(19) United States
(12) Patent Application Publication
(10) Pub. No.: US 2021/0163451 A1
(43) Pub. Date: Jun. 3, 2021

(54) METHODS AND COMPOSITIONS FOR SUBSTITUTED 2,5-DIKETOPIPERAZINE ANALOGS

(71) Applicant: University of Florida Research Foundation, Inc., Gainesville, FL (US)

(72) Inventors: Yousong Ding, Gainesville, FL (US); Guangde Jiang, Gainesville, FL (US)

(21) Appl. No.: 17/049,708

(22) PCT Filed: May 16, 2019
(86) PCT No.: PCT/US2019/032745

§ 371 Date: Oct. 22, 2020

Related U.S. Application Data
(60) Provisional application No. 62/734,878, filed on Sep. 21, 2018.
(60) Provisional application No. 62/672,455, filed on May 16, 2018.

(51) Int. Cl. C07D 403/06; A01N 43/60

(52) U.S. Cl. CPC C07D 403/06; A01N 43/60

(57) ABSTRACT

Thaxtomins are phytotoxic secondary metabolites produced in plant pathogenic Streptomyces strains and have received considerable interests as bioherbicides. A cell-free, biocombinatorial approach was developed to produce a thaxtomin library for herbicide development. Combination of biosynthetic enzymes led to the production of 136 substituted 2,5-diketopiperazines, thaxtomin D, thaxtomin B and thaxtomin A analogs in a single pot. Furthermore, rational engineering of TxtA allowed the synthesis of azido-containing thaxtomin analog. Selected unnatural thaxtomins demonstrated improved herbicidal activities. This abstract is intended as a scanning tool for purposes of searching in the particular art and is not intended to be limiting of the present disclosure.

### CROSS-REFERENCE TO RELATED APPLICATIONS

This application is the 35 U.S.C. § 371 national stage application of PCT Application No. PCT/US2019/032745, filed May 16, 2019, where the PCT claims priority to, and the benefit of, U.S. Provisional Application No. 62/672,455, filed on May 16, 2018, and U.S. Provisional Application No. 62/734,878, filed on Sep. 21, 2018, each of which is incorporated herein by reference in its entirety.

### BACKGROUND

Natural products have a wide variety of applications in health and agriculture. However, their structural complex challenges the use of combinatorial synthesis strategies to create their analogs. Thaxtomins are phytotoxic secondary metabolites produced in plant pathogenic Streptomyces strains and have received considerable interests as bioherbicides. Given their impressive potency and a new model of action, thaxtomins have been pursued as herbicides for crop protection and approved by the EPA. However, conventional synthetic routes to thaxtomin analogues for herbicide development often suffer from the lack of stereocontrol and low overall yield.

Despite advances in research directed to utilization of thaxtomins as herbicides, there is still a scarcity of suitably facile synthetic methods to generate analogs. Moreover, there are ar scarcity of thaxtomin analogues that are potent, efficacious, and selective herbicides. These needs and other needs are satisfied by the present disclosure.

### SUMMARY

In accordance with the purpose(s) of the disclosure, as embodied and broadly described herein, the disclosure, in one aspect, relates to thaxtomins that are useful as bioherbicides. In various aspects, disclosed herein are cell-free, biocombinatorial synthetic methods comprisiung recombinant TxtA and TxtB, two single-module NRPSs, and one pathway-specific P450 TxtC prepared from E. coli. As disclosed herein, biochemical characterization of these enzymes demonstrated their substrate promiscuity and catalytic versatility. The disclosed methods comprising the foregoing, along with TxtE, a nitration promoting P450, the combination of these biosynthetic allows facile preparation of substituted 2,5-diketopiperazines, thaxtomin D, thaxtomin B and thaxtomin A analogs in a single pot. In a further aspect, disclosed herein is the rational engineering of TxtA that allowes for the synthesis of azido-containing thaxtomin analog. In some aspects, the disclosed compounds comprise unnatural thaxtomins that demonstrate improved herbicidal activities. The present disclosure demonstrates the feasibility of biocombinatorial synthesis in generating natural products-like libraries and provided useful insights into the biosynthetic logic leading to expanded chemical diversity in biological systems.

Disclosed are compounds having formula represented by a structure:

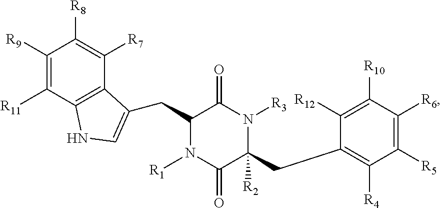

wherein each of R₁ and R₃ is independently selected from hydrogen and C1-C3 alkyl; wherein R₂ is selected from hydrogen and hydroxy; and wherein each of R₄, R₅, R₆, R₇, R₈, R₉, R₁₀, R₁₁, and R₁₂ is independently selected hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Also disclosed are compounds having a formula represented by a structure:

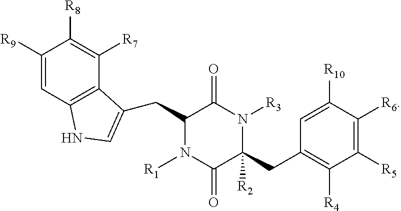

wherein each of R₁ and R₃ is independently selected from hydrogen and C1-C3 alkyl; wherein each of R₂ and R₁₀ is independently selected from hydrogen and hydroxy; wherein each of R₄, R₅, and R₆ is independently selected from hydrogen, halo, hydroxy, azido, and C1-C3 alkyl; wherein R₇ is selected from hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl; wherein each of R₈ and R₉ is independently selected from hydrogen and halo; and wherein R₁₀ is selected from hydrogen and hydroxyl.

Also disclosed are enzyme preparations, including recombinant enzyme preparations of TxtA, TxtB, TxtD, and TxtE enzymes, constructs for recombinant TxtA, TxtB, TxtD, and TxtE enzymes, and bacterial expression systems for TxtA, TxtB, TxtD, and TxtE enzymes.

Also disclosed are methods of preparing the disclosed compounds utilizing TxtA, TxtB, TxtD, and/or TxtE enzymes in cell-free biosynthetic methods.

Also disclosed are herbicidal compositions comprising an herbicidally effective amount of a disclosed compound in a mixture with an agriculturally acceptable adjuvant or carrier.

Also disclosed are herbicidal compositions comprising an herbicidally effective amount of a disclosed compound and at least one further herbicidal agent in a mixture with an agriculturally acceptable adjuvant or carrier.

Also disclosed are herbicidal compositions comprising an herbicidally effective amount of a disclosed compound and at least agent selected from (i) a fungicide, (ii) a herbicide, (iii) an insecticide, (iv) a bactericide, (v) an acaricide, (vi) a nematicide and/or (vii) a plant growth regulator; wherein the the disclosed compound and the at least one agent are formed in a mixture with an agriculturally acceptable adjuvant or carrier.

Also disclosed are methods of using the disclosed compounds and disclosed herbicidal compositions to mitigate the growth of an undesirable plant, e.g., a weed.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A. The representative bioactive 2,5-diketopiperazine compounds. FIG. 1B. Biosynthetic gene cluster of thaxtomins; FIG. 1C. All previously reported thaxtomins and non-nitrated analogue.

FIG. 2A. SDS-PAGE of TxtAH, TxtBH and TxtH. FIG. 2B. HPLC analysis of TxtABH reaction. FIG. 2C. Thermostability of TxtA/B reaction. FIG. 2D. PH dependence of TxtABH reaction.

FIG. 3A. The influence of SAM concentrations to TxtA/B reaction. FIG. 3B. Yields of 4-nitro-L-tryptophan and L-phenylalanine substrates for TxtABH reaction. FIG. 3C. Yields of L-tryptophan analogues and L-phenylalanine analogues for TxtA/B reaction.

FIG. 4. Yields of thaxtomin D analogues generated by TxtEABHC reactions.

FIG. 5. 1D and 2D NMR spectra of 4Me-thaxtomin (60). FIG. 5A. 1H NMR (600 MHz, CD₃OD). FIG. 5B. ¹³C NMR (125 MHz, CD₃OD). FIG. 5C. COSY. FIG. 5D. HSQC. FIG. 5E. HMBC.

FIG. 6A. Substrate selectivity of TxtA-W225S. FIG. 6B. UV spectroscopic analysis of TCB14. FIG. 6C. The influence of NADPH and its regeneration system to TCB14 reaction. FIG. 6D. The influence of methyl group of the substrate for TCB14 reaction.

FIG. 7. Yields of thaxtomin A/B analogues generated by TxtEABHC reactions.

FIG. 8. SDS-PAGE gel of TCB14.

FIG. 9A) Chemical structures of select bioactive compounds with a 2,5-diketopiperazine core. FIG. 9B) The biosynthesis of thaxtomins requires five enzymes to produce multiple analogues.

FIG. 10A) HPLC analysis revealed the production of thaxtomin D (4) in the reaction of TB14, TxtA and TxtB, which missed in control with heat-inactivated TxtA and TxtB. FIG. 10B) SAM availability influenced the distribution of compound 5, thaxtomin C (3) and thaxtomin D (4) in the reaction of TB14, TxtA and TxtB.

FIG. 11A) TxtAW255A mutant utilized p-azido-L-Phe to synthesize p-azido-thaxtomin D and showed a strong preference over L-Phe in the reaction with TB14, TxtA and TxtB. FIG. 11B) TxtA and TxtB synthesized 30 out of 60 possible desnitro thaxtomin D analogues from 5 L-Trp analogues and 12 L-Phe analogues with varying conversion rates. FIG. 11C) TB14, TxtA and TxtB together synthesized 36 thaxtomin D analogues from 3 L-Trp analogues and 12 L-Phe analogues with varying conversion rates. The data represent means of at least two independent experiments with details shown in Tables 8-9.

FIG. 12A) The catalytic performance of TCB14 was influenced by different NADPH regeneration systems. FIG. 12B) TB14, TxtA, TxtB and TCB14 together synthesized 58 mono-(solid bar) and dihydroxylated (sliced bar) thaxtomin analogues from 3 L-Trp analogues and 12 L-Phe analogues with varying conversion rates. The data represent means of at least two independent experiments with details shown in Tables 10-11.

FIG. 13. Characterization of herbicidal activity of select thaxtomin analogues synthesized in this work in radish seedling assay. DMSO was used as the negative control. Data represent mean±SD (n=4).

FIG. 14. Additional natural thaxtomin analogues produced by Streptomyces pathogens.

FIG. 15. SDS-PAGE analysis of purified recombinant proteins used in this work. M: protein marker; Lane 1-8: TxtA, TxtB, TxtH, TxtAW225S, TCB14, GDH, FDH, and PTDH, respectively. All proteins showed calculated molecular weights in the SDS-PAGE analysis.

FIG. 16A-16B) HRMS and MS/MS spectra of thaxtomin D (4) in the reaction. FIG. 16C) MS/MS spectrum of standard thaxtomin D.

FIGS. 17A-17D. The MS fragmentation patterns of key thaxtomin analogues. FIG. 17A) MS/MS fragmentation of N-dimethylated DKPs; FIG. 17B) MS/MS fragmentation of thaxtomin D analogues; FIG. 17C) MS/MS fragmentation of thaxtomin B analogues; FIG. 17D) MS/MS fragmentation of thaxtomin A analogues.

FIG. 18. HPLC traces of the TxtA and TxtB reactions with purified 4-nitro-L-tryptophan as the substrate of TxtB. A significant amount of 4-nitro-L-tryptophan was not consumed, indicating it to be a less effective substrate of TxtB compared with the in situ generated one.

FIG. 19A) HR-MS spectra of compound 5; FIG. 19B) HR-MS/MS spectra of compound 5.

FIG. 20. HPLC traces of the TB14, TxtA and TxtB reactions with different SAM concentrations. The production of one molecule of thaxtomin D consumes two molecules of SAM.

FIG. 21A-21B) HRMS and MS/MS spectra of thaxtomin C (3) in the reaction. FIG. 21C) MS/MS spectrum of standard thaxtomin C (3).

FIG. 22A) Relative substrate selectivity of TxtA; FIG. 22B) Relative substrate selectivity of TxtB. The enzyme activity toward the most active substrate was set as 100% and its activities toward other substrates were then normalized.

FIG. 23. Engineering TxtA's A domain substrate binding pocket. FIG. 23A) Ribbon diagram of a homology model of TxtA's A domain (cyan) superimposed on the homology model of TxtA A domain W225S variant (magenta). The substrate binding pose of phenylalanine was directly imported from the template (grsA, PDB code 1AMU) and was highlighted in CPK representation. FIG. 23B) Residues lining the L-Phe binding pocket are shown as stick models. The overlapped amino acids (Ala222, Trp225, Thr264, Val285, Ala287, Ala308, Val316, and Cys317) were highlighted in cyan and Ser225 on the engineered protein was rendered magenta.

FIG. 24A) HR-MS spectrum of p-N3-Thx D; FIG. 24B) HR-MS/MS spectrum of p-N3-Thx D.

FIG. 25. 1D and 2D NMR spectra of p-N3-thaxtomin D. FIG. 25A). 1H NMR (600 MHz, CD3OD); FIG. 25B) 13C NMR (125 MHz, CD3OD); FIG. 25C) COSY; FIG. 25D) HSQC; FIG. 25E) HMBC.

FIGS. 26A and 26B. HR-MS (FIG. 26A) and MS/MS (FIG. 26B) spectra of desnitro-Thx D.

FIGS. 27A and 27B. HR-MS (FIG. 27A) and MS/MS (FIG. 27B) spectra of 4′-Me-Thx D.

FIGS. 28A to 28E. 1D and 2D NMR spectra of 4′Me-thaxtomin D. FIG. 28A). ¹H NMR (600 MHz, CD₃OD); FIG. 28B) ¹³C NMR (125 MHz, CD₃OD); FIG. 28C) COSY; FIG. 28D) HSQC; FIG. 28E) HMBC.

FIG. 29. UV-Vis spectroscopic analysis of TCB14. Red lines: CO-oxidized spectrum; red line: CO-reduced spectrum; green lines: CO-reduced difference spectrum.

FIGS. 30A to 30C. HR-MS (FIG. 30A) and MS/MS (FIG. 30B) spectra of thaxtomin B produced in TCB14 reaction in comparison with MS/MS spectrum of standard thaxtomin B (FIG. 30C).

FIGS. 31A to 31E. 1D and 2D NMR spectra of 4′Me-thaxtomin A. FIG. 31A). ¹H NMR (600 MHz, CD₃OD); FIG. 31B)¹³C NMR (125 MHz, CD₃OD); FIG. 31C) COSY; FIG. 31D) HSQC; FIG. 31E) HMBC.

FIG. 32A to 32C. HR-MS (FIG. 32A) and MS/MS (FIG. 32B) spectra of thaxtomin A produced in the one-pot reaction in comparison with MS/MS spectrum of standard thaxtomin A (FIG. 32C).

FIG. 33A to 33F. HR-MS (FIGS. 33A, 33B, and 33C) and MS/MS (FIGS. 33D, 33E, and 33F) spectra of 4′Me-Thx A, 6F-3′F-Thx D and desnitro-4Me-Thx D, respectively.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound,” “an herbicidal composition,” or “an agriculturally acceptable carrier,” includes, but is not limited to, two or more such “compounds,” “herbicidal compositions,” or “agriculturally acceptable carriers.”

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

The term “agriculturally acceptable” is used herein to include agricultural, industrial and residential uses which are compatible with plants.

As used herein, the terms “controlling” and “combating” are synonyms.

As used herein, the terms “undesirable vegetation”, “harmful plants” and “weeds” are synonyms.

As used herein, the term “herbicide resistant” refers to plants that are resistant to herbicides for example, but not limited to, glyphosate, dicamba, 2,4-dichlorophenoxyethanoic acid, glufosinate, ACCase inhibitors, HPPD inhibitors and/or acetohydroxyacid synthase inhibitors.

“Adjuvants” are materials that facilitate the activity of herbicides or that facilitate or modify characteristics of herbicide formulations or spray solutions.

As used throughout this application, the term “agriculturally acceptable salt” refers to a salt comprising a cation that is known and accepted in the art for the formation of salts for agricultural or horticultural use. In one aspect, the salt is a water-soluble salt.

The “crops of useful plants” to be protected typically comprise, for example, the following species of plants: cereals (wheat, barley, rye, oats, maize (including field corn, pop corn and sweet corn), rice, sorghum and related crops); beet (sugar beet and fodder beet); leguminous plants (beans, lentils, peas, soybeans); oil plants (rape, mustard, sunflowers); cucumber plants (marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), caneberries, cranberries, peppermint, rhubarb, spearmint, sugar cane and turf grasses including, for example, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annus L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutate Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)).

The term “useful plants” also includes also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” also includes useful plants which have been so transformed by the use of recombinant DNA techniques that they are capable of synthesising one or more selectively acting toxins, such as are known, for example, from toxin-producing bacteria, especially those of the genus Bacillus.

The term “useful plants” also includes useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The terms “halo,” “halogen” or “halide,” as used herein can be used interchangeably and refer to F, Cl, Br, or I.

The term “hydroxyl” or “hydroxy” as used herein can be used interchangeably and is represented by the formula —OH.

The term “nitro” as used herein is represented by the formula —NO₂.

The term “azido” as used herein is represented by the formula —N≡N.

The term “nitrile” or “cyano” as used herein is represented by the formula —CN.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

As used herein, the term “effective amount” refers to an amount that is sufficient to achieve the desired modification of a physical property of the composition or material. For example, an “effective amount” of as herbicidal composition refers to an amount that is sufficient to achieve the desired control of target weeds. The specific level in terms of grams per acre in a composition required as an effective amount will depend upon a variety of factors including the amount and type of level of weed growth, stage of weed growth, and stage of crop plant growth.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

It is also appreciated that certain compounds described herein can be present as an equilibrium of tautomers. For example, ketones with an α-hydrogen can exist in an equilibrium of the keto form and the enol form.

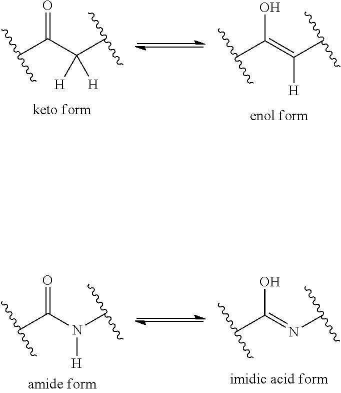 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

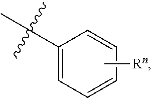

which is understood to be equivalent to a formula:

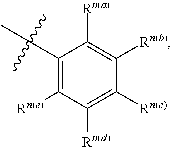

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Introduction

Natural products and their derivatives have contributed significantly to human health and the development of many scientific fields. On the other hand, the structural complexity of natural products continues to be a major challenge in natural products-based drug discovery and development. Elegantly, nature synthesizes these structurally diverse chemicals from simple building blockings using a set of enzymes, providing potentially viable solutions to chemical synthesis. Non-ribosomal peptides (NRPs) are one major family of natural products with diverse bioactivities and pharmacophores. They can be assembled from a pool of over 500 amino acid monomers by NRP synthetases (NRPSs). The substrate specificity, order, and number of NRPS modules and a variety of potential tailoring modifications together create enormous structural diversity, positively correlating with their broad therapeutic space. Importantly, NRPs are feasible targets in creating natural products-like libraries. Indeed, biosynthetic engineering has demonstrated notable successes in producing NRPs and their analogs directly by fermentation. However, the limited understanding of NRPSs (e.g., enzyme dynamics, substrate specificity and protein/protein interactions) and their associated pathways renders most approaches currently not practical. In this regard, in vitro reconstitution of NRP biosynthesis is an attractive approach to delineate useful principles guiding the generation of chemical diversity, informing effective biosynthetic engineering.

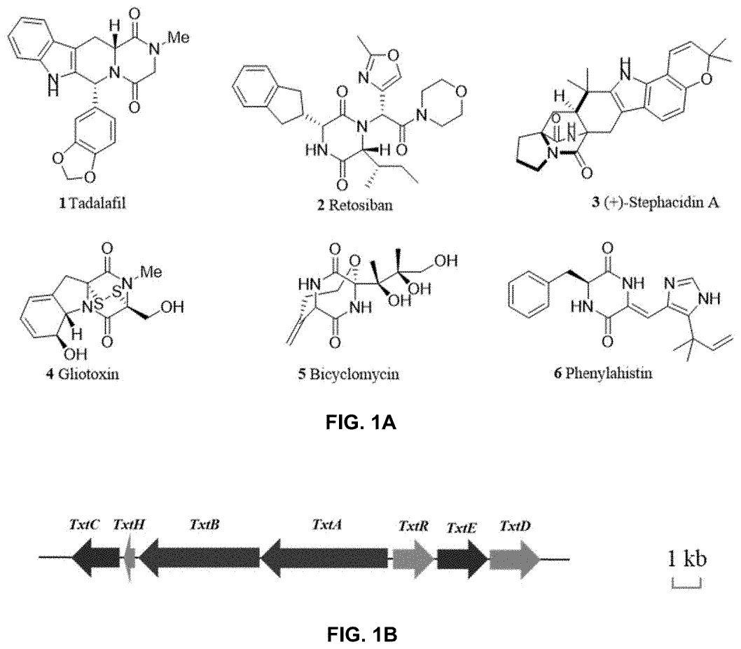

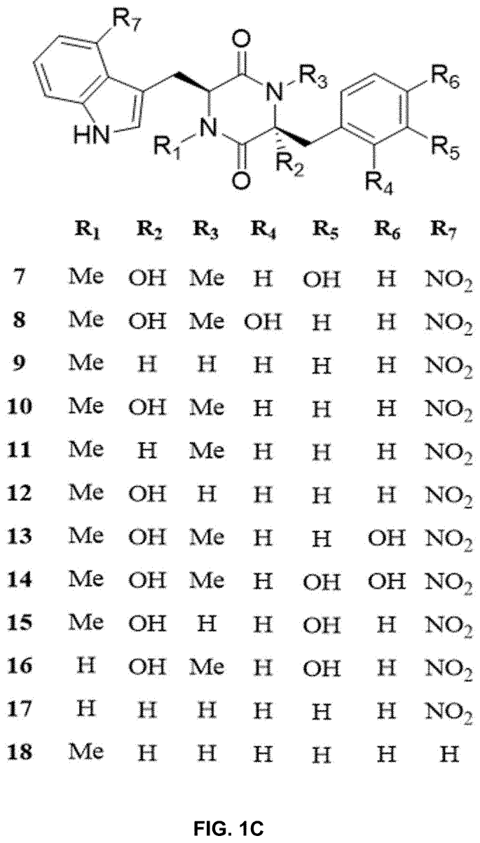

2,5-Diketopiperazines (DKPs) are a family of smallest cyclopeptides made of two amino acid monomers. The DKP scaffold is a privileged structure for drug discovery as it is metabolically stable, structurally constrained, and amenable to multiple stereo-specific modifications. Diversification around this scaffold is a proven strategy to create therapeutically important drug-like molecules, e.g., the PDES inhibitor Tadalafil and the oxytocin receptor antagonist Retosiban (FIG. 1A). Chemical diversification is often achieved using unnatural amino acid building blocks and further decorations after cyclization, which requires special synthetic routes to starting materials and needs to address selectivity issues. By contrast, a variety of organisms employ either two NRPS modules or one cyclodipeptide synthase along with tailoring enzymes to produce a large number of structurally diverse DKP-containing natural products with a broad range of bioactivities (bicyclomycin, gliotoxin, verticillin, phenylahistin, etc (FIG. 1A). Harnessing the catalytic power and versatility of these biosynthetic machineries to propel the development of DKP-containing drug molecules is an enticing alternative strategy to chemical approaches.

Thaxtomins are phytotoxic secondary metabolites produced in plant pathogenic Streptomyces strains and have received considerable interests as bioherbicides. To produce a thaxtomin library for herbicide development, a cell-free, biocombinatorial approach was developed. Recombinant TxtA and TxtB, two single-module NRPSs, and one pathway-specific P450 TxtC were prepared from E. coli. Biochemical characterization of these enzymes revealed their substrate promiscuity and catalytic versatility. Along with TxtE, a nitration promoting P450, the combination of these biosynthetic enzymes led to the production of 136 substituted 2,5-diketopiperazines, thaxtomin D, thaxtomin B and thaxtomin A analogs in a single pot. Furthermore, rational engineering of TxtA allowed the synthesis of azido-containing thaxtomin analog. Selected unnatural thaxtomins demonstrated improved herbicidal activities. This work demonstrated the feasibility of biocombinatorial synthesis in generating natural products-like libraries and provided useful insights into the biosynthetic logic leading to expanded chemical diversity in biological systems.

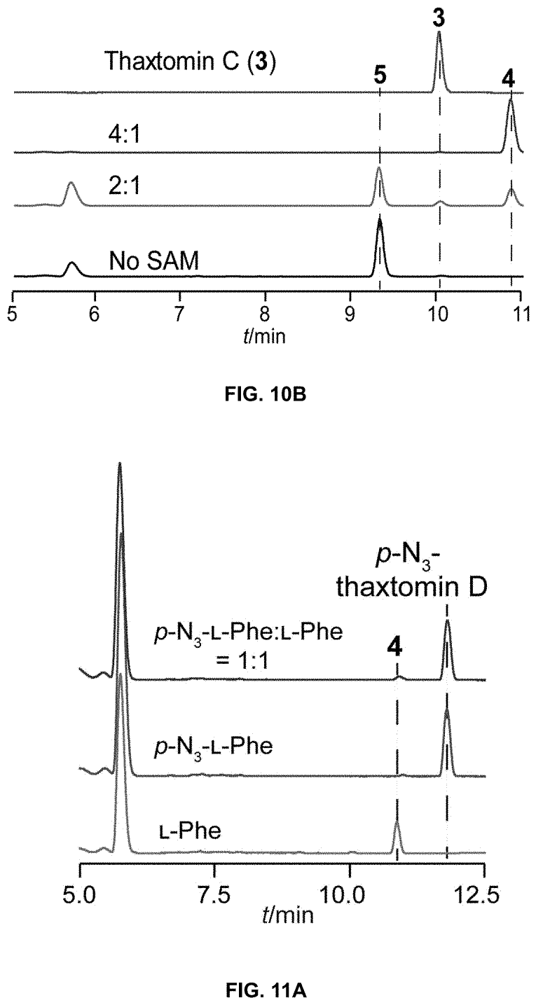

Disclosed herein is a cell-free combinatorial biosynthesis approach to expand the molecular diversity and utility of thaxtomin-like DKP compounds by elucidating and engaging the catalytic versatility of four thaxtomin biosynthetic enzymes (FIG. 1B). Thaxtomins are the virulence factor of the plant pathogen Streptomyces scabiei and tens of other pathogenic Streptomyces strains, and are a novel class of bioherbicides that inhibit cellulose biosynthesis in the nM range. Given their attractive agricultural significance, several chemical routes to thaxtomins and their analogs have been developed but suffered from the lack of stereo-control and low overall yield, resulting in the limited exploration of chemical space of the thaxtomin scaffold in herbicide development. On the other hand, the thaxtomin biosynthetic pathway produces thaxtomin A, the major metabolite, and 11 other analogs using five enzymes (FIGS. 1B and 10). This pathway starts with the production of 4-NO2-I-tryptophan (4-NO2-I-Trp) catalyzed by one P450 enzyme TxtE using co-substrate nitric oxide (NO) which is generated by TxtD from 1-arginine. TxtA and TxtB, two single-module NRPSs, then form the DKP thaxtomin D through the activation of I-phenylalanine (I-Phe) and 4-NO2-I-Trp, dipeptide cyclization, and two N-methylations. TxtC, the other pathway-specific P450 enzyme, may sequentially hydroxylate α-position and the C-3 of the aryl group of the 1-phenylalanine moiety to produce thaxtomin A (FIG. 1B). These biosynthetic enzymes provide biocatalytic opportunities to construct and functionalize the DKP scaffold. Previously, biochemical characterization of TxtE revealed its relaxing substrate scope, which then guided the in vivo production of herbicidal 5-F-thaxtomin A. Recent in vivo studies also suggested the tolerance of TxtB and TxtC to several 1-tryptophan analogs with C-4 substitutions. To fully exploit the synthetic potential of thaxtomin biosynthetic enzymes, the present work systematically characterized the substrate scopes of TxtA, TxtB and TxtC, sequentially reconstructed the thaxtomin biosynthesis and synthesized 132 thaxtomin-like DKP analogs.

### C. Compounds

In one aspect, the disclosure relates to 2,5-diketopiperazines, including thaxtomin D, thaxtomin B and thaxtomin A analogs.

In various aspects, a compound has a formula represented by a structure:

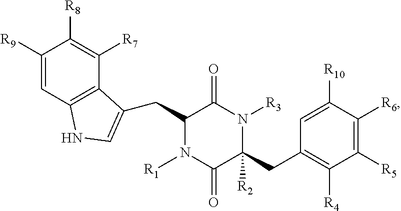

wherein each of R₁ and R₃ is independently selected from hydrogen and C1-C3 alkyl; wherein R₂ and R₁₀ is selected from hydrogen and hydroxy; wherein each of R₄, R₅, and R₆ is independently selected from hydrogen, halo, hydroxy, and C1-C3 alkyl; wherein R₇ is selected from hydrogen, hydroxy, and nitro; and wherein each of R₈ and R₉ is independently selected from hydrogen and halo; or an agriculturally acceptable salt thereof.

In various aspects, a compound has a formula represented by a structure:

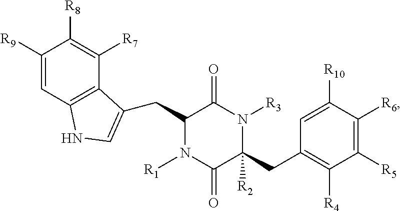

wherein each of R₁ and R₃ is independently selected from hydrogen and C1-C3 alkyl; wherein R₂ and R₁₀ is selected from hydrogen and hydroxy; wherein each of R₄, R₅, and R₆ is independently selected from hydrogen, halo, hydroxy, and C1-C3 alkyl; wherein R₇ is selected from hydrogen, hydroxy, and nitro; and wherein each of R₈ and R₉ is independently selected from hydrogen and halo.

In various aspects, a compound has a formula represented by a structure:

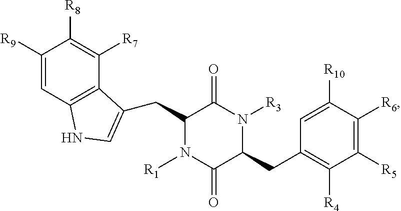

wherein the substituents groups have the meaning as disclosed herein throughout.

In various aspects, a compound has a formula represented by a structure:

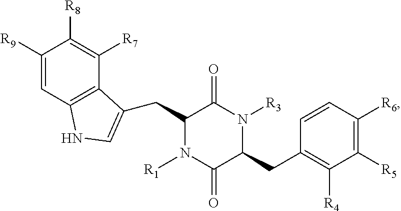

wherein the substituents groups have the meaning as disclosed herein throughout.

In various aspects, a compound has a formula represented by a structure:

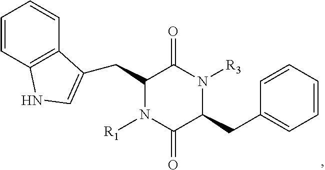

wherein the substituents groups have the meaning as disclosed herein throughout.

In various aspects, a compound has a formula represented by a structure:

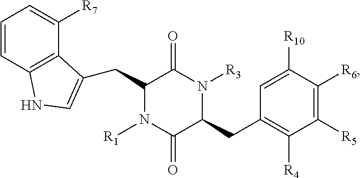

wherein the substituents groups have the meaning as disclosed herein throughout.

In various aspects, a compound has a formula represented by a structure:

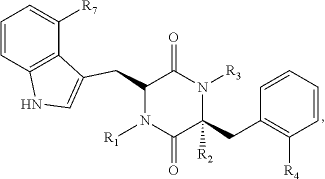

wherein the substituents groups have the meaning as disclosed herein throughout.

In various aspects, a compound has a formula represented by a structure:

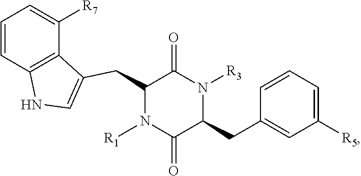

wherein the substituents groups have the meaning as disclosed herein throughout.

In various aspects, a compound has a formula represented by a structure:

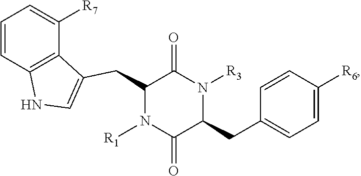

wherein the substituents groups have the meaning as disclosed herein throughout.

In various aspects, a compound has a formula represented by a structure:

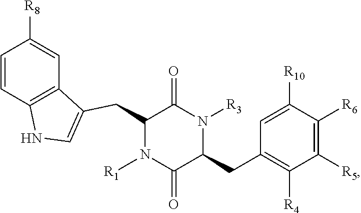

wherein the substituents groups have the meaning as disclosed herein throughout.

In various aspects, a compound has a formula represented by a structure:

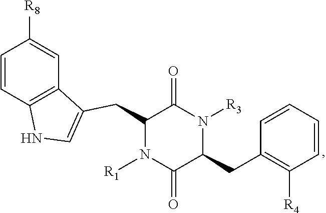

wherein the substituents groups have the meaning as disclosed herein throughout.

In various aspects, a compound has a formula represented by a structure:

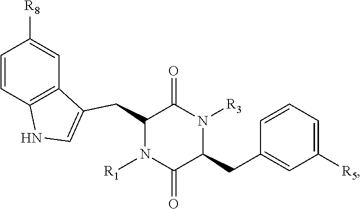

wherein the substituents groups have the meaning as disclosed herein throughout.

In various aspects, a compound has a formula represented by a structure:

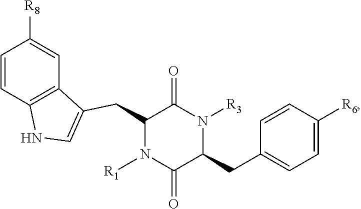

wherein the substituents groups have the meaning as disclosed herein throughout.

In various aspects, a compound has a formula represented by a structure:

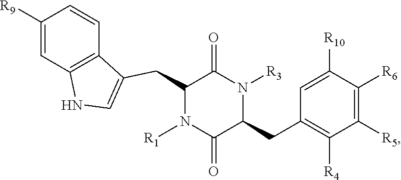

wherein the substituents groups have the meaning as disclosed herein throughout.

In various aspects, a compound has a formula represented by a structure:

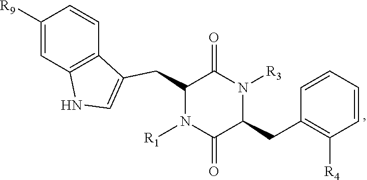

wherein the substituents groups have the meaning as disclosed herein throughout.

In various aspects, a compound has a formula represented by a structure:

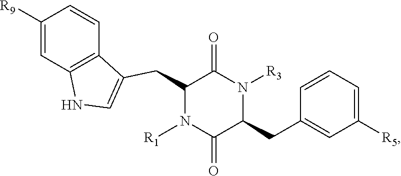

wherein the substituents groups have the meaning as disclosed herein throughout.

In various aspects, a compound has a formula represented by a structure:

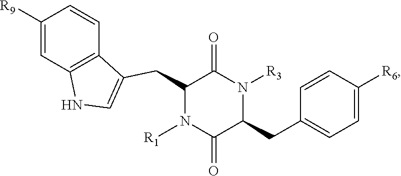

wherein the substituents groups have the meaning as disclosed herein throughout.

In various aspects, a compound has a formula represented by a structure:

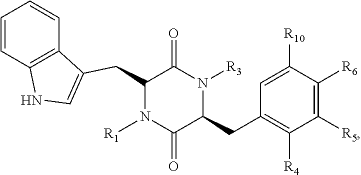

wherein the substituents groups have the meaning as disclosed herein throughout.

In various aspects, a compound has a formula represented by a structure:

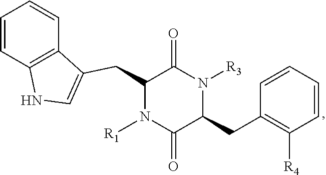

wherein the substituents groups have the meaning as disclosed herein throughout.

In various aspects, a compound has a formula represented by a structure:

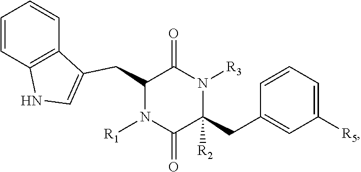

wherein the substituents groups have the meaning as disclosed herein throughout.

In various aspects, a compound has a formula represented by a structure:

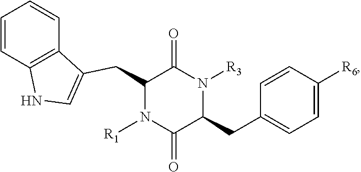

wherein the substituents groups have the meaning as disclosed herein throughout.

In various aspects, a compound has a formula represented by a structure:

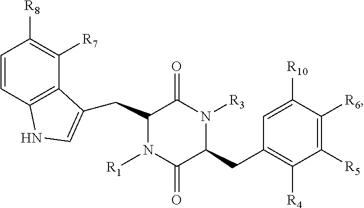

wherein the substituents groups have the meaning as disclosed herein throughout.

In various aspects, a compound has a formula represented by a structure:

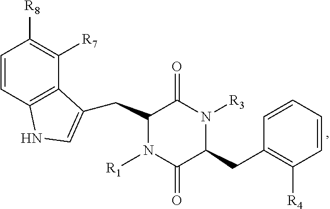

wherein the substituents groups have the meaning as disclosed herein throughout.

In various aspects, a compound has a formula represented by a structure:

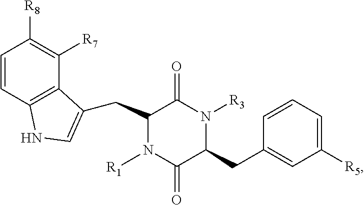

wherein the substituents groups have the meaning as disclosed herein throughout.

In various aspects, a compound has a formula represented by a structure:

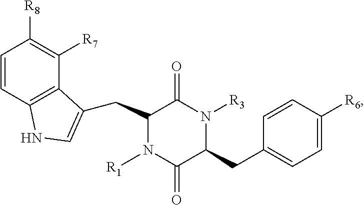

wherein the substituents groups have the meaning as disclosed herein throughout.

In various aspects, a compound has a formula represented by a structure:

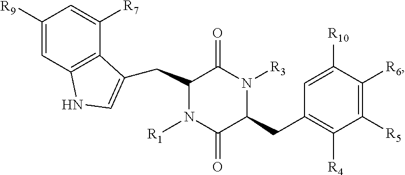

wherein the substituents groups have the meaning as disclosed herein throughout.

In various aspects, a compound has a formula represented by a structure:

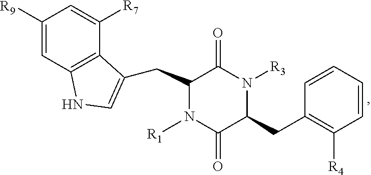

wherein the substituents groups have the meaning as disclosed herein throughout.

In various aspects, a compound has a formula represented by a structure:

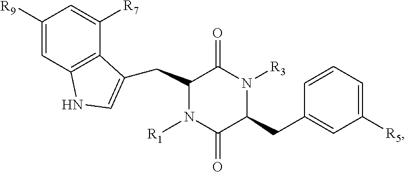

wherein the substituents groups have the meaning as disclosed herein throughout.

In various aspects, a compound has a formula represented by a structure:

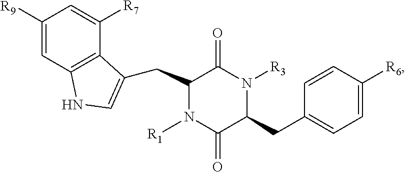

wherein the substituents groups have the meaning as disclosed herein throughout.

In various aspects, a compound has a formula represented by a structure:

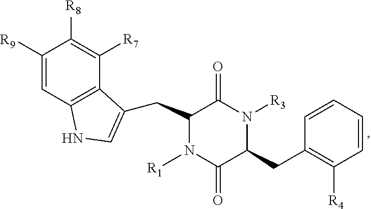

wherein the substituents groups have the meaning as disclosed herein throughout.

In various aspects, a compound has a formula represented by a structure:

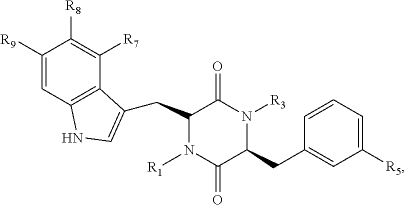

wherein the substituents groups have the meaning as disclosed herein throughout.

In various aspects, a compound has a formula represented by a structure:

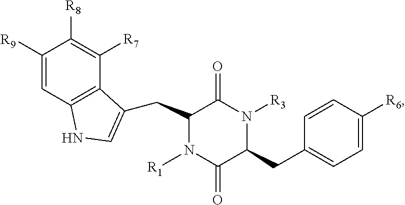

wherein the substituents groups have the meaning as disclosed herein throughout.

In various aspects, a compound has a formula represented by a structure:

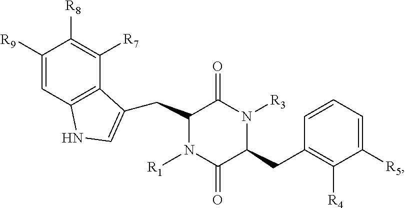

wherein the substituents groups have the meaning as disclosed herein throughout.

In various aspects, a compound has a formula represented by a structure:

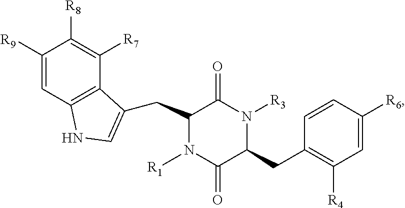

wherein the substituents groups have the meaning as disclosed herein throughout.

In various aspects, a compound has a formula represented by a structure:

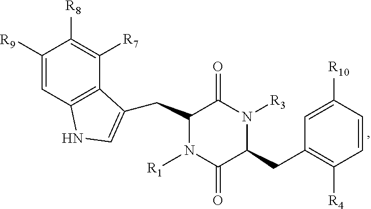

wherein the substituents groups have the meaning as disclosed herein throughout.

In various aspects, a compound has a formula represented by a structure:

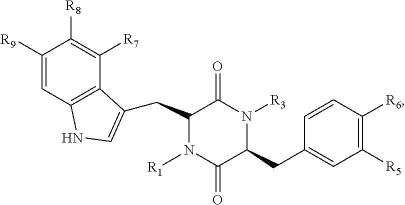

wherein the substituents groups have the meaning as disclosed herein throughout.

In various aspects, a compound has a formula represented by a structure:

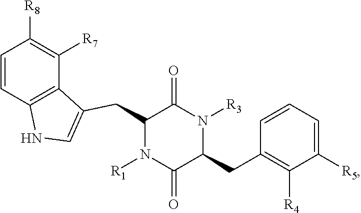

wherein the substituents groups have the meaning as disclosed herein throughout.

In various aspects, a compound has a formula represented by a structure:

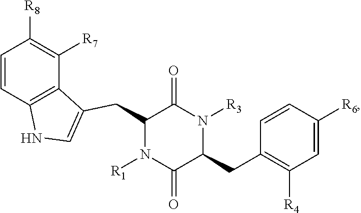

wherein the substituents groups have the meaning as disclosed herein throughout.

In various aspects, a compound has a formula represented by a structure:

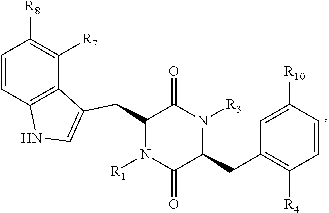

wherein the substituents groups have the meaning as disclosed herein throughout.

In various aspects, a compound has a formula represented by a structure:

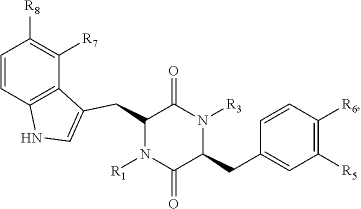

wherein the substituents groups have the meaning as disclosed herein throughout.

In various aspects, a compound has a formula represented by a structure:

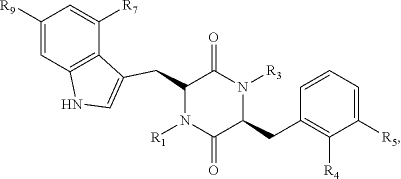

wherein the substituents groups have the meaning as disclosed herein throughout.

In various aspects, a compound has a formula represented by a structure:

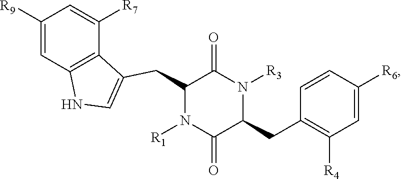

wherein the substituents groups have the meaning as disclosed herein throughout.

In various aspects, a compound has a formula represented by a structure:

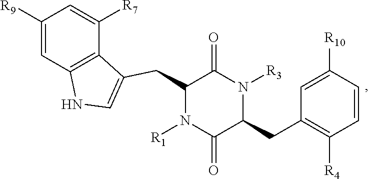

wherein the substituents groups have the meaning as disclosed herein throughout.

In various aspects, a compound has a formula represented by a structure:

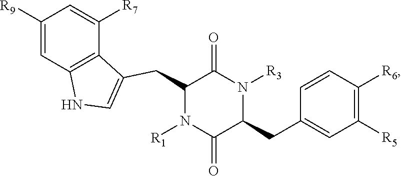

wherein the substituents groups have the meaning as disclosed herein throughout.

In a further aspect, the disclosed compound can be selected from one or more of the following:

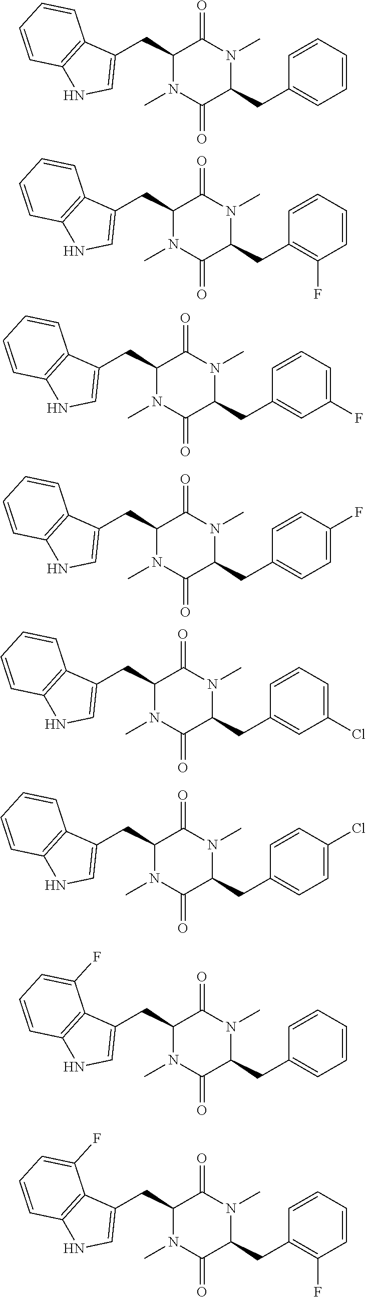

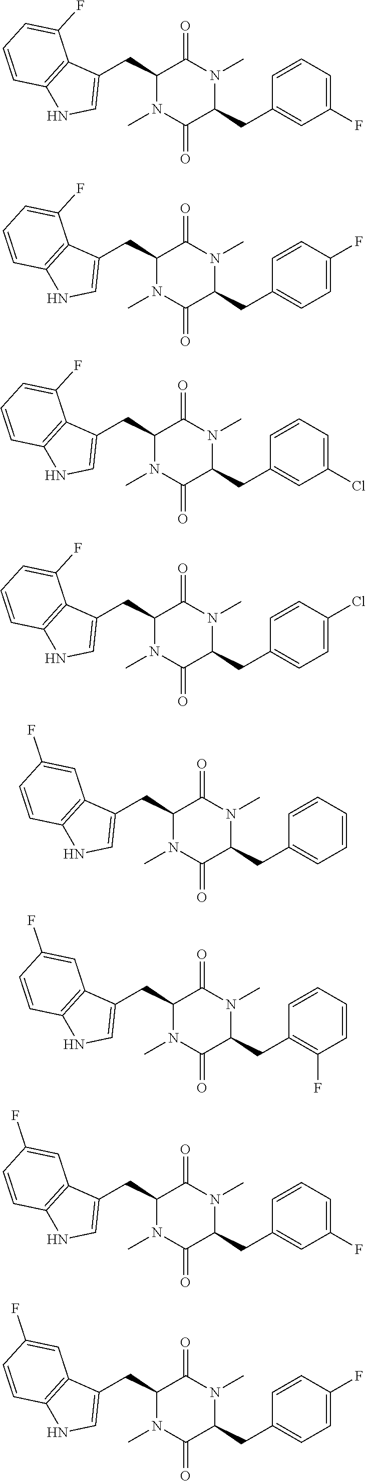

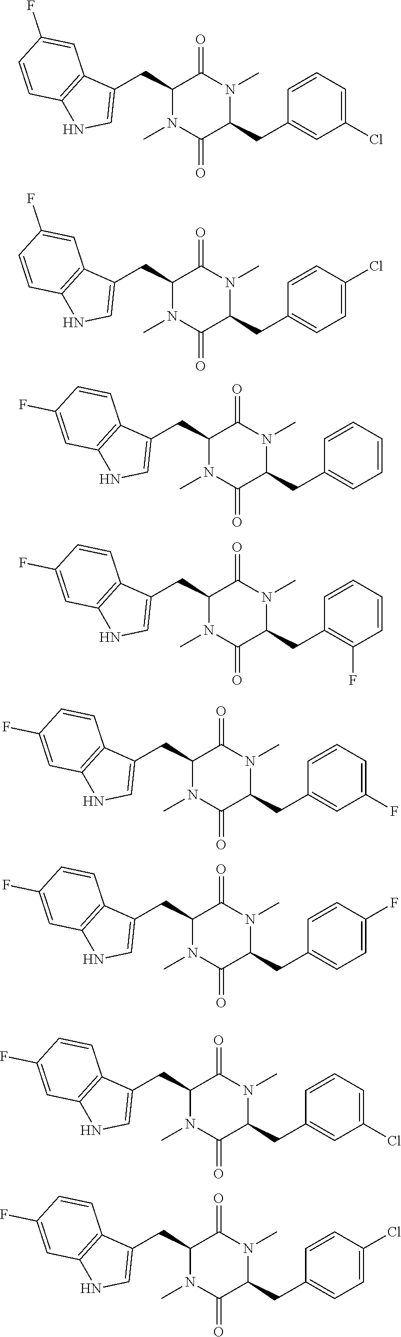

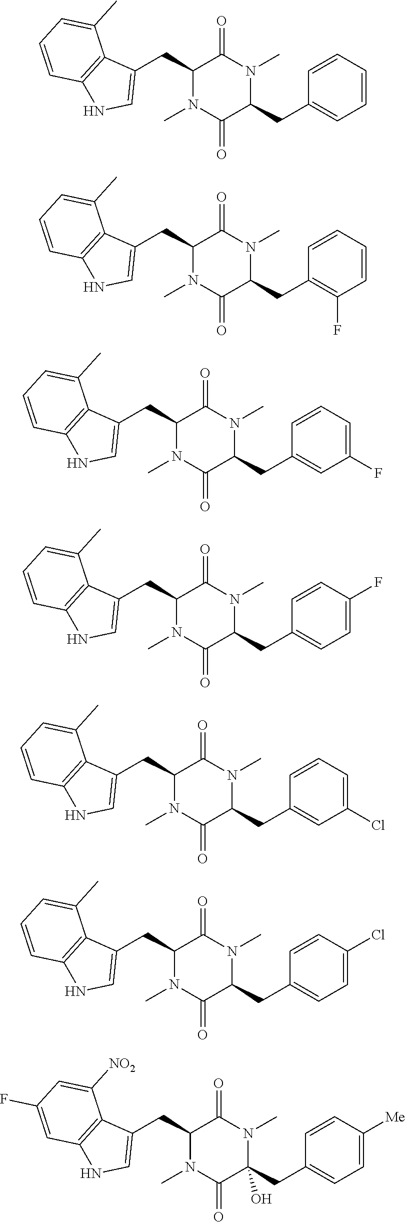

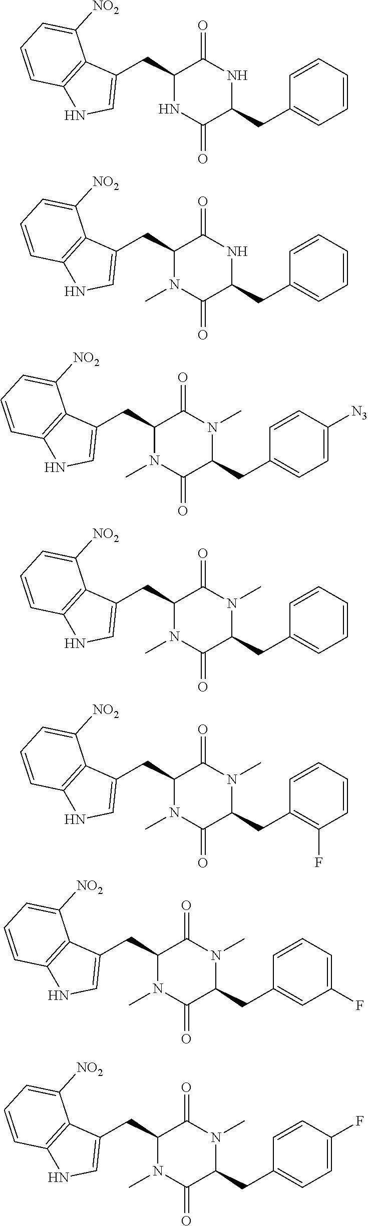

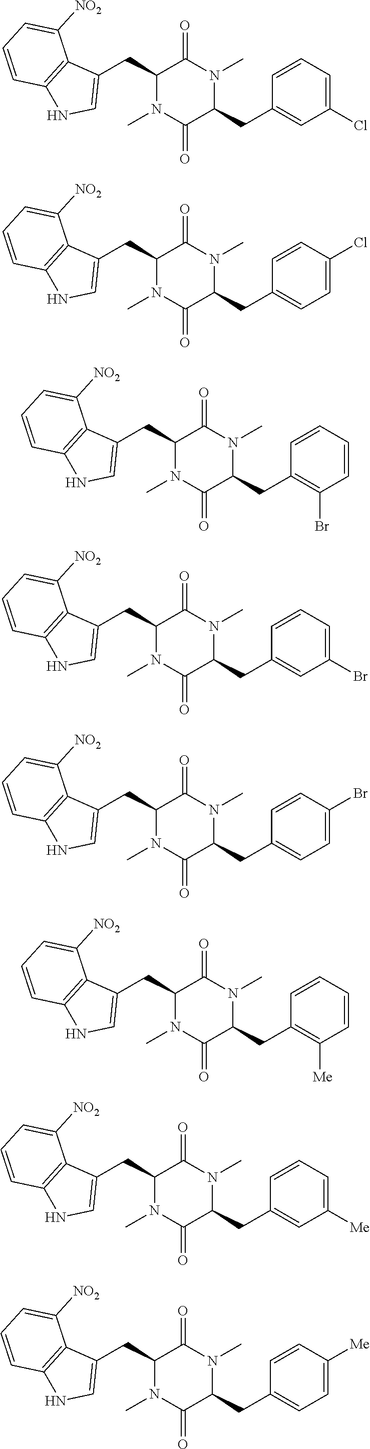

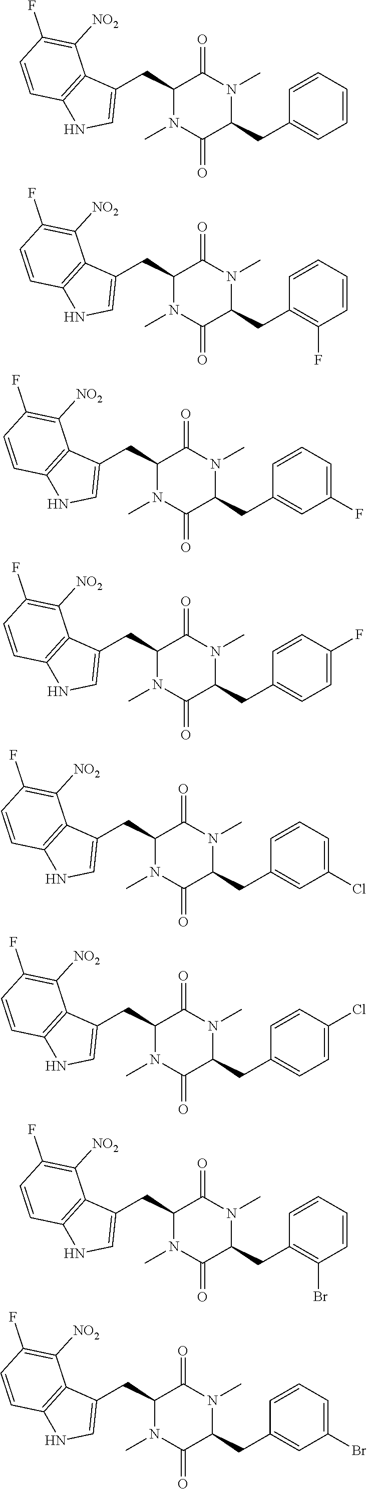

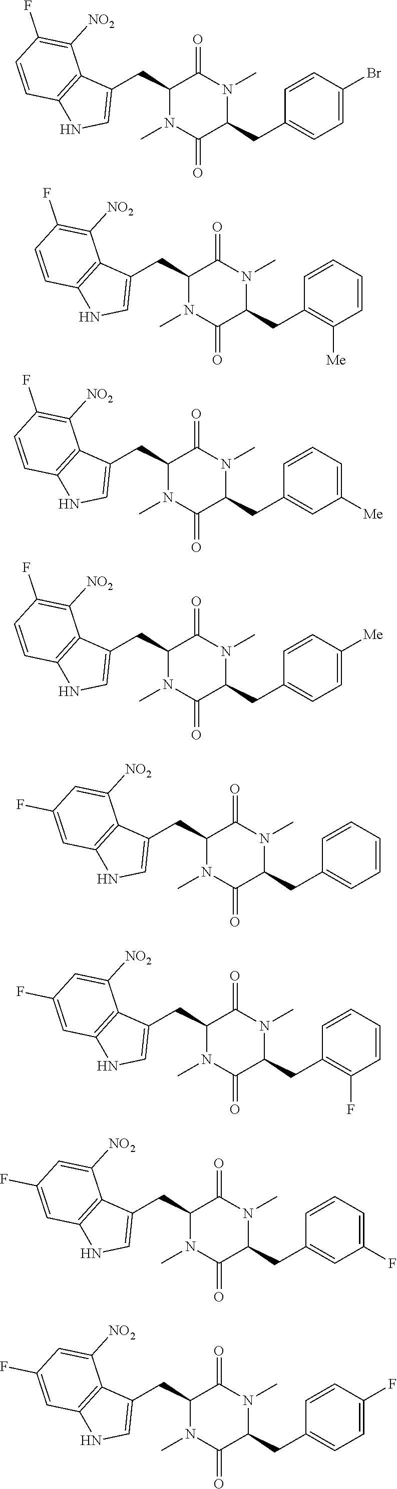

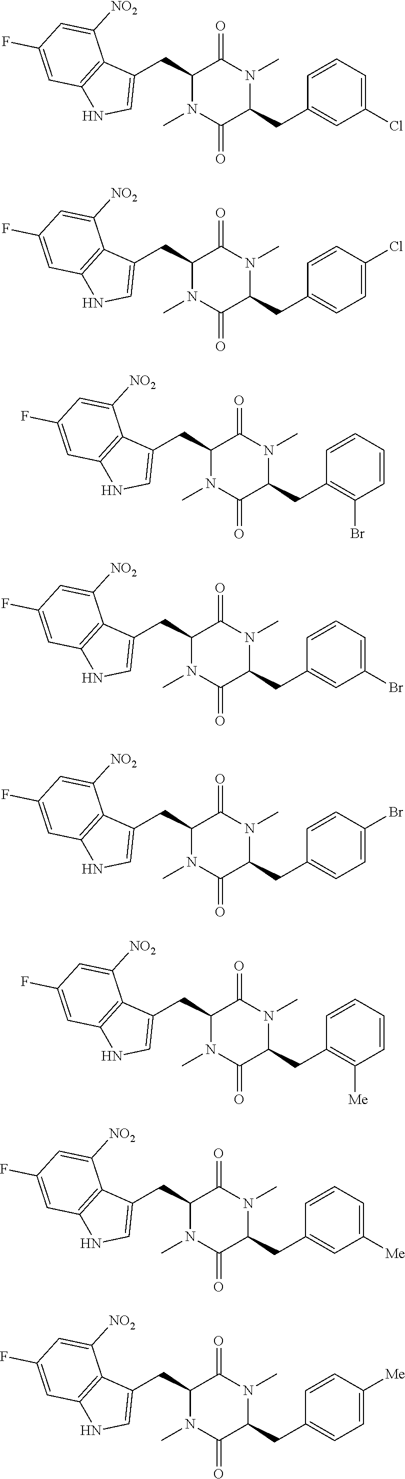

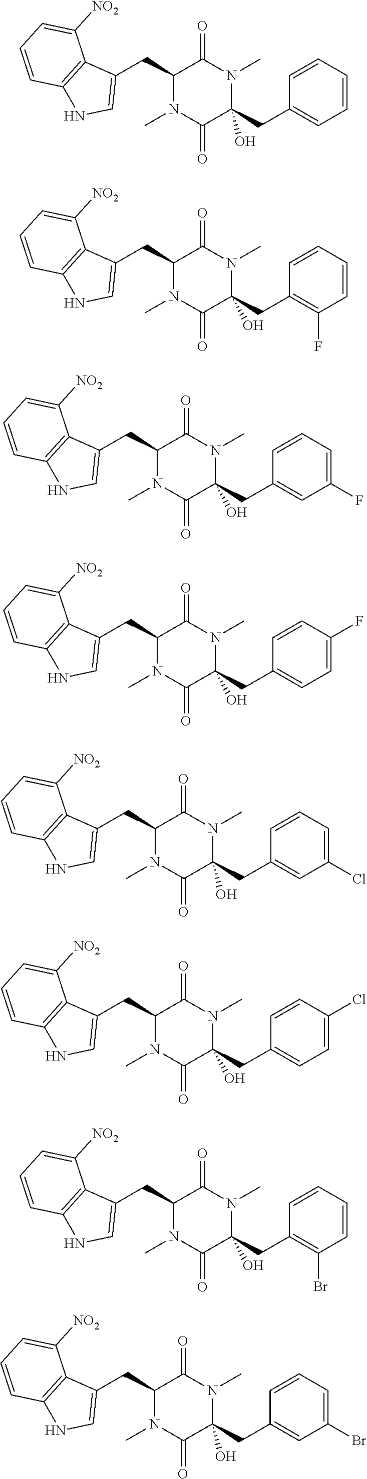

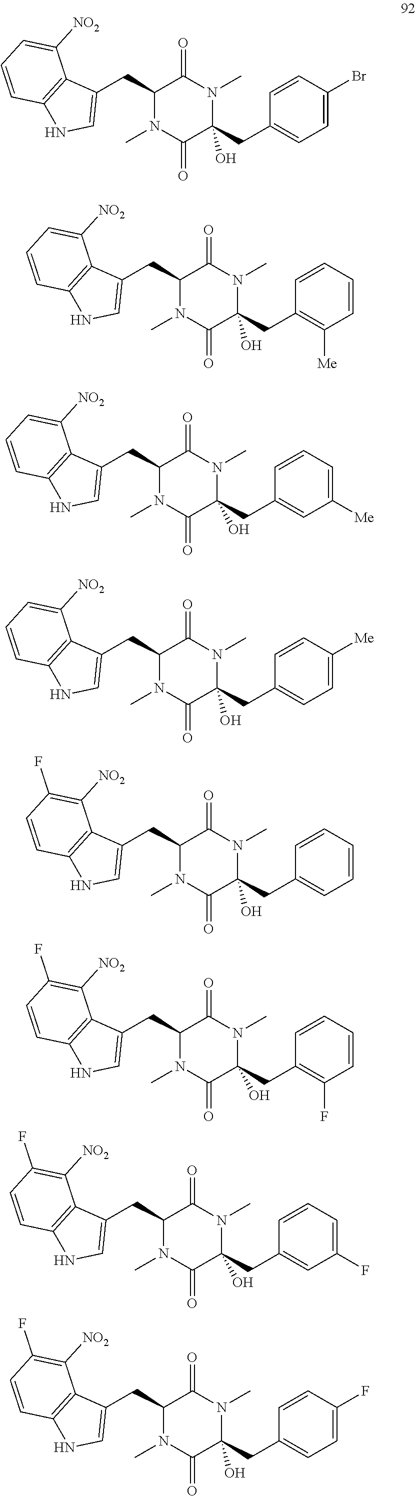

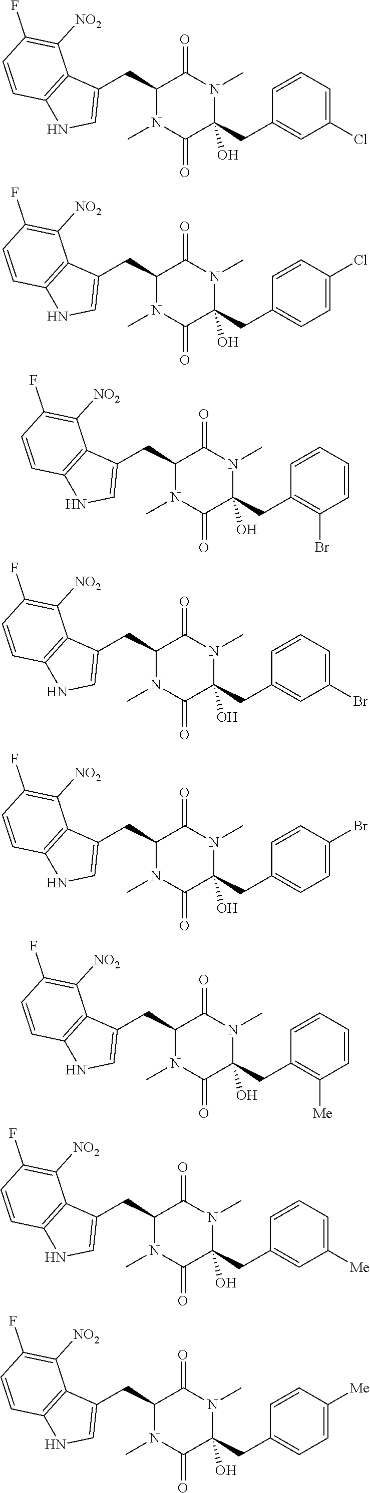

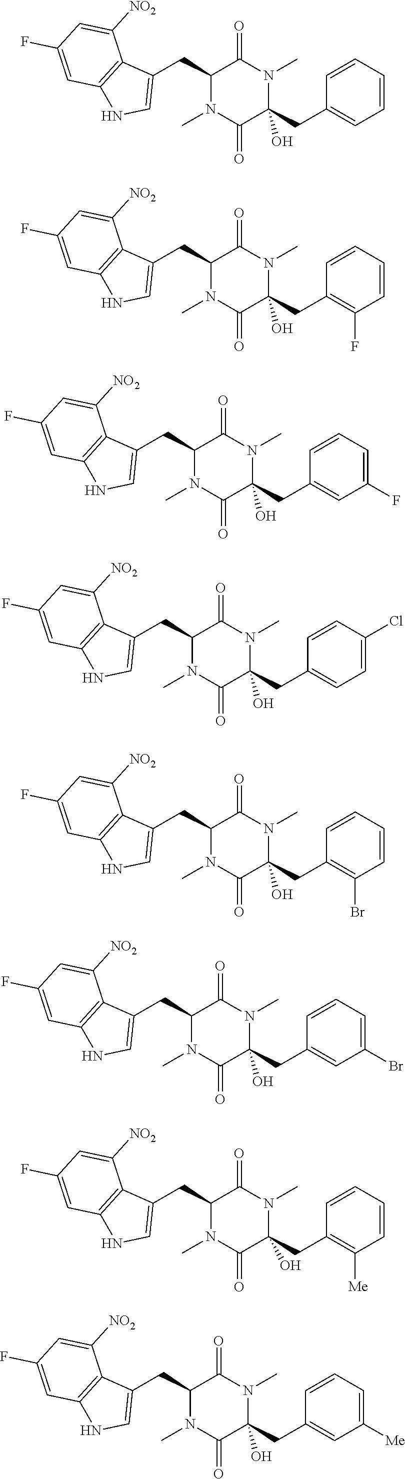

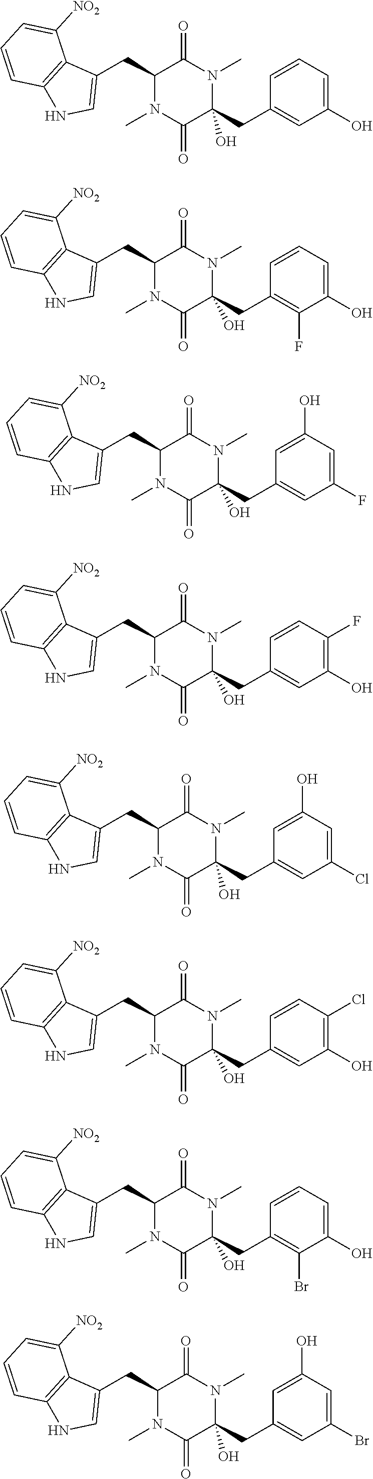

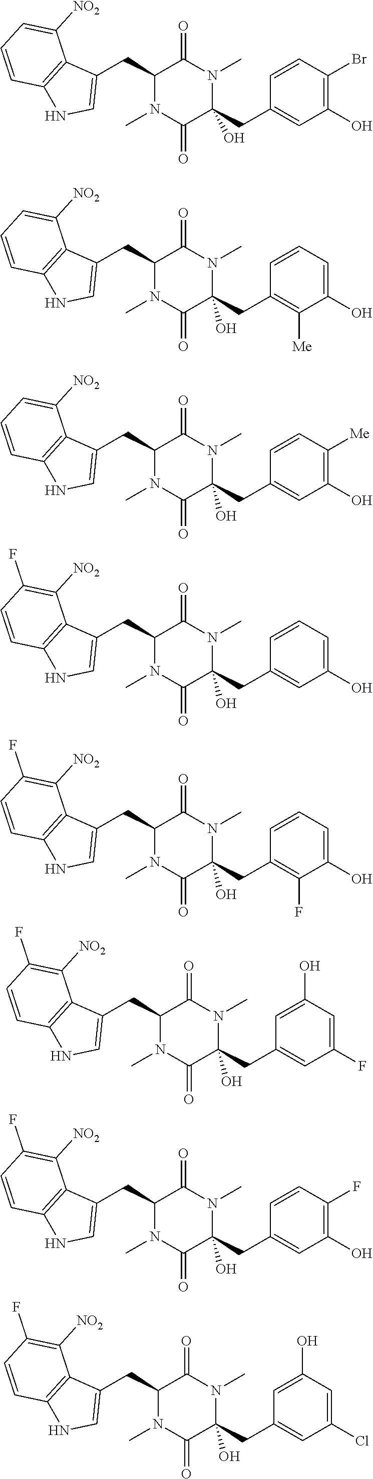

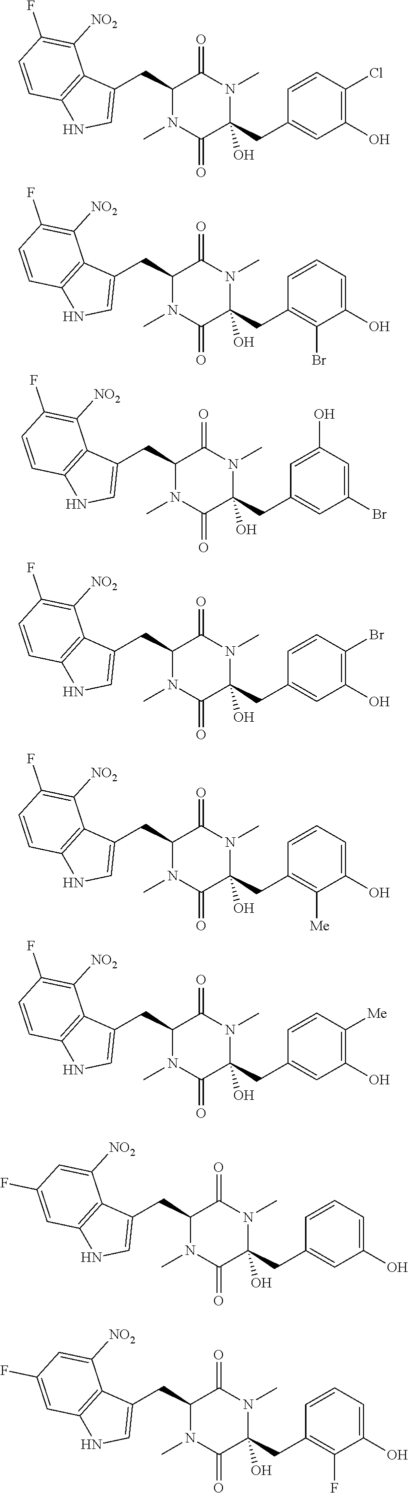

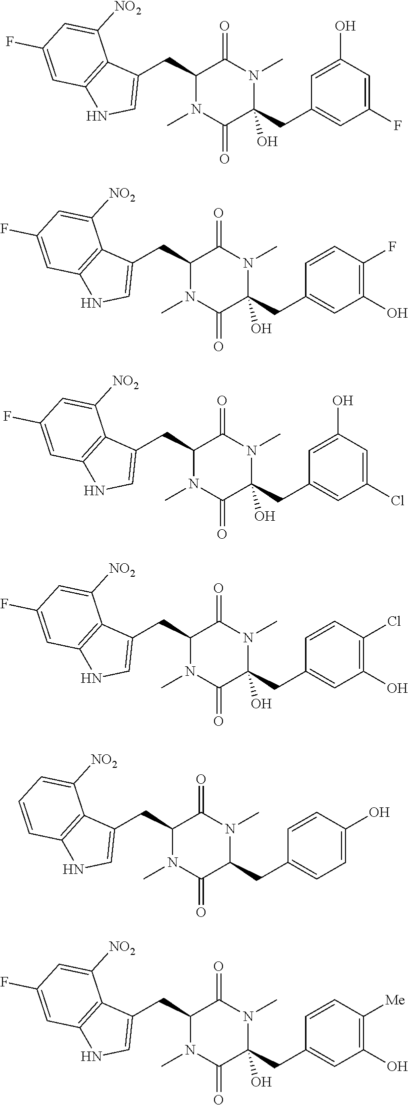 [SMILES: CN1C(=O)[C@](O)(Cc2cc(O)cc(F)c2)N(C)C(=O)[C@@H]1Cc1c[nH]c2cc(F)cc([N+](=O)[O-])c12 ; CN1C(=O)[C@](O)(Cc2ccc(F)c(O)c2)N(C)C(=O)[C@@H]1Cc1c[nH]c2cc(F)cc([N+](=O)[O-])c12 ; CN1C(=O)[C@](O)(Cc2cc(O)cc(Cl)c2)N(C)C(=O)[C@@H]1Cc1c[nH]c2cc(F)cc([N+](=O)[O-])c12 ; CN1C(=O)[C@](O)(Cc2ccc(Cl)c(O)c2)N(C)C(=O)[C@@H]1Cc1c[nH]c2cc(F)cc([N+](=O)[O-])c12 ; CN1C(=O)[C@H](Cc2c[nH]c3cccc([N+](=O)[O-])c23)N(C)C(=O)[C@@H]1Cc1ccc(O)cc1 ; Cc1ccc(C[C@@]2(O)C(=O)N(C)[C@@H](Cc3c[nH]c4cc(F)cc([N+](=O)[O-])c34)C(=O)N2C)cc1O]

### D. Methods of Making

In various aspects, the disclosed methods of making the disclosed compounds comprise the biosynthetic schemes as follows:

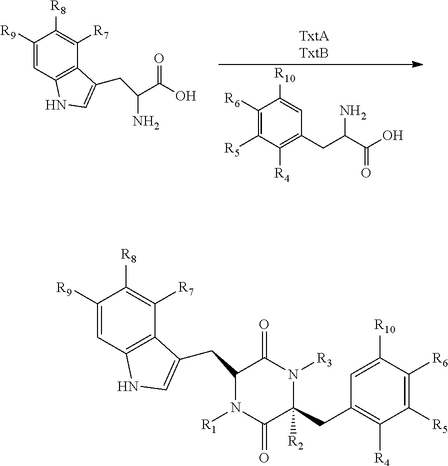

In various aspects, the disclosed methods of making the disclosed compounds comprise the biosynthetic schemes as follows:

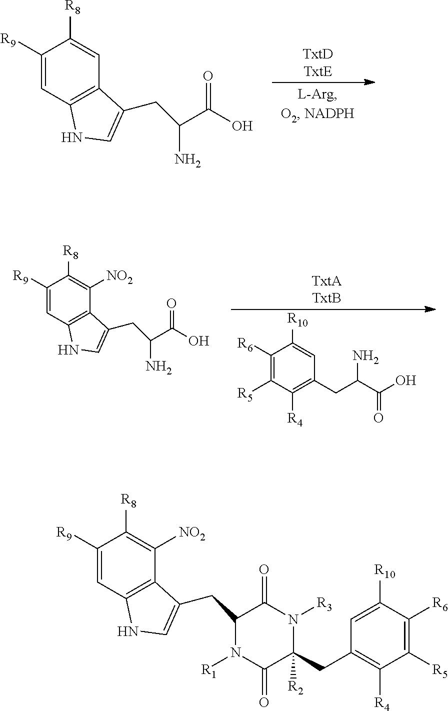

The TxtA, TxtB, TxtD, and TxtE enzymes are as disclosed herein below. In some aspects, the TxtA, TxtB, TxtD, and/or TxtE enzymes can be codon optimized. In a further aspect, the TxtA, TxtB, TxtD, and/or TxtE enzymes can be engineered as described herein below. Although suitable reaction conditions are provided below in the Examples, the skilled artisan can appreciate that the specifically disclosed reaction conditions can be modified or further optimized depending upon the substrates used.

### E. Agricultural Compositions

The present disclosure also concerns agricultural compositions comprising or consisting essentially of an active compound as described herein in combination with a suitable carrier (e.g., an agricultural carrier) or adjuvant (e.g., and agricultural adjuvant). In some aspects, the disclosed agricultural compositions are useful as herbicidal compositions for use with commercially useful plants and crops, including crops of useful plants.

The agricultural composition of the present disclosure, e.g., a disclosed herbicidal composition, can contain from 0.1 to 99% by weight, e.g., from 0.1 to 95% by weight, of a disclosed compound, 99.9 to 1% by weight, e.g., 99.8 to 5% by weight, of a solid or liquid adjuvant, and optionally from 0 to 25% by weight, e.g., from 0.1 to 25% by weight, of a surfactant.

The agricultural composition of the present disclosure, e.g., a disclosed herbicidal composition, can contain from 0.1 to 99% by weight, e.g., from 0.1 to 95% by weight, of a disclosed compound, 99.9 to 1% by weight, e.g., 99.8 to 5% by weight, of a solid or liquid carrier, and optionally from 0 to 25% by weight, e.g., from 0.1 to 25% by weight, of a surfactant.

Suitably, an agricultural composition of the present disclosure, e.g., a disclosed herbicidal composition, can be applied at any suitable developmental stage of the crop or plant. Rates and frequency of use of the formulations are those conventionally used in the art and factors such as the developmental stage of the plant and on the location, timing and application method, and density and development stage of the undesirable plant, e.g., a weed. Advantageous rates of application can range from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds.

In practice, as indicated above, an agricultural composition of the present disclosure, e.g., a disclosed herbicidal composition, can be applied as a formulation containing the various adjuvants and carriers known to or used in the industry. They may thus be formulated as granules, as wettable or soluble powders, as emulsifiable concentrates, as coatable pastes, as dusts, as flowables, as solutions, as suspensions or emulsions, or as controlled release forms such as microcapsules. These formulations are described in more detail below and may contain as little as about 0.5% to as much as about 95% or more by weight of the active ingredient. The optimum amount will depend on formulation, application equipment and nature of the plant to be treated. In addition, the disclosed agricultural compositions, e.g., an herbicidal composition, can optionally further comprise conventional additives such as surfactants, drift reduction agents, safeners, solubility enhancing agents, thickening agents, flow enhancers, foam-moderating agents, freeze protectants, UV protectants, preservatives, antimicrobials, and/or other additives that are necessary or desirable to improve the performance, crop safety, or handling of the composition.

Suitable agricultural adjuvants and carriers that are useful in formulating the compositions of the disclosure in the formulation types described above are well known to those skilled in the art. Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like. Suitable examples of the different classes are found in the non-limiting list below.

Exemplary agriculturally acceptable adjuvants include, but are not limited to, antifreeze agents, antifoam agents, compatibilizing agents, sequestering agents, neutralizing agents and buffers, corrosion inhibitors, colorants, odorants, penetration aids, wetting agents, spreading agents, dispersing agents, thickening agents, freeze point depressants, antimicrobial agents, crop oil, safeners, adhesives (for instance, for use in seed formulations), surfactants, protective colloids, emulsifiers, tackifiers, and mixtures thereof. Exemplary agriculturally acceptable adjuvants include, but are not limited to, crop oil concentrate (mineral oil (85%)+emulsifiers (15%)) or less, nonylphenol ethoxylate or less, benzylcocoalkyldimethyl quaternary ammonium salt or less, blend of petroleum hydrocarbon, alkyl esters, organic acid, and anionic surfactant or less, C9-C11 alkylpolyglycoside or less, phosphate alcohol ethoxylate or less, natural primary alcohol (C12-C16) ethoxylate or less, di-sec-butylphenol EO-PO block copolymer or less, polysiloxane-methyl cap or less, nonylphenol ethoxylate+urea ammonium nitrate or less, emulsified methylated seed oil or less, tridecyl alcohol (synthetic) ethoxylate (8 EO) or less, tallow amine ethoxylate (15 EO) or less, and PEG(400) dioleate-99.

In some aspects, the additive is a safener that is an organic compound leading to better crop plant compatibility when applied with a herbicide. In some aspects, the safener itself is herbicidally active. In some, the safener acts as an antidote or antagonist in the crop plants and can reduce or prevent damage to the crop plants. Exemplary safeners include, but are not limited to, AD-67 (MON 4660), benoxacor, benthiocarb, brassinolide, cloquintocet (mexyl), cyometrinil, cyprosulfamide, daimuron, dichlormid, dicyclonon, dietholate, dimepiperate, disulfoton, fenchlorazole, fenchlorazole-ethyl, fenclorim, flurazole, fluxofenim, furilazole, harpin proteins, isoxadifen-ethyl, jiecaowan, jiecaoxi, mefenpyr, mefenpyr-diethyl, mephenate, naphthalic anhydride, 2,2,5-trimethyl-3-(dichloroacetyl)-1,3-oxazolidine, 4-(dichloroacetyl)-1-oxa-4-azaspiro[4.5]decane, oxabetrinil, R29148, and N-phenyl-sulfonylbenzoic acid amides, as well as agriculturally acceptable salts and, provided they have a carboxyl group, their agriculturally acceptable derivatives thereof. In some aspects, the safener can be cloquintocet or an ester or salt thereof, such as cloquintocet (mexyl). For example, cloquintocet can be used to antagonize harmful effects of the compositions on rice and cereals.

Liquid carriers that can be employed include water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc. ethylene glycol, propylene glycol, glycerine, N-methyl-2-pyrrolidinone, and the like. Water is generally the carrier of choice for the dilution of concentrates.

Suitable solid carriers include talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour, lignin and the like.

A broad range of surface-active agents are advantageously employed in both said liquid and solid compositions, especially those designed to be diluted with carrier before application, including surfactants. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C 18 ethoxylate; alcohol-alkylene oxide addition products, such as tridecyl alcohol-C 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Exemplary surfactants (e.g., wetting agents, tackifiers, dispersants, emulsifiers) include, but are not limited to, the alkali metal salts, alkaline earth metal salts and ammonium salts of aromatic sulfonic acids, for example lignosulfonic acids, phenolsulfonic acids, naphthalenesulfonic acids, and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalene sulfonic acids with phenol and formaldehyde, polyoxyethylene octylphenol ether, ethoxylated isooctyl-, octyl- or nonylphenol, alkylphenyl or tributylphenyl polyglycol ether, alkyl aryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g., methylcellulose), hydrophobically modified starches, polyvinyl alcohol, polycarboxylates, polyalkoxylates, polyvinyl amine, polyethyleneimine, polyvinylpyrrolidone and copolymers thereof.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient, i.e., one or more disclosed compound, may range from about 0.5% to about 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain about 5% to about 95% of the active ingredient plus a small amount of wetting, dispersing or emulsifying agent. Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from about 0.5% to about 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain about 5% to about 25% active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically about 1 to 50 microns in diameter. The enclosed liquid typically constitutes about 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

In addition, further, other biocidally active ingredients or compositions may be combined with the disclosed compound and used in the methods of the disclosure and applied simultaneously or sequentially with the disclosed compound. When applied simultaneously, these further active ingredients may be formulated together with the disclosed compound or mixed in, for example, the spray tank. These further biocidally active ingredients may be fungicides, herbicides, insecticides, bactericides, acaricides, nematicides and/or plant growth regulators.

Accordingly, the present disclosure provides for the use of a composition in the methods of the present disclosure, said composition comprising (i) a disclosed compound and (i) a fungicide, (ii) a herbicide, (iii) an insecticide, (iv) a bactericide, (v) an acaricide, (vi) a nematicide and/or (vii) a plant growth regulator.

The herbicidal compositions of the present disclosure optionally can further comprise at least one non-auxin herbicide. The term “non-auxin herbicide” refers to a herbicide having a primary mode of action other than as an auxin herbicide. Representative examples of non-auxin herbicides include acetyl CoA carboxylase (ACCase) inhibitors, acetolactate synthase (ALS) inhibitors, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvyl shikimate-3-phosphate (EPSP) synthase inhibitor, glutamine synthetase inhibitor, dihydropteroate synthetase inhibitor, mitosis inhibitors, and nucleic acid inhibitors; salts and esters thereof; racemic mixtures and resolved isomers thereof; and combinations thereof.

Representative examples of ACCase inhibitors include clethodim, clodinafop, fenoxaprop-P, fluazifop-P, quizalofop-P, and sethoxydim.

Representative examples of ALS or AHAS inhibitors include flumetsulam, imazamethabenz-m, imazamox, imazapic, imazapyr, imazaquin, imazethapyr, metsulfuron, prosulfuron, and sulfosulfuron.

Representative examples of photosystem I inhibitors include diquat and paraquat.

Representative examples of photosystem II inhibitors include atrazine, cyanazine, diuron, and metibuzin.

Representative examples of PPO inhibitors include acifluorofen, butafenacil, carfentrazone-ethyl, flufenpyr-ethyl, fluthiacet, flumiclorac, flumioxazin, fomesafen, lactofen, oxadiazon, oxyfluorofen, and sulfentrazone.

Representative examples of carotenoid biosynthesis inhibitors include aclonifen, amitrole, diflufenican, mesotrione, and sulcotrione.

A representative example of an EPSP inhibitor is N-phosphonomethyl glycine (glyphosate).

A representative example of a glutamine synthetase inhibitor is glufosinate.

A representative example of a dihydropteroate synthetase inhibitor is asulam.

Representative examples of mitosis inhibitors include acetochlor, alachlor, dithiopyr, S-metolachlor, and thiazopyr.

Representative examples of nucleic acid inhibitors include difenzoquat, fosamine, metham, and pelargonic acid.

In one aspect, the herbicidal compositions of the present disclosure further comprise a non-auxin herbicide selected from the group consisting of acetochlor, glyphosate, glufosinate, flumioxazin, fomesafen, and agriculturally acceptable salts thereof.

In one aspect, the herbicidal compositions of the present disclosure further comprise glyphosate, or an agriculturally acceptable salt thereof. Suitable glyphosate salts include, for example, the ammonium, diammonium, dimethylammonium, monoethanolamine, isopropylamine, and potassium salts, and combinations thereof. In one aspect, the glyphosate salts are selected from the group consisting of monoethanolamine, isopropylamine, and potassium salts, and combinations thereof.

In one aspect, the herbicidal compositions of the present disclosure further comprise glufosinate, or an agriculturally acceptable salt thereof.

In one aspect, the herbicidal compositions of the present disclosure can further comprise dicamba, or an agriculturally acceptable salt or ester thereof, and glyphosate, or an agriculturally acceptable salt thereof. In another aspect, the herbicidal compositions of the present disclosure comprise dicamba, or an agriculturally acceptable salt thereof; glyphosate, or an agriculturally acceptable salt thereof; and a non-ammoniated, agriculturally acceptable acetate salt. Commercially available sources of glyphosate, and its agriculturally acceptable salts, include those products sold under the trade names DURANGO® DMA®, HONCHO PLUS®, ROUNDUP POWERMAX®, ROUNDUP WEATHERMAX®, TRAXION®, and TOUCHDOWN®.

In one aspect, the herbicidal compositions of the present disclosure can further comprise 2,4-D, or an agriculturally acceptable salt or ester thereof, and glyphosate, or an agriculturally acceptable salt thereof. In another aspect, the herbicidal compositions of the present disclosure comprise 2,4-D, or an agriculturally acceptable salt or ester thereof; glyphosate, or an agriculturally acceptable salt thereof; and a non-ammoniated, agriculturally acceptable acetate salt.

In some aspects, the disclosed herbicidal compositions can further comprise an additive such as a pesticide. Exemplary pesticides include, but are not limited to, 2,4-D, acetochlor, aclonifen, amicarbazone, 4-aminopicolinic acid based herbicides, such as halauxifen, halauxifen-methyl, and those described in U.S. Pat. Nos. 7,314,849 and 7,432,227 to Balko, et al., amidosulfuron, aminocyclopyrachlor, aminopyralid, aminotriazole, ammonium thiocyanate, anilofos, asulam, azimsulfuron, atrazine, beflubutamid, benazolin, benfuresate, bensulfuron-methyl, bentazon-sodium, benzofenap, bifenox, bispyribac-sodium, bromobutide, bromacil, bromoxynil, butachlor, butafenacil, butralin, butroxydim, carbetamide, cafenstrole, carfentrazone, carfentrazone-ethyl, chlormequat, clopyralid, chlorsulfuron, chlortoluron, cinidon-ethyl, clethodim, clodinafop-propargyl, clomeprop, clomazone, cloransulam-methyl, cyanazine, cyclosulfamuron, cycloxydim, cyhalofop-butyl, daimuron, dicamba, dichlobenil, dichlorprop-P, diclofop-methyl, diclosulam, diflufenican, diflufenzopyr, dimefuron, dimethachlor, diquat, diuron, S-ethyl dipropylcarbamothioate (EPTC), esprocarb, ethoxysulfuron, etobenzanid, fenoxaprop, fenoxaprop-ethyl, fenoxaprop-ethyl+isoxadifen-ethyl, fenoxaprop-P-ethyl, fenoxasulfone, fenquinotrione, fentrazamide, flazasulfuron, florasulam, fluazifop, fluazifop-P-butyl, flucarbazone, flucarbazone-sodium, flucetosulfuron (LGC-42153), flufenacet, flumetsulam, flumioxazin, flupyrsulfuron, flurochloridone, fluroxypyr, fluroxypyr-meptyl, flurtamone, glufosinate, glufosinate-ammonium, glyphosate, halosulfuron-methyl, haloxyfop-methyl, haloxyfop-R-methyl, hexazinone, imazamethabenz, imazamox, imazapic, imazapyr, imazaquin, imazethapyr, imazosulfuron, indanofan, indaziflam, iodosulfuron, iodosulfuron-ethyl-sodium, iofensulfuron, ioxynil, ipfencarbazone, isoproturon, isoxaben, isoxaflutole, lactofen, linuron, MCPA, MCPB, mecoprop-P, mefenacet, mesosulfuron, mesosulfuron-ethyl sodium, mesotrione, metamifop, metazochlor, metazosulfuron, metosulam, metribuzin, metsulfuron, metsulfuron-methyl, molinate, MSMA, napropamide, napropamide-M, orfurazon, orthosulfamuron, oryzalin, oxadiargyl, oxadiazon, oxazichlomefone, oxyfluorfen, paraquat, pendimethalin, penoxsulam, pentoxazone, pethoxamid, picloram, picolinafen, pinoxaden, pretilachlor, primisulfuron, profluazol, profoxydim, propanil, propaquizafop, propyrisulfuron, propoxycarbazone, propyzamide, prosulfocarb, prosulfuron, pyraclonil, pyraflufen-ethyl, pyrasulfotole, pyrazosulfuron-ethyl, pyrazolynate, pyribenzoxim (LGC-40863), pyributicarb, pyridate, pyriftalid, pyrimisulfan, pyroxsulam, pyroxasulfone, quinclorac, quinmerac, quizalofop-ethyl-D, quizalofop-P-ethyl, quizalofop-P-tefuryl, rimsulfuron, sethoxydim, simazine, sulfentrazone, sulfometuron, sulfosate, sulfosulfuron, tebuthiuron, tefuryltrione, tepraloxidim, terbacil, terbuthylazine, terbutryn, thenylchlor, thiazopyr, thifensulfuron, thifensulfuron-methyl, thiobencarb, topramezone, tralkoxydim, triafamone, triasulfuron, tribenuron, tribenuron-methyl, triafamone, triclopyr, and trifluralin, and agriculturally acceptable salts, choline salts, esters and mixtures thereof. In certain aspects, the additional pesticide includes benzofenap, cyhalofop, daimuron, pentoxazone, esprocarb, pyrazosulfuron, butachlor, pretilachlor, metazosulfuron, bensulfuron-methyl, imazosulfuron, azimsulfuron, bromobutide, benfuresate, mesotrione, oxazichlomefone, and agriculturally acceptable salts or esters thereof, or combinations thereof. In certain aspects, the additional pesticide includes triclopyr choline salt.

### F. Methods of Using

The agricultural compositions disclosed herein can be applied in any known technique for applying herbicides. Exemplary application techniques include, but are not limited to, spraying, atomizing, dusting, spreading, or direct application into water (in-water). The method of application can vary depending on the intended purpose. In some aspects, the method of application can be chosen to ensure the finest possible distribution of the compositions disclosed herein.

The agricultural compositions disclosed herein can be applied pre-emergence (before the emergence of undesirable vegetation) or post-emergence (i.e., during and/or after emergence of the undesirable vegetation). The composition can be applied, for example, to the vegetation as an in-water application to a flooded rice field.

When the agricultural compositions are used in crops, the compositions can be applied after seeding and before or after the emergence of the crop plants. In some aspects, the compositions disclosed herein show good crop tolerance even when the crop has already emerged, and can be applied during or after the emergence of the crop plants. In some aspects, when the compositions are used in crops, the compositions can be applied before seeding of the crop plants.

In some aspects, the compositions disclosed herein are applied to vegetation or an area adjacent the vegetation, or applied to soil, or applied to/into water, for example to/into flooded rice fields, to prevent the emergence or growth of vegetation by spraying (e.g., foliar spraying or spraying into the water of a flooded rice field). In some aspects, the spraying techniques use, for example, water as carrier and spray liquor rates of from 10 liters per hectare (L/ha) to 2000 L/ha (e.g., from 50 L/ha to 1000 L/ha, or from 100 to 500 L/ha). In some aspects, the compositions disclosed herein are applied by the low-volume or the ultra-low-volume method, wherein the application is in the form of micro granules. In some aspects, the compositions disclosed herein can be applied as dry formulations (e.g., granules, WDGs) into water.

The compositions and methods disclosed herein can be used to control undesired vegetation in a variety of crop and non-crop applications. In some aspects, the compositions and methods disclosed herein can be used for controlling undesired vegetation in rice (e.g., in direct-seeded rice, water-seeded rice, transplanted rice, or rice seedbeds prior to planting rice seeds or rice transplants).

### G. Aspects

The following listing of exemplary aspects supports and is supported by the disclosure provided herein.

Aspect 1. A compound, wherein the compound has a formula represented by a structure:

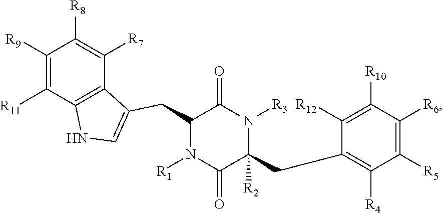

wherein each of R₁ and R₃ is independently selected from hydrogen and C1-C3 alkyl; wherein R₂ is selected from hydrogen and hydroxy; and wherein each of R₄, R₅, R₆, R₇, R₈, R₉, R₁₀, R₁₁, and R₁₂ is independently selected hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 2. The compound of Aspect 1, wherein R₁ is hydrogen.

Aspect 3. The compound of Aspect 1, wherein R₁ is selected from hydrogen, methyl, and ethyl.

Aspect 4. The compound of Aspect 1, wherein R₁ is selected from hydrogen and methyl.

Aspect 5. The compound of Aspect 1, wherein R₁ is selected from methyl, ethyl, propyl, and isopropyl.

Aspect 6. The compound of Aspect 5, wherein R₁ is methyl.

Aspect 7. The compound of Aspect 5, wherein R₁ is ethyl.

Aspect 8. The compound of Aspect 5, wherein R₁ is propyl or isopropyl.

Aspect 9. The compound of any one of Aspect 1-Aspect 8, wherein R₂ is hydrogen.

Aspect 10. The compound of any one of Aspect 1-Aspect 8, wherein R₂ is hydroxy.

Aspect 11. The compound of any one of Aspect 1-Aspect 10, wherein R₃ is hydrogen.

Aspect 12. The compound of any one of Aspect 1-Aspect 10, wherein R₃ is selected from hydrogen, methyl, and ethyl.

Aspect 13. The compound of any one of Aspect 1-Aspect 10, wherein R₃ is selected from hydrogen and methyl.

Aspect 14. The compound of any one of Aspect 1-Aspect 10, wherein R₃ is selected from methyl, ethyl, propyl, and isopropyl.

Aspect 15. The compound of Aspect 14, wherein R₃ is methyl.

Aspect 16. The compound of Aspect 14, wherein R₃ is ethyl.

Aspect 17. The compound of Aspect 14, wherein R₃ is propyl or isopropyl.

Aspect 18. The compound of any one of Aspect 1-Aspect 17, wherein R₄ is hydrogen.

Aspect 19. The compound of any one of Aspect 1-Aspect 17, wherein R₄ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 20. The compound of Aspect 19, wherein R₄ is selected from azido, hydroxy, hydrazino, sulfhydryl, amino, isocyano, cyano, nitro, —F, —Cl, —Br, methyl, ethyl, —CH₂F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 21. The compound of Aspect 20, wherein R₄ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 22. The compound of Aspect 20, wherein R₄ is selected from hydroxy, azido, —F, —Cl, —Br, methyl, and ethyl.

Aspect 23. The compound of any one of Aspect 1-Aspect 17, wherein R₄ is selected from hydrogen, halo, hydroxy, azido, and C1-C3 alkyl.

Aspect 24. The compound of Aspect 23, wherein R₄ is selected from hydrogen, halo, hydroxy, azido, and methyl.

Aspect 25. The compound of Aspect 23, wherein R₄ is selected from hydrogen, —F, —Cl, —Br, hydroxy, azido, and methyl.

Aspect 26. The compound of Aspect 23, wherein R₄ is selected from hydrogen, hydroxy, azido, and methyl.

Aspect 27. The compound of Aspect 23, wherein R₄ is selected from hydrogen, —F, —Cl, —Br, hydroxy, and methyl.

Aspect 28. The compound of Aspect 23, wherein R₄ is selected from hydrogen, —F, —Cl, —Br, azido, and methyl.

Aspect 29. The compound of Aspect 23, wherein R₄ is selected from hydrogen, —F, —Cl, —Br, and methyl.

Aspect 30. The compound of Aspect 23, wherein R₄ is selected from hydrogen, azido, and methyl.

Aspect 31. The compound of Aspect 23, wherein R₄ is selected from hydrogen, hydroxy, azido, and methyl.

Aspect 32. The compound of Aspect 23, wherein R₄ is selected from —F, —Cl, —Br, hydroxy, azido, and methyl.

Aspect 33. The compound of Aspect 23, wherein R₄ is selected from —F, —Cl, —Br, hydroxy, and methyl.

Aspect 34. The compound of Aspect 23, wherein R₄ is selected from hydrogen, —F, —Cl, —Br, hydroxy, and methyl.

Aspect 35. The compound of Aspect 23, wherein R₄ is selected from hydrogen, —F, —Cl, —Br, hydroxy, and azido.

Aspect 36. The compound of any one of Aspect 1-Aspect 35, wherein R₅ is hydrogen.

Aspect 37. The compound of any one of Aspect 1-Aspect 35, wherein R₅ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 38. The compound of Aspect 37, wherein R₅ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 39. The compound of Aspect 38, wherein R₅ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 40. The compound of Aspect 38, wherein R₅ is selected from hydroxy, azido, —F, —Cl, —Br, methyl, and ethyl.

Aspect 41. The compound of any one of Aspect 1-Aspect 35, wherein R₅ is selected from hydrogen, halo, hydroxy, azido, and C1-C3 alkyl.

Aspect 42. The compound of Aspect 41, wherein R₅ is selected from hydrogen, halo, hydroxy, azido, and methyl.

Aspect 43. The compound of Aspect 41, wherein R₅ is selected from hydrogen, —F, —Cl, —Br, hydroxy, azido, and methyl.

Aspect 44. The compound of Aspect 41, wherein R₅ is selected from hydrogen, hydroxy, azido, and methyl.

Aspect 45. The compound of Aspect 41, wherein R₅ is selected from hydrogen, —F, —Cl, —Br, hydroxy, and methyl.

Aspect 46. The compound of Aspect 41, wherein R₅ is selected from hydrogen, —F, —Cl, —Br, azido, and methyl.

Aspect 47. The compound of Aspect 41, wherein R₅ is selected from hydrogen, —F, —Cl, —Br, and methyl.

Aspect 48. The compound of Aspect 41, wherein R₅ is selected from hydrogen, azido, and methyl.

Aspect 49. The compound of Aspect 41, wherein R₅ is selected from hydrogen, hydroxy, azido, and methyl.

Aspect 50. The compound of Aspect 41, wherein R₅ is selected from —F, —Cl, —Br, hydroxy, azido, and methyl.

Aspect 51. The compound of Aspect 41, wherein R₅ is selected from —F, —Cl, —Br, hydroxy, and methyl.

Aspect 52. The compound of Aspect 41, wherein R₅ is selected from hydrogen, —F, —Cl, —Br, hydroxy, and methyl.

Aspect 53. The compound of Aspect 41, wherein R₅ is selected from hydrogen, —F, —Cl, —Br, hydroxy, and azido.

Aspect 54. The compound of any one of Aspect 1-Aspect 53, wherein R₆ is hydrogen.

Aspect 55. The compound of any one of Aspect 1-Aspect 53, wherein R₆ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 56. The compound of Aspect 55, wherein R₆ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 57. The compound of Aspect 56, wherein R₆ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 58. The compound of Aspect 56, wherein R₆ is selected from hydroxy, azido, —F, —Cl, —Br, methyl, and ethyl.

Aspect 59. The compound of any one of Aspect 1-Aspect 53, wherein R₆ is selected from hydrogen, halo, hydroxy, azido, and C1-C3 alkyl.

Aspect 60. The compound of Aspect 59, wherein R₆ is selected from hydrogen, halo, hydroxy, azido, and methyl.

Aspect 61. The compound of Aspect 59, wherein R₆ is selected from hydrogen, —F, —Cl, —Br, hydroxy, azido, and methyl.

Aspect 62. The compound of Aspect 59, wherein R₆ is selected from hydrogen, hydroxy, azido, and methyl.

Aspect 63. The compound of Aspect 59, wherein R₆ is selected from hydrogen, —F, —Cl, —Br, hydroxy, and methyl.

Aspect 64. The compound of Aspect 59, wherein R₆ is selected from hydrogen, —F, —Cl, —Br, azido, and methyl.

Aspect 65. The compound of Aspect 59, wherein R₆ is selected from hydrogen, —F, —Cl, —Br, and methyl.

Aspect 66. The compound of Aspect 59, wherein R₆ is selected from hydrogen, azido, and methyl.

Aspect 67. The compound of Aspect 59, wherein R₆ is selected from hydrogen, hydroxy, azido, and methyl.

Aspect 68. The compound of Aspect 59, wherein R₆ is selected from —F, —Cl, —Br, hydroxy, azido, and methyl.

Aspect 69. The compound of Aspect 59, wherein R₆ is selected from —F, —Cl, —Br, hydroxy, and methyl.

Aspect 70. The compound of Aspect 59, wherein R₆ is selected from hydrogen, —F, —Cl, —Br, hydroxy, and methyl.

Aspect 71. The compound of Aspect 59, wherein R₆ is selected from hydrogen, —F, —Cl, —Br, hydroxy, and azido.

Aspect 72. The compound of any one of Aspect 1-Aspect 71, wherein R₇ is hydrogen.

Aspect 73. The compound of any one of Aspect 1-Aspect 71, wherein R₇ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 74. The compound of Aspect 73, wherein R₇ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 75. The compound of Aspect 74, wherein R₇ is selected from azido, hydroxy, hydrazino, sulfhydryl, amino, isocyano, cyano, nitro, —F, —Cl, —Br, methyl, —CH₂F, —CH2Cl, —CH₂Br, —CHF2, —CF3, —CHCl2, —CCl3, —CHBr2, —CBr3, —OCH₃, —NHCH3, —N(CH3)2, —CH2OH, —CH2OCH3, —CH2OCH2CH3, —CH2CH2OCH3, —CH2NH2, and —CH2NHCH3.

Aspect 76. The compound of Aspect 74, wherein R₇ is selected from hydroxy, azido, —F, —Cl, —Br, methyl, and ethyl.

Aspect 77. The compound of any one of Aspect 1-Aspect 71, wherein R₇ is selected from hydrogen, halo, hydroxy, azido, and C1-C3 alkyl.

Aspect 78. The compound of Aspect 77, wherein R₇ is selected from hydrogen, halo, hydroxy, azido, and methyl.

Aspect 79. The compound of Aspect 77, wherein R₇ is selected from hydrogen, —F, —Cl, —Br, hydroxy, azido, and methyl.

Aspect 80. The compound of Aspect 77, wherein R₇ is selected from hydrogen, hydroxy, azido, and methyl.

Aspect 81. The compound of Aspect 77, wherein R₇ is selected from hydrogen, —F, —Cl, —Br, hydroxy, and methyl.

Aspect 82. The compound of Aspect 77, wherein R₇ is selected from hydrogen, —F, —Cl, —Br, azido, and methyl.

Aspect 83. The compound of Aspect 77, wherein R₇ is selected from hydrogen, —F, —Cl, —Br, and methyl.

Aspect 84. The compound of Aspect 77, wherein R₇ is selected from hydrogen, azido, and methyl.

Aspect 85. The compound of Aspect 77, wherein R₇ is selected from hydrogen, hydroxy, azido, and methyl.

Aspect 86. The compound of Aspect 77, wherein R₇ is selected from —F, —Cl, —Br, hydroxy, azido, and methyl.

Aspect 87. The compound of Aspect 77, wherein R₇ is selected from —F, —Cl, —Br, hydroxy, and methyl.

Aspect 88. The compound of Aspect 77, wherein R₇ is selected from hydrogen, —F, —Cl, —Br, hydroxy, and methyl.

Aspect 89. The compound of Aspect 77, wherein R₇ is selected from hydrogen, —F, —Cl, —Br, hydroxy, and azido.

Aspect 90. The compound of any one of Aspect 1-Aspect 89, wherein R₈ is hydrogen.

Aspect 91. The compound of any one of Aspect 1-Aspect 89, wherein R₈ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 92. The compound of Aspect 91, wherein R₈ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 93. The compound of Aspect 92, wherein R₈ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 94. The compound of Aspect 92, wherein R₈ is selected from hydroxy, azido, —F, —Cl, —Br, methyl, and ethyl.

Aspect 95. The compound of any one of Aspect 1-Aspect 89, wherein R₈ is selected from hydrogen, halo, hydroxy, azido, and C1-C3 alkyl.

Aspect 96. The compound of Aspect 95, wherein R₈ is selected from hydrogen, halo, hydroxy, azido, and methyl.

Aspect 97. The compound of Aspect 95, wherein R₈ is selected from hydrogen, —F, —Cl, —Br, hydroxy, azido, and methyl.

Aspect 98. The compound of Aspect 95, wherein R₈ is selected from hydrogen, hydroxy, azido, and methyl.

Aspect 99. The compound of Aspect 95, wherein R₈ is selected from hydrogen, —F, —Cl, —Br, hydroxy, and methyl.

Aspect 100. The compound of Aspect 95, wherein R₈ is selected from hydrogen, —F, —Cl, —Br, azido, and methyl.

Aspect 101. The compound of Aspect 95, wherein R₈ is selected from hydrogen, —F, —Cl, —Br, and methyl.

Aspect 102. The compound of Aspect 95, wherein R₈ is selected from hydrogen, azido, and methyl.

Aspect 103. The compound of Aspect 95, wherein R₈ is selected from hydrogen, hydroxy, azido, and methyl.

Aspect 104. The compound of Aspect 95, wherein R₈ is selected from —F, —Cl, —Br, hydroxy, azido, and methyl.

Aspect 105. The compound of Aspect 95, wherein R₈ is selected from —F, —Cl, —Br, hydroxy, and methyl.

Aspect 106. The compound of Aspect 95, wherein R₈ is selected from hydrogen, —F, —Cl, —Br, hydroxy, and methyl.

Aspect 107. The compound of Aspect 95, wherein R₈ is selected from hydrogen, —F, —Cl, —Br, hydroxy, and azido.

Aspect 108. The compound of any one of Aspect 1-Aspect 107, wherein R₉ is hydrogen.

Aspect 109. The compound of any one of Aspect 1-Aspect 107, wherein R₉ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 110. The compound of Aspect 109, wherein R₉ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 111. The compound of Aspect 110, wherein R₉ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 112. The compound of Aspect 110, wherein R₉ is selected from hydroxy, azido, —F, —Cl, —Br, methyl, and ethyl.

Aspect 113. The compound of any one of Aspect 1-Aspect 107, wherein R₉ is selected from hydrogen, halo, hydroxy, azido, and C1-C3 alkyl.

Aspect 114. The compound of Aspect 113, wherein R₉ is selected from hydrogen, halo, hydroxy, azido, and methyl.

Aspect 115. The compound of Aspect 113, wherein R₉ is selected from hydrogen, —F, —Cl, —Br, hydroxy, azido, and methyl.

Aspect 116. The compound of Aspect 113, wherein R₉ is selected from hydrogen, hydroxy, azido, and methyl.

Aspect 117. The compound of Aspect 113, wherein R₉ is selected from hydrogen, —F, —Cl, —Br, hydroxy, and methyl.

Aspect 118. The compound of Aspect 113, wherein R₉ is selected from hydrogen, —F, —Cl, —Br, azido, and methyl.

Aspect 119. The compound of Aspect 113, wherein R₉ is selected from hydrogen, —F, —Cl, —Br, and methyl.

Aspect 120. The compound of Aspect 113, wherein R₉ is selected from hydrogen, azido, and methyl.

Aspect 121. The compound of Aspect 113, wherein R₉ is selected from hydrogen, hydroxy, azido, and methyl.

Aspect 122. The compound of Aspect 113, wherein R₉ is selected from —F, —Cl, —Br, hydroxy, azido, and methyl.

Aspect 123. The compound of Aspect 113, wherein R₉ is selected from —F, —Cl, —Br, hydroxy, and methyl.

Aspect 124. The compound of Aspect 113, wherein R₉ is selected from hydrogen, —F, —Cl, —Br, hydroxy, and methyl.

Aspect 125. The compound of Aspect 113, wherein R₉ is selected from hydrogen, —F, —Cl, —Br, hydroxy, and azido.

Aspect 126. The compound of any one of Aspect 1-Aspect 125, wherein R₁₀ is hydrogen.

Aspect 127. The compound of any one of Aspect 1-Aspect 125, wherein R₁₀ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 128. The compound of Aspect 127, wherein R₁₀ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 129. The compound of Aspect 128, wherein R₁₀ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 130. The compound of Aspect 128, wherein R₁₀ is selected from hydroxy, azido, —F, —Cl, —Br, methyl, and ethyl.

Aspect 131. The compound of any one of Aspect 1-Aspect 125, wherein R₁₀ is selected from hydrogen, halo, hydroxy, azido, and C1-C3 alkyl.

Aspect 132. The compound of Aspect 131, wherein R₁₀ is selected from hydrogen, halo, hydroxy, azido, and methyl.

Aspect 133. The compound of Aspect 131, wherein R₁₀ is selected from hydrogen, —F, —Cl, —Br, hydroxy, azido, and methyl.

Aspect 134. The compound of Aspect 131, wherein R₁₀ is selected from hydrogen, hydroxy, azido, and methyl.

Aspect 135. The compound of Aspect 131, wherein R₁₀ is selected from hydrogen, —F, —Cl, —Br, hydroxy, and methyl.

Aspect 136. The compound of Aspect 131, wherein R₁₀ is selected from hydrogen, —F, —Cl, —Br, azido, and methyl.

Aspect 137. The compound of Aspect 131, wherein R₁₀ is selected from hydrogen, —F, —Cl, —Br, and methyl.

Aspect 138. The compound of Aspect 131, wherein R₁₀ is selected from hydrogen, azido, and methyl.

Aspect 139. The compound of Aspect 131, wherein R₁₀ is selected from hydrogen, hydroxy, azido, and methyl.

Aspect 140. The compound of Aspect 131, wherein R₁₀ is selected from —F, —Cl, —Br, hydroxy, azido, and methyl.

Aspect 141. The compound of Aspect 131, wherein R₁₀ is selected from —F, —Cl, —Br, hydroxy, and methyl.

Aspect 142. The compound of Aspect 131, wherein R₁₀ is selected from hydrogen, —F, —Cl, —Br, hydroxy, and methyl.

Aspect 143. The compound of Aspect 131, wherein R₁₀ is selected from hydrogen, —F, —Cl, —Br, hydroxy, and azido.

Aspect 144. The compound of any one of Aspect 1-Aspect 143, wherein R₁₁ is hydrogen.

Aspect 145. The compound of any one of Aspect 1-Aspect 143, wherein R₁₁ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 146. The compound of Aspect 145, wherein R₁₁ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and —CH2CH2NHCH3.

Aspect 147. The compound of Aspect 146, wherein R₁₁ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 148. The compound of Aspect 146, wherein R₁₁ is selected from hydroxy, azido, —F, —Cl, —Br, methyl, and ethyl.

Aspect 149. The compound of any one of Aspect 1-Aspect 143, wherein R₁₁ is selected from hydrogen, halo, hydroxy, azido, and C1-C3 alkyl.

Aspect 150. The compound of Aspect 149, wherein R₁₁ is selected from hydrogen, halo, hydroxy, azido, and methyl.

Aspect 151. The compound of Aspect 149, wherein R₁₁ is selected from hydrogen, —F, —Cl, —Br, hydroxy, azido, and methyl.

Aspect 152. The compound of Aspect 149, wherein R₁₁ is selected from hydrogen, hydroxy, azido, and methyl.

Aspect 153. The compound of Aspect 149, wherein R₁₁ is selected from hydrogen, —F, —Cl, —Br, hydroxy, and methyl.

Aspect 154. The compound of Aspect 149, wherein R₁₁ is selected from hydrogen, —F, —Cl, —Br, azido, and methyl.

Aspect 155. The compound of Aspect 149, wherein R₁₁ is selected from hydrogen, —F, —Cl, —Br, and methyl.

Aspect 156. The compound of Aspect 149, wherein R₁₁ is selected from hydrogen, azido, and methyl.

Aspect 157. The compound of Aspect 149, wherein R₁₁ is selected from hydrogen, hydroxy, azido, and methyl.

Aspect 158. The compound of Aspect 149, wherein R₁₁ is selected from —F, —Cl, —Br, hydroxy, azido, and methyl.

Aspect 159. The compound of Aspect 149, wherein R₁₁ is selected from —F, —Cl, —Br, hydroxy, and methyl.

Aspect 160. The compound of Aspect 149, wherein R₁₁ is selected from hydrogen, —F, —Cl, —Br, hydroxy, and methyl.

Aspect 161. The compound of Aspect 149, wherein R₁₁ is selected from hydrogen, —F, —Cl, —Br, hydroxy, and azido.

Aspect 162. The compound of any one of Aspect 1-Aspect 161, wherein R₁₂ is hydrogen.

Aspect 163. The compound of any one of Aspect 1-Aspect 161, wherein R₁₂ is selected from azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl.

Aspect 164. The compound of Aspect 163, wherein R₁₂ is selected from azido, hydroxy, hydrazino, sulfhydryl, amino, isocyano, cyano, nitro, —F, —Cl, —Br, methyl, ethyl, —CH2F, —CH2Cl, —CH2Br, —CH2CH2F, —CH2CH2Cl, —CH2CH2Br, —CHF2, —CF3, —CHCl2, —CCl3, —CHBr2, —CBr3, —CH2CHF2, —CH2CF3, —CH2CHCl2, —CH2CCl3, —CH2CHBr2, —CH2CBr3, —OCH3, —OCH2CH3, —NHCH3, —NHCH2CH3, —N(CH3)2, —N(CH3)CH2CH3, —CH2OH, —(CH2)2OH, —(CHOH)CH3, —(CHOH)CH2CH3, —CH2OCH3, —CH2OCH2CH3, —CH2CH2OCH3, —CH2NH2, —CH2NHCH3, —CH2NHCH2CH3, and CH2CH2NHCH3.

Aspect 165. The compound of Aspect 164, wherein R₁₂ is selected from azido, hydroxy, hydrazino, sulfhydryl, amino, isocyano, cyano, nitro, —F, —Cl, —Br, methyl, —CH2F, —CH2Cl, —CH2Br, —CHF2, —CF3, —CHCl2, —CCl3, —CHBr2, —CBr3, —OCH3, —NHCH3, —N(CH3)2, —CH2OH, —CH2OCH3, —CH2OCH2CH3, —CH2CH2OCH3, —CH2NH2, and —CH2NHCH3.

Aspect 166. The compound of Aspect 164, wherein R₁₂ is selected from hydroxy, azido, —F, —Cl, —Br, methyl, and ethyl.

Aspect 167. The compound of any one of Aspect 1-Aspect 161, wherein R₁₂ is selected from hydrogen, halo, hydroxy, azido, and C1-C3 alkyl.

Aspect 168. The compound of Aspect 167, wherein R₁₂ is selected from hydrogen, halo, hydroxy, azido, and methyl.

Aspect 169. The compound of Aspect 167, wherein R₁₂ is selected from hydrogen, —F, —Cl, —Br, hydroxy, azido, and methyl.

Aspect 170. The compound of Aspect 167, wherein R₁₂ is selected from hydrogen, hydroxy, azido, and methyl.

Aspect 171. The compound of Aspect 167, wherein R₁₂ is selected from hydrogen, —F, —Cl, —Br, hydroxy, and methyl.

Aspect 172. The compound of Aspect 167, wherein R₁₂ is selected from hydrogen, —F, —Cl, —Br, azido, and methyl.

Aspect 173. The compound of Aspect 167, wherein R₁₂ is selected from hydrogen, —F, —Cl, —Br, and methyl.

Aspect 174. The compound of Aspect 167, wherein R₁₂ is selected from hydrogen, azido, and methyl.

Aspect 175. The compound of Aspect 167, wherein R₁₂ is selected from hydrogen, hydroxy, azido, and methyl.

Aspect 176. The compound of Aspect 167, wherein R₁₂ is selected from —F, —Cl, —Br, hydroxy, azido, and methyl.

Aspect 177. The compound of Aspect 167, wherein R₁₂ is selected from —F, —Cl, —Br, hydroxy, and methyl.

Aspect 178. The compound of Aspect 167, wherein R₁₂ is selected from hydrogen, —F, —Cl, —Br, hydroxy, and methyl.

Aspect 179. The compound of Aspect 167, wherein R₁₂ is selected from hydrogen, —F, —Cl, —Br, hydroxy, and azido.

Aspect 180. The compound of Aspect 1, wherein R₁ is hydrogen; and wherein R₃ is selected from hydrogen and C1-C3 alkyl.

Aspect 181. The compound of Aspect 1, wherein R₁ is hydrogen; and wherein R₃ is C1-C3 alkyl.

Aspect 182. The compound of Aspect 181, wherein R₃ is methyl or ethyl.

Aspect 183. The compound of Aspect 181, wherein R₃ is methyl.

Aspect 184. The compound of Aspect 1, wherein R₃ is hydrogen; and wherein R₁ is selected from hydrogen and C1-C3 alkyl.

Aspect 185. The compound of Aspect 184, wherein R₁ is methyl or ethyl.

Aspect 186. The compound of Aspect 184, wherein R₁ is methyl.

Aspect 187. The compound of Aspect 1, wherein R₃ is hydrogen; and wherein R₁ is C1-C3 alkyl.

Aspect 188. The compound of Aspect 1, wherein each of R₁ and R₃ is hydrogen.

Aspect 189. The compound of Aspect 1, wherein each of R₁ and R₃ is C1-C3 alkyl.

Aspect 190. The compound of any one of Aspect 180-Aspect 189, wherein each of R₂ and R₁₀ is independently selected from hydrogen and hydroxy.

Aspect 191. The compound of Aspect 190, wherein R₂ is hydrogen; and wherein R₁₀ is selected from hydrogen and hydroxy.

Aspect 192. The compound of Aspect 190, wherein R₁₀ is hydrogen; and wherein R₂ is selected from hydrogen and hydroxy.

Aspect 193. The compound of Aspect 190, wherein each of R₂ and R₁₀ is hydrogen.

Aspect 194. The compound of Aspect 190, wherein each of R₂ and R₁₀ is hydroxy.

Aspect 195. The compound of Aspect 1, wherein the compound has a formula represented by a structure:

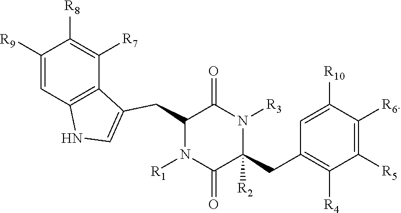

Aspect 196. The compound of Aspect 195, wherein each of R₁ and R₃ is independently selected from hydrogen and C1-C3 alkyl; wherein each of R₂ and R₁₀ is independently selected from hydrogen and hydroxy; wherein each of R₄, R₅, and R₆ is independently selected from hydrogen, halo, hydroxy, azido, and C1-C3 alkyl; wherein R₇ is selected from hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl; wherein each of R₈ and R₉ is independently selected from hydrogen and halo; and wherein R₁₀ is selected from hydrogen and hydroxyl.

Aspect 197. The compound of Aspect 196, wherein each of R₁ and R₃ is independently selected from hydrogen or methyl.

Aspect 198. The compound of Aspect 196, wherein R₁ is methyl; and wherein R₃ is selected from hydrogen or methyl.

Aspect 199. The compound of any one of Aspect 196-Aspect 198, wherein R₂ is hydrogen.

Aspect 200. The compound of any one of Aspect 196-Aspect 198, wherein R₂ is hydroxy.

Aspect 201. The compound of any one of Aspect 196-Aspect 200, wherein each of R₄, R₅, and R₆ is independently selected from hydrogen, fluoro, chloro, bromo, hydroxy, and methyl.

Aspect 202. The compound of Aspect 201, wherein each of R₄, R₅, and R₆ is independently selected from hydrogen and hydroxy.

Aspect 203. The compound of any one of Aspect 196-Aspect 200, wherein each of R₄, R₅, and R₆ is hydrogen.

Aspect 204. The compound of any one of Aspect 196-Aspect 200, wherein R₄ is hydrogen; and where each of R₅ and R₆ is independently selected from hydrogen, fluoro, chloro, bromo, hydroxy, and methyl.

Aspect 205. The compound of any one of Aspect 196-Aspect 200, wherein R₅ is hydrogen; and where each of R₅ and R₆ is independently selected from hydrogen, fluoro, chloro, bromo, hydroxy, and methyl.

Aspect 206. The compound of any one of Aspect 196-Aspect 200, wherein R₆ is hydrogen; and where each of R₄ and R₅ is independently selected from hydrogen, fluoro, chloro, bromo, hydroxy, and methyl.

Aspect 207. The compound of any one of Aspect 196-Aspect 206, wherein R₇ is selected from hydrogen, nitro, and fluoro.

Aspect 208. The compound of Aspect 207, wherein R₇ is nitro.

Aspect 209. The compound of any one of Aspect 196-Aspect 208, wherein each of R₈ and R₉ is independently selected from hydrogen and fluoro.

Aspect 210. The compound of any one of Aspect 196-Aspect 208, wherein each of R₈ and R₉ is hydrogen.

Aspect 211. The compound of any one of Aspect 196-Aspect 208, wherein each of R₈ and R₉ is fluoro.

Aspect 212. The compound of Aspect 1, wherein the compound is selected from one or more of the following:

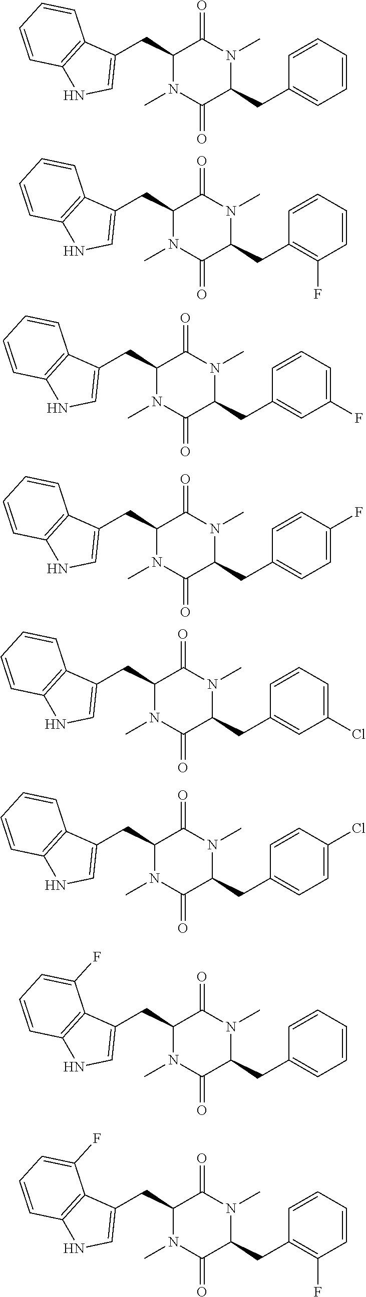

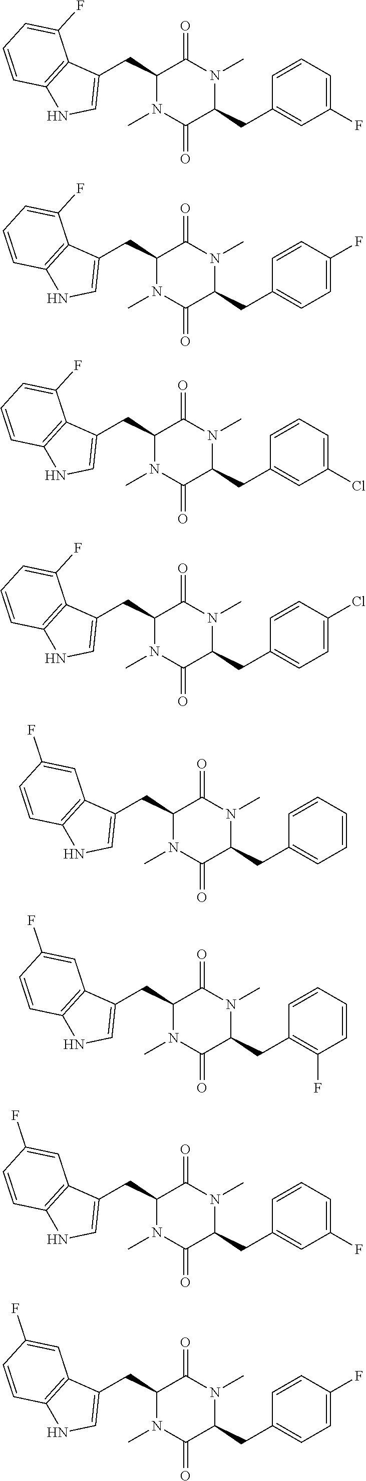

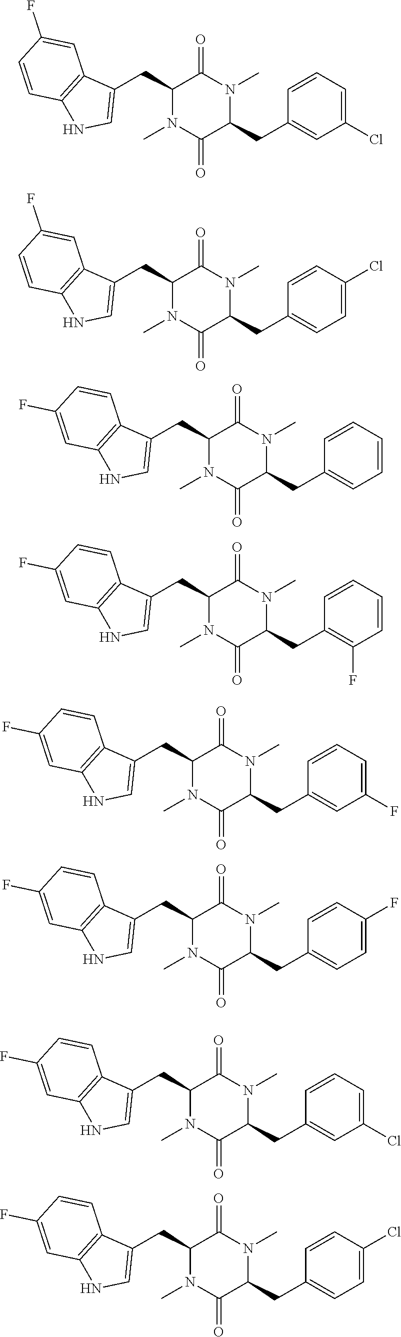

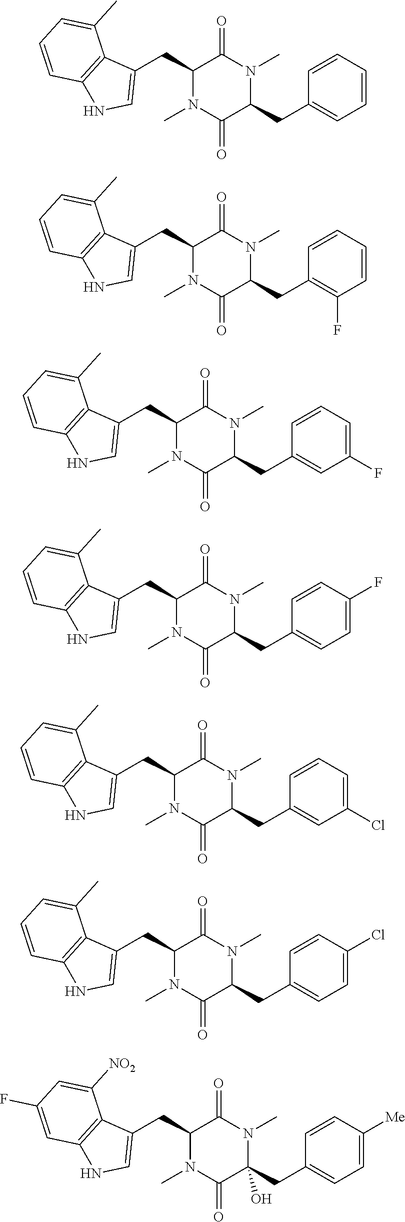

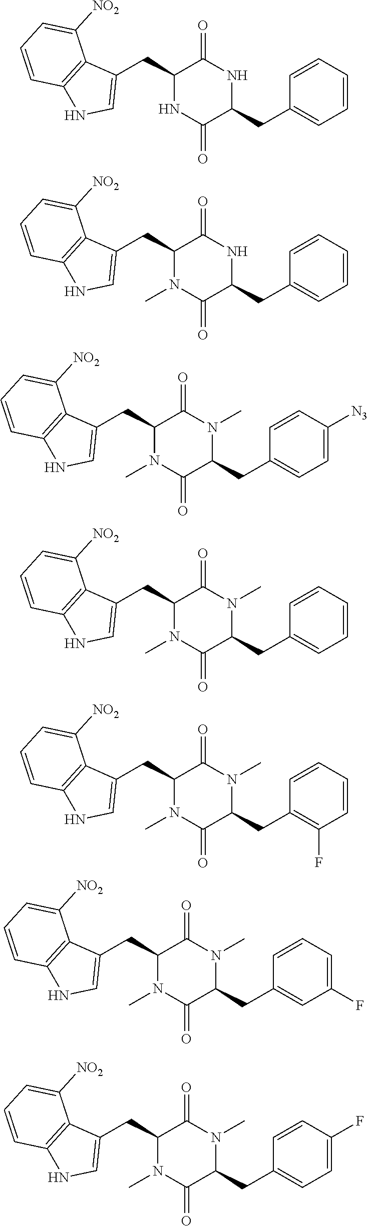

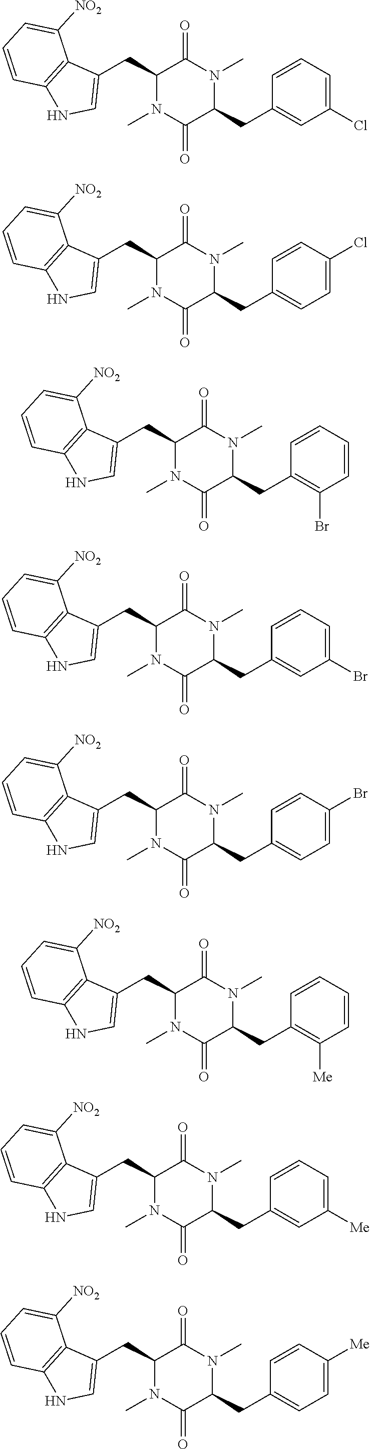

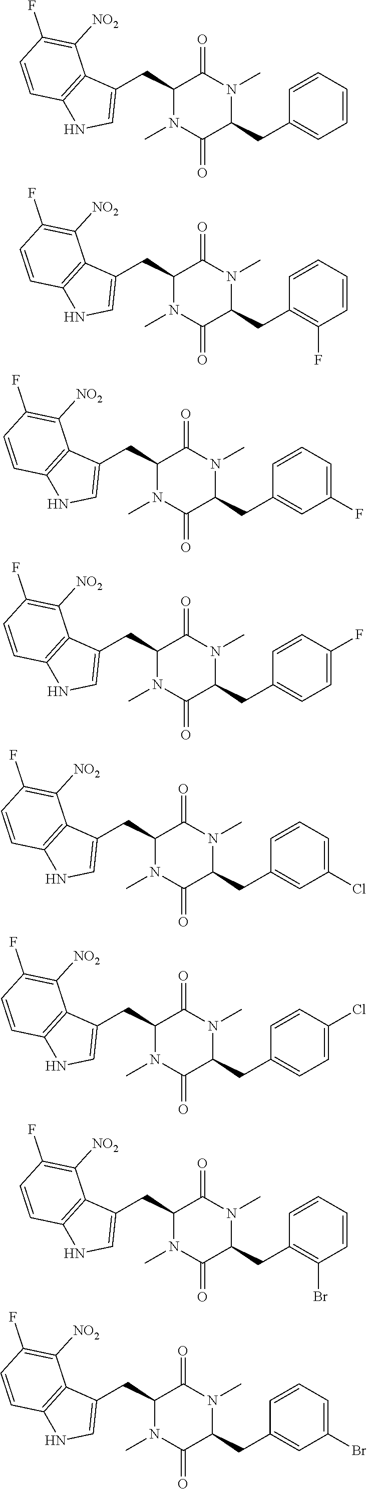

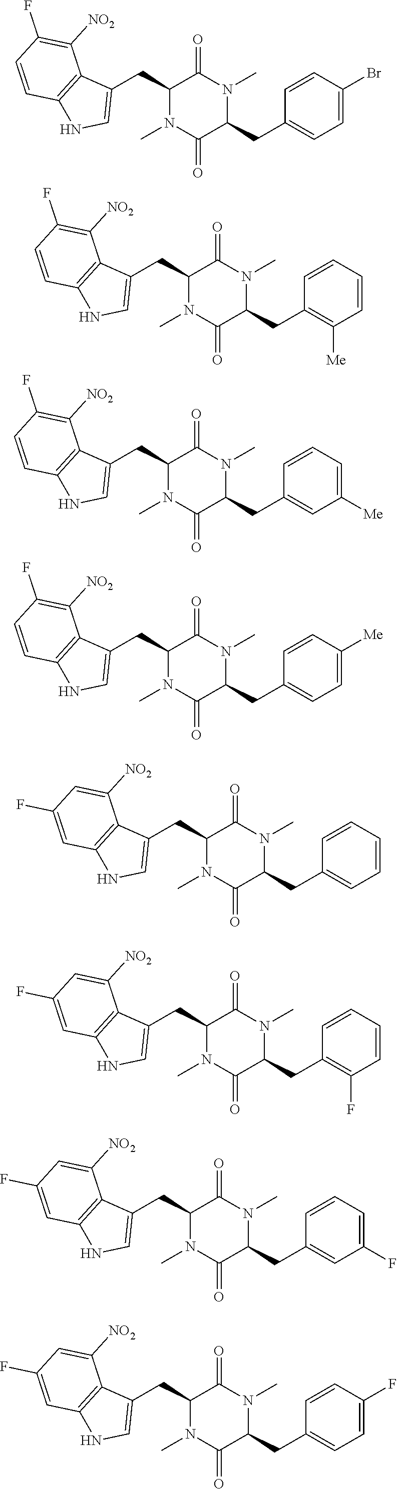

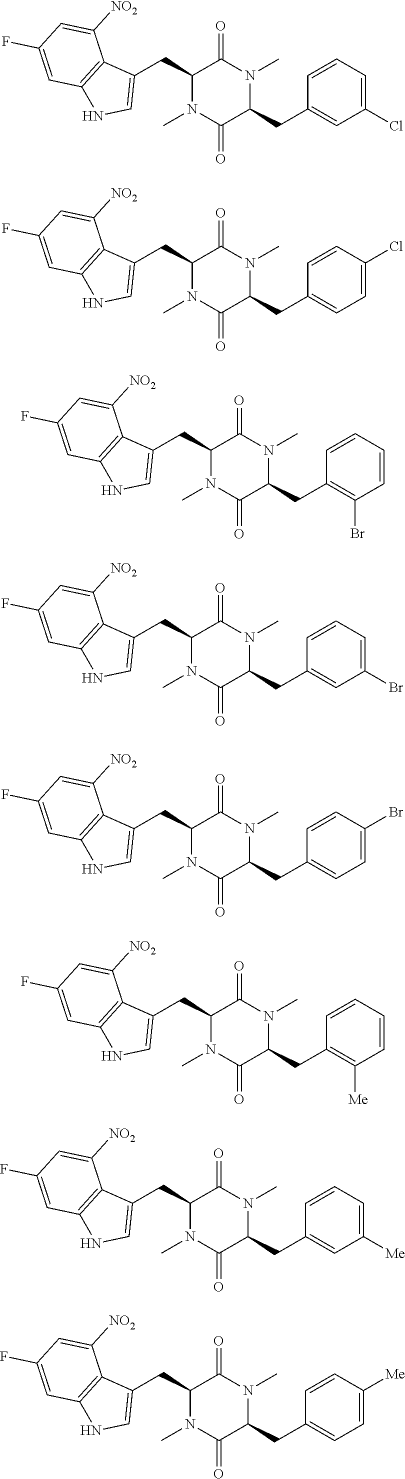

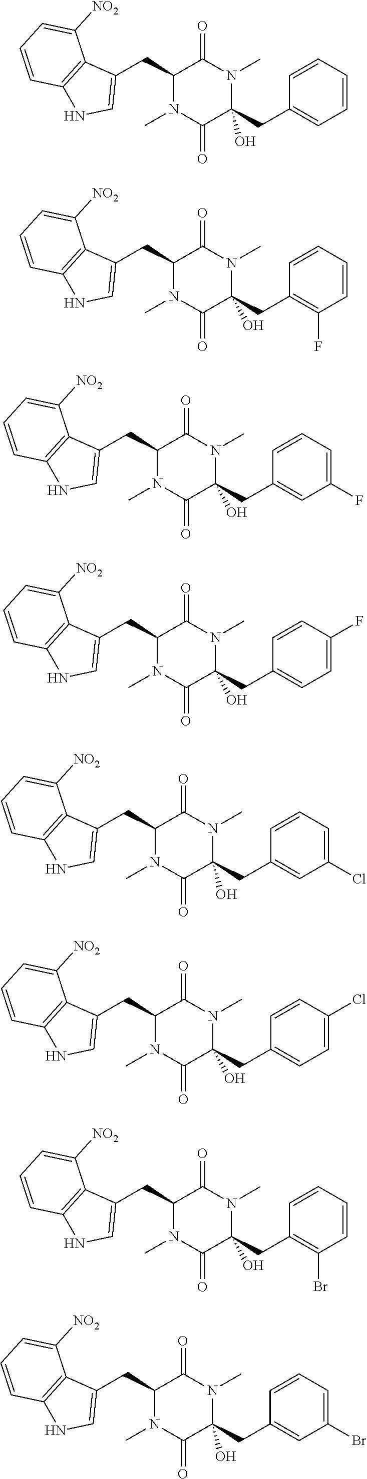

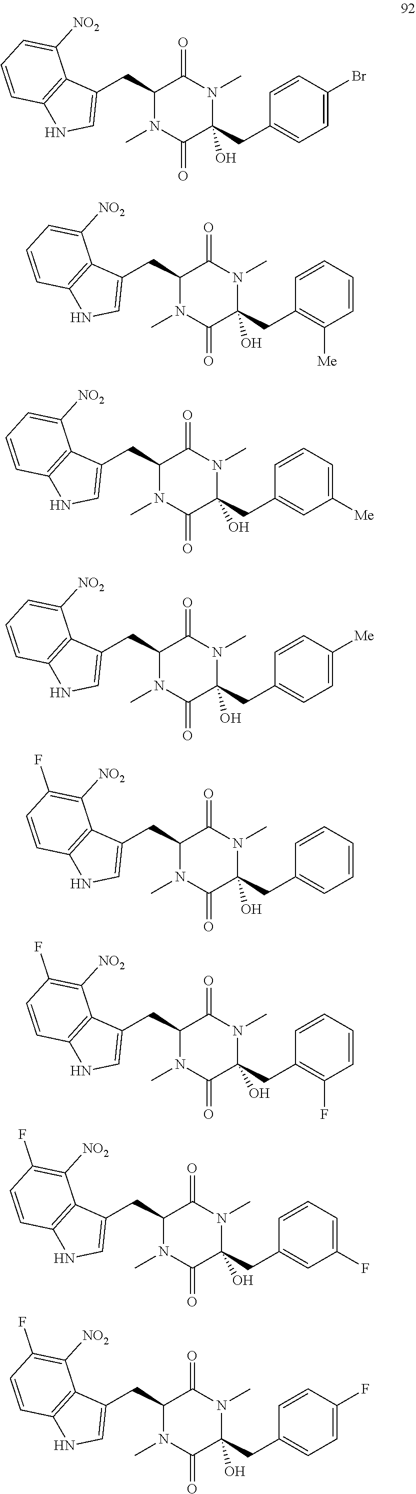

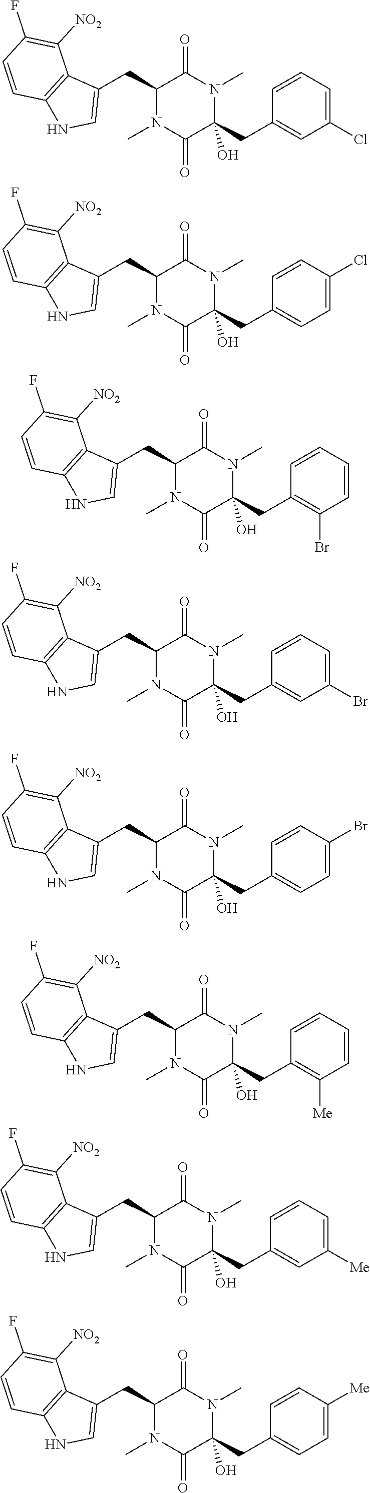

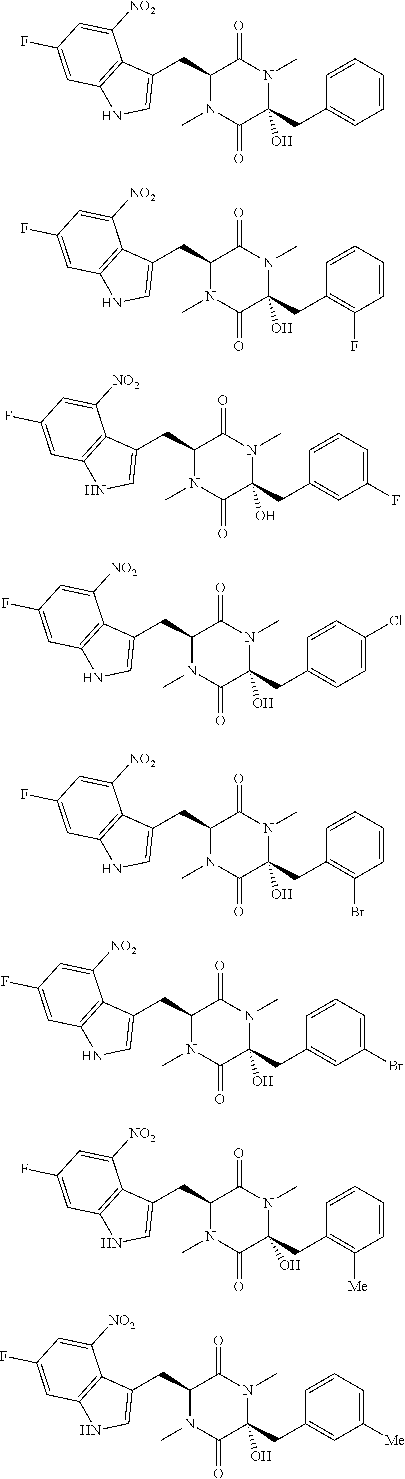

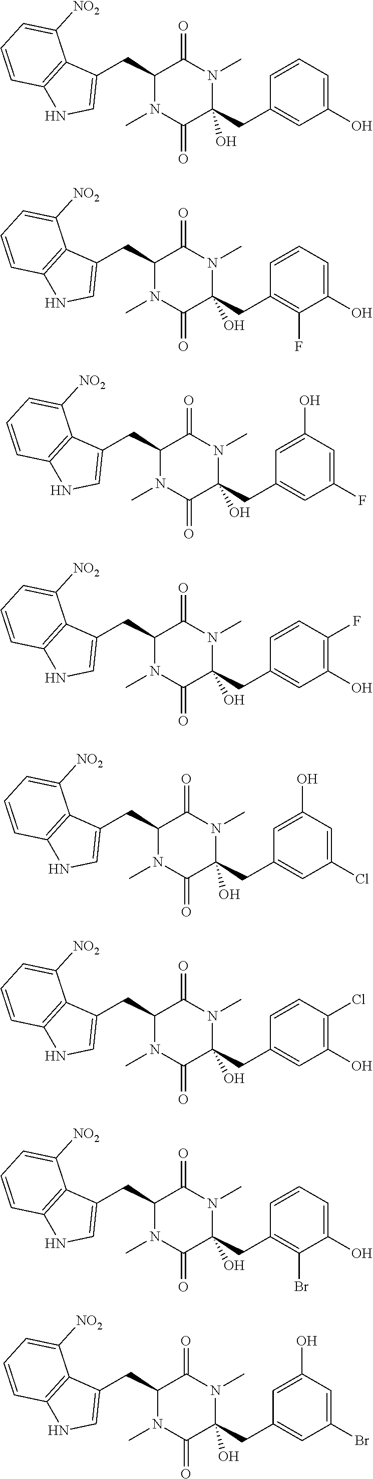

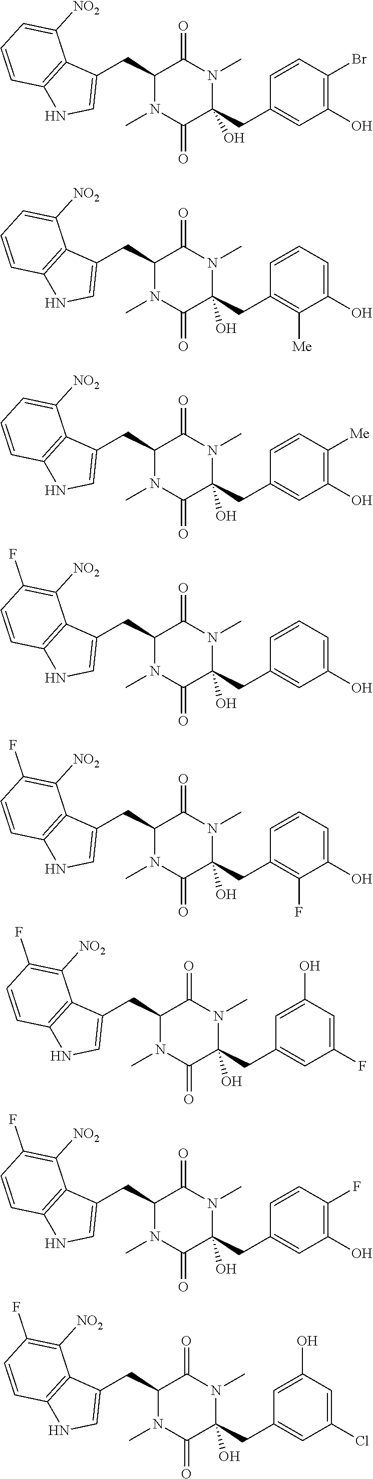

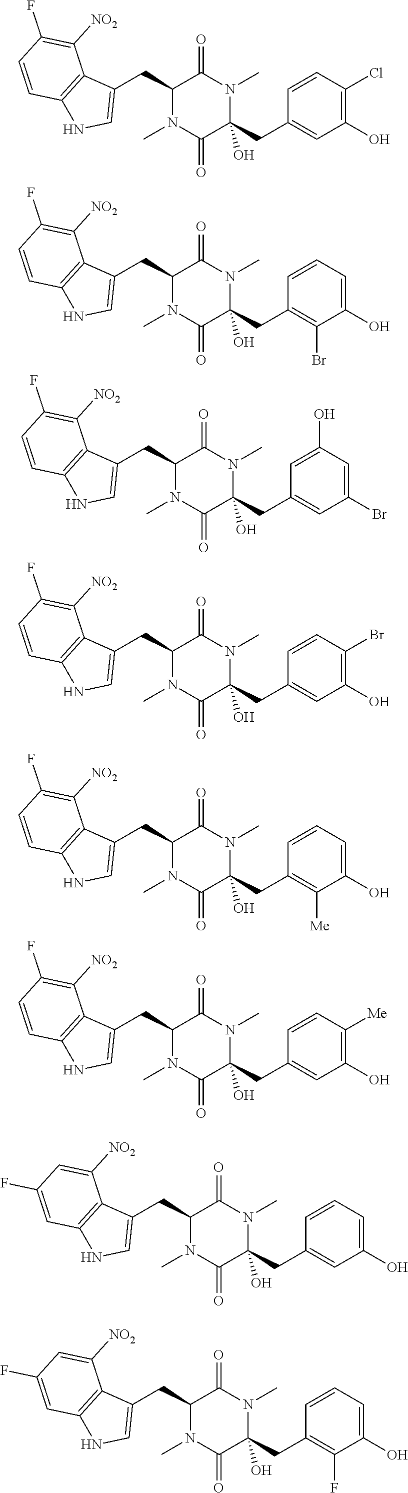

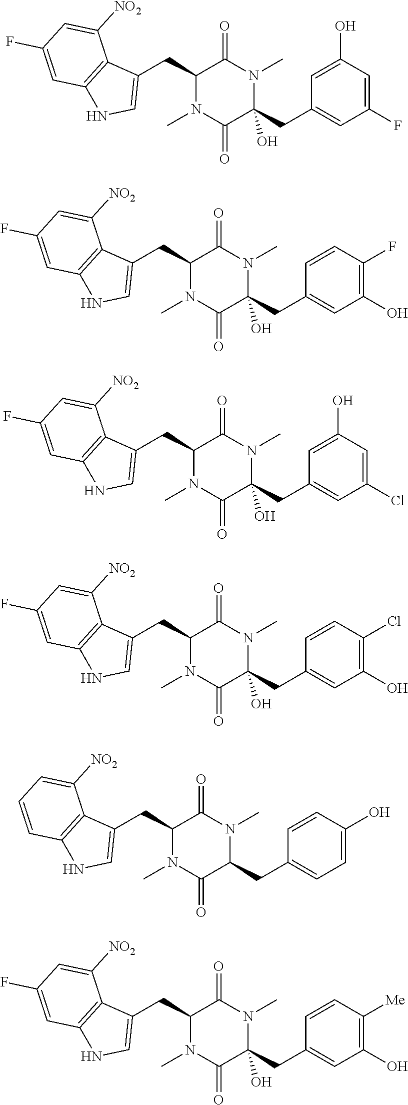 [SMILES: CN1C(=O)[C@](O)(Cc2cc(O)cc(F)c2)N(C)C(=O)[C@@H]1Cc1c[nH]c2cc(F)cc([N+](=O)[O-])c12 ; CN1C(=O)[C@](O)(Cc2ccc(F)c(O)c2)N(C)C(=O)[C@@H]1Cc1c[nH]c2cc(F)cc([N+](=O)[O-])c12 ; CN1C(=O)[C@](O)(Cc2cc(O)cc(Cl)c2)N(C)C(=O)[C@@H]1Cc1c[nH]c2cc(F)cc([N+](=O)[O-])c12 ; CN1C(=O)[C@](O)(Cc2ccc(Cl)c(O)c2)N(C)C(=O)[C@@H]1Cc1c[nH]c2cc(F)cc([N+](=O)[O-])c12 ; CN1C(=O)[C@H](Cc2c[nH]c3cccc([N+](=O)[O-])c23)N(C)C(=O)[C@@H]1Cc1ccc(O)cc1 ; Cc1ccc(C[C@@]2(O)C(=O)N(C)[C@@H](Cc3c[nH]c4cc(F)cc([N+](=O)[O-])c34)C(=O)N2C)cc1O]

Aspect 213. A herbicidal composition comprising an herbicidally effective amount of a compound of any one of Aspect 1-Aspect 212, in a mixture with an agriculturally acceptable adjuvant or carrier.

Aspect 214. The herbicidal composition of Aspect 213, further comprising at least one herbicidal agent.

Aspect 215. The herbicidal composition of Aspect 214, wherein the at least one herbicidal agent is selected from an amide herbicide, anilide herbicide, arylalanine herbicide, chloroacetanilide herbicide, sulfonanilide herbicide, sulfonamide herbicide, antibiotic herbicide, benzoic acid herbicide, pyrimidinyloxybenzoic acid herbicide, pyrimidinylthiobenzoic acid herbicide, quinolinecarboxylic acid herbicide, arsenical herbicide, benzoylcyclohexanedione herbicide, benzofuranyl alkylsulfonate herbicide, carbamate herbicide, carbanilate herbicide, cyclohexene oxime herbicide, cyclopropylisoxazole herbicide, dicarboximide herbicide, dinitroaniline herbicide, dinitrophenol herbicide, diphenyl ether herbicide, nitrophenyl ether herbicide, dithiocarbamate herbicide, halogenated aliphatic herbicide, imidazolinone herbicide, inorganic herbicide, nitrile herbicide, organophosphorus herbicide, phenoxy herbicide, phenoxyacetic herbicide, phenoxybutyric herbicide, phenoxypropionic herbicide, aryloxyphenoxypropionic herbicide, phenylenediamine herbicide, pyrazolyl herbicide, pyrazolylphenyl herbicide, pyridazine herbicide, pyridazinone herbicide, pyridine herbicide, pyrimidinediamine herbicide, quaternary ammonium herbicide, thiocarbamate herbicide, thiocarbonate herbicide, triazine herbicide, chlorotriazine herbicide, methoxytriazine herbicide, methylthiotriazine herbicide, triazinone herbicide, triazole herbicide, triazolone herbicide, triazolopyrimidine herbicide, urea herbicide, phenylurea herbicide, pyrimidinylsulfonylurea herbicide, triazinylsulfonylurea herbicide, thiadiazolylurea herbicide, and unclassified herbicide, and combinations thereof.

Aspect 216. The herbicidal composition of Aspect 215, wherein the amide herbicide is selected from allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid, tebutam, and combinations thereof.

Aspect 217. The herbicidal composition of Aspect 215, wherein the anilide herbicide is selected from chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen, propanil, and combinations thereof.

Aspect 218. The herbicidal composition of Aspect 215, wherein the arylalanine herbicide is selected from benzoylprop, flamprop, flamprop-M, and combinations thereof.

Aspect 219. The herbicidal composition of Aspect 215, wherein the chloroacetanilide herbicide is selected from acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, xylachlor, and combinations thereof.

Aspect 220. The herbicidal composition of Aspect 215, wherein the sulfonanilide herbicide is selected from benzofluor, perfluidone, pyrimisulfan, profluazol, and combinations thereof.

Aspect 221. The herbicidal composition of Aspect 215, wherein the sulfonamide herbicide is selected from asulam, carbasulam, fenasulam, oryzalin, and combinations thereof.

Aspect 222. The herbicidal composition of Aspect 215, wherein the antibiotic herbicide is bilanafos.

Aspect 223. The herbicidal composition of Aspect 215, wherein the benzoic acid herbicide is selected from chloramben, dicamba, 2,3,6-TBA, tricamba, and combinations thereof.

Aspect 224. The herbicidal composition of Aspect 215, wherein the pyrimidinyloxybenzoic acid herbicide is selected from bispyribac, pyriminobac, and combinations thereof.

Aspect 225. The herbicidal composition of Aspect 215, wherein the pyrimidinylthiobenzoic acid herbicide is pyrithiobac.

Aspect 226. The herbicidal composition of Aspect 215, wherein the phthalic acid herbicide is chlorthal.

Aspect 227. The herbicidal composition of Aspect 215, wherein the picolinic acid herbicide is selected from aminopyralid, clopyralid, picloram, and combinations thereof.

Aspect 228. The herbicidal composition of Aspect 215, wherein the quinolinecarboxylic acid herbicide is selected from quinclorac, quinmerac, and a combination thereof.

Aspect 229. The herbicidal composition of Aspect 215, wherein the arsenical herbicide is selected from cacodylic acid, CMA, DSMA, hexaflurate, MAA, MAMA, MSMA, potassium arsenite, sodium arsenite, and combinations thereof.

Aspect 230. The herbicidal composition of Aspect 215, wherein the benzoylcyclohexanedione herbicide is selected from mesotrione, sulcotrione, tefuryltrione, tembotrione, and combinations thereof.

Aspect 231. The herbicidal composition of Aspect 215, wherein the benzofuranyl alkylsulfonate herbicide is selected from benfuresate, ethofumesate, and a combination thereof.

Aspect 232. The herbicidal composition of Aspect 215, wherein the carbamate herbicide is selected from asulam, carboxazole chlorprocarb, dichlormate, fenasulam, karbutilate, terbucarb, and combinations thereof.

Aspect 233. The herbicidal composition of Aspect 215, wherein the carbanilate herbicide is selected from barban, BCPC, carbasulam, carbetamide, CEPC, chlorbufam, chlorpropham, CPPC, desmedipham, phenisopham, phenmedipham, phenmedipham-ethyl, propham, and combinations thereof.

Aspect 234. The herbicidal composition of Aspect 215, wherein the cyclohexene oxime herbicide is selected from alloxydim, butroxydim, clethodim, cloproxydim, cycloxydim, profoxydim, sethoxydim, tepraloxydim, tralkoxydim, and combinations thereof.

Aspect 235. The herbicidal composition of Aspect 215, wherein the cyclopropylisoxazole herbicide is selected from isoxachlortole, isoxaflutole, and a combination thereof.

Aspect 236. The herbicidal composition of Aspect 215, wherein the dicarboximide herbicide is selected from benzfendizone, cinidon-ethyl, flumezin, flumiclorac, flumioxazin, flumipropyn, and combinations thereof.

Aspect 237. The herbicidal composition of Aspect 215, wherein the dinitroaniline herbicide is selected from benfluralin, butralin, dinitramine, ethalfluralin, fluchloralin, isopropalin, methalpropalin, nitralin, oryzalin, pendimethalin, prodiamine, profluralin, trifluralin, and combinations thereof.

Aspect 238. The herbicidal composition of Aspect 215, wherein the dinitrophenol herbicide is selected from dinofenate, dinoprop, dinosam, dinoseb, dinoterb, DNOC, etinofen, medinoterb, and combinations thereof.

Aspect 239. The herbicidal composition of Aspect 215, wherein the diphenyl ether herbicide is ethoxyfen.

Aspect 240. The herbicidal composition of Aspect 215, wherein the nitrophenyl ether herbicide is selected from acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen, oxyfluorfen, and combinations thereof.

Aspect 241. The herbicidal composition of Aspect 215, wherein the dithiocarbamate herbicide is selected from dazomet, metam, and a combination thereof.

Aspect 242. The herbicidal composition of Aspect 215, wherein the halogenated aliphatic herbicide is selected from alorac, chloropon, dalapon, flupropanate, hexachloroacetone, iodomethane, methyl bromide, monochloroacetic acid, SMA, TCA, and combinations thereof.

Aspect 243. The herbicidal composition of Aspect 215, wherein the imidazolinone herbicide is selected from imazamethabenz, imazamox, imazapic, imazapyr, imazaquin, imazethapyr, and combinations thereof.

Aspect 244. The herbicidal composition of Aspect 215, wherein the inorganic herbicide is selected from ammonium sulfamate, borax, calcium chlorate, copper sulfate, ferrous sulfate, potassium azide, potassium cyanate, sodium azide, sodium chlorate, sulfuric acid, and combinations thereof.

Aspect 245. The herbicidal composition of Aspect 215, wherein the nitrile herbicide is selected from bromobonil, bromoxynil, chloroxynil, dichlobenil, iodobonil, ioxynil, pyraclonil, and combinations thereof.

Aspect 246. The herbicidal composition of Aspect 215, wherein the organophosphorus herbicide is selected from amiprofos-methyl, anilofos, bensulide, bilanafos, butamifos, 2,4-DEP, DMPA, EBEP, fosamine, glufosinate, glyphosate, piperophos, and combinations thereof.

Aspect 247. The herbicidal composition of Aspect 215, wherein the phenoxy herbicide is selected from bromofenoxim, clomeprop, 2,4-DEB, 2,4-DEP, difenopenten, disul, erbon, etnipromid, fenteracol, trifopsime, and combinations thereof.

Aspect 248. The herbicidal composition of Aspect 215, wherein the phenoxyacetic herbicide is selected from 4-CPA, 2,4-D, 3,4-DA, MCPA, MCPA-thioethyl, 2,4,5-T, and combinations thereof.

Aspect 249. The herbicidal composition of Aspect 215, wherein the phenoxybutyric herbicide is selected from 4-CPB, 2,4-DB, 3,4-DB, MCPB, 2,4,5-TB, and combinations thereof.

Aspect 250. The herbicidal composition of Aspect 215, wherein the phenoxypropionic herbicide is selected from cloprop, 4-CPP, dichlorprop, dichlorprop-P, 3,4-DP, fenoprop, mecoprop, mecoprop-P, and combinations thereof.

Aspect 251. The herbicidal composition of Aspect 215, wherein the aryloxyphenoxypropionic herbicide is selected from chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop, and combinations thereof.

Aspect 252. The herbicidal composition of Aspect 215, wherein the phenylenediamine herbicide is selected from dinitramine, prodiamine, and a combination thereof.

Aspect 253. The herbicidal composition of Aspect 215, wherein the pyrazolyl herbicide is selected from benzofenap, pyrazolynate, pyrasulfotole, pyrazoxyfen, pyroxasulfone, topramezone, and combinations thereof.

Aspect 254. The herbicidal composition of Aspect 215, wherein the pyrazolylphenyl herbicide is selected from fluazolate, pyraflufen, and a combination thereof.

Aspect 255. The herbicidal composition of Aspect 215, wherein the pyridazine herbicide is selected from credazine, pyridafol, pyridate, and combinations thereof.

Aspect 256. The herbicidal composition of Aspect 215, wherein the pyridazinone herbicide is selected from brompyrazon, chloridazon, dimidazon, flufenpyr, metflurazon, norflurazon, oxapyrazon, pydanon, and combinations thereof.

Aspect 257. The herbicidal composition of Aspect 215, wherein the pyridine herbicide is selected from aminopyralid, cliodinate, clopyralid, dithiopyr, fluoroxypyr, haloxydine, picloram, picolinafen, pyriclor, thiazopyr, triclopyr, and combinations thereof.

Aspect 258. The herbicidal composition of Aspect 215, wherein the pyrimidinediamine herbicide is selected from iprymidam, tioclorim, and a combination thereof.

Aspect 259. The herbicidal composition of Aspect 215, wherein the quaternary ammonium herbicide is selected from cyperquat, diethamquat, difenzoquat, diquat, morfamquat, paraquat, and combinations thereof.

Aspect 260. The herbicidal composition of Aspect 215, wherein the thiocarbamate herbicide is selected from butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, vernolate, and combinations thereof.

Aspect 261. The herbicidal composition of Aspect 215, wherein the thiocarbonate herbicide is selected from dimexano, EXD, proxan, and combinations thereof.

Aspect 262. The herbicidal composition of Aspect 215, wherein the thiourea herbicide is methiuron.

Aspect 263. The herbicidal composition of Aspect 215, wherein the triazine herbicide is selected from dipropetryn, triaziflam, trihydroxytriazine, and combinations thereof.

Aspect 264. The herbicidal composition of Aspect 215, wherein the chlorotriazine herbicide is selected from atrazine, chlorazine, cyanazine, cyprazine, eglinazine, ipazine, mesoprazine, procyazine, proglinazine, propazine, sebuthylazine, simazine, terbuthylazine, trietazine, and combinations thereof.

Aspect 265. The herbicidal composition of Aspect 215, wherein the methoxytriazine herbicide is selected from atraton, methometon, prometon, secbumeton, simeton, terbumeton, and combinations thereof.

Aspect 266. The herbicidal composition of Aspect 215, wherein the methylthiotriazine herbicide is selected from ametryn, aziprotryne, cyanatryn, desmetryn, dimethametryn, methoprotryne, prometryn, simetryn, terbutryn, and combinations thereof.

Aspect 267. The herbicidal composition of Aspect 215, wherein the triazinone herbicide is selected from ametridione, amibuzin, hexazinone, isomethiozin, metamitron, metribuzin, and combinations thereof.

Aspect 268. The herbicidal composition of Aspect 215, wherein the triazole herbicide is selected from amitrole, cafenstrole, epronaz, flupoxam, and combinations thereof.

Aspect 269. The herbicidal composition of Aspect 215, wherein the triazolone herbicide is selected from amicarbazone, bencarbazone, carfentrazone, flucarbazone, propoxycarbazone, sulfentrazone, thiencarbazone-methyl, and combinations thereof.

Aspect 270. The herbicidal composition of Aspect 215, wherein the triazolopyrimidine herbicide is selected from cloransulam, diclosulam, florasulam, flumetsulam, metosulam, penoxsulam, pyroxsulam, and combinations thereof.

Aspect 271. The herbicidal composition of Aspect 215, wherein the uracil herbicide is selected from butafenacil, bromacil, flupropacil, isocil, lenacil, terbacil, 3-phenyluracils, and combinations thereof.

Aspect 272. The herbicidal composition of Aspect 215, wherein the urea herbicide is selected from benzthiazuron, cumyluron, cycluron, dichloralurea, diflufenzopyr, isonoruron, isouron, methabenzthiazuron, monisouron, noruron, and combinations thereof.

Aspect 273. The herbicidal composition of Aspect 215, wherein the phenylurea herbicide is selected from anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron, thidiazuron, and combinations thereof.

Aspect 274. The herbicidal composition of Aspect 215, wherein the pyrimidinylsulfonylurea herbicide is selected from amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron, trifloxysulfuron, and combinations thereof.

Aspect 275. The herbicidal composition of Aspect 215, wherein the triazinylsulfonylurea herbicide is selected from chlorsulfuron, cinosulfuron, ethametsulfuron, iodosulfuron, metsulfuron, prosulfuron, thifensulfuron, triasulfuron, tribenuron, triflusulfuron and tritosulfuron.

Aspect 276. The herbicidal composition of Aspect 215, wherein the thiadiazolylurea herbicide is selected from buthiuron, ethidimuron, tebuthiuron, thiazafluoron, thidiazuron, and combinations thereof.

Aspect 277. The herbicidal composition of Aspect 215, wherein the unclassified herbicide is selected from acrolein, allyl alcohol, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, fluorochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan tritac, and combinations thereof.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

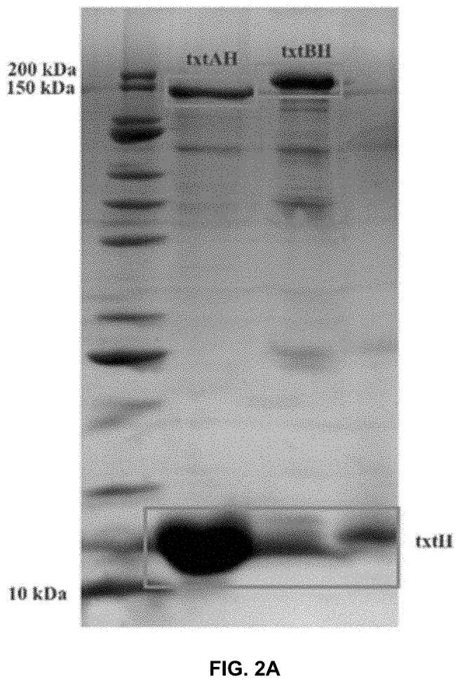

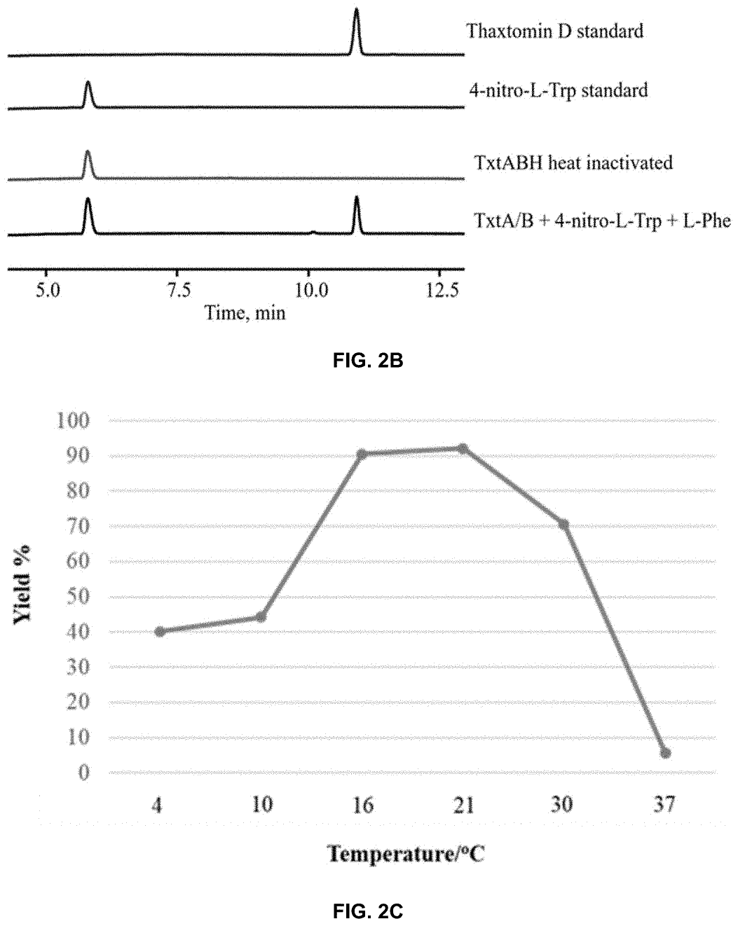

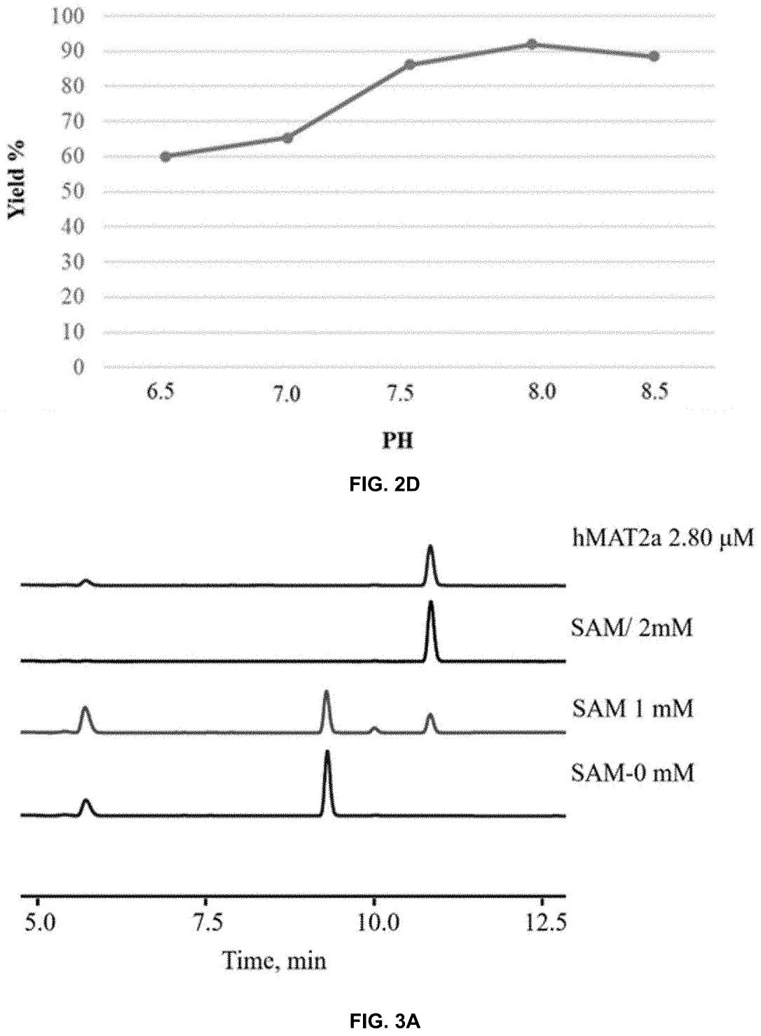

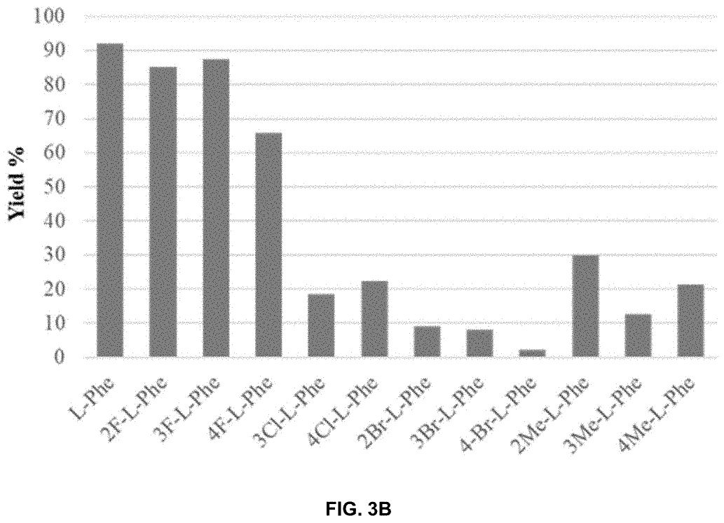

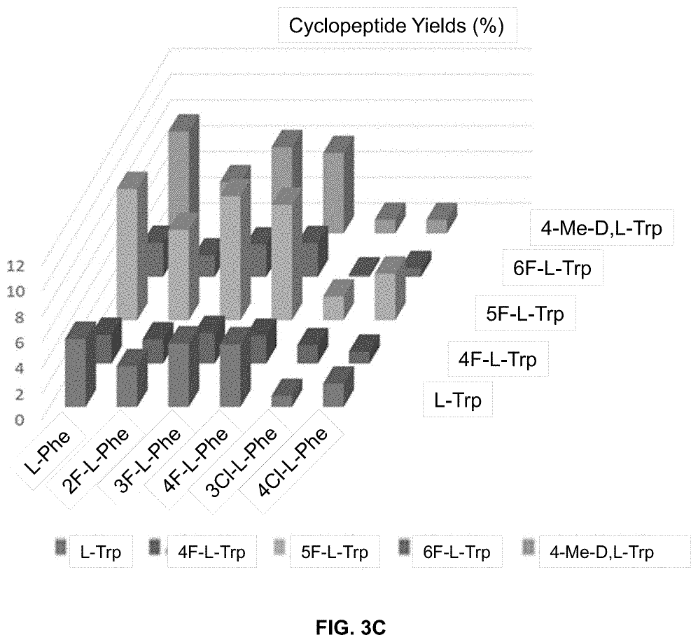

Initial attempts to prepare recombinant C-terminal His6-tagged TxtA and TxtB in E. coli BAP1 failed due to the low soluble protein level and no enzyme activities with I-Phe and 4-NO2-I-Trp as substrates. The expression problem was successfully addressed by co-expressing txtA and txtB with txtH (FIG. 2A). TxtH is a predicted MtbH-like protein that is known to be essential to the expression and function of many bacterial NRPSs. Purified recombinant TxtA/B were incubated with I-Phe, 4-NO2-I-Trp and S-adenosyl methionine (SAM). HPLC analysis revealed a single product with the same retention time and m/z value (407.1710, Δ 2.2 ppm) as the standard thaxtomin D (FIG. 2B, Table 3). The reaction conditions were further optimized, and the two enzymes showed the highest activity at pH 8.0 and 16 to 21° C. (FIGS. 2C and 2D). Notably, the enzymes completely lost the activities at 37° C. Under the optimal conditions, TxtA/B (14.5 μM) consumed 30% amino acid substrates (0.5 mM) after 1 h and 90% after 15 hs (FIG. 2E), indicating the efficient synthesis of DKP. In addition to the formation of DKP core, TxtA/B both carry one methyltransferase (MT) domain that expectedly methylate two —NH— groups of the DKP using SAM as co-substrate. Omission of SAM in the TxtA/B reaction produced nonmethylated thaxtomin 17, confirming the function of two N-MT domains (FIG. 3A). Increasing SAM concentrations in the reactions gradually shifted thaxtomin 17 into thaxtomin D with minor accumulation of monomethylated intermediate thaxtomin C. When the molar ratio of SAM:amino acid substrates was 4:1, thaxtomin D was the only product in the reaction, indicating the high catalytic activity of MT domains (FIG. 3A). Furthermore, hMAT2a was used to generate SAM from 1-methionine in situ and effectively synthesized thaxtomin D in the three-enzyme system (FIG. 3A), creating a cost-effective approach to synthesize methylated DKPs.

To explore whether TxtA/B are versatile to generate a library of thaxtomin D analogs, enzymes' substrate tolerance was then examined in the reactions. When using 4-NO2-I-Trp as the substrate of TxtB, only I-Phe among all 20 natural amino acids in the TxtA/B reactions was accepted by TxtA to produce thaxtomin D as confirmed in HPLC, HR-MS (FIG. 3A, Table 1 and 4). Subsequently, the substrate scope of TxtA with 17 I-Phe analogs was assessed with various substituents at α-position or C2, C3, and C4 of the aryl group (Table 1). Remarkably, TxtA used 11 unnatural substrates bearing aryl F-, Cl-, Br- and Me-substituents to synthesize thaxtomin D analogs, presenting valuable opportunities to expand chemical diversity of DKP compounds. TxtA showed the same level of activity toward 2F- and 3F-I-Phe as I-Phe, followed by 4F-I-Phe (˜72% of I-Phe) (FIG. 3B). Bulkier substituents like —Cl, —Br, and -Me on the I-Phe aryl group significantly decreased enzyme activity in the synthesis of thaxtomin D analogs. For example, 3-OMe-I-Phe was not the substrate of TxtA while 3-Me-I-Phe retained about 12% relative activity of I-Phe in the TxtA/B reaction (FIG. 3C, Table 1).

Using the above strategy, it was determined that TxtB accepted only I-Trp among all 20 natural amino acids in the reaction (FIG. 2B, Table 1). When incubating with 15 I-Trp analogs with various substituents at α-position or C4, C5, C6 and C7 of I-Trp indole (Table 1), TxtB was able to use 4-F, 4-Me, 5-F, and 6-F-tryptophan as substrates in the DKP synthesis (Table 1). Compared with 4-NO2-I-Trp, these noncognate substrates showed lowered activity in the TxtA/B reaction (FIG. 3C). This result indicated that TxtB has a relatively limited substrate flexibility and is more tolerant to modifications on C4 than other positions of I-Trp indole. Indeed, the Micklefield group recently produced several thaxtomin analogs with C4-H, F, Cl, Br, or Me when expressing txtA/B in S. albus. The advanced understanding of substrate scopes of TxtA/B led to enzymatic creation of a library of 30 DKPs with 5 I-Trp analogs and 6 I-Phe analogs (FIG. 3C). The production of all DKPs were confirmed in HPLC analysis and verified in high resolution LC-MS analysis (Table 2). Notably, compared with the reactions with only one substrate variation, TxtA/B retained similar substrate preference patterns in the combinatorial synthesis. This result indicated that TxtA/B independently contributed to diversity generation.

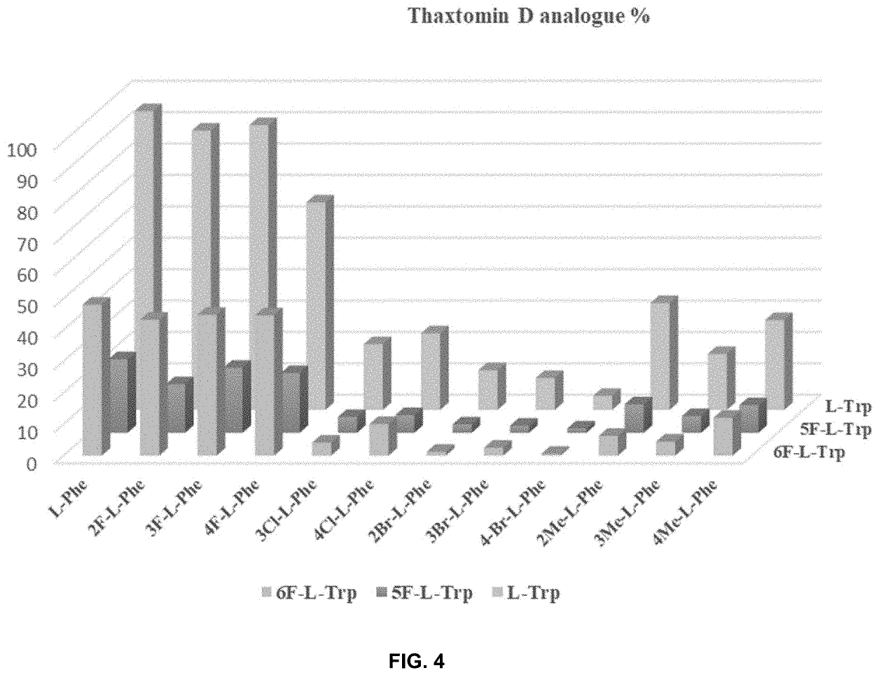

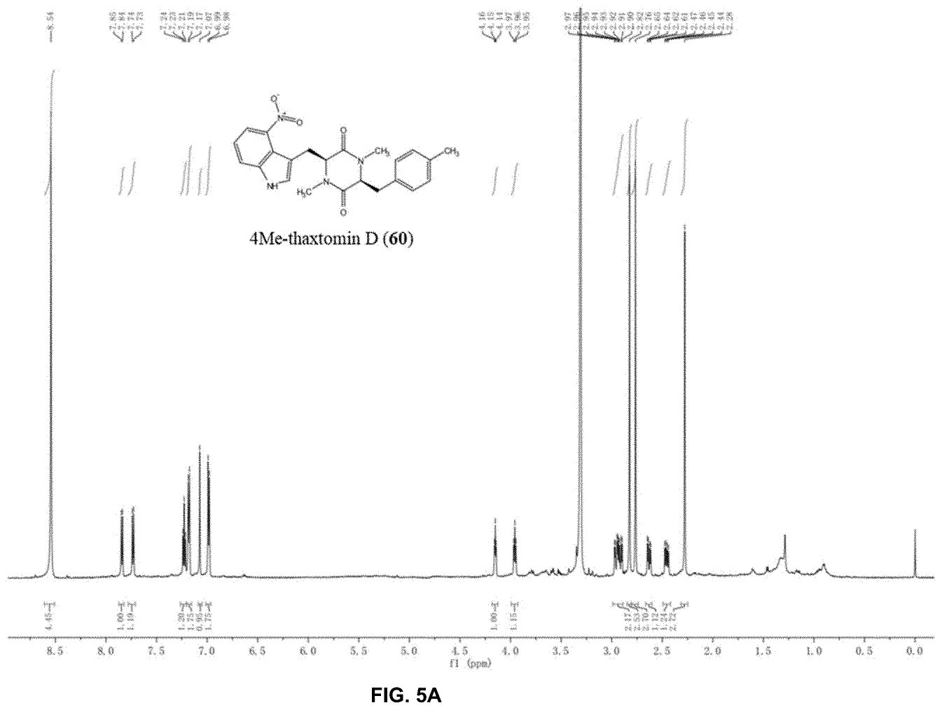

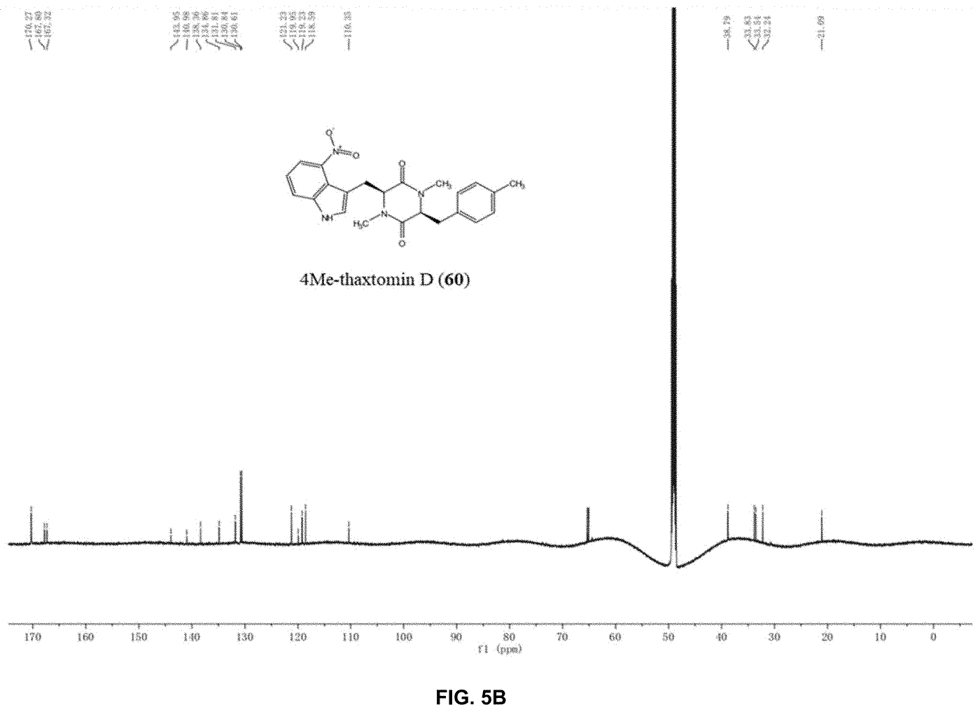

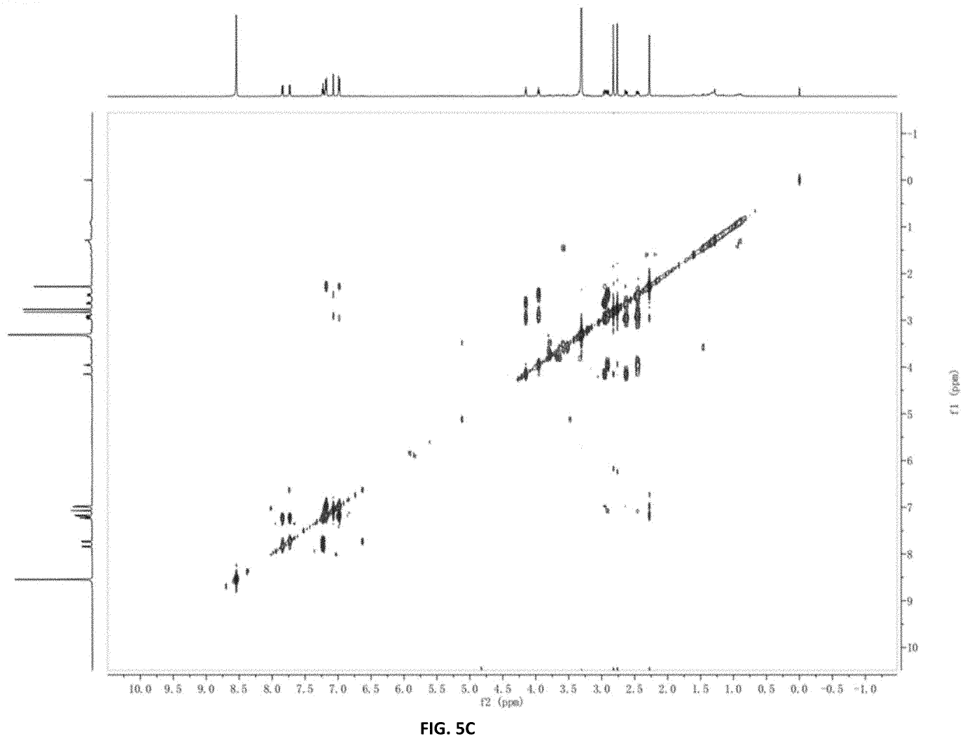

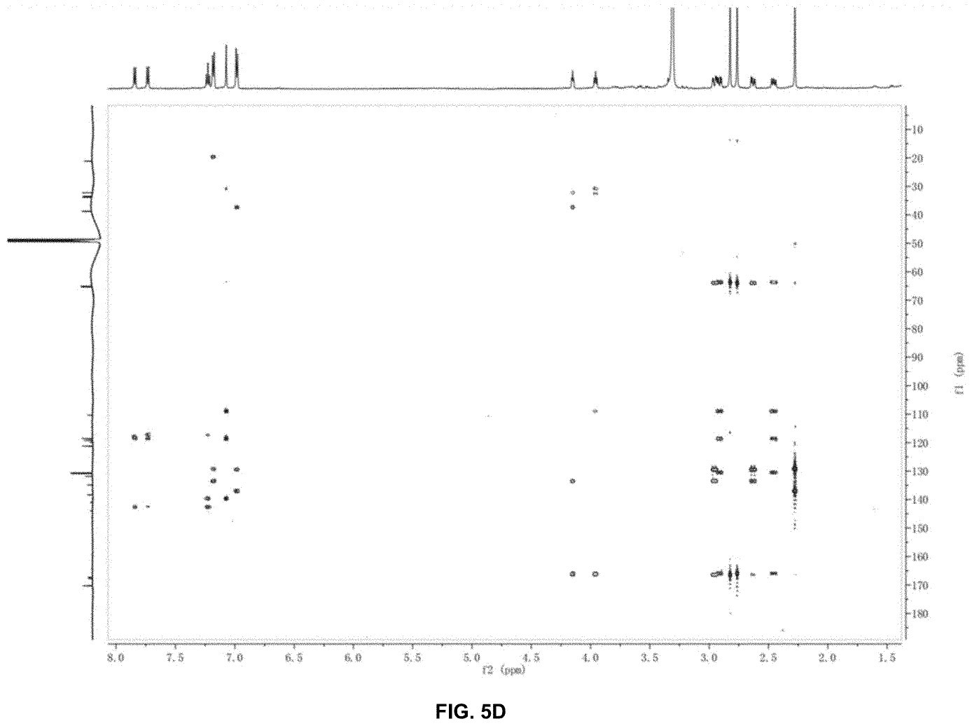

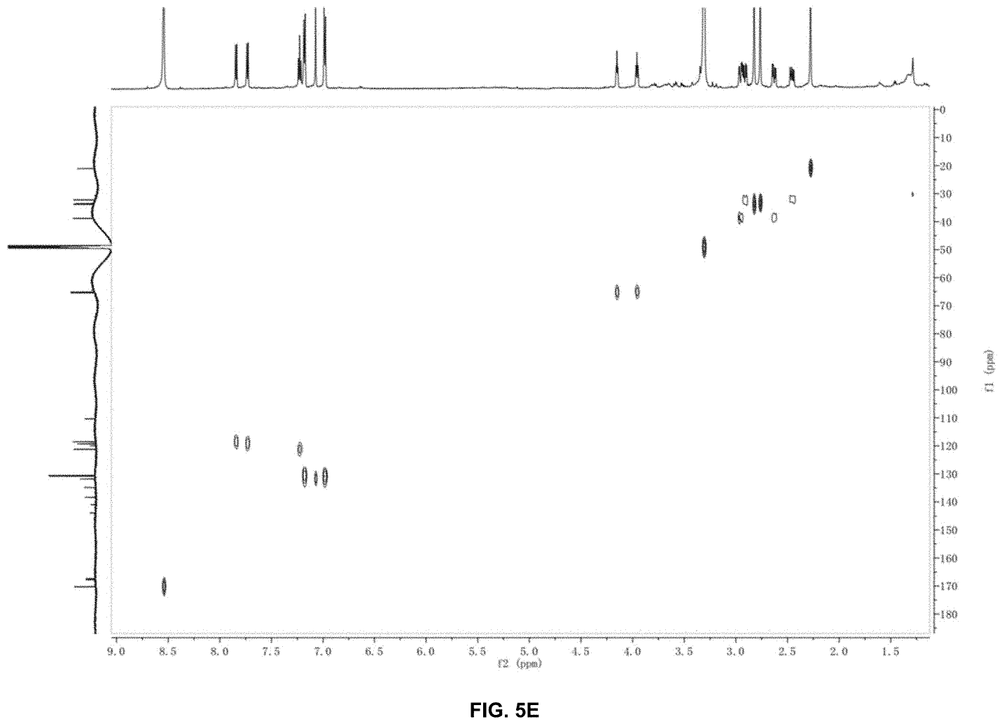

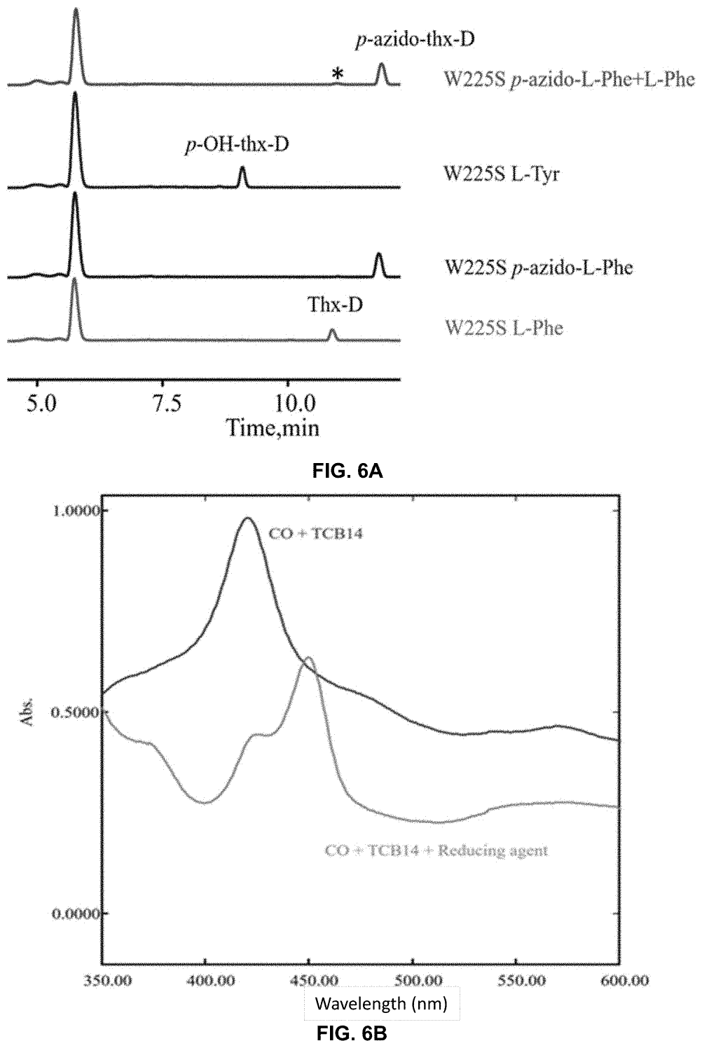

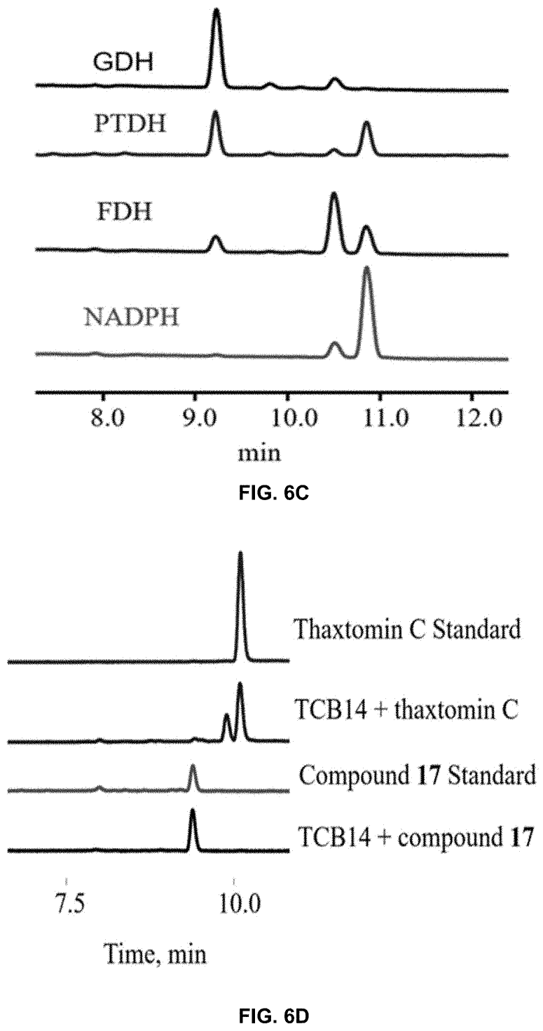

The nitro group of thaxtomins is essential for herbicidal activity, and is installed by TxtE. The next goal was to further diversify the DKP structure for enriching bioactivities by coupling TxtE with TxtA/B. The engineered self-sufficient TxtE TB14 was the most active enzyme ever reported for directly nitrating I-Trp analogs. In the present work, one-pot reaction of TB14 and TxtA/B converted I-Trp and I-Phe into thaxtomin D with a total conversion rate of 94±3.1%. NADPH, NOC-5 as NO donor and SAM were included in the reaction. In addition, freshly regenerated NADPH by formate dehydrogenase (FDH) fully supported the three-enzyme reaction, thereby reducing the synthesis cost. Next, this validated approach was extended to synthesize 36 nitro-containing DKP analogs from 3 I-Trp analogs and 12 I-Phe analogs (FIG. 4). All nitrated DKPs were verified in HR-MS analysis (Table 3). In addition, the reaction was scaled up and 1.1 mg 4-Me-thaxtomin D was isolated from the 10-mL reaction of TB14 and TxtA/B (22.1% isolation yield). The structure of thaxtomin analog was elucidated by NMR and HR-MS analysis (FIG. 5, Table 3). Of note, the activity of TxtB toward in situ generated 4-NO2-I-Trp analogs was similar to I-Trp analogs (FIG. 2B). This result supported the diversity generation model in which each enzyme acts independently. To further expand the substrate scope of TxtA, Trp225 was mutated into Ser in TxtA. The same mutation previously expanded the substrate scope of I-Phe-specific GrsA. The resultant TxtAW225S mutant showed a strong preference to p-azido-I-Phe over I-Phe (FIG. 6A) and also accepted I-Tyr as its substrate, albeit with a lower activity.

In the thaxotmin biosynthetic pathway, the DKP scaffold is further diversified with two —OH groups to produce thaxtomin A that possesses the highest potent herbicidal activity (FIG. 1B). One recent genetic study supported that TxtC catalyzes these two hydroxylation reactions. However, biochemical characterization of TxtC remains elusive. To this end, recombinant TxtC was prepared for in vitro studies. However, the expression of codon-optimized txtC in E. coli led to the protein with no catalytic activity toward its physiological substrate thaxtomin D. Inspired by the successful design of TB14, a self-sufficient TxtC variant was then engineered by fusing the C-terminus of TxtC with the N-terminus of reductase domain of P450BM3 through a 14-amino acid linker, named as TCB14. When co-expressed with chaperone proteins, recombinant TCB14 in E. coli exhibited a Soret peak at 450 nm in its reduced form when saturated with carbon monoxide (FIG. 6B), a diagnostic feature of properly folded P450s. Indeed, this enzyme was able to convert thaxtomin D into thaxtomin B by hydroyxlating α-position of its I-Phe fragment, but the overall conversion rate was only about 12% (FIG. 6C). In addition, LC-MS analysis did not detect thaxtomin A in the TCB14 reaction even when the molar ratio of NADPH/substrate was over 20. Three NADPH regeneration systems driven by FDH, phosphite dehydrogenase (PTDH) and glucose dehydrogenase (GDH) were then screened to examine their effects on the TCB14 reaction. Remarkably, the TCB14 reaction supplemented with the GDH system achieved the complete conversion of thaxtomin D into 94% thaxtomin A and 6% thaxtomin B (FIG. 6C). The FDH system led to about 70% conversion of thaxtomin D in the TCB14 reaction but the major product was thaxtomin B. By contrast, the PTDH system was least effective in supporting the TCB14 reaction but the major product was thaxtomin A. It remains unclear how these NADPH regeneration systems influence the overall activity and product distribution of TCB14. Nonetheless, this work for the first time biochemically confirmed the catalytic function of TxtC and uncovered the order of its two hydroxylation reactions, first on the tertiary aliphatic carbon and then on the C3 of phenyl ring. Functionalization of two C—H bonds with different structure and reactivity by a single P450 enzyme is distinct with other multi-functional P450s.

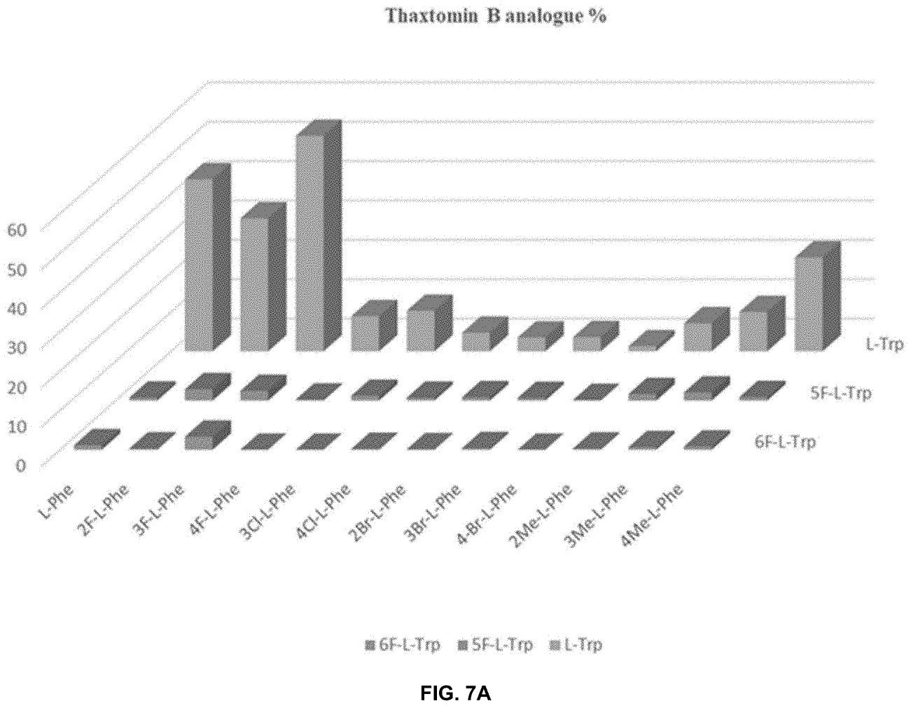

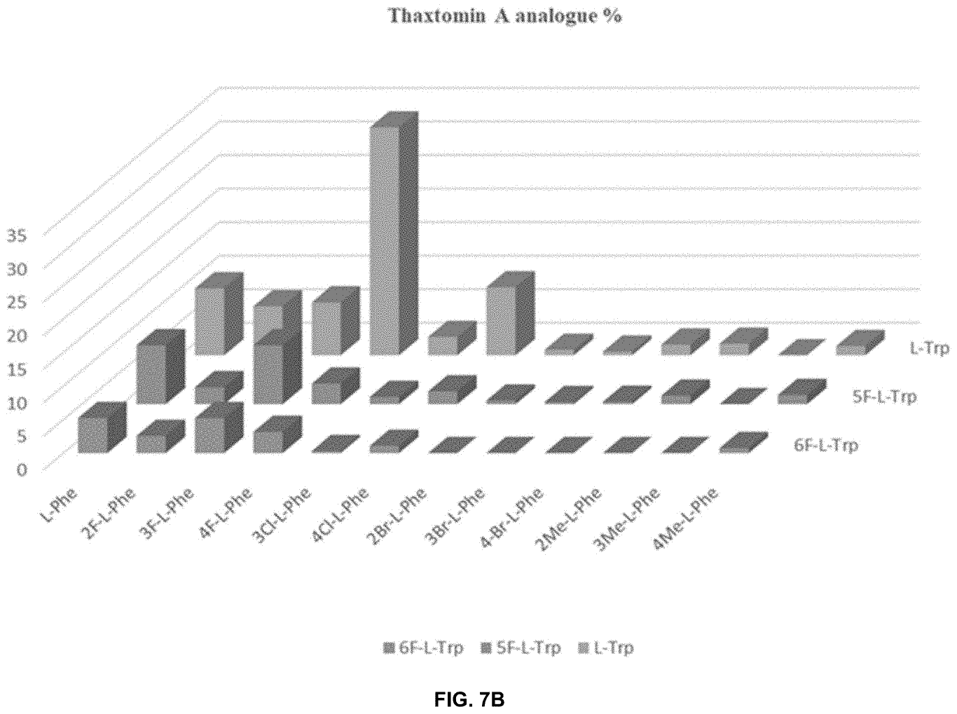

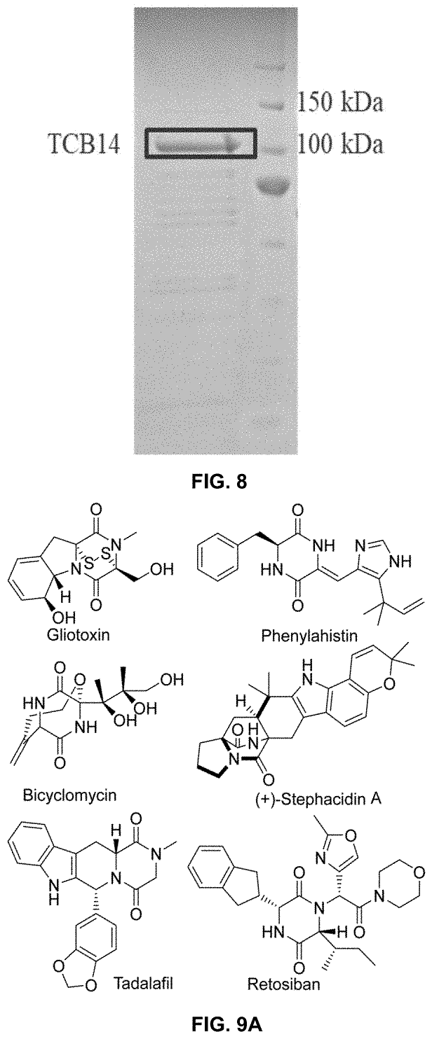

To employ TCB14 to diversify other DKPs, its substrate scope was first examined (FIG. 6D). TCB14 showed no activity toward non-methylated thaxtomin 17 [cyclo(4-NO2-I-Trp-I-Phe)] and only produced monohydroxylated from thaxtomin C, but produced monohydroxylated and dihydroxylated products from thaxtomin D analogs (FIG. 6D). These results suggested that methylation of the DKP core is prerequisite to the TCB14 reactions, illustrating that TxtC provides a control mechanism of chemical diversity of thaxtomins. The next goal was to create one-pot reactions with TB14, TxtA/B and TCB14 to systematically examine TCB14's substrate scope and to synthesize advanced DKP analogs. However, neither NADPH nor any of three NADPH regeneration systems supported the efficient synthesis of thaxtomin B or A directly from I-Trp and I-Phe (typically 0.5 to 9% overall yield). a two-step reaction system was eventually developed that first produced thaxtomin D by TB14 and TxtA/B with the FAD-driven NADPH regeneration system. After 20 h, TCB14 and the GDH-driven NADPH regeneration system were added to the reaction mixture, which was incubated for another 20 h. Using this design, the overall conversion rate of thaxtomin A reached 10% while the conversion rate thaxtomin B reached to 43.7%. this one-pot two-step strategy was then employed to create a library of 62 thaxomin A/B analogues (FIG. 7). All new thaxtomin analogs were confirmed in HRMS analysis (Tables 4 and 5). This result revealed the considerable tolerance of TCB14 to small substituents on the indole and aryl moieties of thaxtomin analogs and provided a biocombinatorial approach to synthesize functionalized DKPs.

In summary, a biocombinatorial approach for the synthesis of a library of structurally diversified thaxtomin-like DKPs directly from amino acid building blocks was have developed. The diversity of DKPs is first determined independently by TxtA and TxtB and can be expanded by protein engineering and the use of single-module NRPS present in other NRP biosynthetic pathways. Importantly, the incorporation of modifying enzymes TxtE and TCB14 enabled the stepwise exploration of significantly broader chemical space of DKPs, providing new insights into the diversity-generating metabolic model that numerous biosynthetic pathways follow. Additional tailoring enzymes can be used to further enrich the structural and functional diversities. The approach can readily provide sufficient amounts of chemicals for further characterization, and led to the discovery of unnatural thaxtomin analogs with improved herbicidal activities. In addition, a number of new DKPs prepared in this work were equipped with naturally unavailable, medicinally important functional groups that can be utilized for further structural diversification. Enzymatic synthesis of DKPs has been used as a model system to investigate the biosynthetic logic and evolution of NRPSs; however, biocombinatorial routes to highly diversified DKPs have not been reported. The approach developed here presents a feasible strategy for generating many NRPs for basic and translational applications.

Materials and General Methods.

Molecular biology reagents and enzymes were purchased from Fisher Scientific. Primers were ordered from Sigma-Aldrich. 4-Me-D,L-Tryptophan was from MP Biomedical (Santa Ana, Calif.), while NOC-5 (3-(Aminopropyl)-1-hydroxy-3-isopropyl-2-oxo-1-triazene) was purchased from EMD Millipore. Escherichia coli DH5a, BL21-GOLD (DE3) (Agilent) and BAP1 were used for routine molecular biology studies and protein expression, respectively, and were grown in Luria-Bertani broth or Terric broth. Other chemicals and solvents were purchased from Sigma-Aldrich and Fisher Scientific. Oligos were ordered from Sigma-Aldrich. DNA sequencing was performed at Eurofins. The primers used in this study were listed in Table 6. A Shimadzu Prominence UHPLC system (Kyoto, Japan) fitted with an Agilent Poroshell 120 EC-C18 column (2.7 μm, 3.0×50 mm), coupled with a PDA detector was used for HPLC analysis. For semi-preparative HPLC, Agilent ZORBAX SB-C18 (5 μm, 9.4×250 mm), and YMC-Pack Ph (5 μm, 4.6×250 mm) columns were used. 1D and 2D NMR spectra of compounds were recorded in DMSO-d6 CD3OD on a Bruker 600 MHz spectrometer using a 5 mm TXI Cryoprobe in the AMRIS facility at the University of Florida, Gainesville, Fla., USA. Spectroscopy data were collected using Topspin 3.5 software. HRMS data were obtained using a Thermo Fisher Q Exactive Focus mass spectrometer equipped with electrospray probe on Universal Ion Max API source. The quantitation of the yields of txtA/B library reactions were based on the direct comparison of product concentration to the initial substrate concentration. The quantitation of the yields of txtA/B and TB14 library reactions were based on the standard curve of thaxtomin D. The quantitation of thaxtomin A and thaxtomin B yields from txtA/B, TB14 and TCB14 library reactions was based on the standard curve of thaxtomin A.

Overexpression of TxtA, TxtB, TxtH, TCB14, GDH, FDH and hMAT2A.

TxtC gene was amplified from genomic DNA of S. scabies 87.22 (NRRL B-24449) using a pair of TX-C-NcoI-F and C-SacI-R primers in PCR reactions (Table 3). The PCR product was analyzed by agarose gel and extracted with a GeneJET Gel Extraction Kit (Thermo). Purified PCR products and TB14 plasmid were digested with the restriction enzymes NcoI and SacI, and corresponding linear DNAs were ligated to generate expression construct pET28b-TxtEs. To further create the TxtE-BM3R variants with variable linker length, BM3R domain with selected linker lengths was amplified from P450BM3 gene by a set of primer pairs (Table 3). Purified PCR products and pET28b-TxtE construct were then digested with the restriction enzymes SacI and XhoI, and corresponding linear DNAs were ligated to generate pET26b-txtC14-BM3Rd-C-Histag expression constructs. TxtA, txtB, txtH, TCB14, GDH, FDH and hMAT2A were following similar procedures of TCB14 construction. All the constructed plasmids were sequenced to exclude mutations introduced during PCR amplification and gene manipulation. Protein expression and purification followed the previous protocols. The purified proteins were exchanged into storage buffer (25 mM Tris-HCl, pH 8.0, 100 mM NaCl, 3 mM βME, and 10% glycerol) by PD-10 column, aliquoted and stored at −80° C. until needed. CO difference spectroscopy was used to measure the concentrations of functional P450s.

Spectral Analysis of Chimeric TCB14.

Purified TB14 and TCB14 were spectrally analyzed following a previous protocol. Briefly, the absorbance spectra (400-600 nm) of TB14 and TCB14 in Tris-HCl (25 mM, pH 8) buffer were recorded by a Shimadzu UV2700 dual beam UV-Vis spectrophotometer. The ferric heme of enzymes was then saturated by bubbling carbon monoxide (Airgas) and the spectra of the saturated enzyme solutions were recorded. Solid sodium dithionite was then added to reduce ferric ion, and reduced spectra were taken subsequently. CO reduced difference spectra of all P450s were created by subtracting the CO binding spectra from the reduced spectra. Data were further analyzed by Excel. Substrate binding affinities to P450s were measured using 1.5 μM of enzyme solutions in 25 mM Tris-HCl, pH 8.0. The changes in absorbance (ΔA) were determined by subtracting the absorbance at ˜420 nm from that at ˜390 nm. Data were then fitted to the equation of ΔA=ΔAmax[L]/(Kd+[L]) using GraphPad Prism 4.

Enzymatic Reaction Conditions.

TxtA/B reaction: 100 mM Tris-Cl (PH=8.0), 0.5 mM Nitro-L-tryptophan or L-tryptophan, 0.55 mM L-Phenylalanine, 3.2 μM txtA, 14.5 μM txtB, 2.5 mM ATP and 2 mM SAM in 100 μL reaction. 21° C. shaken with 400 rpm for 20 hours.

TB14 and txtA/B coupling reaction: 100 mM Tris-Cl (PH=8.0), 0.5 mM L-tryptophan, 0.55 mM L-Phenylalanine, 1.5 mM NAPDH, 0.75 mM NOC-5, 3.2 μM txtA, 14.5 μM txtB, 2.5 mM ATP, 2 mM SAM and 11.1 μM TB14 in 100 μL reaction. Opened to air, 21° C. shaken with 400 rpm for 30 hours.

TB14, hMAT2A and txtA/B coupling reaction: 100 mM Tris-Cl (PH=8.0), 0.5 mM Nitro-L-tryptophan or L-tryptophan, 0.55 mM L-Phenylalanine, 1.5 mM NAPDH, 0.75 mM NOC-5, 100 mM KCl, 5 mM L-Methionine, 3.2 μM txtA, 14.5 μM txtB, 2.5 mM ATP, 3.2 μM hMAT2A, and 11.1 μM TB14 in 100 μL reaction. Opened to air, 21° C. shaken with 400 rpm for 30 hours.

TCB14 reaction: 100 mM Tris-Cl (PH=8.0), thaxtomin D (0.3 mM), TCB14 (16.7 μM), GDH (2.1 μM), NADP+ (0.75 μM) and glucose (16.6 mM). Opened to air, 21° C. shaken with 400 rpm for 20 hours.

TB14, txtA/B and TCB14 coupling reaction: TB14 and txtA/B coupling reaction was carried out as described above. Then add txtC14BM3Rd (16.7 μM), GDH (2.1 μM), NADP+/NAD+ (0.75 μM) and glucose (16.6 mM). Opened to air, 21° C. shaken with 400 rpm for 20 hours.

Large-Scale Enzymatic Synthesis of Thaxtomin Analogues.

To isolate sufficient amount of nitrated 4-Me-thxD (60) for structural determination, 14 μM TCB14, 3.2 μM txtA, 14.5 μM txtB was used in a 10-mL reaction mixture containing 0.5 mM 4-Me-Trp, 1.5 mM NADPH, 3 mM NOC-5, 2.5 mM ATP and 2 mM SAM in 100 mM Tris-HCl buffer (pH 8.0). The reactions were incubated at 21° C., 250 rpm overnight, and then terminated by 20 mL ethyl acetate (EA). After repeatedly extraction by EA, the supernatants were concentrated in vacuo and then freeze-dried. The products were redissolved in 3 ml methanol for semi-preparation.

HPLC and LC-MS Analysis.

For analytic purpose, the HPLC program included the column elution first with 10% solvent B (acetonitrile with 0.1% formic acid, FA) for 2 min and then with a linear gradient of 10-50% solvent B in 8 min, followed by another linear gradient of 50-99% solvent B in 5 min. The column was further cleaned with 99% solvent B for 3 min and then re-equilibrated with 10% solvent B for 1 min. Solvent A was water with 0.1% FA. The flow rate was set as 0.5 mL/min, and the products were detected at 254 nm with a PDA detector. For semi-preparative HPLC analysis, the column at 40° C. was first eluted with 10% solvent B (acetonitrile with 0.1% FA) for 2 min and then with a linear gradient of 10-50% solvent B for 8 min, followed by a linear gradient of 50-99% solvent B for 5 min. The column was then cleaned by 99% solvent B for 1 min and re-equilibrated with 10% solvent B for 1 min. The flow rate was set at 3 mL/min, and the products were detected at 380 nm with a PDA detector. All metabolites were well separated and corresponding fractions were combined, concentrated, dried, and then weighed. The fraction containing the targeted compound was collected and dried for further purification with one analytical column (YMC-Pack Ph column, 5 μm, 4.6×250 mm). The column at 30° C. was eluted with 50% solvent B (methanol with 0.1% FA) for 13.5 min and then with a linear gradient of 50-99% solvent B in 0.5 min, followed by another linear gradient of 50-99% solvent B in 0.5 min. After eluting in 99% solvent B for 0.5 min, the liner gradient of 99-50% solvent B in 1.0 min was used. The flow rate was set at 1 mL/min, and the product was detected at 380 nm with a PDA detector. The compound 60 was eluted and collected for NMR analysis.

A SHIMADZU Prominence UPLC system fitted with an Agilent Poroshell 120 EC-C18 column (2.7 μm, 4.6×50 mm) coupled with a Linear Ion Trap Quadrupole LC/MS/MS Mass Spectrometer system was used in the studies. The HPLC conditions were the same as the above. For MS detection, the turbo spray conditions included curtain gas: 30 psi; ion spray voltage: 5500 V; temperature: 600° C.; ion source gas 1: 50 psi; ion source gas 2: 60 psi). For MS/MS analysis, the collision energy was 12 eV. LC-HR-MS analysis was performed on a Thermo Fisher Q Exactive Focus mass spectrometer. Acetonitrile (B)/water (A) containing 0.1% FA were used as mobile phases with a linear gradient program (10-90% solvent B over 15 min) to separate chemicals at a flow rate of 0.3 mL/min. A pre-wash phase of 15 min with 10% solvent B was added at the beginning of each run, in which the elute was diverted to the waste by a diverting valve. MS1 were acquired under Full Scan mode of Orbitrap, in which a mass range of m/z 150-2000 was covered and data were collected in the positive ion mode. Fragmentation was introduced by HCD technique with optimized collision energy ranging from 6 to 15 eV. Other settings for the Orbitrap scan were as follow: resolution 15000, AGC target 5×10⁵. Full scan mass spectra and targeted MS/MS spectra for each of the pre-selected parental ion were extracted from the raw files of the HPLC-MS/MS Experiment II using Xcalibur™ 2.1 (Thermo Scientific).

TABLE 1
Amino acid analogs used to characterize the substrate scopes of TxtA/B.

| | | | | |
|---|---|---|---|---|
| | | TxtA | TxtA-W225S | TxtB |
| Chemicals | Abbreviation | substrate | substrate | substrate |

20 Amino acids

| | | | | |
|---|---|---|---|---|
| L-alanine | L-Ala | | | |
| L-arginine | L-Arg | | | |
| L-asparagine | L-Asn | | | |
| L-aspartic acid | L-Asp | | | |
| L-cysteine | L-Cys | | | |
| L-glutamine | L-Gln | | | |
| L-glutamic acid | L-Glu | | | |
| L-glycine | L-Gln | | | |
| L-histidine | L-His | | | |
| L-isoleucine | L-Ile | | | |
| L-leucine | L-Leu | | | |
| L-lysine | L-Lys | | | |
| L-methionine | L-Met | | | |
| L-phenylalanine | L-Phe | +++++^{a} | + | |
| L-proline | L-Pro | | | |
| L-serine | L-Ser | | | |
| L-threonine | L-Thr | | | |
| L-tryptophan | L-Trp | | | + |
| L-tyrosine | L-Tyr | | ++ | |
| L-valine | L-Val | | | |

L-tryptophan analogs

| | | |
|---|---|---|
| 4-nitro-L-Tryptophan | 4-nitro-Trp | +++++ |
| D-Tryptophan | D-Trp | |
| α-Me-L-Tryptophan | α-Me-L-Trp | |
| 5OH-Tryptophan | 5OH-Trp | |
| 5OMe-Tryptophan | 5OM-Trp | |
| 7-Aza-Tryptophan | 7-Aza-Trp | |
| 4F--Tryptophan | 4F--Trp | + |
| 5F-Tryptophan | 5F-Trp | ++ |
| 6F-Tryptophan | 6F-Trp | ++ |
| 4Me-D,L-Tryptophan | 4Me-D,L-Trp | ++ |
| 5Me-D,L-Tryptophan | 5Me-D,L-Trp | |
| 6Me-D,L-Tryptophan | 6Me-D,L-Trp | |
| 7Me-D,L-Tryptophan | 7Me-D,L-Trp | |
| 5Cl-L-Tryptophan | 5Cl-L-Trp | |
| 5Br-L-Tryptophan | 5Br-L-Trp | |
| 1M-L-Tryptophan | 1M-L-Trp | |

L-phenylalanine analogs

| | | | |
|---|---|---|---|
| 2F-L-phenylalanine | 2F-L-Phe | ++++ | |
| 3F-L-phenylalanine | 3F-L-Phe | +++++ | |
| 4F-L-phenylalanine | 4F-L-Phe | +++ | |
| 3Cl-L-phenylalanine | 3Cl-L-Phe | ++ | |
| 4Cl-L-phenylalanine | 4Cl-L-Phe | ++ | |
| 2Br-L-phenylalanine | 2Br-L-Phe | + | |
| 3Br-L-phenylalanine | 3Br-L-Phe | + | |
| 4Br-L-phenylalanine | 4Br-L-Phe | + | |
| 2Me-L-phenylalanine | 2Me-L-Phe | +++ | |
| 3Me-L-phenylalanine | 3Me-L-Phe | ++ | |
| 4Me-L-phenylalanine | 4Me-L-Phe | +++ | |
| 3OMe-L-phenylalanine | 3OMe-L-Phe | | + |
| α-Me-L-phenylalanine | α-Me-L-Phe | | |
| 3-(4-Pyridyl)-L-alanine | Pyri-L-Phe | | |
| 4-Azidophenylalanine | 4-N₃-L-Phe | | ++ |
| 4-Aminophenylalanine | 4-NH₂-L-Phe | | |

^{a}The number of ‘+’ represents the reactivities of the enzymes to the substrate. Five ‘+’ represents the strong reactivity and one represent very weak reactivities.

TABLE 2
HR-MS data and conversion rate of cyclopeptides generated through txtA/B
enzymatic reactions in optimized conditions.

| | | Substrate | Substrate | | |
|---|---|---|---|---|---|
| | Conversion | L-Phe or L-Phe | L-Trp or | Calculated | Identified |
| Compound # | rate % | analog | L-Trp analog | [M + H]⁺ | [M + H]⁺ |
| 18 | 5.28% | L-Phe | L-Trp | 362.1869 | 362.1848 |
| 19 | 3.16% | 2F-L-Phe | L-Trp | 380.1774 | 380.1757 |
| 20 | 4.93% | 3F-L-Phe | L-Trp | 380.1774 | 380.1756 |
| 21 | 4.86% | 4F-L-Phe | L-Trp | 380.1774 | 380.1758 |
| 22 | 0.85% | 3Cl-L-Phe | L-Trp | 396.1479 | 396.1464 |
| 23 | 1.81% | 4Cl-L-Phe | L-Trp | 396.1479 | 396.1462 |
| 24 | 2.23% | L-Phe | 4F-L-Trp | 380.1774 | 380.1757 |
| 25 | 1.87% | 2F-L-Phe | 4F-L-Trp | 398.1680 | 398.1666 |
| 26 | 2.37% | 3F-L-Phe | 4F-L-Trp | 398.1680 | 398.1666 |
| 27 | 2.16% | 4F-L-Phe | 4F-L-Trp | 398.1680 | 398.1664 |
| 28 | 1.42% | 3Cl-L-Phe | 4F-L-Trp | 414.1385 | 414.1367 |
| 29 | 0.92% | 4Cl-L-Phe | 4F-L-Trp | 414.1385 | 414.1367 |
| 30 | 10.18% | L-Phe | 5F-L-Trp | 380.1774 | 380.1757 |
| 31 | 7.00% | 2F-L-Phe | 5F-L-Trp | 398.1680 | 398.1664 |
| 32 | 9.63% | 3F-L-Phe | 5F-L-Trp | 398.1680 | 398.1662 |
| 33 | 8.95% | 4F-L-Phe | 5F-L-Trp | 398.1680 | 398.1663 |
| 34 | 1.84% | 3Cl-L-Phe | 5F-L-Trp | 414.1385 | 414.1366 |
| 35 | 3.60% | 4Cl-L-Phe | 5F-L-Trp | 414.1385 | 414.1366 |
| 36 | 2.63% | L-Phe | 6F-L-Trp | 380.1774 | 380.1762 |
| 37 | 1.66% | 2F-L-Phe | 6F-L-Trp | 398.1680 | 398.1665 |
| 38 | 2.51% | 3F-L-Phe | 6F-L-Trp | 398.1680 | 398.1669 |
| 39 | 2.62% | 4F-L-Phe | 6F-L-Trp | 398.1680 | 398.1667 |
| 40 | 0.18% | 3Cl-L-Phe | 6F-L-Trp | 414.1385 | 414.1375 |
| 41 | 0.67% | 4Cl-L-Phe | 6F-L-Trp | 414.1385 | 414.1373 |
| 42 | 7.91% | L-Phe | 4Me-D,L-Trp | 376.2025 | 376.2018 |
| 43 | 3.97% | 2F-L-Phe | 4Me-D,L-Trp | 394.1931 | 394.1915 |
| 44 | 6.69% | 3F-L-Phe | 4Me-D,L-Trp | 394.1931 | 394.1417 |
| 45 | 6.24% | 4F-L-Phe | 4Me-D,L-Trp | 394.1931 | 394.1920 |
| 46 | 1.06% | 3Cl-L-Phe | 4Me-D,L-Trp | 410.1635 | 410.1624 |
| 47 | 1.04% | 4Cl-L-Phe | 4Me-D,L-Trp | 410.1635 | 410.1623 |

TABLE 3
HR-MS data and conversion rate of thaxtomin D analogs generated through txtA/B
andTB14 coupling enzymatic reactions in optimized conditions.

| | | Substrate | Substrate | | |
|---|---|---|---|---|---|
| | Conversion | L-Phe or L-Phe | L-Trp or | Calculated | Identified |
| Compound # | rate % | analog | L-Trp analog | [M + H]⁺ | [M + H]⁺ |
| 17 | 43.44% | L-Phe | L-Trp | 379.1406 | 379.1396 |
| 9 | 24.51% | L-Phe | L-Trp | 393.1563 | 393.1552 |
| 48a | 9.55% | 4N3-L-Phe | L-Trp | 448.1733 | 448.1714 |
| 49a | 8.45% | L-Tyr | L-Trp | 423.1668 | 423.1646 |
| 11 | 95.15% | L-Phe | L-Trp | 407.1719 | 407.1710 |
| 50 | 88.95% | 2F-L-Phe | L-Trp | 425.1625 | 425.1615 |
| 51 | 90.68% | 3F-L-Phe | L-Trp | 425.1625 | 425.1603 |
| 52 | 66.09% | 4F-L-Phe | L-Trp | 425.1625 | 425.1604 |
| 53 | 20.95% | 3Cl-L-Phe | L-Trp | 441.1330 | 441.1309 |
| 54 | 24.41% | 4Cl-L-Phe | L-Trp | 441.1330 | 441.1314 |
| 55 | 12.67% | 2Br-L-Phe | L-Trp | 485.0824 | 485.0807 |
| 56 | 10.17% | 3Br-L-Phe | L-Trp | 485.0824 | 485.0802 |
| 57 | 4.49% | 4Br-L-Phe | L-Trp | 485.0824 | 485.0807 |
| 58 | 34.05% | 2Me-L-Phe | L-Trp | 421.1876 | 421.1862 |
| 59 | 17.85% | 3Me-L-Phe | L-Trp | 421.1876 | 421.1856 |
| 60 | 28.62% | 4Me-L-Phe | L-Trp | 421.1876 | 421.1856 |
| 61 | 23.41% | L-Phe | 5F-L-Trp | 425.1625 | 425.1607 |
| 62 | 15.45% | 2F-L-Phe | 5F-L-Trp | 443.1531 | 443.1510 |
| 63 | 20.69% | 3F-L-Phe | 5F-L-Trp | 443.1531 | 443.1512 |
| 64 | 19.02% | 4F-L-Phe | 5F-L-Trp | 443.1531 | 443.1510 |
| 65 | 5.03% | 3Cl-L-Phe | 5F-L-Trp | 459.1235 | 459.1215 |
| 66 | 5.63% | 4Cl-L-Phe | 5F-L-Trp | 459.1235 | 459.1218 |
| 67 | 2.67% | 2Br-L-Phe | 5F-L-Trp | 503.0730 | 503.0712 |
| 68 | 2.26% | 3Br-L-Phe | 5F-L-Trp | 503.0730 | 503.0712 |
| 69 | 1.37% | 4Br-L-Phe | 5F-L-Trp | 503.0730 | 503.0710 |
| 70 | 9.07% | 2Me-L-Phe | 5F-L-Trp | 439.1782 | 439.1764 |
| 71 | 5.33% | 3Me-L-Phe | 5F-L-Trp | 439.1782 | 439.1765 |
| 72 | 9.79% | 4Me-L-Phe | 5F-L-Trp | 439.1782 | 439.1766 |
| 73 | 48.07% | L-Phe | 6F-L-Trp | 425.1625 | 425.1605 |
| 74 | 43.31% | 2F-L-Phe | 6F-L-Trp | 443.1531 | 443.1512 |
| 75 | 44.86% | 3F-L-Phe | 6F-L-Trp | 443.1531 | 443.1514 |
| 76 | 44.68% | 4F-L-Phe | 6F-L-Trp | 443.1531 | 443.1514 |
| 77 | 4.13% | 3Cl-L-Phe | 6F-L-Trp | 459.1235 | 459.1216 |
| 78 | 10.06% | 4Cl-L-Phe | 6F-L-Trp | 459.1235 | 459.1216 |
| 79 | 1.23% | 2Br-L-Phe | 6F-L-Trp | 503.0730 | 503.0714 |
| 80 | 2.53% | 3Br-L-Phe | 6F-L-Trp | 503.0730 | 503.0713 |
| 81 | 0.6% | 4Br-L-Phe | 6F-L-Trp | 503.0730 | 503.0711 |
| 82 | 6.29% | 2Me-L-Phe | 6F-L-Trp | 439.1782 | 439.1766 |
| 83 | 4.47% | 3Me-L-Phe | 6F-L-Trp | 439.1782 | 439.1765 |
| 84 | 12.07% | 4Me-L-Phe | 6F-L-Trp | 439.1782 | 439.1764 |

a Catalyzed by TxtB and TxtA-W225S

TABLE 4
HR-MS data and conversion rate of thaxtomin B analogs generated through txtA/B,
TCB14 and TB14 enzymatic reactions in optimized conditions.

| | | Substrate | Substrate | | |
|---|---|---|---|---|---|
| | Conversion | L-Phe or L-Phe | L-Trp or | Calculated | Identified |
| Compound # | rate % | analog | L-Trp analog | [M + H]⁺ | [M + H]⁺ |
| 10 | 43.70% | L-Phe | L-Trp | 423.1668 | 423.1656 |
| 85 | 33.78% | 2F-L-Phe | L-Trp | 441.1574 | 441.1568 |
| 86 | 54.75% | 3F-L-Phe | L-Trp | 441.1574 | 441.1569 |
| 87 | 8.94% | 4F-L-Phe | L-Trp | 441.1574 | 441.1569 |
| 88 | 10.3% | 3Cl-L-Phe | L-Trp | 457.1279 | 457.1274 |
| 89 | 4.74% | 4Cl-L-Phe | L-Trp | 457.1279 | 457.1272 |
| 90 | 3.53% | 2Br-L-Phe | L-Trp | 501.0774 | 501.0764 |
| 91 | 3.61% | 3Br-L-Phe | L-Trp | 501.0774 | 501.0763 |
| 92 | 1.3% | 4Br-L-Phe | L-Trp | 501.0774 | 501.0769 |
| 93 | 7.15% | 2Me-L-Phe | L-Trp | 437.1825 | 437.1810 |
| 94 | 10.02% | 3Me-L-Phe | L-Trp | 437.1825 | 437.1808 |
| 95 | 23.86% | 4Me-L-Phe | L-Trp | 437.1825 | 437.1807 |
| 96 | 0.77% | L-Phe | 5F-L-Trp | 441.1574 | 441.1559 |
| 97 | 2.87% | 2F-L-Phe | 5F-L-Trp | 459.1480 | 459.1462 |
| 98 | 2.45% | 3F-L-Phe | 5F-L-Trp | 459.1480 | 459.1463 |
| 99 | 0.27% | 4F-L-Phe | 5F-L-Trp | 459.1480 | 459.1466 |
| 100 | 1.22% | 3Cl-L-Phe | 5F-L-Trp | 475.1185 | 475.1167 |
| 101 | 0.72% | 4Cl-L-Phe | 5F-L-Trp | 475.1185 | 475.1169 |
| 102 | 0.86% | 2Br-L-Phe | 5F-L-Trp | 519.0679 | 519.0661 |
| 103 | 0.67% | 3Br-L-Phe | 5F-L-Trp | 519.0679 | 519.0661 |
| 104 | 0.15% | 4Br-L-Phe | 5F-L-Trp | 519.0679 | 519.0662 |
| 105 | 1.66% | 2Me-L-Phe | 5F-L-Trp | 455.1731 | 455.1712 |
| 106 | 2.06% | 3Me-L-Phe | 5F-L-Trp | 455.1731 | 455.1712 |
| 107 | 0.9% | 4Me-L-Phe | 5F-L-Trp | 455.1731 | 455.1713 |
| 108 | 1.14% | L-Phe | 6F-L-Trp | 441.1574 | 441.1560 |
| 109 | 0.34% | 2F-L-Phe | 6F-L-Trp | 459.1480 | 459.1463 |
| 110 | 3.32% | 3F-L-Phe | 6F-L-Trp | 459.1480 | 459.1461 |
| 111 | 0.23% | 4Cl-L-Phe | 6F-L-Trp | 475.1185 | 475.1166 |
| 112 | 0.12% | 2Br-L-Phe | 6F-L-Trp | 519.0679 | 519.0663 |
| 113 | 0.41% | 3Br-L-Phe | 6F-L-Trp | 519.0679 | 519.0662 |
| 114 | 0.32% | 2Me-L-Phe | 6F-L-Trp | 455.1731 | 455.1714 |
| 115 | 0.67% | 3Me-L-Phe | 6F-L-Trp | 455.1731 | 455.1531 |
| 116 | 0.83% | 4Me-L-Phe | 6F-L-Trp | 455.1731 | 455.1712 |

TABLE 5
HR-MS data and conversion rate of thaxtomin A analogs generated through txtA/B,
TCB14 and TB14 enzymatic reactions in optimized conditions.

| | | Substrate | Substrate | | |
|---|---|---|---|---|---|
| | Conversion | L-Phe or L-Phe | L-Trp or | Calculated | Identified |
| Compound # | rate % | analog | L-Trp analog | [M + H]⁺ | [M + H]⁺ |
| 1 | 9.98% | L-Phe | L-Trp | 439.1618 | 439.1607 |
| 117 | 7.31% | 2F-L-Phe | L-Trp | 457.1523 | 457.1517 |
| 118 | 7.89% | 3F-L-Phe | L-Trp | 457.1523 | 457.1517 |
| 119 | 33.97% | 4F-L-Phe | L-Trp | 457.1523 | 457.1515 |
| 120 | 2.74% | 3Cl-L-Phe | L-Trp | 473.1228 | 473.1223 |
| 121 | 10.14% | 4Cl-L-Phe | L-Trp | 473.1228 | 473.1222 |
| 122 | 0.82% | 2Br-L-Phe | L-Trp | 517.0723 | 517.0714 |
| 123 | 0.55% | 3Br-L-Phe | L-Trp | 517.0723 | 517.0720 |
| 124 | 1.6% | 4Br-L-Phe | L-Trp | 517.0723 | 517.0717 |
| 125 | 1.72% | 2Me-L-Phe | L-Trp | 453.1774 | 453.1756 |
| 126 | 1.37% | 4Me-L-Phe | L-Trp | 453.1774 | 453.1757 |
| 127 | 8.78% | L-Phe | 5F-L-Trp | 457.1523 | 457.1505 |
| 128 | 2.53% | 2F-L-Phe | 5F-L-Trp | 475.1429 | 475.1412 |
| 129 | 8.78% | 3F-L-Phe | 5F-L-Trp | 475.1429 | 475.1420 |
| 130 | 3.07% | 4F-L-Phe | 5F-L-Trp | 475.1429 | 475.1414 |
| 131 | 1.08% | 3Cl-L-Phe | 5F-L-Trp | 491.1134 | 459.1466 |
| 132 | 1.91% | 4Cl-L-Phe | 5F-L-Trp | 491.1134 | 491.1115 |
| 133 | 0.54% | 2Br-L-Phe | 5F-L-Trp | 535.0629 | 535.0612 |
| 134 | 0.31% | 3Br-L-Phe | 5F-L-Trp | 535.0629 | 535.0618 |
| 135 | 0.36% | 4Br-L-Phe | 5F-L-Trp | 535.0629 | 535.0612 |
| 136 | 1.26% | 2Me-L-Phe | 5F-L-Trp | 471.1680 | 471.1663 |
| 137 | 1.37% | 4Me-L-Phe | 5F-L-Trp | 471.1680 | 471.1662 |
| 138 | 5.22% | L-Phe | 6F-L-Trp | 457.1523 | 457.1507 |
| 139 | 2.55% | 2F-L-Phe | 6F-L-Trp | 475.1429 | 475.1409 |
| 140 | 5.22% | 3F-L-Phe | 6F-L-Trp | 475.1429 | 475.1408 |
| 141 | 3.16% | 4F-L-Phe | 6F-L-Trp | 475.1429 | 475.1416 |
| 142 | 0.3% | 3Cl-L-Phe | 6F-L-Trp | 491.1134 | 491.1116 |
| 143 | 1.08% | 4Cl-L-Phe | 6F-L-Trp | 491.1134 | 491.1116 |
| 144 | 0.74% | 4Me-L-Phe | 6F-L-Trp | 471.1680 | 471.1664 |

TABLE 6
Primers used in the study.

| | | |
|---|---|---|
| | | SEQ |
| Primer | | ID |
| Name | Sequence (5′ → 3′) | No. |

pET28b-TxtA-C-Histag

| | | |
|---|---|---|
| C-Histag- | ATCCCATGGGCATGTCGCACCTGACCGGTGAAGATC | 1 |
| TxtA-F | | |
| C-Histag- | ATCAAGCTTCTGCAGTGGGAGGTCCTGG | 2 |
| TxtA-R | | |

pET28b-TxtB-C-Histag

| | | |
|---|---|---|
| C-Histag- | ATCCCATGGGCATGGTCCACTCGATGTCCATGCTGC | 3 |
| TxtA-F | | |
| C-Histag- | ATC AAGCTT CGGCCGTGGTGAGAAGG | 4 |
| TxtA-R | | |

pET22b-TxtH-C-Histag

| | | |
|---|---|---|
| TxtH-F | ACTGGATCCGGTGCCCTCACCCTTCGACGAC | 5 |
| TxtH-R | ACTGAATTCGATTCACGGACGGACGCCGGGC | 6 |

pET26b-TxtC14-BM3Rd-C-Histag

| | | |
|---|---|---|
| C-NcoI-F | ATACCATGGAATCTCCGGCCACCCAG | 7 |
| C-SadI-R | ATAGAGCTCCCAGGTCATGGGCAGCTCATG | 8 |

pET28b-hMAT2A-C-Histag

| | | |
|---|---|---|
| MAT-F | CATGAATTCGATGAACGGACAGCTCAACGG | 9 |
| MAT-R | AGCGGCCGCTCAATATTTAAGCTTTTTGG | 10 |

pET22b-GDH-C-Histag

| | | |
|---|---|---|
| GDH-F | ATACATATGTATAAAGATCTGGAAGGTA | 11 |
| GDH-R | ATAAAGCTTGCCACGACCTGCCTGAAAGCT | 12 |

pET22b-FDH-C-Histag

| | | |
|---|---|---|
| FDH-F | ATACATATGGCAAAGGTCCTGTGCGTT | 13 |
| FDH-R | ATAAAGCTTGACCGCCTTCTTGAACTTGG | 14 |

### Example 2

Non-ribosomal peptides (NRPs) are one major family of natural products that have a wide array of applications ranging from human health to agriculture [Katz, L. et al. J. Ind. Microbiol. Biotechnol. 2016 43:155-176; Fischbach, M. A. et al. Chem. Rev. 2006 106:3468-3496]. The substrate specificity, order, and number of NRP synthetase (NRPS) modules along with a variety of tailoring modifications together grant the enormous chemical diversity of NRPs [Bozhuyuk, K. A. J. et al. Nat. Chem. 2018 10:275-281; Süssmuth, R. D. et al. Angew. Chem. Int. Ed. Engl. 2017 56:3770-3821; Kries, H. et al. Angew. Chem. Int. Ed. Engl. 2014 53:10105-10108]. The modularity of NPR biosynthesis encourages synthetic biology-type studies to create natural products-like libraries in a combinatorial manner [Kries, H. et al. Angew. Chem. Int. Ed. Engl. 2014 53:10105-10108]. However, despite a few notable successes [Katz, L. et al. J. Ind. Microbiol. Biotechnol. 2016 43:155-176; Niquille, D. L. et al. Nat. Chem. 2018 10:282-287; Williams, G. J. Curr. Opin. Struct. Biol. 2013 23:603-612; Yan, Y. et al. Angew. Chem. Int. Ed. 2013 52:12308-12312; Wang, M. et al. ACS Catal. 2014 4:1219-1225], the scope and extent of these studies remain limited. In this regard, in vitro reconstitution of NRP biosynthesis not only synthesizes peptidic compounds but also delineates useful principles guiding the generation of chemical diversity [von Tesmar, A. et al. Cell Chem. Biol. 2017 24:1216-1227; Goering, A. W. et al. ACS Syn. Biol. 2017 6:39-44; Lowry, B. et al. Synlett 2015 26:1008-1025], aiding NPR biocombinatorial synthesis.

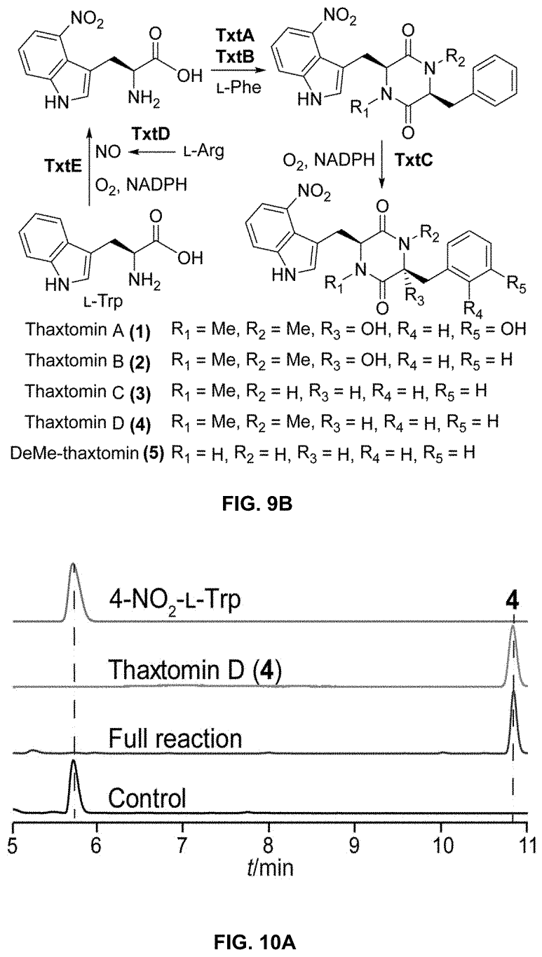

A family of the smallest cyclopeptides (FIG. 9A), 2,5-diketopiperazines (DKPs) are assembled from two amino acid monomers by two NRPS modules or one cyclodipeptide synthase [James, E. D. et al. ACS Synth. Biol. 2016 5:547-553; Scharf, D. H. et al. Appl. Microbiol. Biotechnol. 2012 93:467-472; Gondry, M. et al. Nat. Chem. Biol. 2009 5:414-420]. This scaffold is metabolically stable, structurally constrained, and amenable to multiple stereo-specific modifications [Ortiz, A. et al. Curr. Med. Chem. 2017 24:2773-2780; Borthwick, A. D. Chem. Rev. 2012 112:3641-3716], making it privileged in drug discovery and development (e.g., the PDES inhibitor Tadalafil and the oxytocin receptor antagonist Retosiban, FIG. 9A). The combination of the brevity of DKP biogenesis and structural diversity of final products indicates that the DKP biosynthetic systems can be leveraged to unravel the rules of chemical innovation in nature [Li, L. et al. Chem. Rev. 2018 118:3752-3832; Mori, S. et al. Biochemistry 2017 56:6087-6097].

Thaxtomins (Thxs) are a group of DKP analogues produced by tens of Streptomyces strains causing potato common scab [Loria, R. et al. Antonie Van Leeuwenhoek 2008 94:3-10]. They are virulence factors and inhibit cellulose biosynthesis in the nM range [Fry, B. A. et al. Physiol. Mol. Plant Pathol. 2002 60:1-8]. Given their impressive potency and a new model of action, Thxs have been pursued as herbicides for crop protection. However, synthetic routes to Thx analogues for herbicide development often suffer from the lack of stereo-control and low overall yield [Balalaie, S. et al. J. Org. Chem. 2017 82:12141-12152; Zhang, H. et al. Org. Lett. 2013 15:5670-5673]. On the other hand, nature devises a concise biosynthetic pathway that produces thaxtomin A (1), the dominant metabolite, and 11 analogues with structural variations mainly on N-methylation of the DKP scaffold and hydroxylation on the aryl group (FIG. 9B; see also FIG. 14) [Loria, R. et al. Antonie Van Leeuwenhoek 2008 94:3-10]. This pathway starts with the production of 4NO2-L-tryptophan (4NO2-L-Trp) by one unique P450 enzyme TxtE using co-substrate nitric oxide (NO) generated from L-arginine by one nitric oxide synthase TxtD [Barry, S. M. et al. Nat. Chem. Biol. 2012 8:14-816; Kers, J. A. et al. Nature 2004 429:79-82]. Two single-module NRPSs TxtA and TxtB then use L-phenylalanine (L-Phe) and 4NO2-L-Trp, respectively, to produce thaxtomin D (4). Finally, TxtC, the other pathway-specific P450 enzyme, hydroxylates α-position and the C′3 of the aryl group to produce 1 (FIG. 9B). Among all Thx biosynthetic enzymes, we and others have biochemically characterized the catalytic function and substrate scope of TxtE [Dodani, S. C. et al. Chembiochem 2014 15:2259-2267; Zuo, R. et al. Biotechnol. J. 2016 11:624-632; Zuo, R. et al. Sci. Rep. 2017 7:842], while the rest has been examined only in genetic studies [Wnn, M. et al. Angew. Chem. Int. Ed. Engl. 2018 57:6830-6833; Jiang, G. et al. Appl. Environ. Microbiol. 2018 84:e00164-18], limiting the efforts to utilize them for basic and biotechnological applications. Reported herein are biochemical characterization of the functions and substrate scopes of TxtA-C and the in vitro reconstitution of Thx biosynthesis. These studies led to the production of 124 Thx analogues and offered insights that will be useful to expand the chemical diversity of NRPs in future.

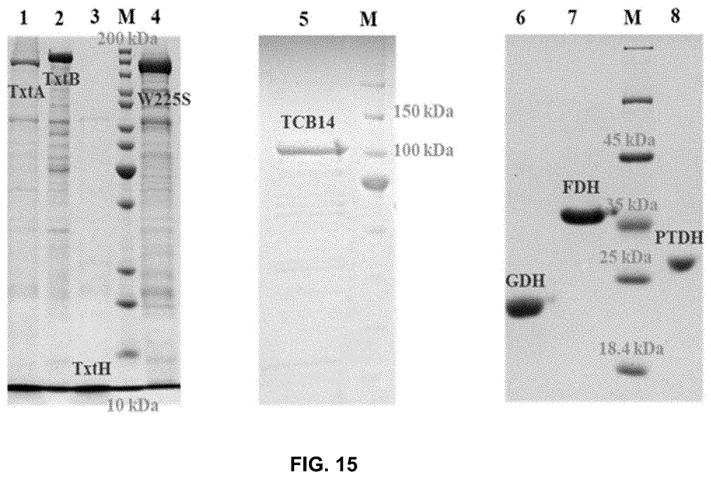

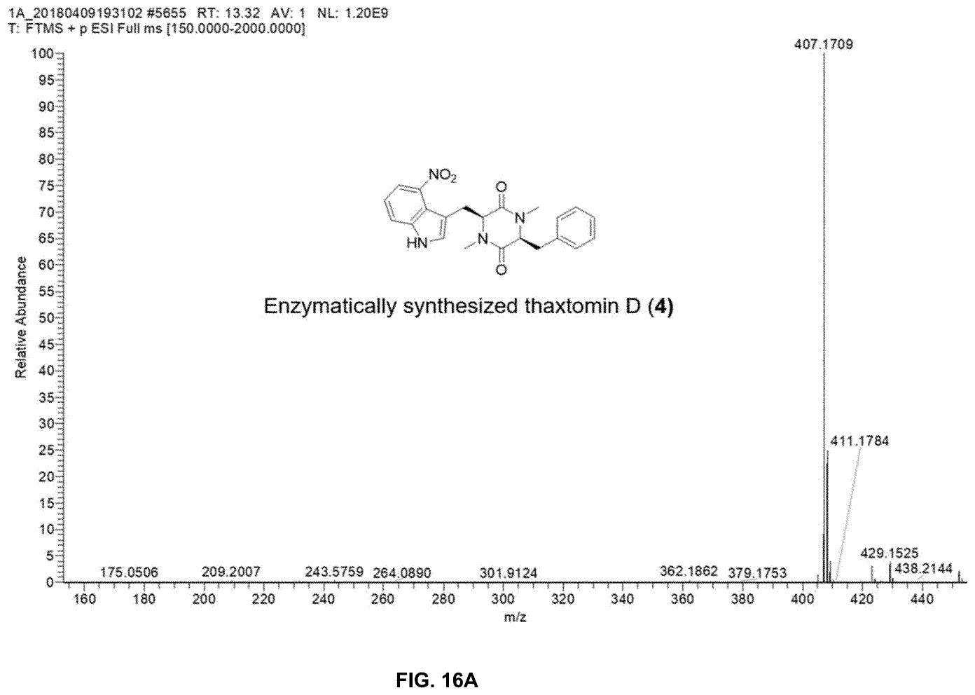

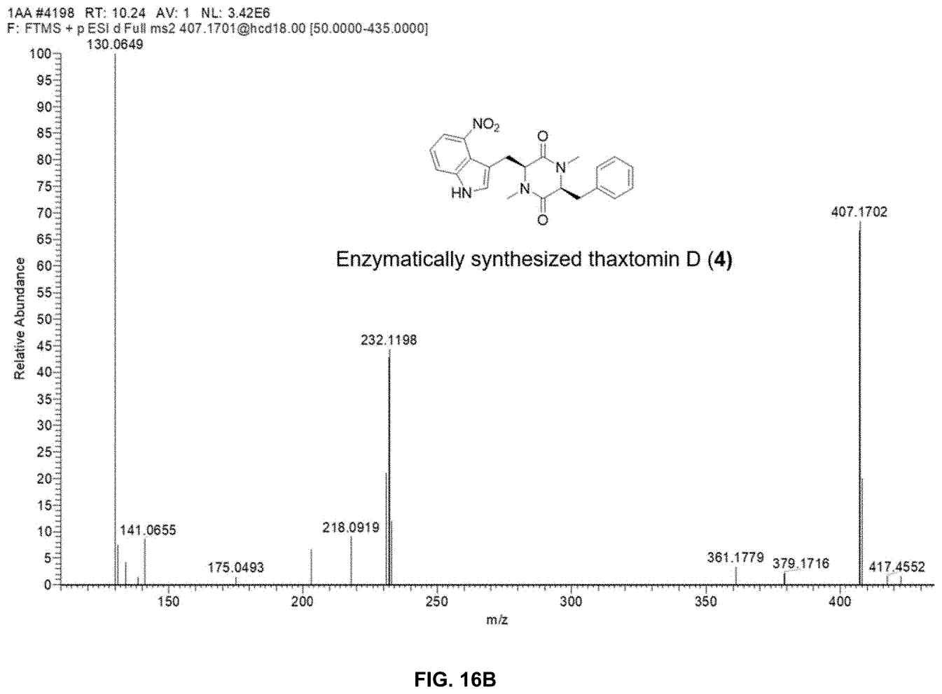

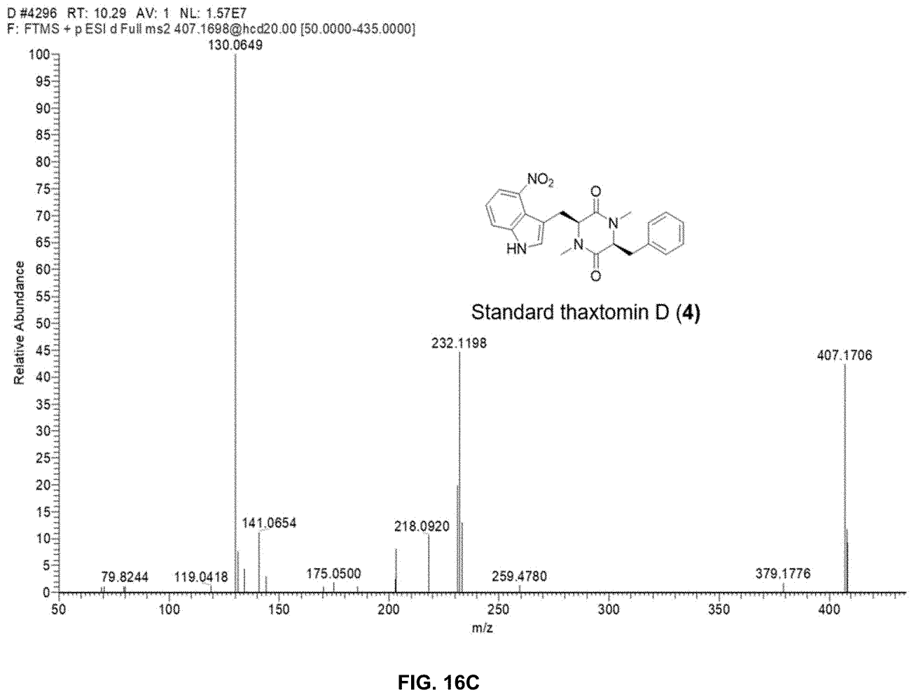

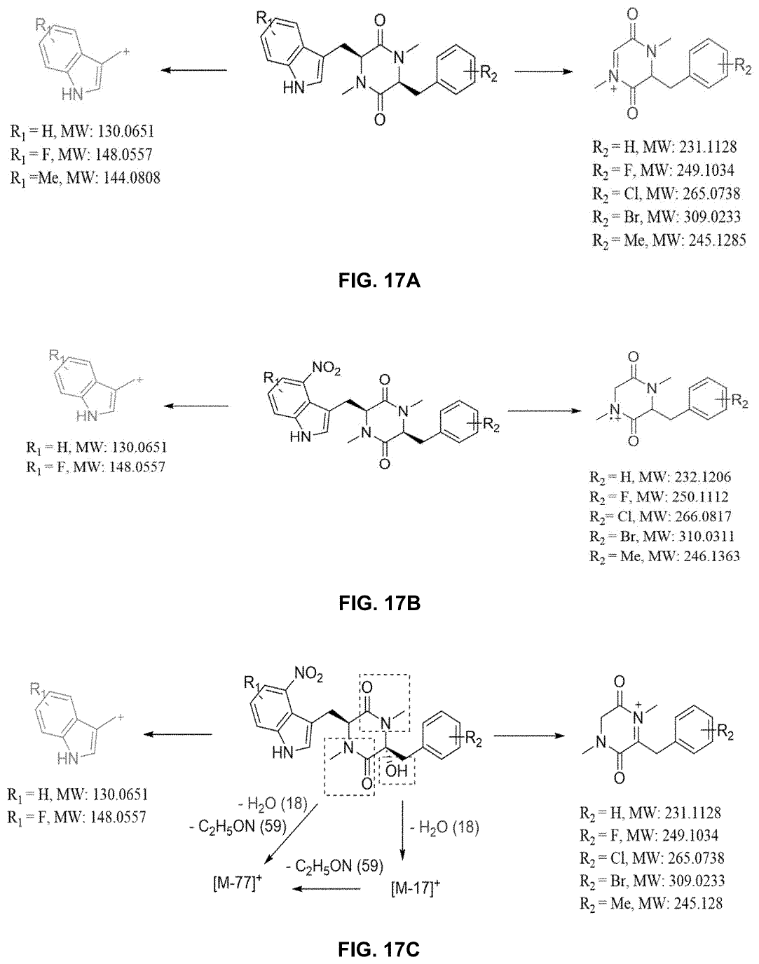

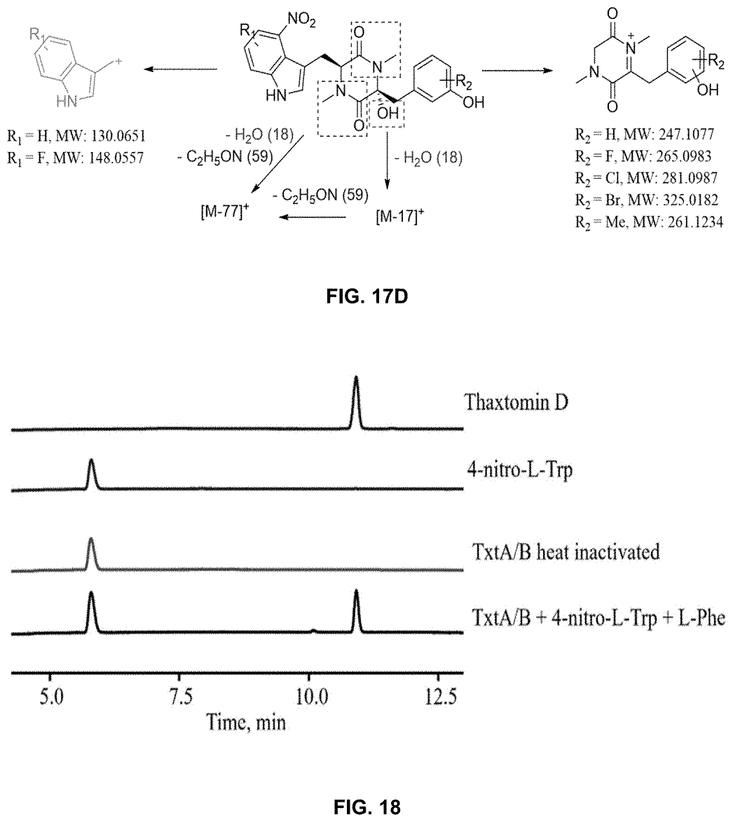

These studies were begun by preparing recombinant TxtA and TxtB in E. coli BAP1. Coexpression with txtH, a pathway-specific MtbH-like protein gene [Felnagle, E. A. et al. Biochemistry 2010 49:8815-8817], significantly enhanced the expression level and solubility of TxtA and TxtB (FIG. 15). The catalytic functions of recombinant TxtA and TxtB were examined in the reaction containing L-Phe, S-adenosyl methionine (SAM), ATP, Mg²⁺, and 4NO2-L-Trp or L-Trp that was effectively nitrated in situ by our previously engineered self-sufficient TxtE variant TB14 [Zuo, R. et al. Sci. Rep. 2017 7:842]. LC-high-resolution MS (LC-HRMS) analysis revealed a single product with the same retention time, m/z value (407.1710, A 1.0 ppm) and tandem MS pattern as the standard thaxtomin D (4) (FIG. 10A; FIG. 16), which missed in the control with heat-inactivated NRPSs. MS/MS fragmentation patterns of 4 and other Thx analogues were depicted in FIG. 17 and produced multiple diagnostic m/z values for structural identification. 4NO2-L-Trp has a low solubility, was less efficient than the in situ generated one for the reaction (FIG. 18). The latter was thus used below.

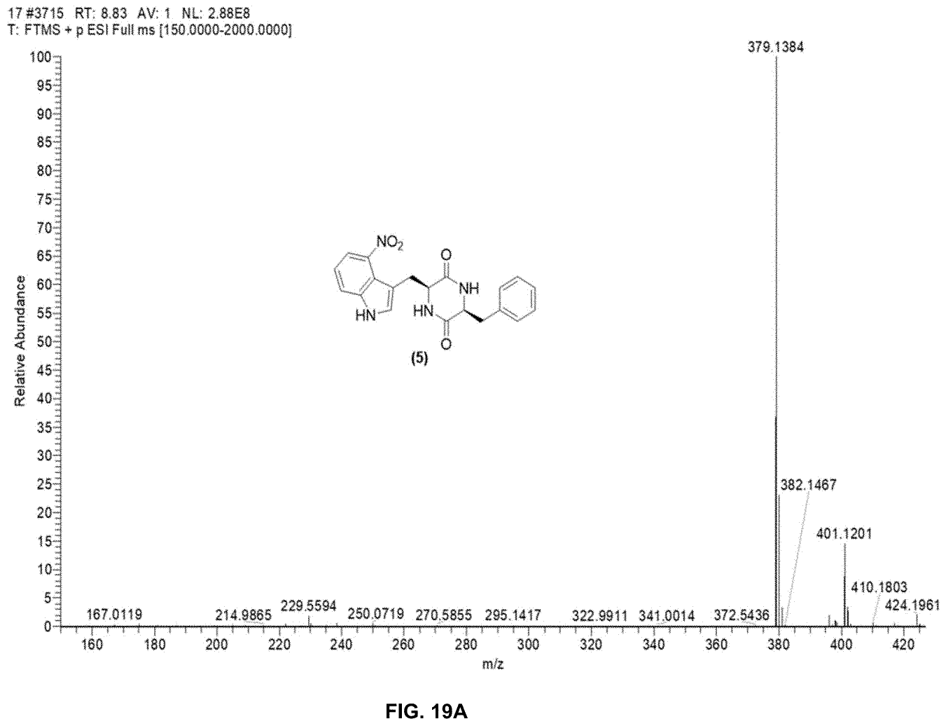

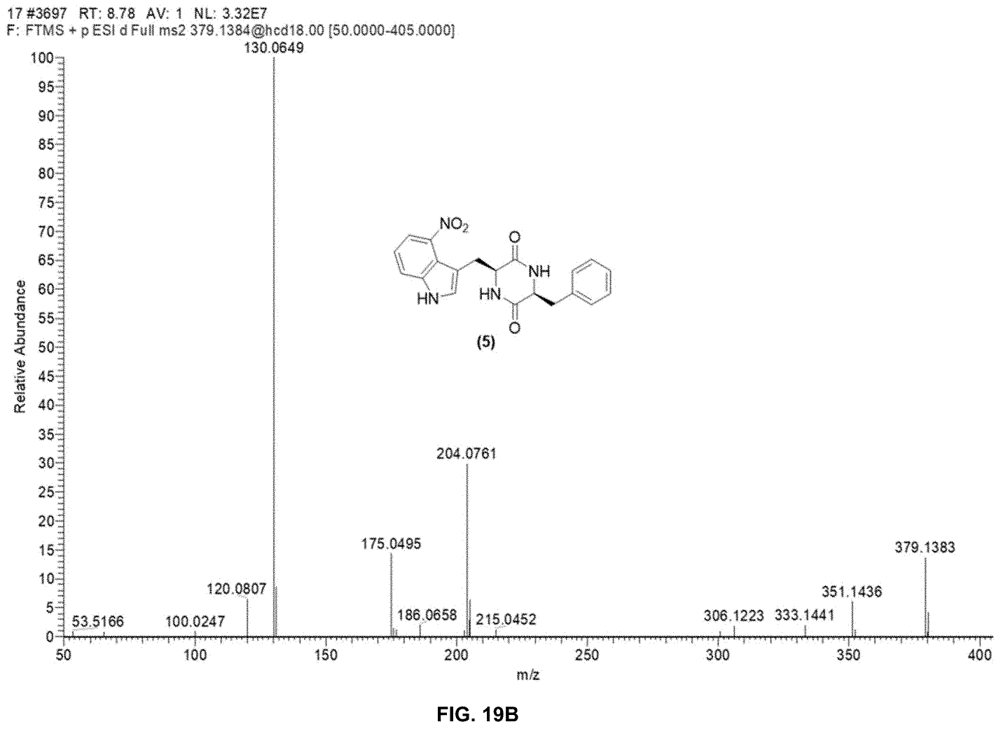

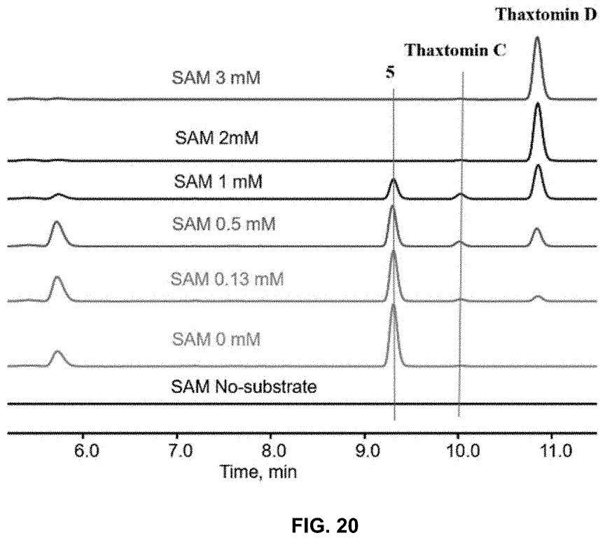

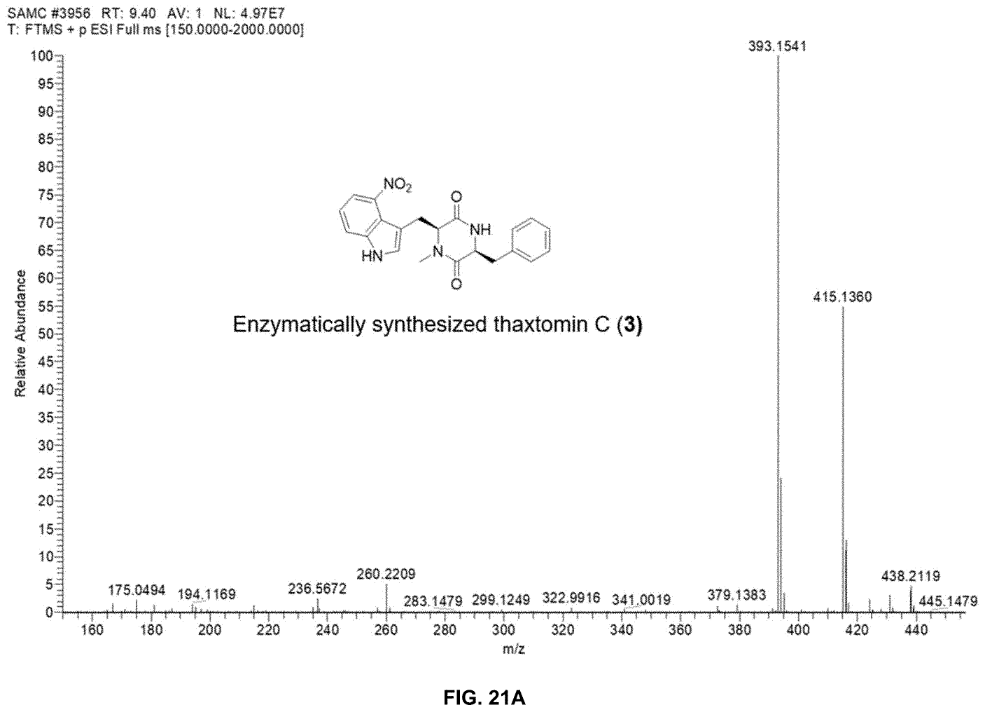

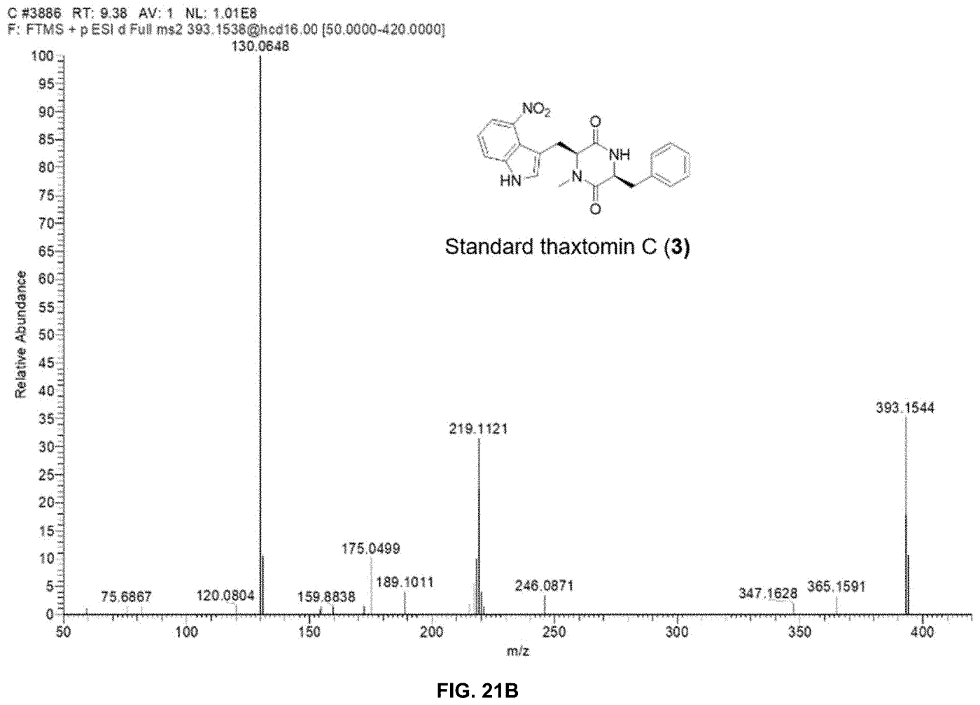

Both TxtA and TxtB carry one N-methyltransferase (MT) domain. The omission of SAM in the reaction mildly affected other reactions of TxtA and TxtB and generated one product with an identical m/z to demethyl-Thx D (5, 379.1396, Δ 1.3 ppm) and expected MS2 fragments (FIG. 19). When setting the ratio of [SAM]:[amino acids] at 2:1, the reaction produced a mixture of compound 5, thaxtomin C (3), and 4 with a molar ratio of 8:1:4, and further increase of the ratio to 4:1 resulted in 4 as the single product (FIG. 10B; FIG. 20). Overall, these results indicated that the N-MT domains of TxtA and TxtB methylate the —NH₂ of substrates tethered to corresponding thiolation domains and significantly favor di- over mono-methylation when the availability of SAM is limiting. Importantly, this work suggested that NRPS domains have a relatively independent control on the generation of DKP chemical diversity as the methylation reactions merely influence the condensation reaction [Mori, S. et al. Nat. Chem. Biol. 2018 14: 428-430].

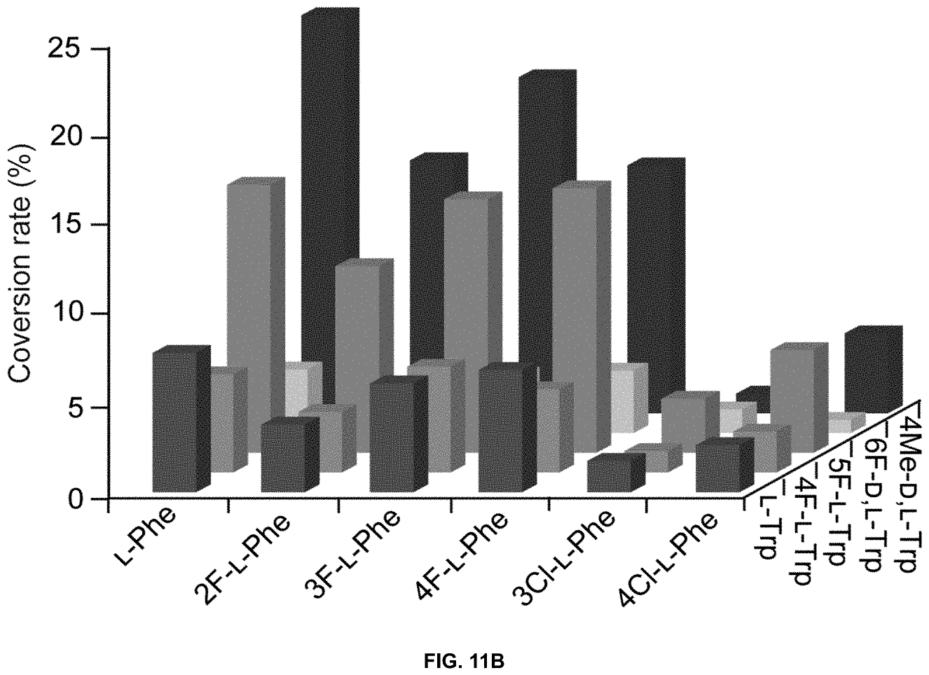

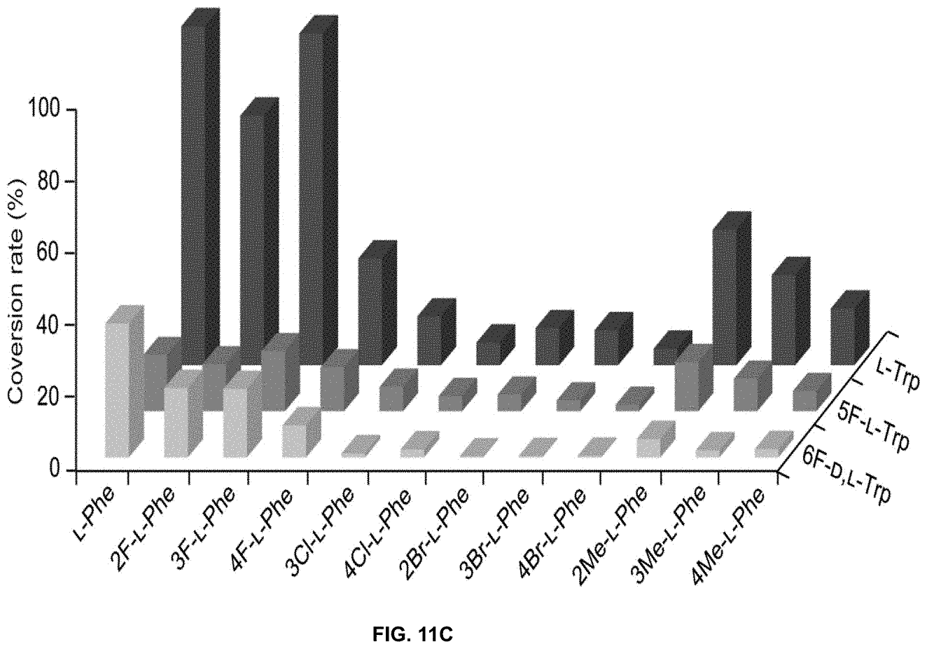

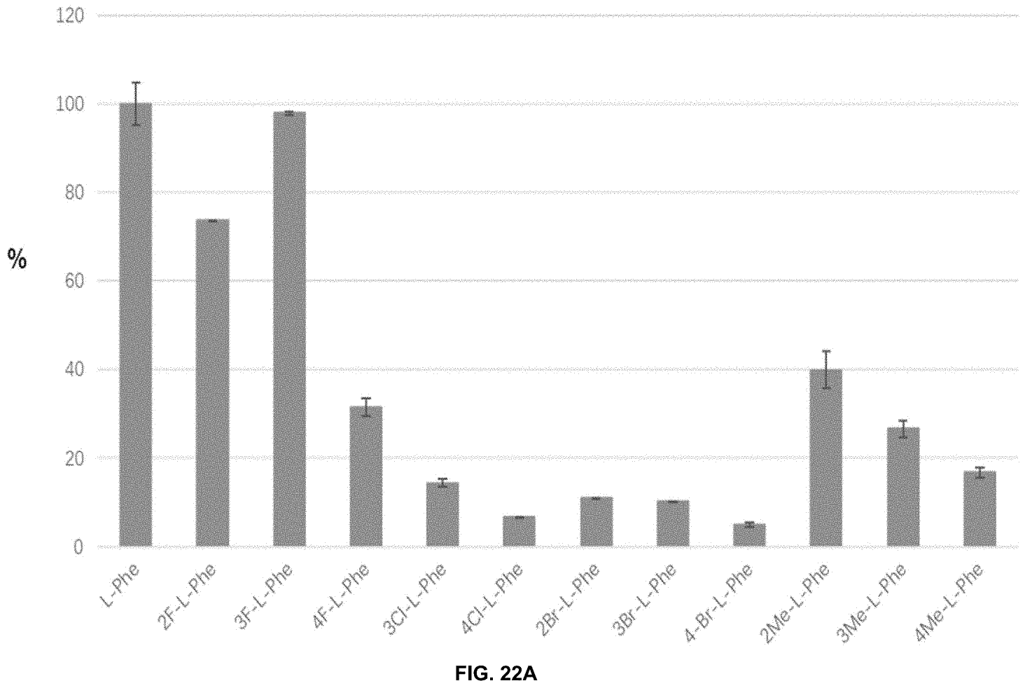

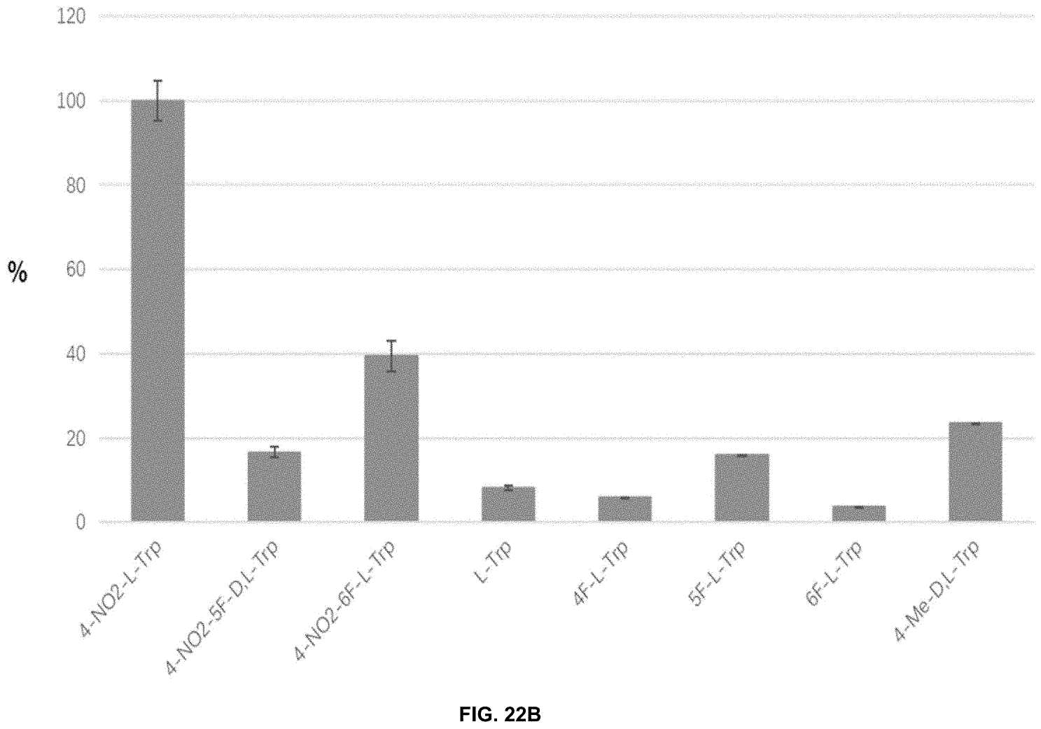

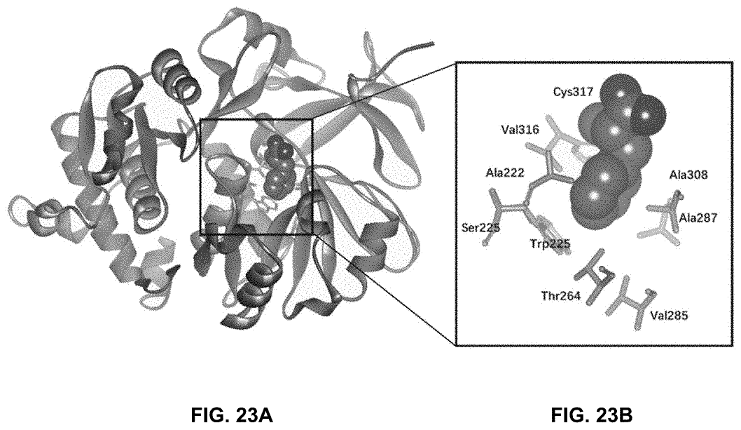

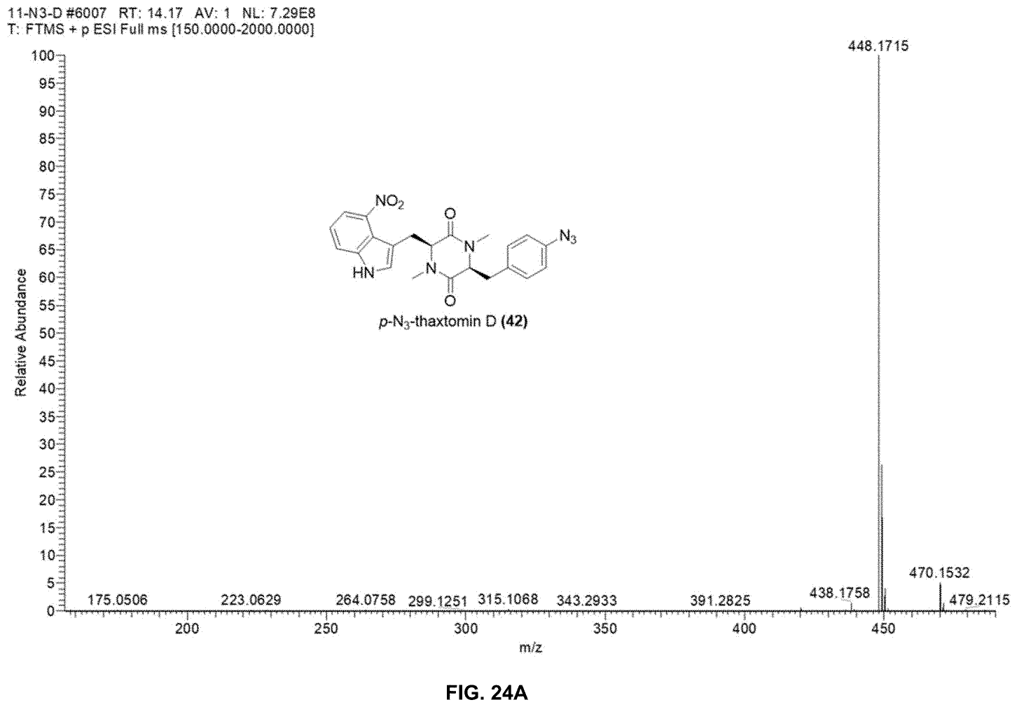

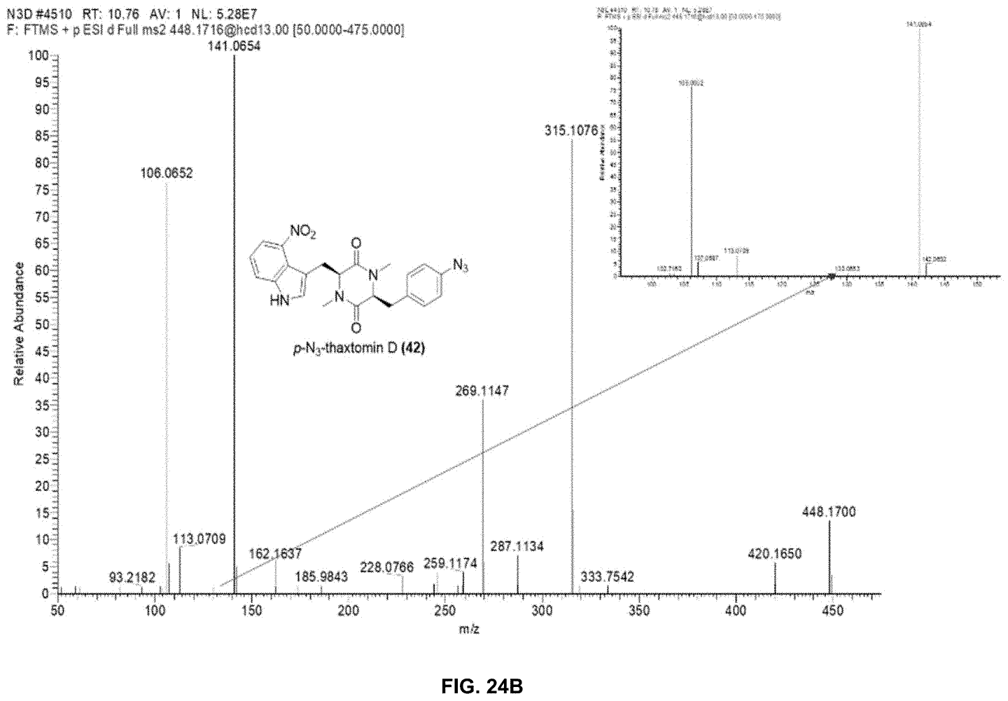

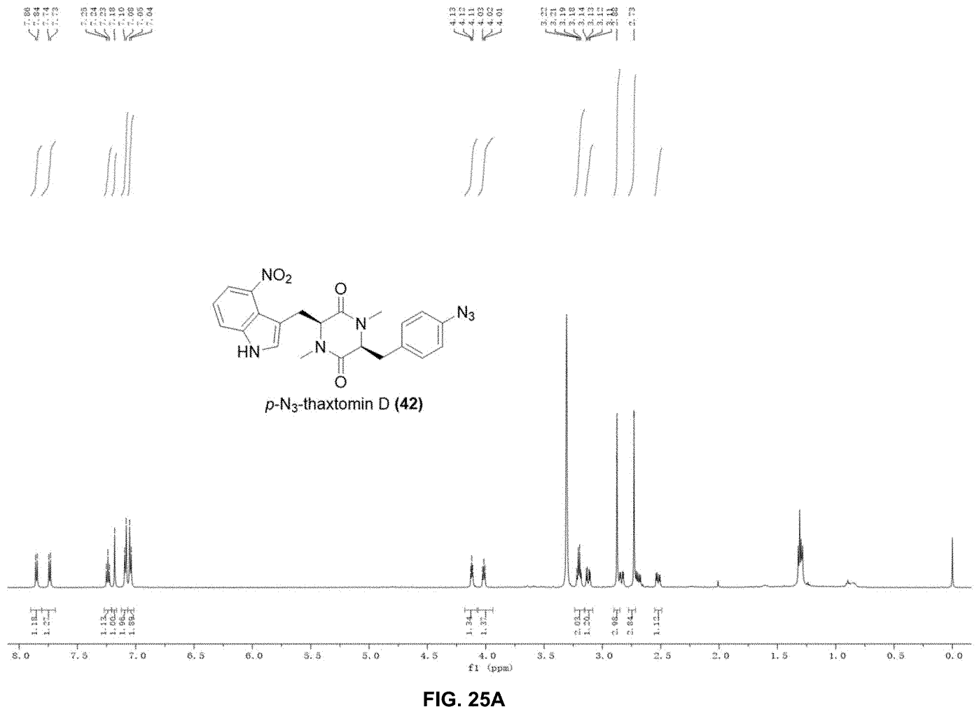

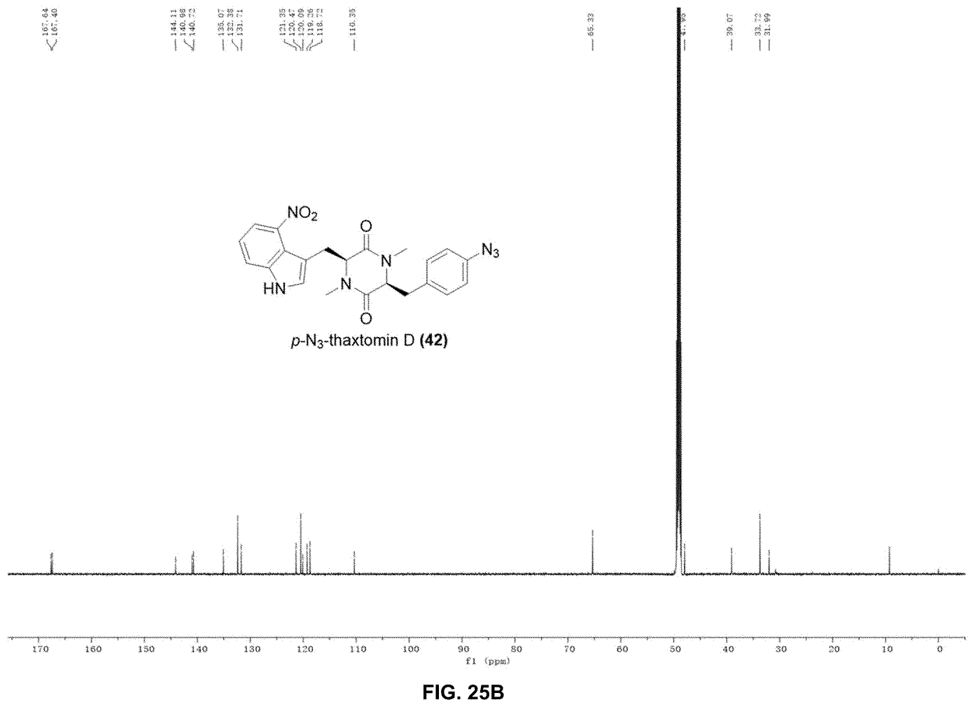

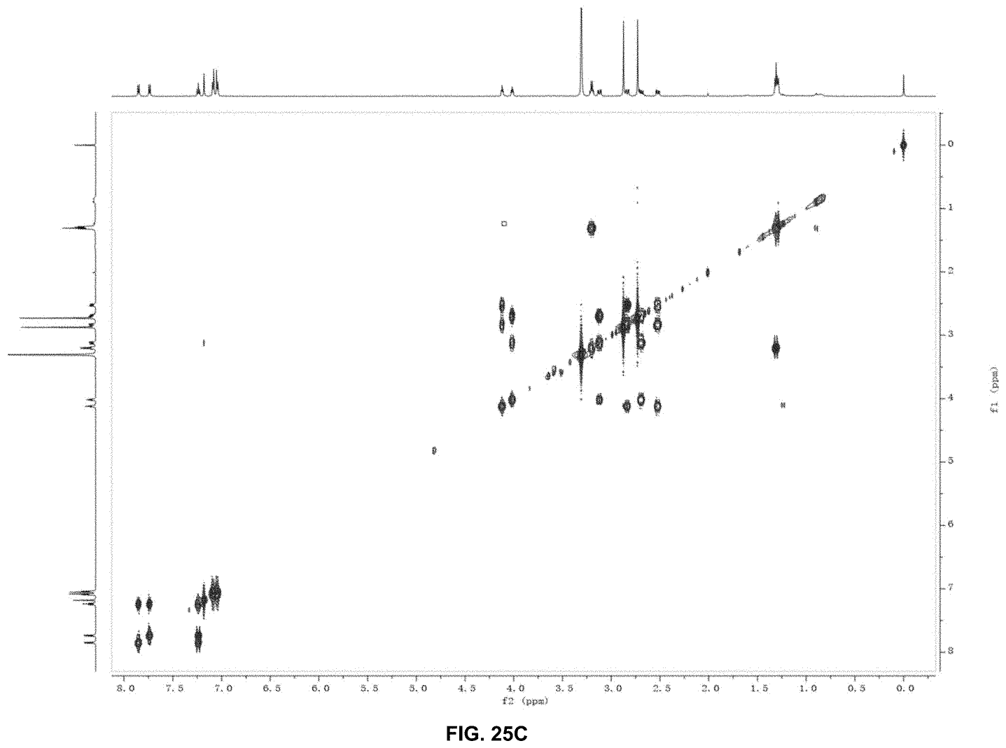

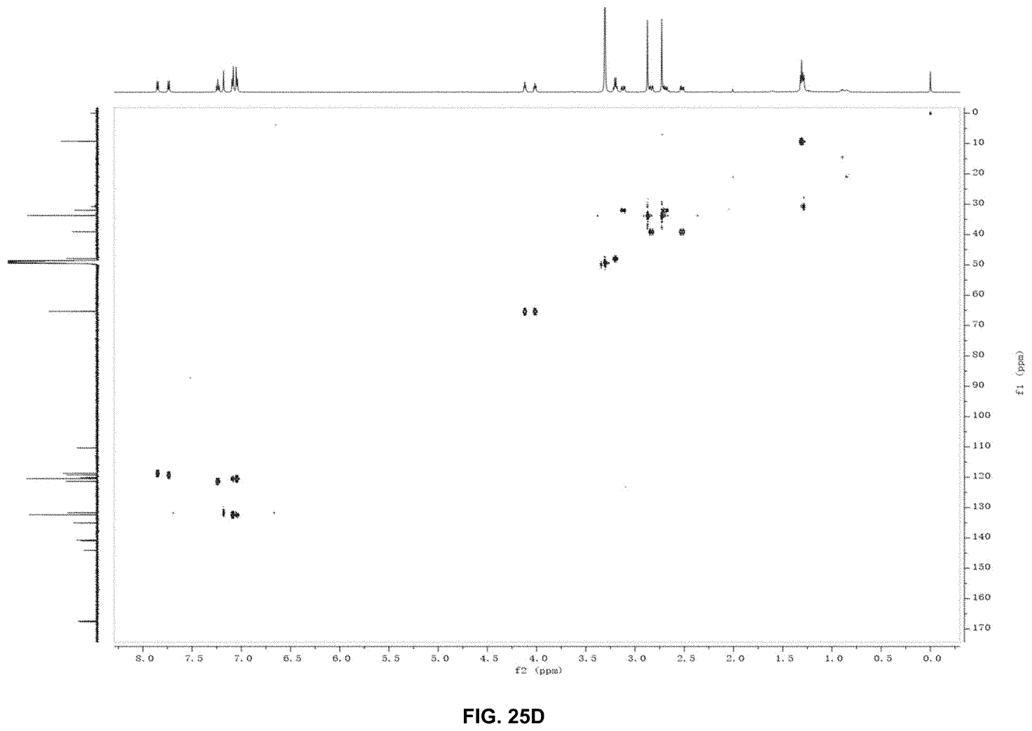

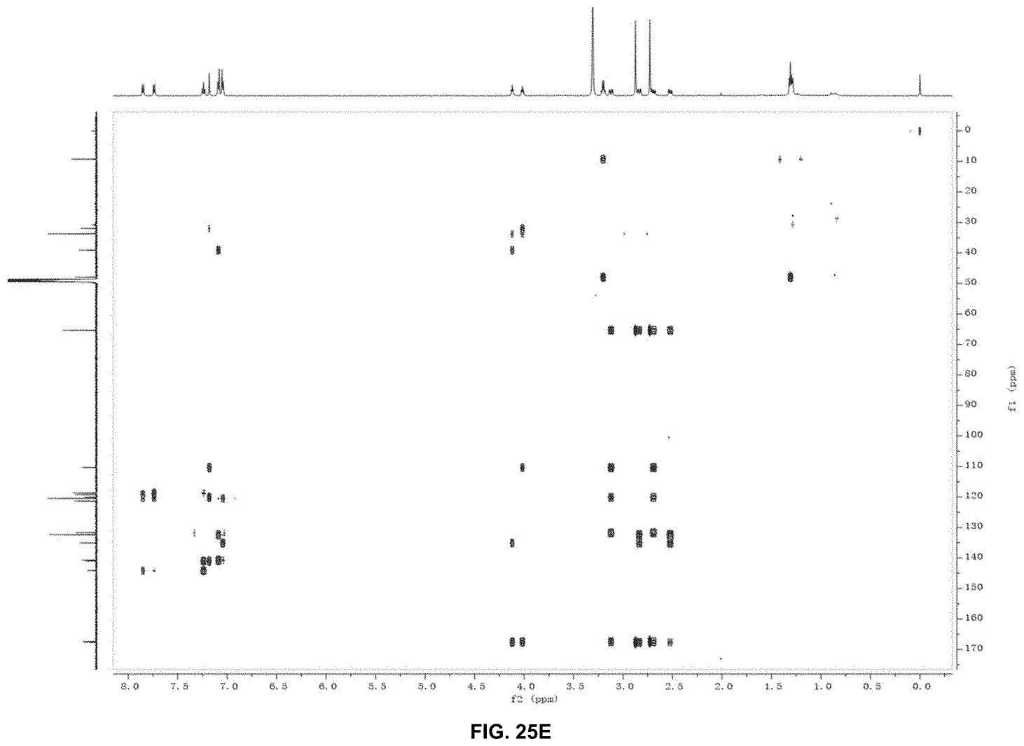

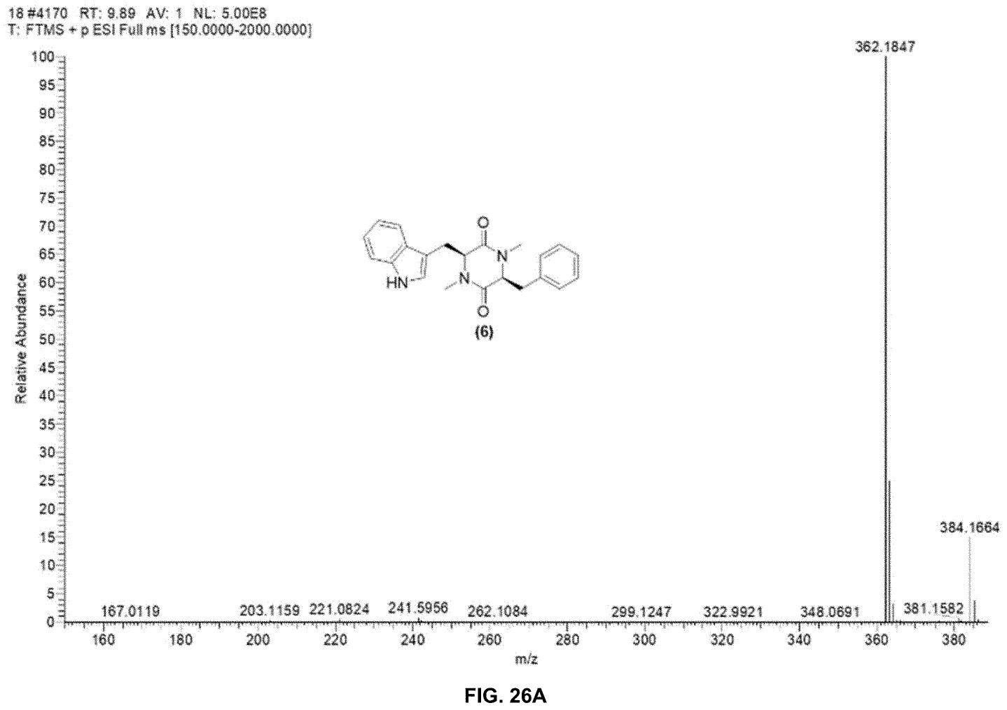

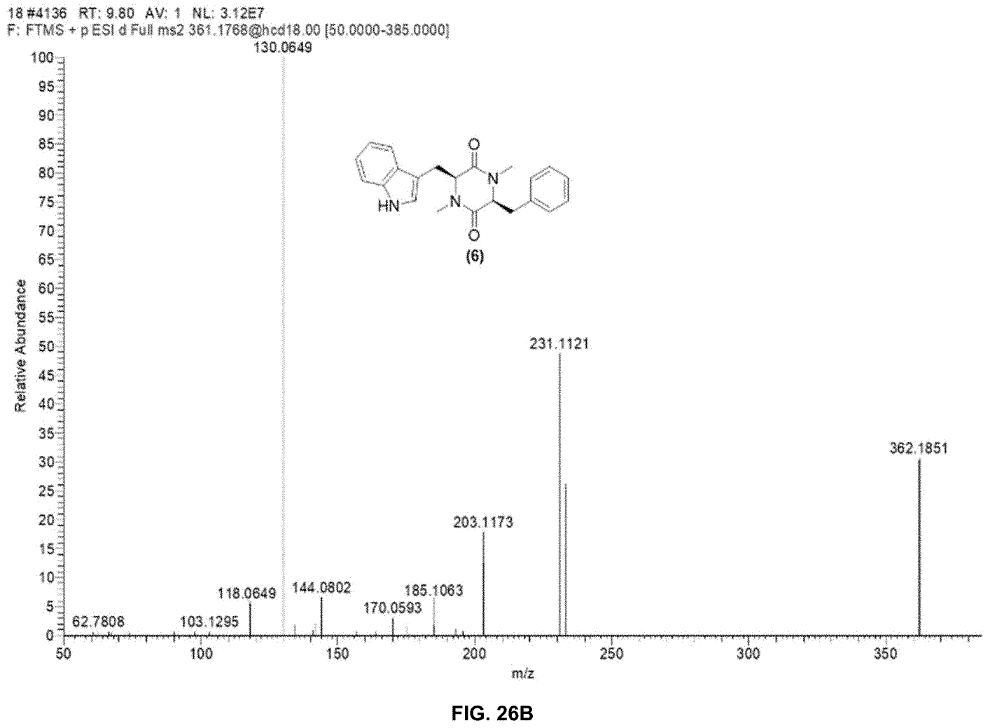

To probe the basis of chemical diversity of Thx DKP scaffold, next characterized was the substrate scopes of TxtA and TxtB. TxtA recognized only L-Phe among 20 natural amino acids to produce 4 but accepted 11 out of 17 L-Phe analogues with aryl F-, Cl-, Br- and Me-substituents to synthesize 4 analogues as revealed in HRMS and MS/MS analysis (FIG. 22A; Table 7). 3F-L-Phe was equally active to L-Phe in the reaction, followed by 2F-L-Phe(˜73% of L-Phe) (FIG. 22A). Bulkier aryl substituents decreased enzyme activity. For example, 3Me-L-Phe retained about 27% relative activity of L-Phe while TxtA did not accept p-azido-L-Phe for the reaction (FIG. 22A; Table 8). From the modelled structure of TxtA adenylation (A) domain (FIG. 23), it was identified that Trp225 likely controls the binding of bulky L-Phe analogues. Indeed, the TxtA W225S mutant [Kries, H. et al. Angew. Chem. Int. Ed. Engl. 2014 53:10105-10108] accepted p-azido-L-Phe and showed a strong preference over L-Phe in the reaction (FIG. 11A). We confirmed the product as p-azido-Thx D by HRMS, MS/MS, 1D and 2D NMR analysis (FIGS. 24-25). The substrate preference of TxtB was then assessed. In the reaction without TB14, TxtB accepted only L-Trp among 20 natural amino acids to synthesize desnitro Thx D as confirmed in HRMS and MS/MS analysis (FIG. 26), but was 12 times less active than 4-NO2-L-Trp. Additionally, TxtB utilized 4F-L-, 5F-L-, 4Me-D,L- and 6F-D,L-Trp out of 15 L-Trp analogues carrying various substituents (Table 7) as substrates to synthesize desnitro dimethylated DKP analogues (FIG. 22). 5F-L-Trp and 4Me-D,L-Trp were over 2 times more active than L-Trp. The tolerance to small modifications on L-Trp indole by TxtB agreed with a recent in vivo study by the Micklefield group [Winn, M. et al. Angew. Chem. Int. Ed. Engl. 2018 57:6830-6833]. These results further revealed the obvious substrate tolerance of MT domains [Mori, S. et al. Nat. Chem. Biol. 2018 14: 428-430].

TABLE 7
Chemicals used to probe the substrate scope of TxtA and TxtB.

| | | | | |
|---|---|---|---|---|
| | | TxtA | TxtAW225S | TxtB |
| Chemicals | Abbreviation | substrate | substrate | substrate |

L-tryptophan analogues

| | | |
|---|---|---|
| 4-nitro-L-tryptophan | 4-nitro-L-Trp | +++++ |
| L-tryptophan | L-Trp | + |
| D-tryptophan | D-Trp | |
| α-Me-L-tryptophan | α-Me-L-Trp | |
| 5OH-L-tryptophan | 5OH-L-Trp | |
| 5OMe-L-tryptophan | 5OM-L-Trp | |
| 7-Aza-L-tryptophan | 7-Aza-L-Trp | |
| 4F-L-tryptophan | 4F-L-Trp | + |
| 5F-L-tryptophan | 5F-L-Trp | ++ |
| 6F-D,L-tryptophan | 6F-D,L-Trp | + |
| 4Me-D,L-tryptophan | 4Me-D,L-Trp | +++ |
| 5Me-D,L-tryptophan | 5Me-D,L-Trp | |
| 6Me-D,L-tryptophan | 6Me-D,L-Trp | |
| 7Me-D,L-tryptophan | 7Me-D,L-Trp | |
| 5Cl-L-tryptophan | 5Cl-L-Trp | |
| 5Br-L-tryptophan | 5Br-L-Trp | |
| 1-Me-L-tryptophan | 1Me-L-Trp | |

L-phenylalanine analogues

| | | | |
|---|---|---|---|
| L-phenylalanine | L-Phe | +++++ | + |
| 2F-L-phenylalanine | 2F-L-Phe | ++++ | |
| 3F-L-phenylalanine | 3F-L-Phe | +++++ | |
| 4F-L-phenylalanine | 4F-L-Phe | ++ | |
| 3Cl-L-phenylalanine | 3Cl-L-Phe | + | |
| 4Cl-L-phenylalanine | 4Cl-L-Phe | + | |
| 2Br-L-phenylalanine | 2Br-L-Phe | + | |
| 3Br-L-phenylalanine | 3Br-L-Phe | + | |
| 4Br-L-phenylalanine | 4Br-L-Phe | + | |
| 2Me-L-phenylalanine | 2Me-L-Phe | ++ | |
| 3Me-L-phenylalanine | 3Me-L-Phe | ++ | |
| 4Me-L-phenylalanine | 4Me-L-Phe | + | |
| 3OMe-phenylalanine | 3OMe-L-Phe | | + |
| α-Me-L-phenylalanine | α-Me-L-Phe | | |
| 3-(4-Pyridyl)-alanine | Pyri-L-Phe | | |
| p-N3-L-phenylalanine | 4-N3-L-Phe | | ++ |
| 4-NH2-L-phenylalanine | 4-NH2-L-Phe | | |
| 4-NO2-L-phenylalanine | 4-NO2-L-Phe | | |

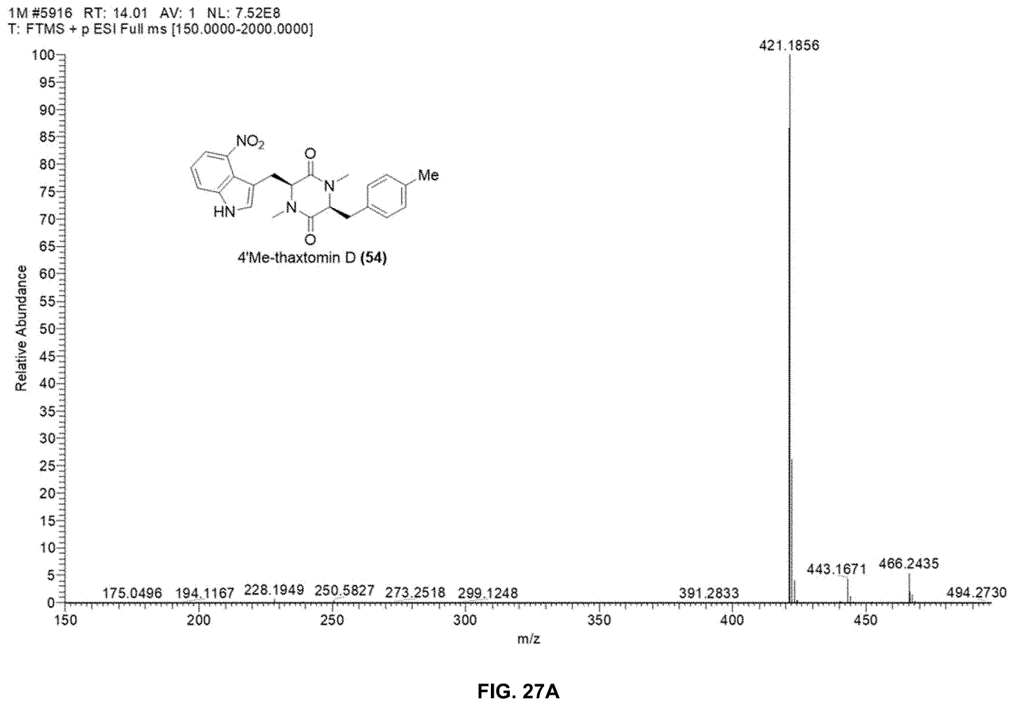

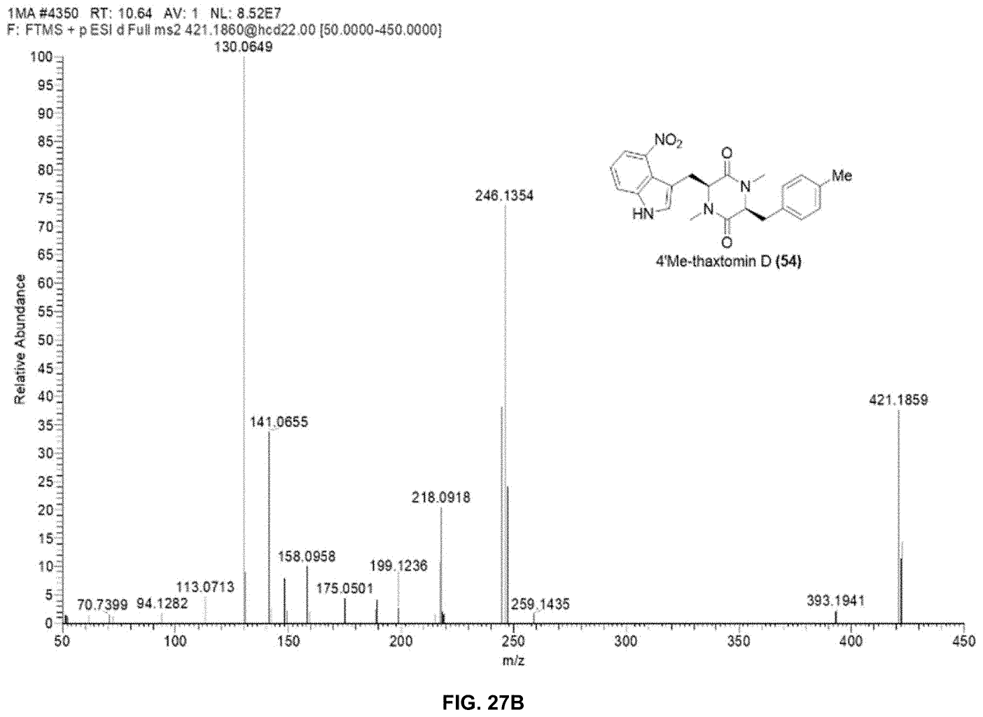

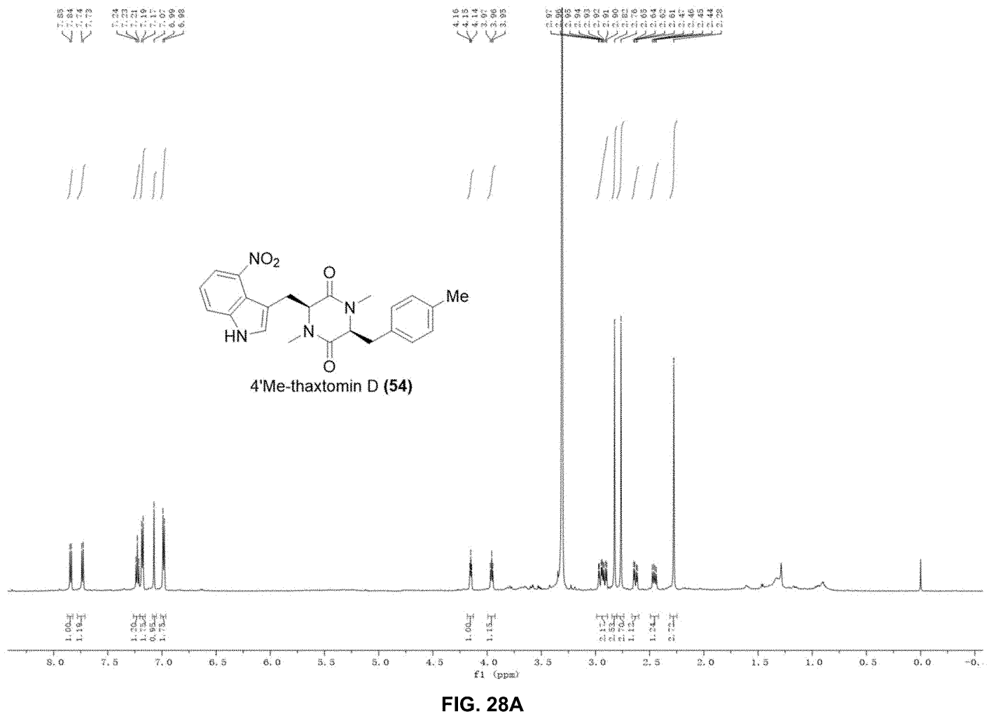

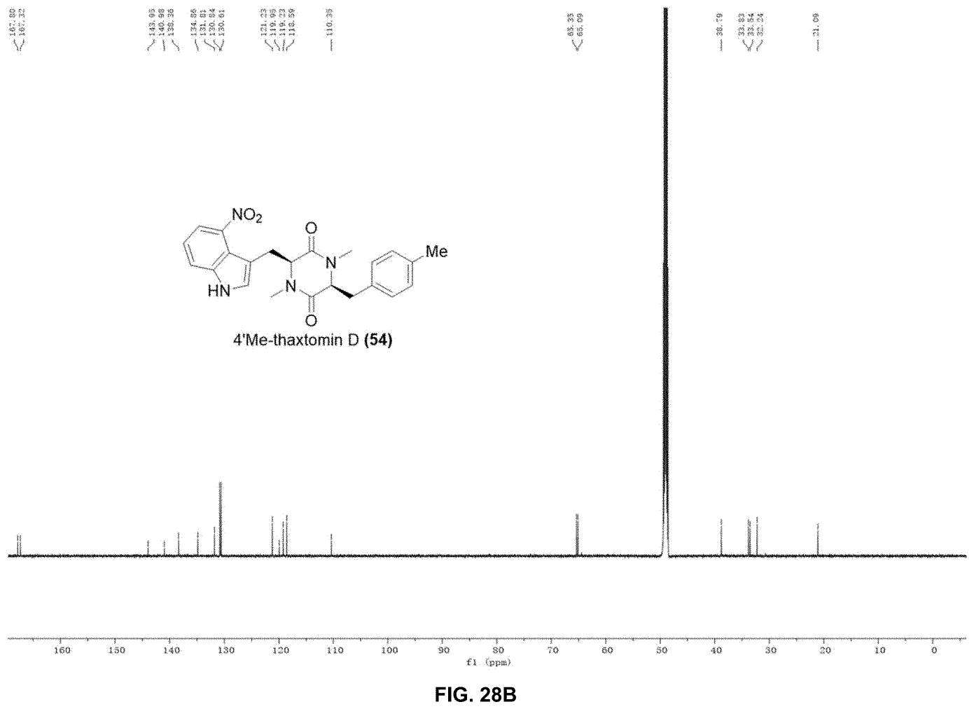

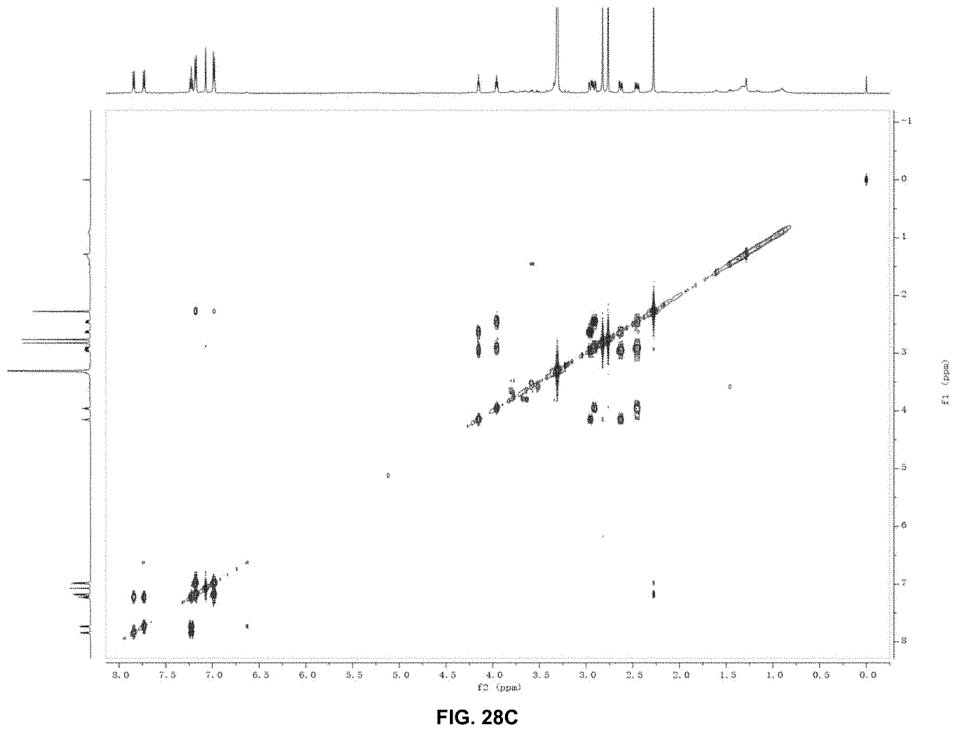

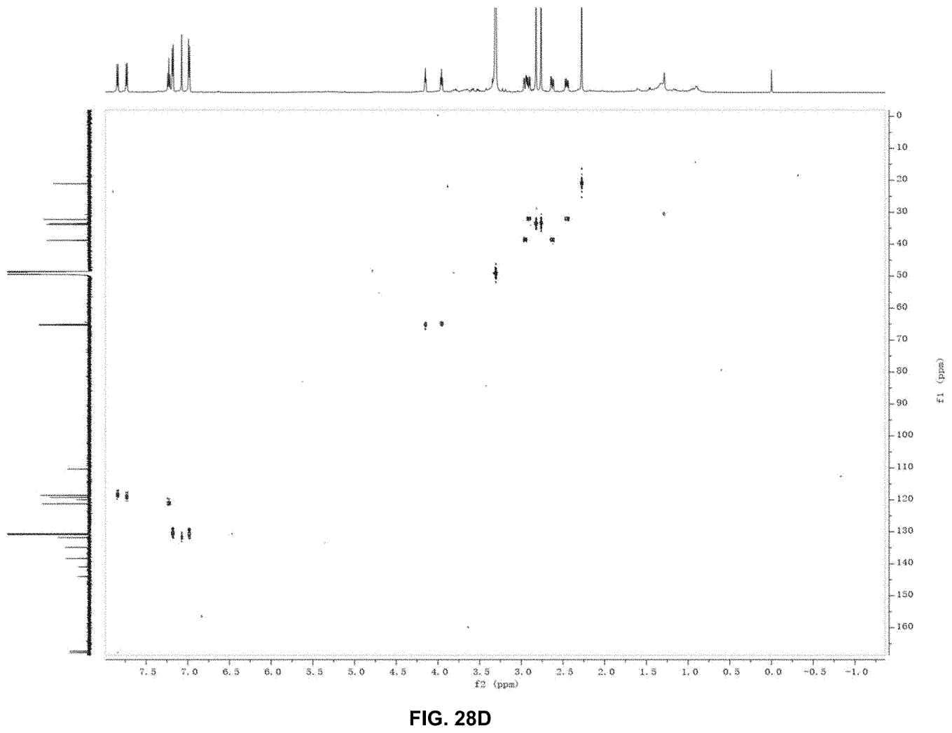

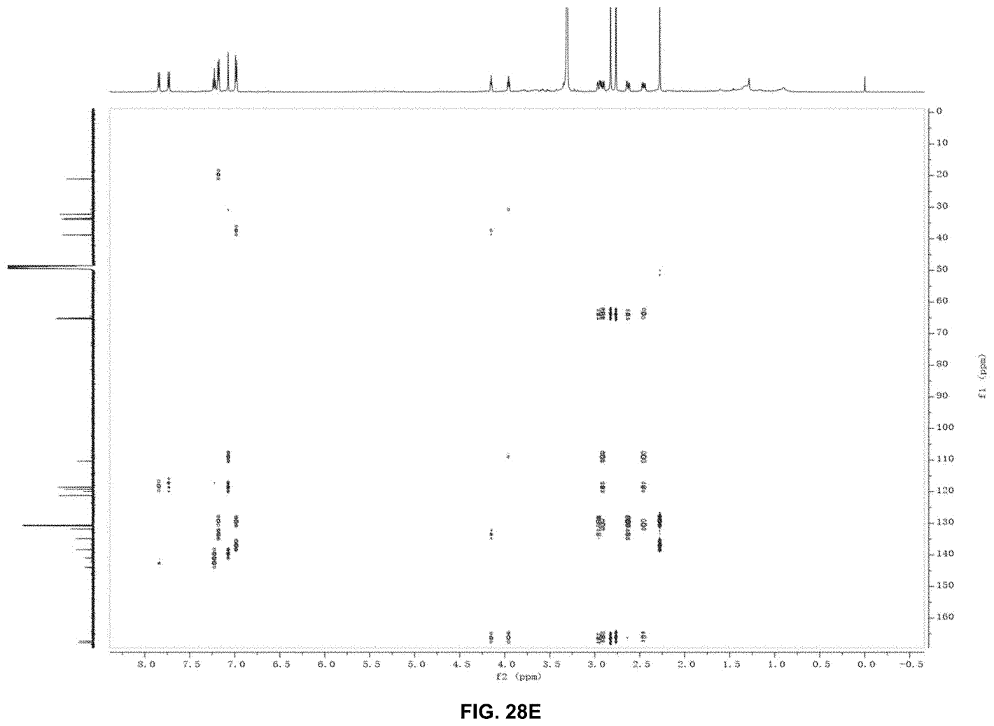

Next examined was the potential interplay of TxtA and TxtB in defining the chemical diversity of the Thx scaffold. The biocombinatorial TxtA-TxtB reactions produced 30 out of 60 potential desnitro Thx D analogues using 12 L-Phe and 5 L-Trp analogues as the substrates (FIG. 11B; Table 8). The overall conversion rates of amino acids into the DKPs ranged from 22% to 0.7%. TxtA was unable to utilize six brominated or methylated L-Phe analogues in the reactions, while the substrate scope of TxtB was unchanged and 5F-L-Trp and 4Me-D,L-Trp tended to give higher conversion rates. This result indicated that TxtB primarily controls DKP diversity generation. Next, TB14 was included in the above biocombinatorial reactions to assess its influence on the generation of DKP diversity. Importantly, the nitro group is critical to the herbicidal activity of Thx analogues [Zhang, H. et al. J. Agric. Food. Chem. 2015 63:3734-3741]. Formate dehydrogenase (FDH) [Seelbach, K. et al. Tetrahedron Lett. 1996 37:1377-1380] was included in these reactions to recycle NADPH, which gave rise to an overall conversion rate of 4 at 94%. From L-Trp, 5F-L- and 6F-D,L-Trp and 12 L-Phe analogues, 36 Thx D analogues were successfully produced with the conversion rates ranging from 94% (4) to 0.3% (FIG. 11C; Table 9). As a representative example, the structure of 4′Me-Thx D (0.32 mg) was validated by a combination of MS and NMR analysis (FIGS. 27-28). Of note, TxtA accepted all 12 L-Phe analogues when nitro-L-Trp analogues were generated in the reactions (FIGS. 11B-C), demonstrating that TxtE is another important control of Thx molecular diversity. On the other hand, TxtB did not accept the nitro products generated from 4F- and 4Me-Trp by TB14 [Zuo, R. et al. Biotechnol. J. 2016 11:624-632; Zuo, R. et al. Sci. Rep. 2017 7:842]. These results together indicated that TxtB provides the primary control of DKP chemical diversity, followed by TxtE, a hierarchical model that may be followed by the biosynthesis of many other secondary metabolites [Fischbach, M. A. et al. Nat. Chem. Biol. 2007 3:353-355; Tianero, M. D. et al. Proc. Natl. Acad. Sci. USA 2016 113:1772-1777].

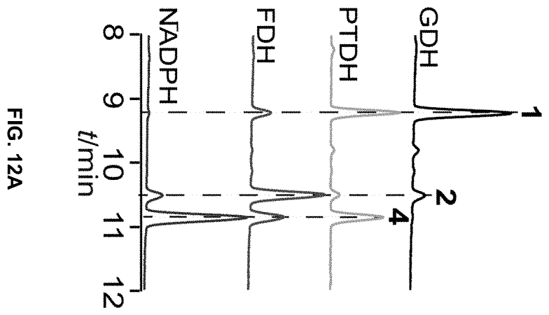

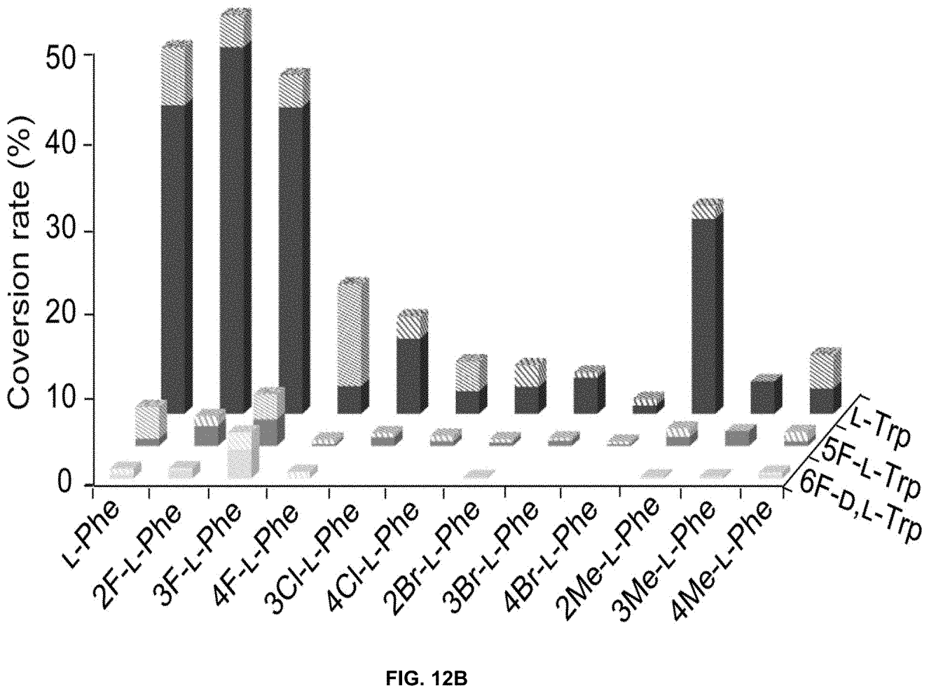

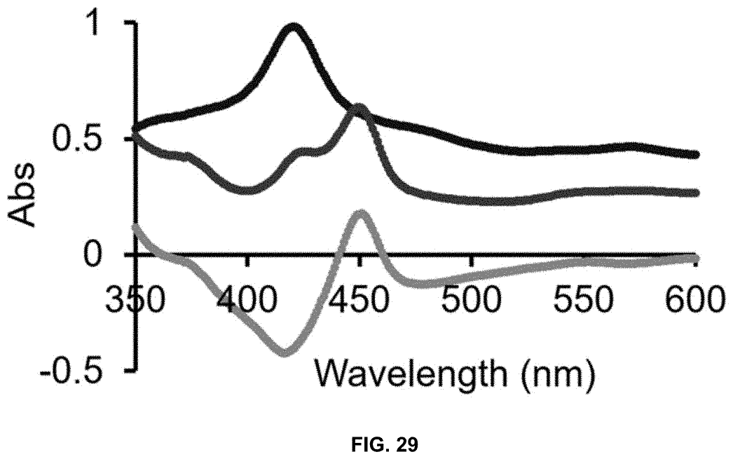

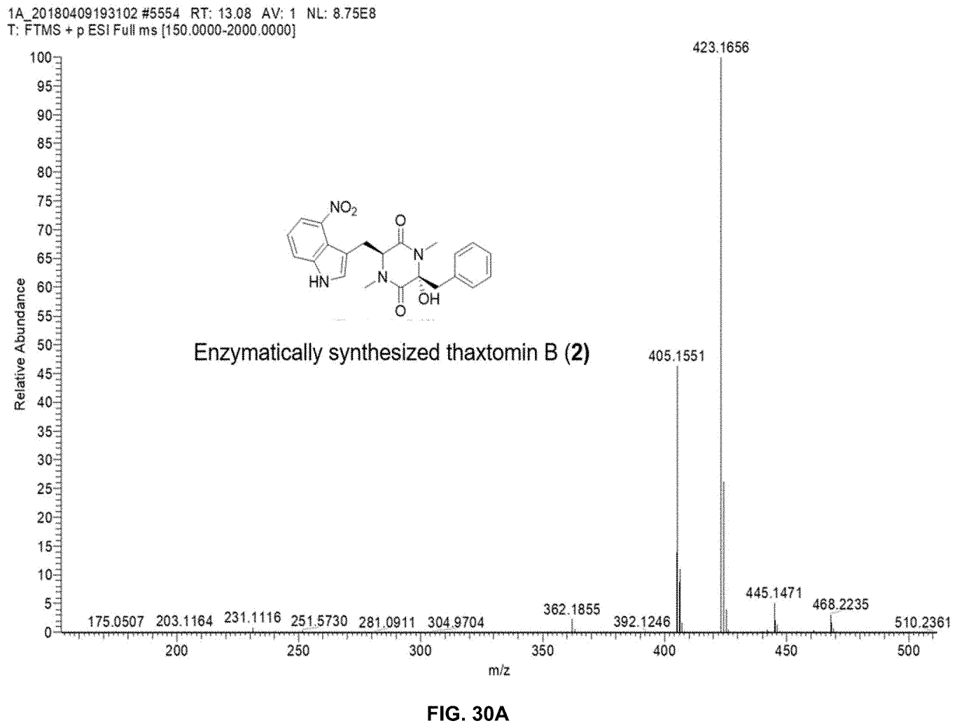

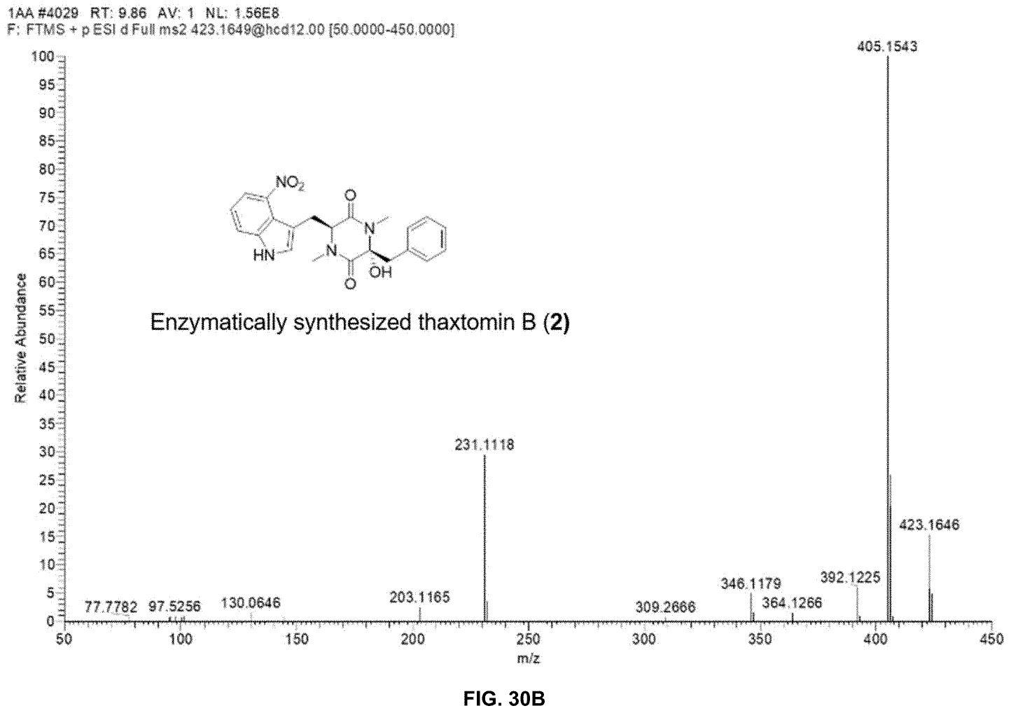

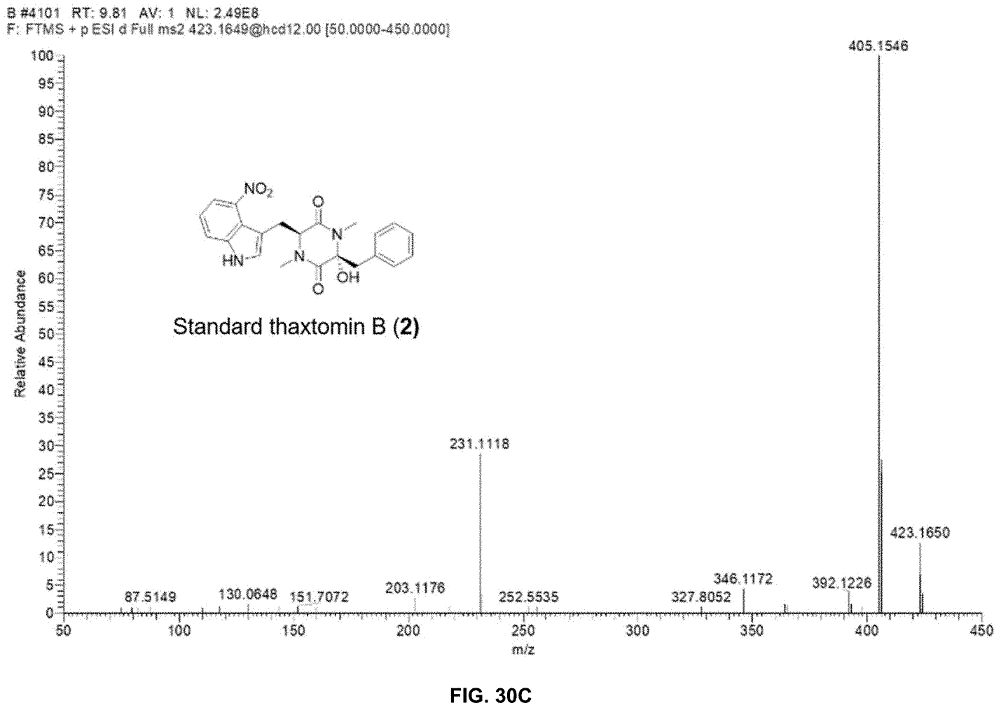

TxtC is predicted to catalyse the last two steps of Thx biosynthesis by sequentially hydroxylating 4 into 2 and then 1 (FIG. 9B), but its catalytic function has not been characterized biochemically [Healy, F. G. et al. J. Bacteriol. 2002 184:2019-2029]. To this end, there was an attempt to recombinantly prepare TxtC in E. coli but yielded an inactive enzyme toward 4. Inspired by the successful construction of TB14 [Zuo, R. et al. Sci. Rep. 2017 7:842], a self-sufficient TxtC variant was then engineered by fusing its C-terminus with the N-terminus of the reductase domain of P450BM3 [Narhi, L. O. et al. J. Biol. Chem. 1986 261:7160-7169] through a 14-amino acid linker, named as TCB14. Recombinant TCB14 was soluble and brownish and exhibited a major Soret peak at 450 nm in its differential CO-reduced spectrum (FIG. 29), a diagnostic feature of properly folded P450s [Omura, T. et al. J. Biol. Chem. 1964 239:2370-2378]. Its catalytic function was next examined, and LC-MS analysis revealed one single product in the reaction containing 4 (FIG. 12A). The product was identified as thaxtomin B (2) by comparing its retention time, m/z and tandem MS fragmentation pattern with the standard (FIG. 30). However, only about 13% of 4 was converted and increasing NADPH concentration to 20 times of 4 led to only a trace amount of 1. To improve the catalytic efficiency of TCB14, three NADPH regeneration systems driven we screened by FDH, phosphite dehydrogenase (PTDH) [Johannes, T. W. et al. Biotech. Bioeng. 2007 96:18-26] and glucose dehydrogenase (GDH) [Wong, C. H. et al. J. Am. Chem. Soc. 1985 107:4028-4031] that all supply fresh NADPH for the TCB14 reaction. The GDH system supported the conversion of 94% of 4 into 90% 1 along with 4% 2 (FIG. 12A). By contrast, the FDH system led to the conversion of 60% of 4 with 2 as the major product, while the PTDH system was least effective (˜44%) but the major product (>75% of hydroxylated products) was 1. It remains unclear how these systems influence the overall activity and product distribution of TCB14. Nonetheless, this work provided the first biochemical evidence of TxtC's function and the order of its two hydroxylations. Functionalization of two types of C—H bonds by a single P450 enzyme is distinct with other multi-functional P450s [Geisler, K. et al. Proc. Natl. Acad. Sci. USA 2013 110:E3360-E3367; Anzai, Y. et al. Chem. Biol. 2008 15:950-959; Carlson, J. C. et al. Nat. Chem. 2011 3:628-633; Tokai, T. et al. Biochem. Biophys. Res. Commun. 2007 353:412-417].

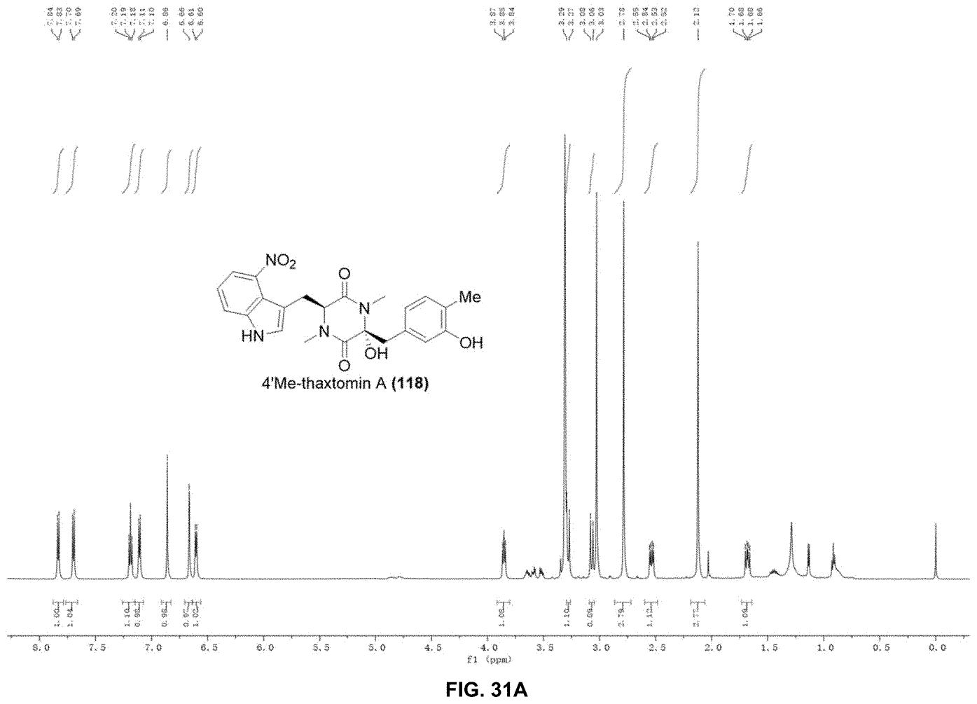

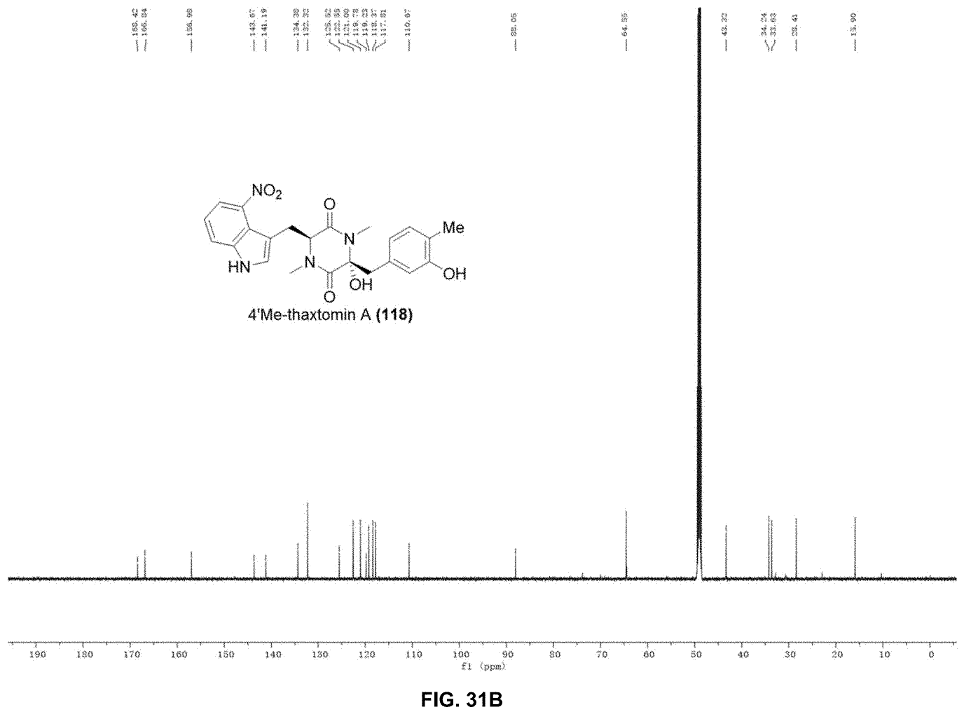

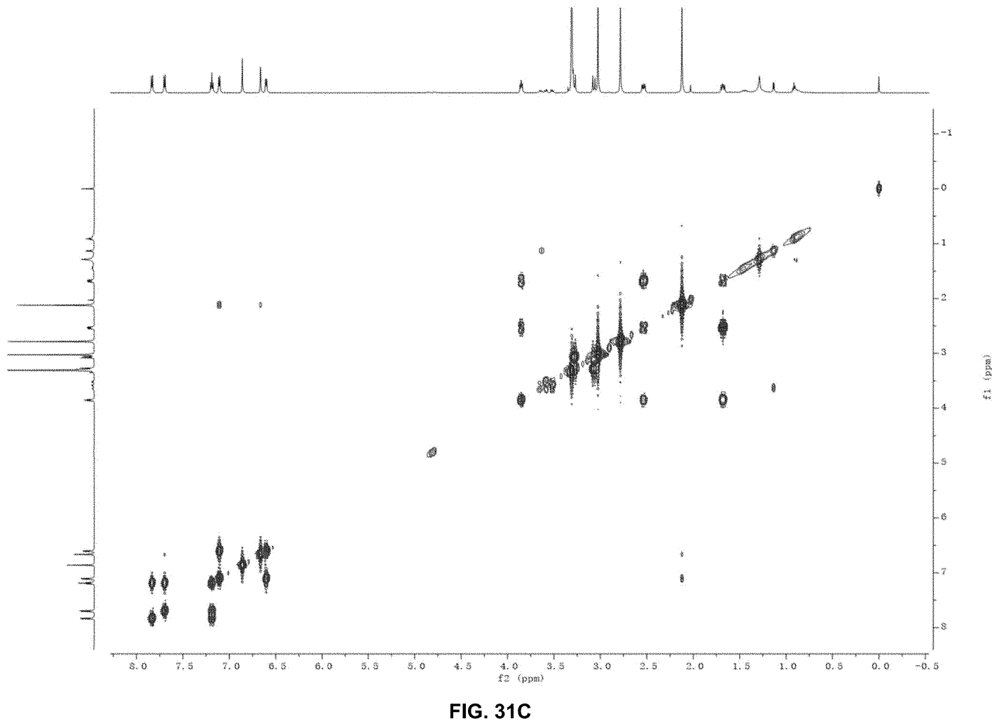

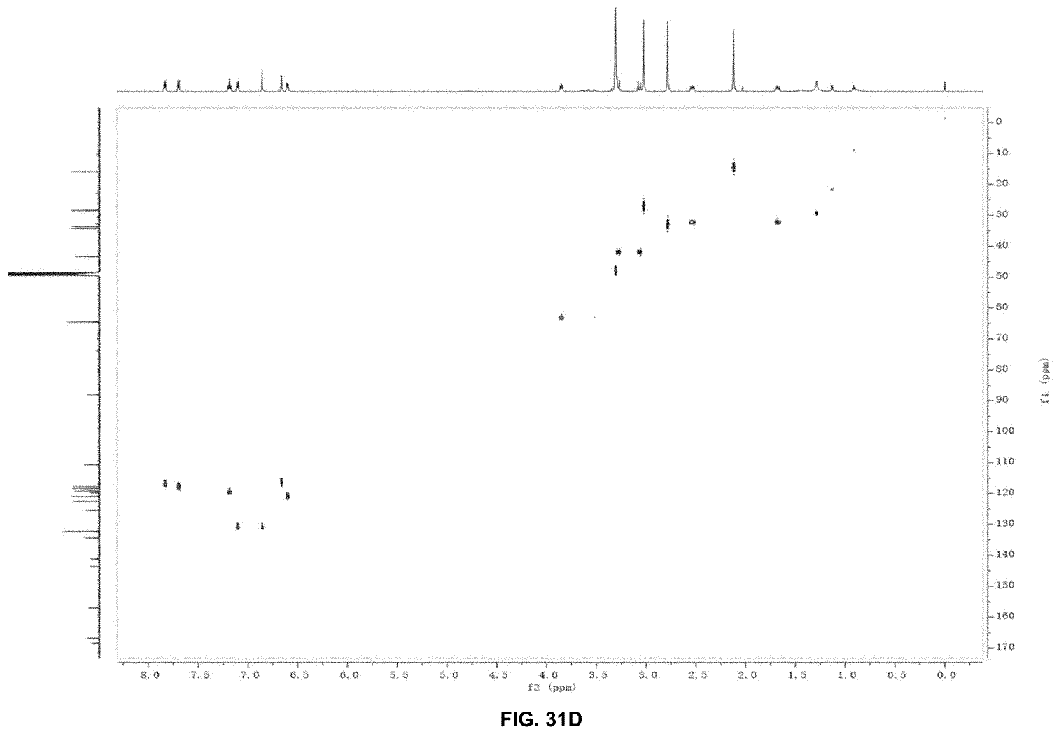

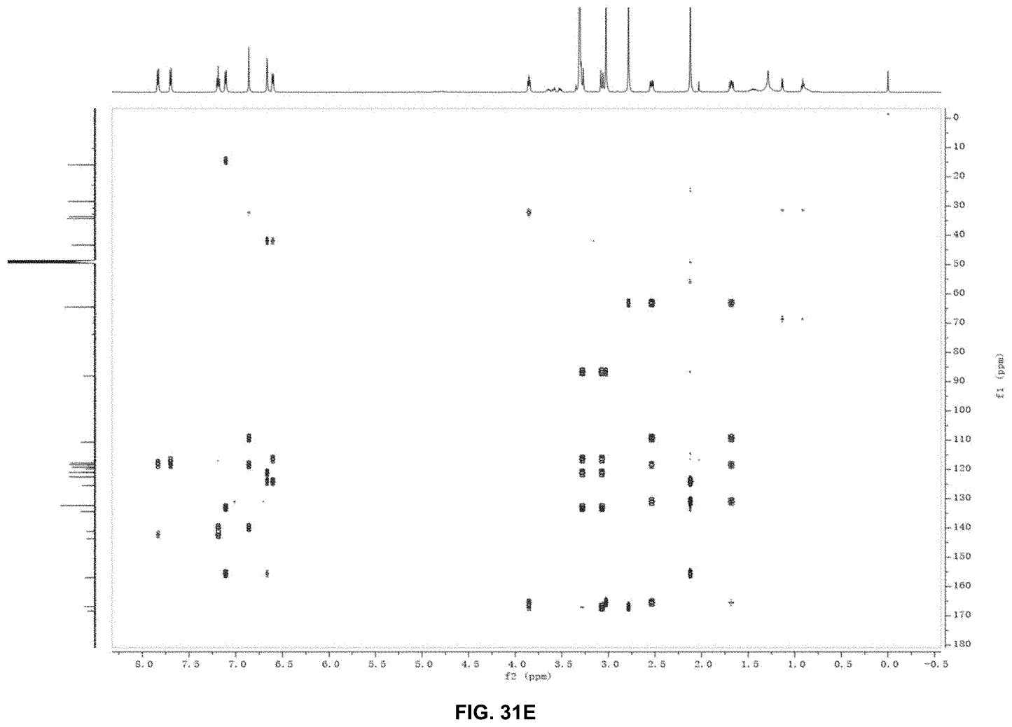

With TB14, TxtA, TxtB and TCB14 in hand, we attempted to fully reconstitute Thx biosynthesis in vitro. The one-pot four-enzyme reaction synthesized 2 but not 1 from L-Trp, L-Phe, NOC-5, SAM, and NADPH with a low overall conversation rate (<9%) even after screening three NADPH regeneration systems. One two-stage strategy was then adopted that separately produced 4 analogues with TB14, TxtA and TxtB and then hydroxylated products with TCB14 and the GDH system. As such, the overall conversation rates of 2 and 1 were 36% and 6.8%, respectively (FIG. 12B), representing the first cell-free total synthesis of Thxs. Employing this strategy, 58 out of 72 possible mono- and di-hydroxylated Thx analogues from L-Trp, 5F-L- and 6F-D,L-Trp and 12 L-Phe analogues were generated as confirmed in HPLC and HRMS analysis (Tables 10-11). Mono- and di-hydroxylated analogues in the reactions were well separated and quantitated in HPLC analysis to calculate the overall conversation rates ranging from 47% to 0.2% (FIG. 12B). As a representative example, the structure of 4′-Me-Thx A (4% conversion rate) was determined in 1D and 2D NMR analysis (FIG. 31). In the cell-free Thx biosynthesis, TCB14 showed low tolerance to substrates with 6F-Trp modification (FIG. 12B; Tables 10-11), and demonstrated different reaction selectivity toward varied structural variations of substrates. For example, 2′F-Thx D was mainly monohydroxylated but TCB14 strongly favoured 4′-F-Thx D for dihydroxylation (12.0% vs. 3.2%). Intriguingly, when the C3 of substrate L-Phe moiety is occupied by an F, Cl, or Br substitution, TCB14 was still able to produce di-hydroxylated products (Table 11), albeit the low abundance (0.3% to 3.8%). Nonetheless, these results suggested the altered, substrate-tuned regio-selectivity of TCB14, a unique catalytic plasticity shared with TxtE [Zuo, R. et al. Sci. Rep. 2017 7:842]. Collectively, this work provided a cell-free biocombinatorial approach to synthesize highly functionalized DKPs and suggested that the chemical diversity of Thxs is further controlled by late-stage tailoring modifications.

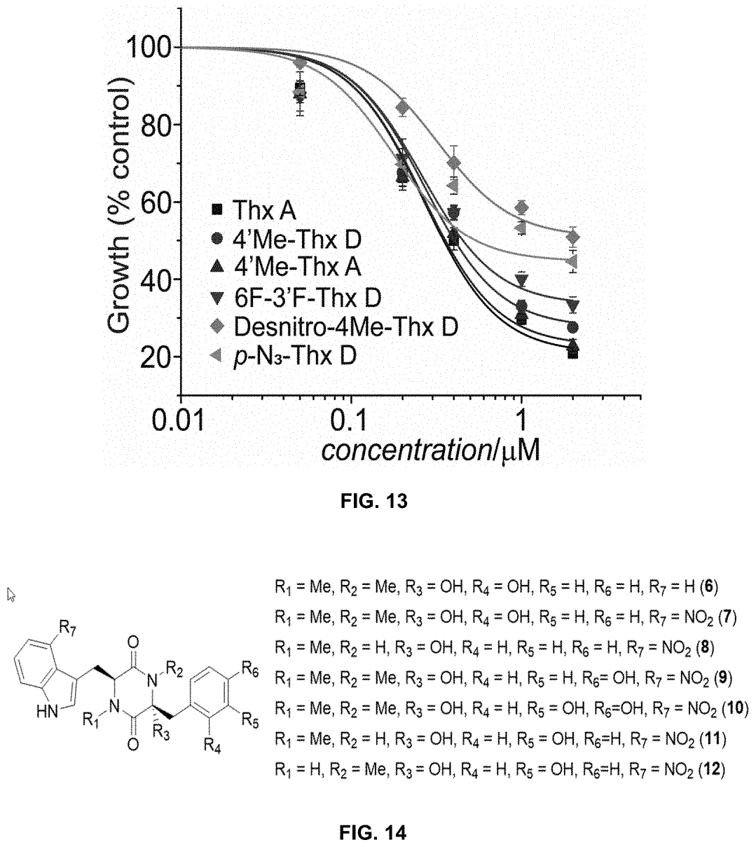

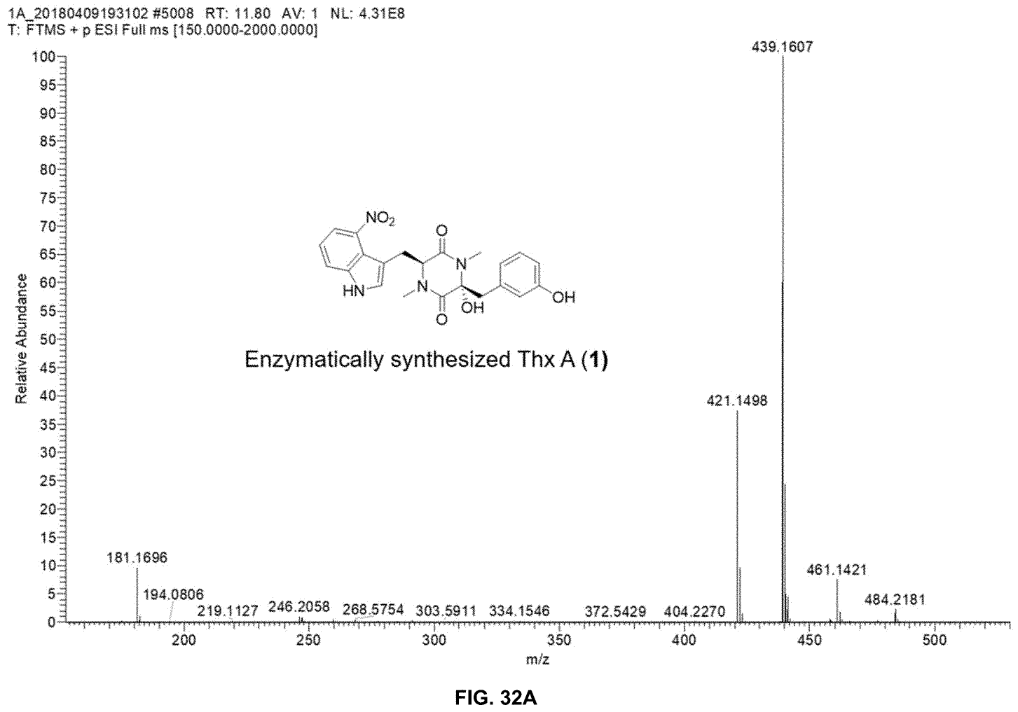

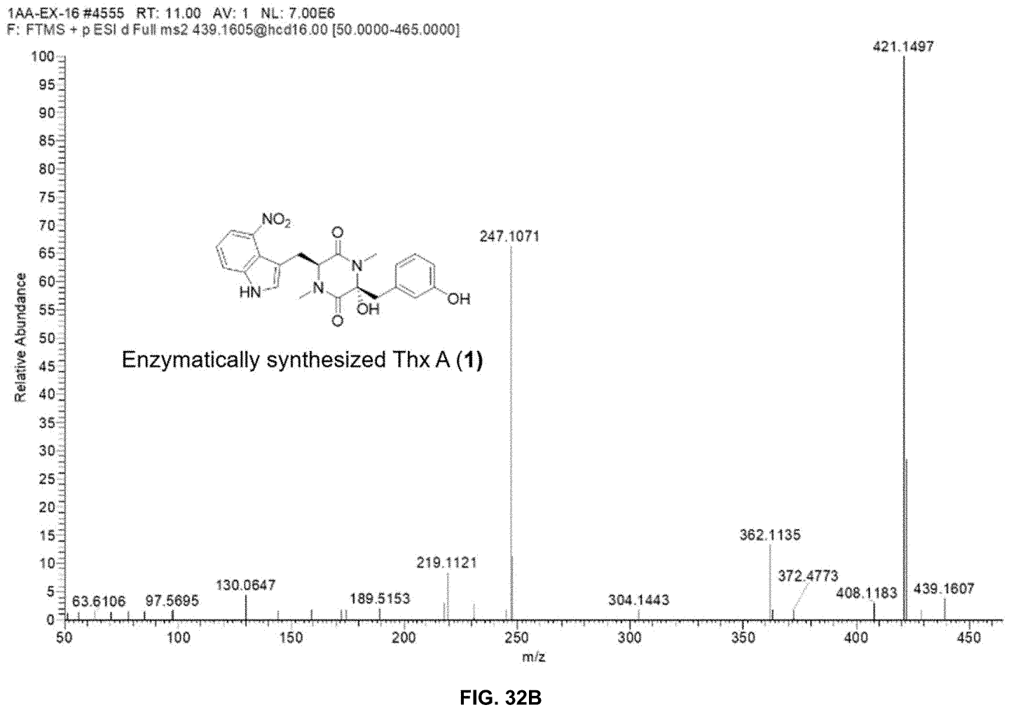

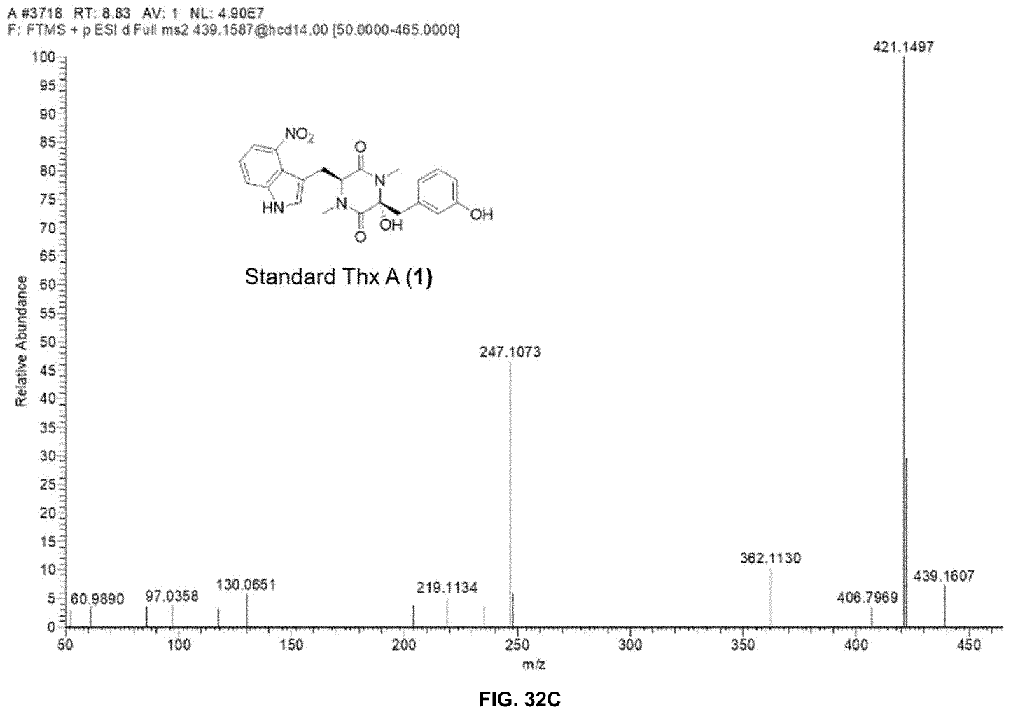

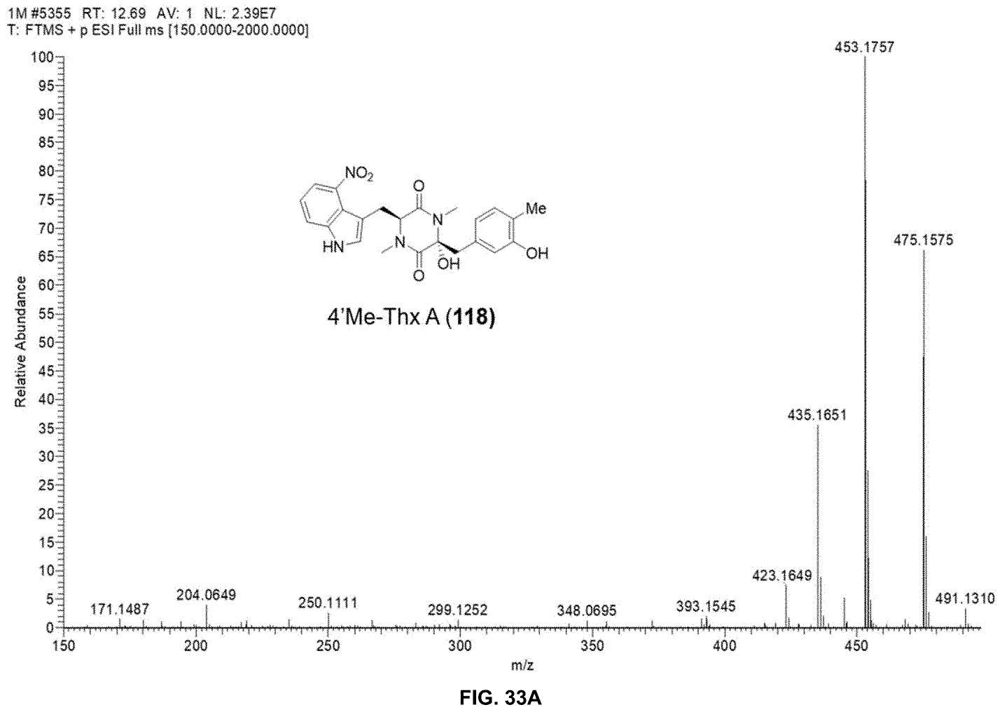

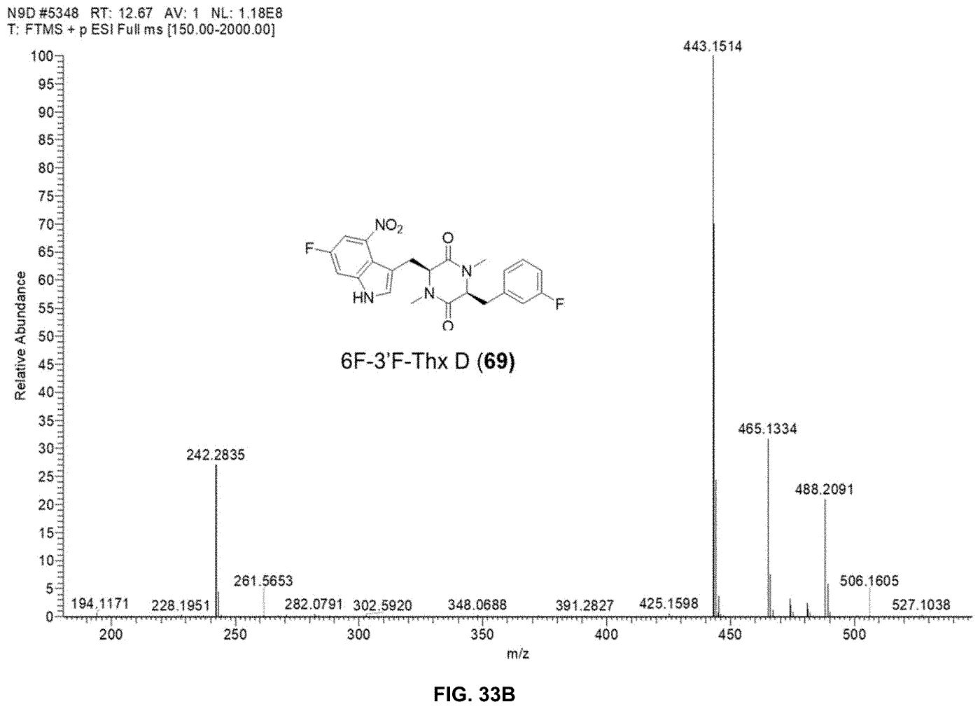

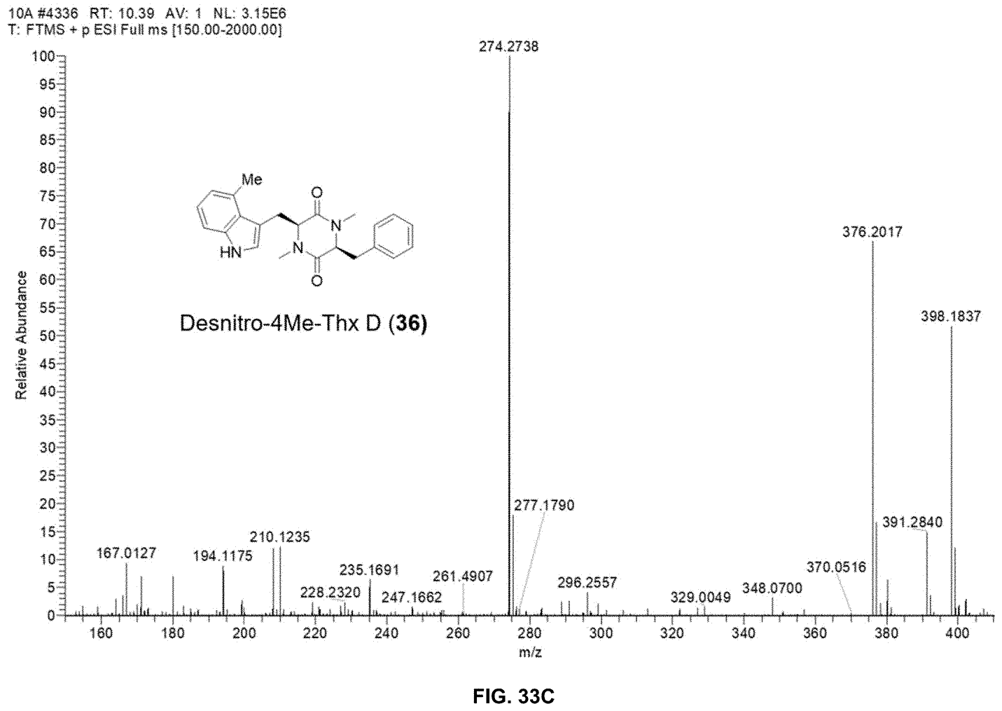

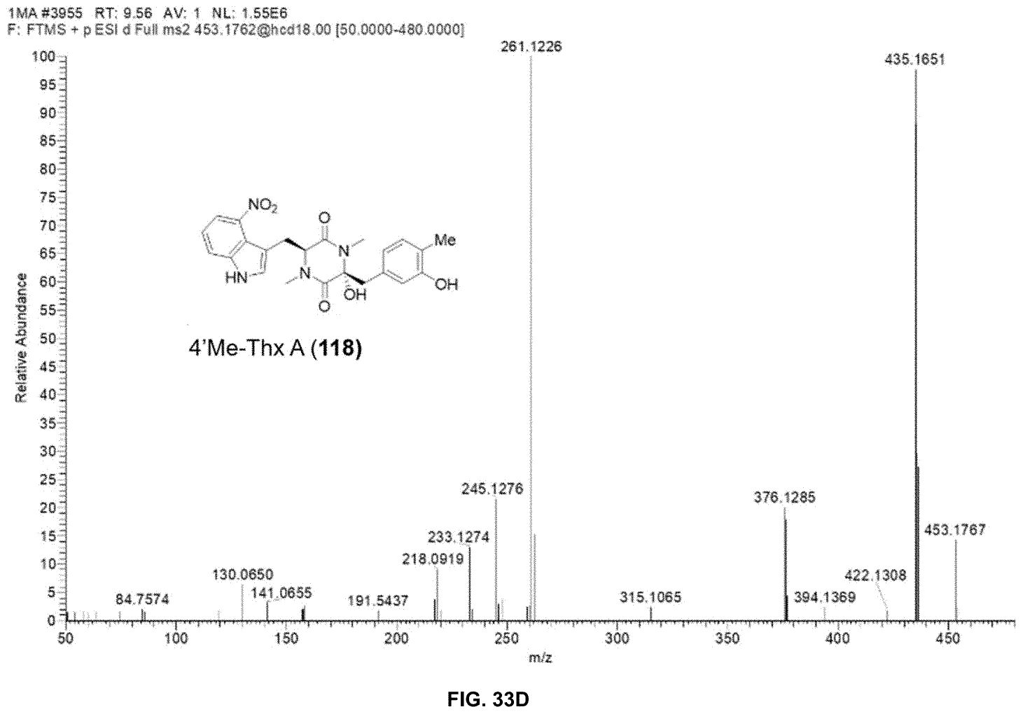

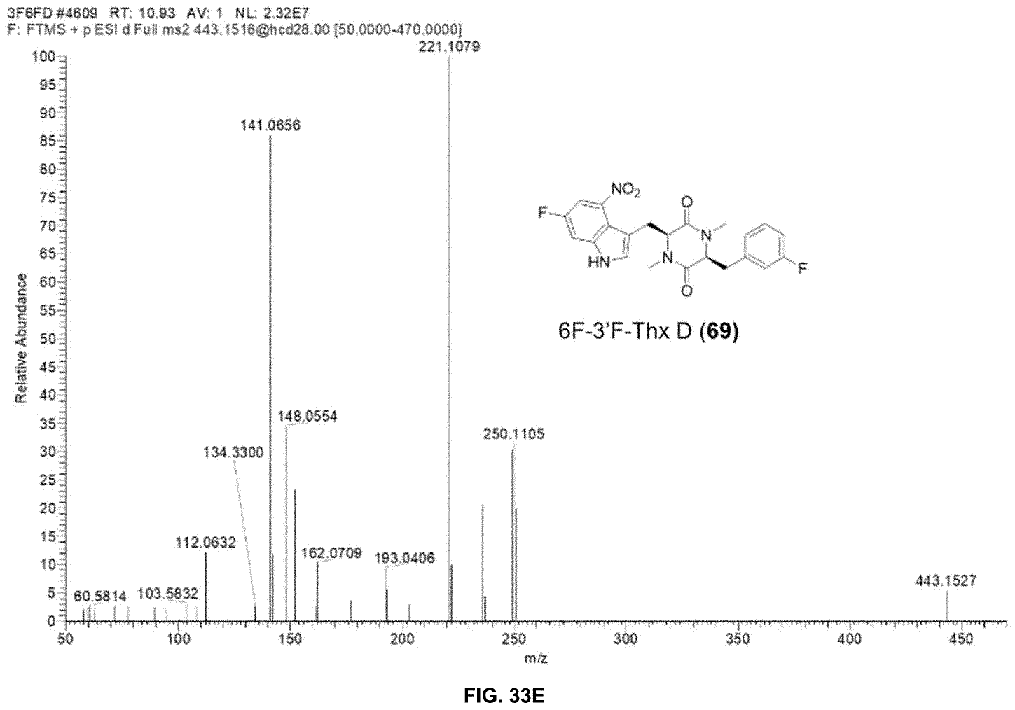

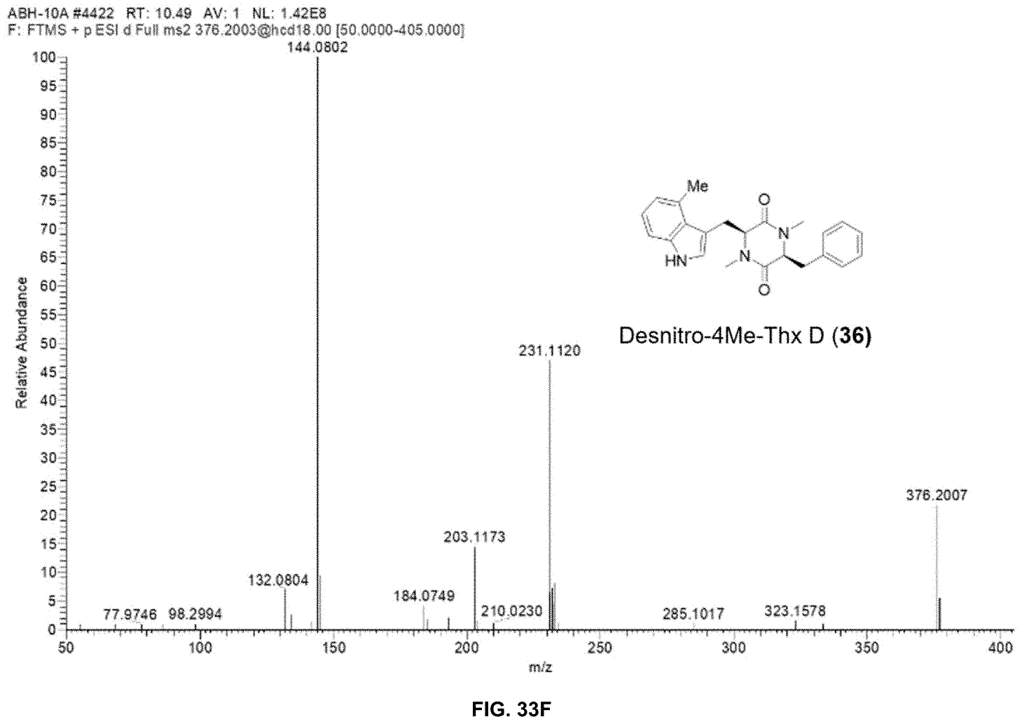

To examine the potential influences of structural variations on Thx herbicidal activity, 1, 4′Me-Thx A, 4′Me-Thx D, desnitro-4-Me-Thx D, 6F-3′F-Thx D and p-N3-Thx D were prepared at the milligram level (FIGS. 32-33). In radish seedling assay [Jiang, G. et al. Appl. Environ. Microbiol. 2018 84:e00164-18], the IC₅₀ of 4′Me-Thx A was the same as 1 (0.41±0.01 μM vs 0.39±0.03 μM, FIG. 13), followed by 4′Me-Thx D (0.48±0.04 μM) and 6F-3′F-Thx D (0.55±0.06 μM). The herbicidal potency of p-N3-Thx D (IC₅₀=1.29±0.21 μM) was modestly weaker than 1 (FIG. 13) but its azido group can be employed to further probe the molecular mechanism of Thx herbicidal activity [Scheible, W. R. et al. Plant Cell 2003 15:1781-1794; Habraken, W. J. et al. Nat. Commun. 2013 4:1507]. Remarkably, desnitro-4Me-Thx D retained a good herbicidal activity (IC50=1.98±0.02 μM), suggesting future exploration of other substituents at the C4 position.

In summary, the biosynthesis of Thxs has been fully reconstituted in vitro. Through detailed characterization of substrate selectivity of three Thx biosynthetic enzymes for the first time, this work suggested that chemical diversity of Thxs is controlled by biosynthetic enzymes in a hierarchical manner, a model that other pathways may follow [Tianero, M. D. et al. Proc. Natl. Acad. Sci. USA 2016 113:1772-1777]. Furthermore, the present work provided a cell-free synthetic biology approach to synthesize 124 structurally diverse Thx analogues directly from simple amino acid building blocks (FIG. 34). These unnatural analogues can be used to develop novel herbicides, obtain an advanced mechanistic understanding of Thx's action, and further diversify their structures using embedded functional groups [Kries, H. et al. Angew. Chem. Int. Ed. Engl. 2014 53:10105-10108; Wang, M. et al. ACS Catal. 2014 4:1219-1225; Durak, L. J. et al. ACS Catal. 2016 6:1451-1454]. In addition, it was envisioned that the diversity of unnatural Thxs can be further expanded with NRPS modules possessing different substrate preference and new tailoring enzymes in other NRP biosynthetic pathways [Chen, R. D. et al. Nat. Chem. Biol. 2017 13:226-234] and using protein engineering approaches.

Materials and General Methods. Molecular biology reagents and chemicals were purchased from Fisher Scientific, Sigma-Aldrich, or New England Biolabs, Inc if not specifically indicated. Primers were ordered from Sigma-Aldrich. 4-Me-D,L-tryptophan was from MP Biomedical (Santa Ana, Calif.), while NOC-5 (3-(Aminopropyl)-1-hydroxy-3-isopropyl-2-oxo-1-triazene) was purchased from EMD Millipore. Escherichia coli DH5a, BL21-GOLD (DE3) (Agilent) and BAP1 were used for routine molecular biology studies (Table 12) and protein expression, respectively, and were grown in Luria-Bertani broth or Terrific broth. DNA sequencing was performed at Eurofins. Primers used in this study were listed in Table 13. A plasmid with PTDH (pET15b-Opt13, Plasmid #61698) was purchased from Addgene. TB14 used in the study was reported previously (Zuo, R. et al. Sci Rep 2017 7:842). 1D and 2D NMR spectra of compounds were recorded in DMSO-d₆ or CD₃OD on a Bruker 600 MHz spectrometer using a 5 mm TXI Cryoprobe in the AMRIS facility at the University of Florida, Gainesville, Fla., USA. Spectroscopy data were collected using Topspin 3.5 software. All ¹H NMR were water suppressed to get rid of high background water peak. HRMS data were obtained using a Thermo Fisher Q Exactive Focus mass spectrometer equipped with an electrospray probe on Universal Ion Max API source. The quantitation of the yields of TxtA and TxtB library reactions (non-nitrated products) were based on the standard curve of cyclo-(L-Phe-L-Trp). The quantitation of thaxtomin D, thaxtomin A and thaxtomin B yields from the reactions of TxtA and TxtB, TB14 and TCB14 libraries were based on the standard curve of thaxtomin A (Jiang, G. et al. Appl Environ Microbiol 2018 84:e00164-18; Wnn, M. et al. Angew Chem Int Ed 2018 57:6830-6833).

TABLE 12
Bacterial strains and plasmids commercially available or created
previously.

| Strain or plasmid | Features^{a} |
|---|---|
| E. coli strains | |
| DH5α | General cloning host |
| BL21-Gold-DE3 | Host for protein expression |
| BAP1 | Host for protein expression |
| Plasmids | |
| pET26b | E. coli cloning vector (kan^{R}) |
| pET26b-TB14 | E. coli cloning vector with fused TxtE with |
| | BM3Rd (kan^{R}) |
| pGro7 | E. coli cloning vector with chaperone gene (cml^{R}) |
| pET28a | E. coli cloning vector (kan^{R}) |
| pET28b | E. coli cloning vector (kan^{R}) |
| pET22b | E. coli cloning vector (amp^{R}) |
| pACYC-DUET | E. coli cloning vector (cmI^{R}) |
| pET15b-Opt13 | E. coli cloning vector with PTDH gene (kan^{R}) |

Overexpression and Purification of Enzymes.

The txtC gene was amplified in PCR reactions using TX-C-NcoI-F and C-SacI-R as primers. After purification from agarose gel with GeneJET Gel Extraction Kit (Thermo), the amplicon and TB14 expression plasmid that we previously prepared¹ were digested with SacI and XhoI for constructing pET26b-txtC14-BM3Rd-C-Histag. Following a similar procedure, txtA, txtB and txtH were amplified from genomic DNA of S. scabies 87.22 (NRRL B-24449) and then txtA and txtB were cloned into pET28b vector while txtH was cloned into a pET22b vector. To construct TxtAW225S mutant, two fragments were prepared from the txtA expression construct in PCR reactions using primers listed in Table 13 and then assembled by HIFI Gibson assembly kit (NEB). Codon-optimized genes of glucose 1-dehydrogenase (GDH) from Bacillus megaterium and formate dehydrogenase (FDH) from Mycobacterium vaccae were cloned into a pET22b vector (Table 14). Inserts in all expression constructs were sequenced to exclude any potential errors introduced during PCR amplification and gene manipulation. E. coli transformation, protein expression and purification followed our previously established protocols (Zuo, R. et al. Sci Rep 2017 7:842). Purified recombinant proteins were exchanged into a storage buffer (25 mM Tris-HCl, pH 8.0, 100 mM NaCl, 3 mM βME, and 10% glycerol) by PD-10 column, aliquoted and stored at −80° C. until needed. Protein concentrations were determined by NanoDrop, while the concentrations of properly folded TB14 and TCB14 were measured by absorbance difference at two wavelengths of ˜420 nm and ˜390 nm after differential CO reduced P450 spectral analysis (Omura, T. et al. J Biol Chem 1964 239:2370-2378; Ding, Y. et al. J Am Chem Soc 2008 130(16):5492-5498). The concentrations of TxtA and TxtB were determined through a standard curve of Bovine serum albumin (BSA).

TABLE 13
List of primers used in the study.

| | | |
|---|---|---|
| Primer | | SEQ ID |
| Name | Sequence (5′ → 3′) | No. |

pET28b-TxtA-C-Histag

| | | |
|---|---|---|
| C-Histag- | ATCCCATGGGCATGTCGCACCTGACCGGTGAAGATC | 1 |
| TxtA-F | | |
| C-Histag- | ATCAAGCTTCTGCAGTGGGAGGTCCTGG | 2 |
| TxtA-R | | |

pET28b-TxtB-C-Histag

| | | |
|---|---|---|
| C-Histag- | ATCCCATGGGCATGGTCCACTCGATGTCCATGCTGC | 3 |
| TxtA-F | | |
| C-Histag- | ATC AAGCTT CGGCCGTGGTGAGAAGG | 4 |
| TxtA-R | | |

pET22b-TxtH-C-Histag

| | | |
|---|---|---|
| TxtH-F | ACTGGATCCGGTGCCCTCACCCTTCGACGAC | 5 |
| TxtH-R | ACTGAATTCGATTCACGGACGGACGCCGGGC | 6 |

pET26b-TxtC14-BM3Rd-C-Histag

| | | |
|---|---|---|
| C-NcoI-F | ATACCATGGAATCTCCGGCCACCCAG | 7 |
| C-SadI-R | ATAGAGCTCCCAGGTCATGGGCAGCTCATG | 8 |

pET28b-hMAT2A-C-Histag

| | | |
|---|---|---|
| MAT-F | CATGAATTCGATGAACGGACAGCTCAACGG | 9 |
| MAT-R | AGCGGCCGCTCAATATTTAAGCTTTTTGG | 10 |

pET22b-GDH-C-Histag

| | | |
|---|---|---|
| GDH-F | ATACATATGTATAAAGATCTGGAAGGTA | 11 |
| GDH-R | ATAAAGCTTGCCACGACCTGCCTGAAAGCT | 12 |

pET22b-FDH-C-Histag

| | | |
|---|---|---|
| FDH-F | ATACATATGGCAAAGGTCCTGTGCGTT | 13 |
| FDH-R | ATAAAGCTTGACCGCCTTCTTGAACTTGG | 14 |

TxtA-W225S-C-Histag

| | | |
|---|---|---|
| W2255-V-F | CCAGCAGTACGAAGTCGTGCTG | 15 |
| W2255-V-R | AGAAACTGGCGTCGAAACTCG | 16 |
| W225S-A-F | CGAGTTTCGACGCCAGTTTCTCGGAGATGTCGATGGCGCTGCTG | 17 |
| W225S-A-R | CAGCACGACTTCGTACTGCTGG | 18 |

TABLE 14
Sequences of GDH and FDH used in the study.

| | | SEQ ID |
|---|---|---|
| Name | Sequence | No. |
| Codon | ATGTATAAAGATCTGGAAGGTAAAGTGGTGGTGATTACCGGC | 19 |
| optimized | AGCAGCACCGGTCTGGGCAAAGCAATGGCGATTCGTTTTGCG | |
| glucose | ACCGAAAAAGCGAAAGTGGTGGTTAACTATCGCAGCAAAGAA | |
| 1- | GAAGAAGCGAACAGCGTTCTGGAAGAAATTAAAAAAGTGGGT | |
| dehydro- | GGCGAAGCGATTGCGGTGAAAGGTGATGTGACCGTGGAAAGC | |
| genase | GATGTGATTAACCTGGTGCAGAGCAGCATTAAAGAATTTGGC | |
| (GDH) | AAACTGGATGTGATGATTAACAATGCGGGTATGGAAAATCCG | |
| | GTGAGCAGCCATGAAATGAGCCTGAGCGATTGGAACAAAGTG | |
| | ATTGATACCAACCTGACCGGTGCGTTTCTGGGCAGCCGTGAA | |
| | GCGATTAAATACTTCGTGGAAAACGATATTAAAGGCACCGTG | |
| | ATTAACATGAGCAGCGTGCATGAAAAAATTCCGTGGCCGCTG | |
| | TTTGTGCATTATGCAGCGAGCAAAGGCGGTATGAAACTGATG | |
| | ACCGAAACCCTGGCCCTGGAATATGCACCGAAAGGCATTCGT | |
| | GTGAACAACATTGGTCCGGGTGCGATTAACACCCCGATTAAC | |
| | GCGGAAAAATTTGCCGATCCGGAACAGCGTGCGGATGTGGAA | |
| | AGCATGATTCCGATGGGCTATATTGGCGAACCGGAAGAAATT | |
| | GCAGCGGTGGCAGCGTGGCTGGCAAGCAGCGAAGCGAGCTAT | |
| | GTGACCGGCATTACCCTGTTTGCGGATGGCGGTATGACCCAG | |
| | TATCCGAGCTTTCAGGCAGGTCGTGGCTAA | |
| Formate | ATGGCAAAGGTCCTGTGCGTTCTTTACGATGATCCGGTCGAC | 20 |
| dehydro- | GGCTACCCGAAGACCTATGCCCGCGACGATCTTACGAAGATC | |
| genase | GACCACTATCCGGGCGGCCAGACCTTGCCGACGCCGAAGGCC | |
| (FDH) | ATCGACTTCACGCCCGGGCAGCTGCTCGGCTCCGTCTCCGGC | |
| | GAGCTCGGCCTGCGCAAATATCTCGAATCCAACGGCCACACC | |
| | CTGGTCGTGACCTCCGACAAGGACGGCCCCGACTCGGTGTTC | |
| | GAGCGCGAGCTGGTCGATGCGGATGTCGTCATCTCCCAGCCC | |
| | TTCTGGCCGGCCTATCTGACGCCCGAGCGCTTCGCCAAGGCC | |
| | AAGAACCTGAAGCTCGCGCTCACCGCCGGCATCGGTTCCGAC | |
| | CACGTCGATCTTCAGTCGGCTGTCGACCGTAACGTCACTGTG | |
| | GCGGAAGTCACCTACTGCAACTCGATCAGCGTCGCCGAGCAT | |
| | GTGGTGATGTTGATCCTGTCGCTGGTGCGCAACTTTTTGCCC | |
| | TCGCACGAATGGGCGCGGAAGGGCGGCTGGAACATCGCCGAC | |
| | TGCGTCTCCCACGCCTACGACCTCGAGGCGATGCATGTCGGC | |
| | ACCGTGGCCGCCGGTCGCATCGGTCTCGCGGTGCTGCGCCGG | |
| | CTGGCGCCGTTCGACGTGCACCTGCACTACACCGACCGTCAC | |
| | CGCCTGCCGGAATCGGTCGAGAAGGAACTCAACCTCACCTGG | |
| | CACGCGACCCGCGAGGACATGTATCCGGTTTGCGACGTGGTG | |
| | ACGCTGAACTGCCCGCTGCACCCAGATACCGAGCACATGATC | |
| | AATGACGAGACGCTGAAGCTGTTCAAGCGCGGCGCCTACATC | |
| | GTCAACACCGCCCGCGGCAAGCTGTGCGACCGCGATGCCGTG | |
| | GCACGTGCGCTCGAATCCGGCCGGCTGGCCGGCTATGCCGGC | |
| | GACGTGTGGTTCCCGCAGCCGGCGCCGAAGGACCACCTCTGG | |
| | CGGACGATGCCCTATAACGCCATGACCCCGCACATCTCCGGC | |
| | ACCACGCTGACCGCGCAGGCGCGTTATGCAGCGGGCACCCGC | |
| | GAGATCCTGGAGTGCTTCTTCGAGGGCCGTCCGATCCGCGAC | |
| | GAATACCTCATCGTGCAGGGCGGCGCTCTTGCCGGCACCGGC | |
| | GCGCATTCCTACTCGAAGGGCAATGCCACTGGCGGTTCGGAA | |
| | GAGGCCGCCAAGTTCAAGAAGGCGGTCTGA | |

Spectral Analysis of Chimeric TCB14.

Purified TB14 and TCB14 were spectrally analyzed following a previous protocol. Briefly, the absorbance spectra (400-600 nm) of TB14 and TCB14 in Tris-HCl (25 mM, pH 9) buffer were recorded using a Shimadzu UV2700 dual beam UV-Vis spectrophotometer. The ferric heme of enzymes was then saturated by bubbling carbon monoxide (Airgas) and the spectra were then recorded. A finite amount of solid sodium dithionite was subsequently added to enzyme solutions to reduce the ferric heme, and reduced spectra were taken. CO reduced differential spectra of both P450s were created by subtracting their CO binding spectra from corresponding reduced spectra. Data were further analyzed by Excel.

In Vitro Reconstitution of TxtA and TxtB Reactions.

The TxtA/B reaction solutions (100 μl) typically contained 100 mM Tris-Cl, pH 9.0, 0.5 mM amino acid substrates, 10 mM MgCl₂, 2.5 mM ATP and 2 mM SAM. The reactions were initiated by adding 1.2 μM TxtA and 1.3 μM TxtB (final concentration) and incubated at 21° C. with shaking at 400 rpm. After 30 h, the reactions were terminated by mixing with 200 μl of methanol. Quenched solutions were centrifuged at 4° C., 16,000×g for 15 min and clear supernatants were collected and subjected to HPLC and LCMS analysis as detailed below. All experiments were repeated independently at least twice. The reaction solutions of TB14 and TxtA and TxtB were the same as those of TxtA and TxtB except the inclusion of 1.5 mM NAPDH, 0.75 mM NOC-5 and 11.1 μM TB14. The reactions were run for 30 h and the products were analyzed and quantitated as described above.

Biochemical Characterization of TCB14 and In Vitro Reconstitution of Thaxtomin Biosynthesis.

The TCB14 reaction solutions (100 μl) typically contained 100 mM Tris-Cl, pH 8.0, 0.3 mM thaxtomin D or other substrates, and 1-6 mM NADPH. Alternatively, the reactions contained the NADPH regeneration systems (e.g., 9.0 μM GDH, 0.75 mM NADP+ and 30.0 mM glucose/9.0 μM FDH with 0.75 mM NADP+ and 10.0 mM sodium formate/9.0 μM PTDH with 0.75 mM NADP+ and 40.0 mM sodium phosphite). The reactions were initiated by adding 16.7 μM TCB14 (final concentration) and incubated at 21° C. with shaking at 400 rpm for 20 h prior to the termination. Hydroxylated thaxtomin analogues were detected as described below. To fully reconstitute thaxtomin biosynthesis, the first stage was the same as the TB14, TxtA and TxtB reactions described above. In the second stage, TCB14 (16.7 μM), GDH (9.0 μM), NADP+ (0.75 mM) and glucose (30.0 mM) were added to the above reaction mixture, which was further incubated at 21° C., 400 rpm for 20 h. The products were analyzed and quantitated as described below.

Large-Scale Enzymatic Synthesis of Thaxtomin Analogues.

To isolate sufficient amounts of thaxtomin analogues for structural determination and herbicidal assay, the enzymatic reactions were scaled up to 10-50 mL. The reactions were incubated at 21° C., 250 rpm overnight, and then terminated by mixing with two volumes of ethyl acetate (EA). The products were extracted twice, and the organic extracts were combined, dried, and concentrated in vacuo. After freeze-drying overnight, the crude extracts were dissolved in methanol for the purification of thaxtomin analogues using semi-preparative HPLC.

HPLC, LC-MS, LC-HRMS and Tandem MS Analysis.

A Shimadzu Prominence UHPLC system (Kyoto, Japan) fitted with an Agilent Poroshell 120 EC-C18 column (2.7 μm, 4.6×50 mm) and coupled with a PDA detector was used for HPLC analysis. Solvent A was water with 0.1% formic acid (FA) and solvent B was acetonitrile with 0.1% FA. The column was equilibrated with 10% solvent B for 2 min and then eluted with a linear gradient of 10-50% in 8 min, followed by the other linear gradient of 50-99% solvent B in 5 min. The column was further eluted with 99% solvent B for 3 min and then re-equilibrated with 10% solvent B for 1 min. The flow rate was 0.5 mL/min. Nitrated products were detected at 380 nm with a PDA detector while desnitro products were detected at 280 nm.

For semi-preparative HPLC analysis, an Agilent ZORBAX SB-C18 (5 μm, 9.4×250 mm) or YMC-Pack Ph (5 μm, 4.6×250 mm) column was used at 40° C. The column was first eluted with 10% solvent B for 2 min and then with a linear gradient of 10-50% solvent B for 8 min, followed by a linear gradient of 50-99% solvent B for 5 min. The column was then cleaned with 99% solvent B for 1 min and re-equilibrated with 10% solvent B for 1 min. The flow rate was 3 mL/min, and the products were detected at 380 nm with a PDA detector. Fractions containing targeted compounds were collected, combined, and dried for further purification with one analytical column (YMC-Pack Ph column, 5 μm, 4.6×250 mm). Specifically, the products were separated on the column with 50% solvent B (methanol with 0.1% FA) for 13.5 min at 30° C. The column was cleaned first with 50-99% solvent B for 0.5 min and then 99% solvent B for 0.5 min, followed with re-equilibration at 50% solvent B for 1 min. The flow rate was 1 mL/min. Fractions containing thaxtomin analogues were collected, combined, dried in a lyophilizer and weighed prior to NMR analysis.

LC-MS was performed on a SHIMADZU Prominence UPLC system fitted with an Agilent Poroshell 120 EC-C18 column (2.7 μm, 4.6×50 mm) coupled with a Linear Ion Trap Quadrupole LC/MS/MS Mass Spectrometer system was used in the studies. The HPLC conditions were the same as described for HPLC analysis. LC-HRMS analysis was performed on a Thermo Fisher Q Exactive Focus mass spectrometer coupled with a SHIMADZU Prominence UPLC system fitted with an Agilent Eclipse Plus C18 column (3.5 μm, 2.1×100 mm, 90 Å). Acetonitrile (B)/water (A) containing 0.1% FA were used as mobile phases with a linear gradient program (10-90% solvent B over 15 min) to separate chemicals at a flow rate of 0.3 mL/min. A pre-wash phase of 15 min with 10% solvent B was added at the beginning of each run, in which the elute was diverted to the waste by a diverting valve. For MS/MS analysis, MS1 was acquired under a Full-Scan mode of Orbitrap, in which a mass range of m/z 150-2000 was covered and data were collected in the positive ion mode. Fragmentation was introduced by HCD technique with optimized collision energy ranging from 6 to 30 eV. Other settings for the Orbitrap scan were as follow: resolution 15000, AGC target 5×10⁵. Full scan mass spectra and targeted MS/MS spectra for each of the pre-selected parental ion were extracted from the raw files of the HPLC-MS/MS Experiment II using Xcalibur™ 2.1 (Thermo Scientific).

Structural Characterization of Three Selected Thaxtomin Analogues.

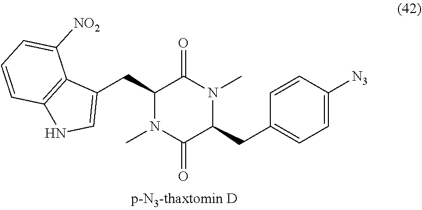 [SMILES: CN1C(=O)[C@H](Cc2c[nH]c3cccc([N+](=O)[O-])c23)N(C)C(=O)[C@@H]1Cc1ccc(N=[N+]=[N-])cc1]

p-N₃-thaxtomin D: yellow solid; HRMS (ESI-TOF) m/z 448.1714 [M+H]⁺ (calcd. for C₂₂H₂₂N₇O₄, 448.1728); ¹H NMR (600 MHz, CD₃OD) δ 7.85 (d, J=7.8 Hz, 1H), 7.74 (d, J=8.1 Hz, 1H), 7.24 (t, J=7.9 Hz, 1H), 7.18 (s, 1H), 7.09 (d, J=8.3 Hz, 2H), 7.05 (d, J=8.4 Hz, 2H), 4.12 (t, J=5.4 Hz, 1H), 4.06-3.94 (m, 1H), 3.24-3.15 (m, 2H), 3.12 (dd, J=13.1, 5.2 Hz, 1H), 2.88 (s, 3H), 2.73 (s, 3H), 2.52 (dd, J=14.2, 6.0 Hz, 1H). ¹³C NMR (150 MHz, CD₃OD): δ 167.64, 167.40, 144.11, 140.98, 140.72, 135.07, 132.38, 131.71, 121.35, 120.47, 120.09, 119.26, 118.72, 110.35, 65.33, 47.95, 39.07, 33.72, 31.99.

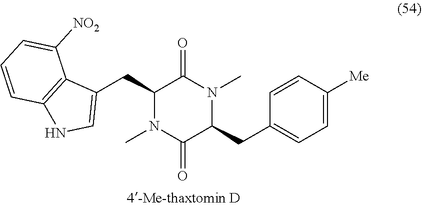 [SMILES: Cc1ccc(C[C@H]2C(=O)N(C)[C@@H](Cc3c[nH]c4cccc([N+](=O)[O-])c34)C(=O)N2C)cc1]

4′Me-thaxtomin D: yellow solid; HRMS (ESI-TOF) m/z 421.1856 [M+H]+(calcd. for C₂₃H₂₅N₄O₄, 421.1870); ¹H NMR (600 MHz, CD₃OD): δ 7.84 (d, J=7.8 Hz, 1H), 7.73 (d, J=8.0 Hz, 1H), 7.23 (t, J=7.9 Hz, 1H), 7.18 (d, J=7.7 Hz, 2H), 7.07 (s, 1H), 6.98 (d, J=7.9 Hz, 2H), 4.15 (t, J=5.0 Hz, 1H), 3.99-3.93 (m, 1H), 2.93 (ddd, J=26.1, 14.4, 5.1 Hz, 2H), 2.82 (s, 3H), 2.76 (s, 3H), 2.63 (dd, J=14.2, 5.6 Hz, 1H), 2.46 (dd, J=14.5, 7.4 Hz, 1H), 2.28 (s, 3H). ¹³C NMR (150 MHz, CD₃OD): δ 167.80, 167.32, 143.95, 140.98, 138.36, 134.86, 131.81, 130.84, 130.61, 121.23, 119.95, 119.23, 118.59, 110.35, 65.09, 65.35, 38.79, 33.83, 33.54, 32.24, 21.09.

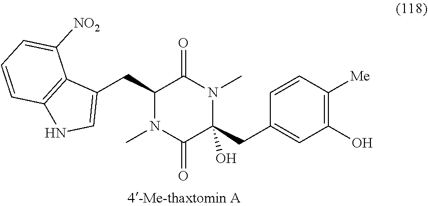 [SMILES: Cc1ccc(C[C@@]2(O)C(=O)N(C)[C@@H](Cc3c[nH]c4cccc([N+](=O)[O-])c34)C(=O)N2C)cc1O]

4′Me-thaxtomin A: yellow solid; HRMS (ESI-TOF) m/z 453.1757 [M+H]+(calcd. for C₂₃H₂₅N₄O₆, 453.1769); ¹H NMR (600 MHz, CD₃OD): δ 7.83 (d, J=7.8 Hz, 1H), 7.70 (d, J=8.0 Hz, 1H), 7.19 (t, J=7.9 Hz, 1H), 7.11 (d, J=7.6 Hz, 1H), 6.86 (s, 1H), 6.66 (s, 1H), 6.60 (d, J=7.5 Hz, 1H), 3.91-3.80 (m, 1H), 3.28 (d, J=13.7 Hz, 1H), 3.07 (d, J=13.6 Hz, 1H), 2.78 (s, 3H), 2.54 (dd, J=14.1, 6.5 Hz, 1H), 2.12 (s, 3H), 1.68 (dd, J=14.1, 8.7 Hz, 1H). ¹³C NMR (150 MHz, CD₃OD): δ 168.42, 166.84, 156.98, 143.67, 141.19, 134.38, 132.32, 125.52, 122.55, 121.00, 119.78, 119.23, 118.37, 117.81, 110.67, 88.05, 64.55, 43.32, 34.24, 33.63, 28.41, 15.90.

Herbicidal Activity Assay of Thaxtomin Analogues

Established protocols were followed to quantitate thaxtomin herbicidal activity in radish seedling assay (Jiang, G. et al. Appl Environ Microbiol 2018 84:e00164-18). In brief, serial concentrations (0 to 2 μM) of thaxtomins in DMSO were added into 20 mL of 1.5% warm agar solution with gentle agitation to prepare agar plates. DMSO was included as the negative control. Radish seeds (Burpee) were surface disinfested, pregerminated and selected when the radicle was 1±2 mm and just emerged from the seed coat. Six radish seedlings were equally located on the surface of each plate with root ends pointed to the same direction. Agar plates were covered, sealed with Parafilm. Seedlings in agar plates grew at room temperature under 14-16 h fluorescent light per day for 7 days, and total seedling lengths were then recorded. The longest and the shorted seedlings would be excluded from the data analysis. Percent inhibition relative to the mean growth response in negative control was then calculated. Dose-response curves were fit to a four-parameter logistic model, and I₅₀ values were determined from these curves.

Modelling a Domain Structure

A crystal structure of grsA (PDB code 1AMU) was used as the crystallographic coordinate template. Homology modelling of AMdnC was performed based on the reference protein model using Protein Modeling module embedded in Discovery Studio 2.5. The optimized model was evaluated by the Ramachandran plot analysis. The alpha carbon based RMSD is 0.22 angstrom.

Following below are Tables 8-11 which are in landscape orientation.

TABLE 8
HR-MS data and conversion rates of desnitro thaxtomin D analogues generated in TxtA and TxtB reactions.

| | Conversion rate | | L-Phe or L-Phe | L-Trp or L-Trp | Calculated | | Key featured MS2 |
|---|---|---|---|---|---|---|---|
| Compound# | % | | analogue | analogue | [M + H]+ | Identified [M + H]+ | fragments |
| 6 | 7.73 | ± 0.54 | L-Phe | L-Trp | 362.1863 | 362.1863 | 130.1, 231.1, 362.2 |
| | | | | | | (Δ 4.4 ppm) | |
| 13 | 3.75 | ± 0 | 2F-L-Phe | L-Trp | 380.1769 | 380.1757 | 130.0, 249.1, 380.2 |
| | | | | | | (Δ 3.2 ppm) | |
| 14 | 6.04 | ± 0.05 | 3F-L-Phe | L-Trp | 380.1769 | 380.1756 | 129.9, 249.1, 380.1 |
| | | | | | | (Δ 3.4 ppm) | |
| 15 | 6.79 | ± 0.42 | 4F-L-Phe | L-Trp | 380.1769 | 380.1758 | 129.9, 249.3, 380.4 |
| | | | | | | (Δ 2.9 ppm) | |
| 16 | 1.74 | ± 0.02 | 3Cl-L-Phe | L-Trp | 396.1473 | 396.1464 | 129.9, 265.5, 396.4 |
| | | | | | | (Δ 2.3 ppm) | |
| 17 | 2.62 | ± 0.10 | 4Cl-L-Phe | L-Trp | 396.1473 | 396.1462 | 130.0, 264.9, 396.0 |
| | | | | | | (Δ 2.8 ppm) | |
| 18 | 5.45 | ± 0.01 | L-Phe | 4F-D,L-Trp | 380.1769 | 380.1757 | 148.2, 231.3, 380.4 |
| | | | | | | (Δ 3.2 ppm) | |
| 19 | 3.36 | ± 0.01 | 2F-L-Phe | 4F-D,L-Trp | 398.1675 | 398.1646 | 148.3, 248.9, 398.2 |
| | | | | | | (Δ 2.2 ppm) | |
| 20 | 5.87 | ± 0.41 | 3F-L-Phe | 4F-D,L-Trp | 398.1675 | 398.1666 | 148.0, 249.3, 398.4 |
| | | | | | | (Δ 2.3 ppm) | |
| 21 | 4.62 | ± 0.01 | 4F-L-Phe | 4F-D,L-Trp | 398.1675 | 398.1664 | 148.0, 249.1, 398.4 |
| | | | | | | (Δ 2.8 ppm) | |
| 22 | 1.19 | ± 0 | 3Cl-L-Phe | 4F-D,L-Trp | 414.1379 | 414.1367 | 148.4, 265.0, 414.3 |
| | | | | | | (Δ 2.9 ppm) | |
| 23 | 2.27 | ± 0.66 | 4Cl-L-Phe | 4F-D,L-Trp | 414.1379 | 414.1367 | 148.2, 265.1, 414.0 |
| | | | | | | (Δ 2.9 ppm) | |
| 24 | 14.87 | ± 0.03 | L-Phe | 5F-L-Trp | 380.1769 | 380.1757 | 148.2, 231.3, 380.4 |
| | | | | | | (Δ 3.2 ppm) | |
| 25 | 10.35 | ± 0.7 | 2F-L-Phe | 5F-L-Trp | 398.1675 | 398.1664 | 148.1, 249.2, 398.2 |
| | | | | | | (Δ 2.8 ppm) | |
| 26 | 14.06 | ± 0.08 | 3F-L-Phe | 5F-L-Trp | 398.1675 | 398.1662 | 148.4, 249.3, 398.1 |
| | | | | | | (Δ 3.3 ppm) | |
| 27 | 14.67 | ± 0.06 | 4F-L-Phe | 5F-L-Trp | 398.1675 | 398.1663 | 148.0, 249.1, 398.3 |
| | | | | | | (Δ 3.0 ppm) | |
| 28 | 3.00 | ± 0.02 | 3Cl-L-Phe | 5F-L-Trp | 414.1379 | 414.1366 | 147.9, 265.0, 414.3 |
| | | | | | | (Δ 3.1 ppm) | |
| 29 | 5.70 | ± 0.01 | 4Cl-L-Phe | 5F-L-Trp | 414.1379 | 414.1366 | 148.1, 265.3, 414.1 |
| | | | | | | (Δ 3.1 ppm) | |
| 30 | 3.53 | ± 0.01 | L-Phe | 6F-D,L-Trp | 380.1769 | 380.1762 | 148.0, 231.1, 380.3 |
| | | | | | | (Δ 1.8 ppm) | |
| 31 | 2.15 | ± 0.13 | 2F-L-Phe | 6F-D,L-Trp | 398.1675 | 398.1665 | 147.8, 249.2, 398.1 |
| | | | | | | (Δ 2.5 ppm) | |
| 32 | 3.23 | ± 0.01 | 3F-L-Phe | 6F-D,L-Trp | 398.1675 | 398.1669 | 148.0, 249.1, 398.0 |
| | | | | | | (Δ 2.5 ppm) | |
| 33 | 3.47 | ± 0.04 | 4F-L-Phe | 6F-D,L-Trp | 398.1675 | 398.1667 | 148.0, 249.3, 398.4 |
| | | | | | | (Δ 2.0 ppm) | |
| 34 | 1.31 | ± 0 | 3Cl-L-Phe | 6F-D,L-Trp | 414.1379 | 414.1375 | 148.4, 265.3, 413.9 |
| | | | | | | (Δ 1.0 ppm) | |
| 35 | 0.73 | ± 0 | 4Cl-L-Phe | 6F-D,L-Trp | 414.1379 | 414.1373 | 148.1, 265.0, 414.1 |
| | | | | | | (Δ 1.4 ppm) | |
| 36 | 22.09 | ± 0.01 | L-Phe | 4Me-DL-Trp | 376.2020 | 376.2018 | 144.0, 231.1, 376.3 |
| | | | | | | (Δ 0.5 ppm) | |
| 37 | 14.04 | ± 0.92 | 2F-L-Phe | 4Me-D,L-Trp | 394.1925 | 394.1915 | 144.2, 249.0, 394.0 |
| | | | | | | (Δ 2.5 ppm) | |
| 38 | 18.65 | ± 0.04 | 3F-L-Phe | 4Me-D,L-Trp | 394.1925 | 394.1919 | 144.3, 249.3, 394.4 |
| | | | | | | (Δ 1.5 ppm) | |
| 39 | 13.76 | ± 0.04 | 4F-L-Phe | 4Me-D,L-Trp | 394.1925 | 394.1920 | 144.2, 249.2, 394.4 |
| | | | | | | (Δ 1.3 ppm) | |
| 40 | 1.10 | ± 0.06 | 3Cl-L-Phe | 4Me-D,L-Trp | 410.1630 | 410.1624 | 144.1, 265.0, 410.0 |
| | | | | | | (Δ 1.4 ppm) | |
| 41 | 4.44 | ± 0.01 | 4Cl-L-Phe | 4Me-D,L-Trp | 410.1630 | 410.1623 | 144.5, 265.0, 410.2 |
| | | | | | | (Δ 1.7 ppm) | |

TABLE 9
HR-MS data and conversion rates of nitro-DKPs in the reactions of TB14, TxtA and TxtB.

| | Conversion rate | | L-Phe or L-Phe | L-Trp or L-Trp | Calculated | Identified | Key featured MS2 |
|---|---|---|---|---|---|---|---|
| Compound# | % | | analogue | analogue | [M + H]+ | [M + H]+ | fragments |
| 5a | 43.06 | ± 13.44 | L-Phe | L-Trp | 379.1401 | 379.1396 (Δ 1.3 | 130.1, 306.1, 333.1, |
| 3b | 5.68 | ± 1.51 | L-Phe | L-Trp | 393.1557 | 393.1552 (Δ 1.3 | 130.1, 219.1, 347.2, |
| 42c | 20.90 | ± 0.91 | p-N3-L-Phe | L-Trp | 448.1728 | 448.1714 (Δ 3.1 | 130.1, 269.1, 315.1, |
| 43c | 11.68 | ± 4.56 | L-Tyr | L-Trp | 423.1663 | 423.1646 (Δ 4.0 | 130.1, 248.0, 423.1 |
| 4 | 93.83 | ± 4.47 | L-Phe | L-Trp | 407.1714 | 407.1710 (Δ 1.0 | 130.1, 232.1, 407.2 |
| 44 | 69.09 | ± 0.05 | 2F-L-Phe | L-Trp | 425.1620 | 425.1615 (Δ 1.2 | 129.9, 250.2, 425.2 |
| 45 | 91.86 | ± 0.46 | 3F-L-Phe | L-Trp | 425.1620 | 425.1603 (Δ 4.0 | 130.1, 250.3, 425.0 |
| 46 | 29.56 | ± 1.92 | 4F-L-Phe | L-Trp | 425.1620 | 425.1604 (Δ 3.8 | 130.4, 249.9, 425.3 |
| 47 | 13.50 | ± 1.89 | 3Cl-L-Phe | L-Trp | 441.1324 | 441.1309 (Δ 3.4 | 130.1, 266.2, 441.2 |
| 48 | 6.29 | ± 0.04 | 4Cl-L-Phe | L-Trp | 441.1324 | 441.1314 (Δ 2.3 | 130.0, 266.2, 441.2 |
| 49 | 10.25 | ± 0.01 | 2Br-L-Phe | L-Trp | 485.0819 | 485.0807 (Δ 2.5 | 130.2, 310.0, 485.2 |
| 50 | 9.68 | ± 0.04 | 3Br-L-Phe | L-Trp | 485.0819 | 485.0802 (Δ 3.5 | 130.2, 310.0, 485.2 |
| 51 | 4.59 | ± 0.43 | 4Br-L-Phe | L-Trp | 485.0819 | 485.0807 (Δ 2.5 | 130.1, 310.2, 485.0 |
| 52 | 37.49 | ± 3.91 | 2Me-L-Phe | L-Trp | 421.1870 | 421.1862 (Δ 1.9 | 130.3, 246.3, 421.2 |
| 53 | 24.98 | ± 1.82 | 3Me-L-Phe | L-Trp | 421.1870 | 421.1856 (Δ 3.3 | 130.2, 246.3, 421.3 |
| 54 | 15.69 | ± 1.11 | 4Me-L-Phe | L-Trp | 421.1870 | 421.1856 (Δ 3.3 | 129.8, 246.2, 421.0 |
| 55 | 15.56 | ± 1.19 | L-Phe | 5F-L-Trp | 425.1620 | 425.1607 (Δ 3.3 | 148.2, 232.3, 424.3 |
| 56 | 12.99 | ± 0.14 | 2F-L-Phe | 5F-L-Trp | 443.1525 | (Δ 3.4 ppm) | 148.2, 249.9, 442.9 |
| 57 | 16.6 | ± 0.16 | 3F-L-Phe | 5F-L-Trp | 443.1525 | 443.1512 (Δ 2.9 | 148.5, 250.0, 443.3 |
| 58 | 12.18 | ± 0.85 | 4F-L-Phe | 5F-L-Trp | 443.1525 | 443.1510 (Δ 3.4 | 148.2, 249.9, 443.3 |
| 59 | 6.77 | ± 0.38 | 3Cl-L-Phe | SF-L-Trp | 459.1230 | 459.1215 (Δ 3.3 | 148.2, 266.1, 459.1 |
| 60 | 4.19 | ± 0.08 | 4Cl-L-Phe | 5F-L-Trp | 459.1230 | 459.1218 (Δ 2.6 | 148.3, 266.3, 458.8 |
| 61 | 4.58 | ± 0.05 | 2Br-L-Phe | 5F-L-Trp | 503.0725 | 503.0712 (Δ 2.6 | 148.1, 310.2, 502.7 |
| 62 | 3.02 | ± 0.14 | 3Br-L-Phe | 5F-L-Trp | 503.0725 | 503.0712 (Δ 2.6 | 147.9, 310.1, 503.1 |
| 63 | 1.81 | ± 0.02 | 4Br-L-Phe | 5F-L-Trp | 503.0725 | 503.0710 (Δ 3.0 | 148.2, 310.2, 502.8 |
| 64 | 13.60 | ± 0.55 | 2Me-L-Phe | 5F-L-Trp | 439.1776 | 439.1764 (Δ 2.7 | 147.9, 246.3, 439.3 |
| 65 | 8.98 | ± 0.19 | 3Me-L-Phe | 5F-L-Trp | 439.1776 | 439.1765 (Δ 2.5 | 148.3, 246.1, 439.2 |
| 66 | 5.60 | ± 0.45 | 4Me-L-Phe | 5F-L-Trp | 439.1776 | 439.1766 (Δ 2.3 | 148.1, 246.3, 439.1 |
| 67 | 37.06 | ± 3.42 | L-Phe | 6F-D,L-Trp | 425.1620 | 425.1605 (Δ 3.5 | 148.1, 232.4, 424.9 |
| 68 | 19.19 | ± 1.52 | 2F-L-Phe | 6F-D,L-Trp | 443.1525 | 443.1512 (Δ 2.9 | 148.2, 250.1, 443.1 |
| 69 | 18.93 | ± 1.17 | 3F-L-Phe | 6F-D,L-Trp | 443.1525 | 443.1514 (Δ 2.5 | 148.1, 250.1, 443.8 |
| 70 | 8.86 | ± 0.12 | 4F-L-Phe | 6F-D,L-Trp | 443.1525 | 443.1514 (Δ 2.5 | 148.2, 250.1, 443.1 |
| 71 | 1.12 | ± 0.03 | 3Cl-L-Phe | 6F-D,L-Trp | 459.1230 | 469.1216 (Δ 3.0 | 147.9, 266.2, 459.1 |
| 72 | 2.21 | ± 0.08 | 4Cl-L-Phe | 6F-D,L-Trp | 459.1230 | 459.1216 (Δ 3.0 | 148.1, 266.1, 459.1 |
| 73 | 0.29 | ± 0.02 | 2Br-L-Phe | 6F-D,L-Trp | 503.0725 | 503.0714 (Δ 2.2 | 148.1, 309.9, 502.8 |
| 74 | 0.38 | ± 0.04 | 3Br-L-Phe | 6F-D,L-Trp | 503.0725 | 503.0713 (Δ 2.4 | 148.1, 309.3, 503.0 |
| 75 | 0.44 | ± 0.03 | 4Br-L-Phe | 6F-D,L-Trp | 503.0725 | 503.0711 (Δ 2.8 | 148.4, 309.8, 503.2 |
| 76 | 5.09 | ± 0.20 | 2Me-L-Phe | 6F-D,L-Trp | 439.1776 | 439.1766 (Δ 2.3 | 147.9, 246.3, 439.3 |
| 77 | 1.95 | ± 0.08 | 3Me-L-Phe | 6F-D,L-Trp | 439.1776 | 439.1765 (Δ 2.5 | 148.4, 246.6, 439.0 |
| 78 | 2.30 | ± 0.05 | 4Me-L-Phe | 6F-D,L-Trp | 439.1776 | 439.1764 (Δ 2.7 | 148.1, 245.9, 439.3 |

aConversion rates were calculated based on the reaction without SAM;
bConversion rates were calculated based on the reaction with 1 mM SAM;
cCatalyzed by TxtB and TxtAW225S

TABLE 10
HR-MS data and conversion rates of thaxtomin B analogues in the reaction of TB14, TCB14, TxtA and TxtB.

| | Conversion | | L-Phe or L-Phe | L-Trp or L-Trp | Calculated | Identified | Key featured MS2 |
|---|---|---|---|---|---|---|---|
| Compound# | rate % | | analogue | analogue | [M + H]+ | [M + H]+ | fragments |
| 2 | 36.29 | ± 2.46 | L-Phe | L-Trp | 423.1663 | 423.1656 (Δ 1.7 ppm) | 130.0, 264.9, 380.2, |
| | | | | | | | 439.2, 457.2 |
| 79 | 43.15 | ± 2.56 | 2F-L-Phe | L-Trp | 441.1569 | 441.1568 (Δ 0.2 ppm) | 130.4, 265.1, 379.9, |
| | | | | | | | 439.3, 457.2. |
| 80 | 36.09 | ± 0.41 | 3F-L-Phe | L-Trp | 441.1569 | 441.1569 (Δ 0.0 ppm) | 130.0, 264.9, 380.2, |
| | | | | | | | 439.2, 457.2 |
| 81 | 3.25 | ± 0.11 | 4F-L-Phe | L-Trp | 441.1569 | 441.1569 (Δ 0.0 ppm) | 130.4, 265.1, 379.9, |
| | | | | | | | 439.3, 457.2 |
| 82 | 8.81 | ± 0.53 | 3Cl-L-Phe | L-Trp | 457.1273 | 457.1274 (Δ 0.2 ppm) | 130.0, 264.9, 380.2, |
| | | | | | | | 439.2, 457.2 |
| 83 | 2.66 | ± 0.08 | 4Cl-L-Phe | L-Trp | 457.1273 | 457.1272 (Δ 0.2 ppm) | 130.4, 265.1, 379.9, |
| | | | | | | | 439.3, 457.2 |
| 84 | 3.14 | ± 0.19 | 2Br-L-Phe | L-Trp | 501.0768 | 501.0764 (Δ 0.8 ppm) | 130.0, 264.9, 380.2, |
| | | | | | | | 439.2, 457.2 |
| 85 | 4.23 | ± 0.15 | 3Br-L-Phe | L-Trp | 501.0768 | 501.0763 (Δ 1.0 ppm) | 130.4, 265.1, 379.9, |
| | | | | | | | 439.3, 457.2 |
| 86 | 0.97 | ± 0.07 | 4Br-L-Phe | L-Trp | 501.0768 | 501.0769 (Δ 0.2 ppm) | 130.0, 264.9, 380.2, |
| | | | | | | | 439.2, 457.2 |
| 87 | 22.94 | ± 0.06 | 2Me-L-Phe | L-Trp | 437.1819 | 437.1810 (Δ 2.0 ppm) | 130.4, 265.1, 379.9, |
| | | | | | | | 439.3, 457.2 |
| 88 | 3.81 | ± 0.31 | 3Me-L-Phe | L-Trp | 437.1819 | 437.1808 (Δ 2.5 ppm) | 130.0, 264.9, 380.2, |
| | | | | | | | 439.2, 457.2 |
| 89 | 2.96 | ± 0.99 | 4Me-L-Phe | L-Trp | 437.1819 | 437.1807 (Δ 2.7 ppm) | 130.4, 265.1, 379.9, |
| | | | | | | | 439.3, 457.2 |
| 90 | 0.86 | ± 0.03 | L-Phe | 5F-L-Trp | 441.1569 | 441.1559 (Δ 2.3 ppm) | 130.0, 264.9, 380.2, |
| | | | | | | | 439.2, 457.2 |
| 91 | 2.35 | ± 0.17 | 2F-L-Phe | 51-L-Trp | 459.1475 | 459.1462 (Δ 2.8 ppm) | 130.4, 265.1, 379.9, |
| | | | | | | | 439.3, 457.2 |
| 92 | 3.13 | ± 0.04 | 3F-L-Phe | 5F-L-Trp | 459.1475 | 459.1461 (Δ 3.0 ppm) | 130.0, 264.9, 380.2, |
| | | | | | | | 439.2, 457.2. |
| 93 | 0.23 | ± 0.01 | 4F-L-Phe | 5F-L-Trp | 459.1475 | 459.1466 (Δ 2.0 ppm) | 130.4, 265.1, 379.9, |
| | | | | | | | 439.3, 457.2 |
| 94 | 1.02 | ± 0.16 | 3Cl-L-Phe | 5F-L-Trp | 475.1179 | 475.1167 (Δ 2.5 ppm) | 130.0, 264.9, 380.2, |
| | | | | | | | 439.2, 457.2 |
| 95 | 0.56 | ± 0 | 4Cl-L-Phe | 51-L-Trp | 475.1179 | 475.1169 (Δ 2.1 ppm) | 130.4, 265.1, 379.9, |
| | | | | | | | 439.3, 457.2 |
| 96 | 0.43 | ± 0.19 | 2Br-L-Phe | 5F-L-Trp | 519.0674 | 519.0661 (Δ 2.5 ppm) | 130.0, 264.9, 380.2, |
| | | | | | | | 439.2, 457.2 |
| 97 | 0.61 | ± 0.08 | 3Br-L-Phe | 5F-L-Trp | 519.0674 | 519.0661 (Δ 2.5 ppm) | 130.4, 265.1, 379.9, |
| | | | | | | | 439.3, 457.2 |
| 98 | 0.22 | ± 0.09 | 4Br-L-Phe | 5F-L-Trp | 519.0674 | 519.0662 (Δ 2.3 ppm) | 130.0, 264.9, 380.2, |
| | | | | | | | 439.2, 457.2 |
| 99 | 1.07 | ± 0.06 | 2Me-L-Phe | 5F-L-Trp | 455.1725 | 455.1712 (Δ 2.9 ppm) | 130.4, 265.1, 379.9, |
| | | | | | | | 439.3, 457.2 |
| 100 | 1.73 | ± 0.24 | 3Me-L-Phe | 5F-L-Trp | 455.1725 | 455.1712 (Δ 2.9 ppm) | 130.0, 264.9, 380.2, |
| | | | | | | | 439.2, 457.2 |
| 101 | 0.56 | ± 0.01 | 4Me-L-Phe | 5F-L-Trp | 455.1725 | 455.1713 (Δ 2.6 ppm) | 130.4., 265.1, 379.9, |
| | | | | | | | 439.3, 457.2 |
| 102 | 0.44 | ± 0.01 | L-Phe | 6F-D,L-Trp | 441.1569 | 441.1560 (Δ 2.0 ppm) | 147.9, 231.1, 364.1, |
| | | | | | | | 423.1, 441.2 |
| 103 | 0.97 | ± 0.03 | 2F-L-Phe | 6F-D,L-Trp | 459.1475 | 459.1463 (Δ 2.6 ppm) | 148.0, 249.1, 382.1, |
| | | | | | | | 441.1, 459.1 |
| 104 | 3.36 | ± 0.07 | 3F-L-Phe | 6F-D,L-Trp | 459.1475 | 459.1461 (Δ 3.0 ppm) | 148.1, 249.1, 382.1, |
| | | | | | | | 441.1, 459.1 |
| 105 | 0.16 | ± 0.06 | 2Br-L-Phe | 6F-D,L-Trp | 519.0674 | 519.0663 (Δ 2.1 ppm) | 147.8, 309.0, 442.0, |
| | | | | | | | 501.1, 519.1 |
| 106 | 0.24 | ± 0.08 | 2Me-L-Phe | 6F-D,L-Trp | 455.1725 | 455.1714 (Δ 2.4 ppm) | 147.8, 245.1, 378.1, |
| | | | | | | | 437.2, 455.2 |
| 107 | 0.25 | ± 0 | 3Me-L-Phe | 6F-D,L-Trp | 455.1725 | 455.1711 (Δ 3.1 ppm) | 148.1, 245.1, 378.1, |
| | | | | | | | 437.2, 455.2 |
| 108 | 0.46 | ± 0.05 | 4Me-L-Phe | 6F-D,L-Trp | 455.1725 | 455.1712 (Δ 2.9 ppm) | 148.1, 245.1, 378.1, |
| | | | | | | | 437.2, 455.2 |

TABLE 11
HR-MS data and conversion rates of thaxtomin A analogues in the reactions of TB14, TCB14, TxtA and TxtB.

| | Conversion | | L-Phe or L-Phe | L-Trp or L-Trp | Calculated | Identified | Key featured MS2 |
|---|---|---|---|---|---|---|---|
| Compound# | rate % | | analogue | analogue | [M + H]+ | [M + H]+ | fragments |
| 1 | 6.84 | ± 2.20 | L-Phe | L-Trp | 439.1612 | 439.1607 (Δ 1.1 ppm) | 130.1, 247.1, 362.1, |
| | | | | | | | 421.1, 439.2 |
| 109 | 3.77 | ± 0.03 | 2F-L-Phe | L-Trp | 457.1518 | 457.1517 (Δ 0.2 ppm) | 130.1, 265.1, 380.1, |
| | | | | | | | 439.1 , 457.2 |
| 110 | 3.77 | ± 1.19 | 3F-L-Phe | L-Trp | 457.1518 | 457.1517 (Δ 0.2 ppm) | 130.1, 265.1, 380.1, |
| | | | | | | | 439.1, 457.2 |
| 111 | 11.98 | ± 0.62 | 4F-L-Phe | L-Trp | 457.1518 | 451.1515 (Δ 0.7 ppm) | 130.1, 265.1, 380.1, |
| | | | | | | | 439.1, 457.2 |
| 112 | 2.64 | ± 0.13 | 3Cl-L-Phe | L-Trp | 473.1222 | 473.1222 (Δ 0.2 ppm) | 129.7, 281.3, 396.1, |
| | | | | | | | 455.2, 473.1 |
| 113 | 3.49 | ± 0.06 | 4Cl-L-Phe | L-Trp | 473.1222 | 473.1222 (Δ 0.0 ppm) | 130.0, 281.3, 396.0, |
| | | | | | | | 455.0, 472.9 |
| 114 | 2.56 | ± 0.48 | 2Br-LPhe | L-Trp | 517.0717 | 517.0714 (Δ 0.6 ppm) | 129.7, 325.0, 439.8, |
| | | | | | | | 499.2, 517.2 |
| 115 | 0.61 | ± 0.01 | 3Br-L-Phe | L-Trp | 517.0717 | 517.0720 (Δ 0.6 ppm) | 129.8, 325.0, 439.8, |
| | | | | | | | 499.1, 517.1 |
| 116 | 0.76 | ± 0.01 | 4Br-L-Phe | L-Trp | 517.0717 | 517.0717 (Δ 0.0 ppm) | 129.9, 325.0, 439.9, |
| | | | | | | | 499.0, 516.5 |
| 117 | 1.55 | ± 0.07 | 2Me-L-Phe | L-Trp | 453.1769 | 453.1756 (Δ 2.9 ppm) | 130.3, 260.8, 375.8, |
| | | | | | | | 435.3, 4513 |
| 118 | 3.98 | ± 0.01 | 4Me-L-Phe | L-Trp | 453.1769 | 451.1757 (Δ 2.6 ppm) | 130.1, 261.1, 376.1, |
| | | | | | | | 435.2, 453.2 |
| 119 | 3.73 | ± 0.05 | L-Phe | 5F-L-Trp | 457.1518 | 457.1505 (Δ 2.8 ppm) | 148.1, 247.1, 380.1, |
| | | | | | | | 439.1, 457.2 |
| 120 | 1.17 | ± 0.28 | 2F-L-Phe | 5F-L-Trp | 475.1424 | 475.1412 (Δ 2.5 ppm) | 148.1, 265.1, 398.1, |
| | | | | | | | 457.1, 475.1 |
| 121 | 2.94 | ± 0.26 | 3F-L-Phe | 5F-L-Trp | 475.1424 | 475.1407 (Δ 3.6 ppm) | 148.3, 265.0, 398.0, |
| | | | | | | | 457.1, 475.1 |
| 122 | 0.53 | ± 0.02 | 4F-L-Phe | 5F-L-Trp | 475.1424 | 475.1414 (Δ 2.1 ppm) | 148.1, 265.1, 398.1, |
| | | | | | | | 457.1, 475.1 |
| 123 | 0.50 | ± 0.05 | 3Cl-L-Phe | 5F-L-Trp | 491.1128 | 491.1116 (Δ 2.4 ppm) | 1481.2811, 414.1, |
| | | | | | | | 473.1, 491.1 |
| 124 | 0.64 | ± 0.05 | 4Cl-L-Phe | 5F-L-Trp | 491.1128 | 491.1115 (Δ 2.6 ppm) | 148.1, 281.1, 414.1, |
| | | | | | | | 473.1, 491.1 |
| 125 | 0.37 | ± 0 | 2Br-L-Phe | 5F-L-Trp | 535.0623 | 535.0612 (Δ 2.1 ppm) | 148.1, 325.0, 458.0, |
| | | | | | | | 517.1, 535.1 |
| 126 | 0.28 | ± 0.01 | 3Br-L-Phe | 5F-L-Trp | 535.0623 | 535.0618 (Δ 0.9 ppm) | 148.1, 325.0, 458.0, |
| | | | | | | | 517.1, 535.1 |
| 127 | 0.30 | ± 0.08 | 4Br-L-Phe | 5F-L-Trp | 535.0623 | 535.0612 (Δ 2.1 ppm) | 148.1, 325.0, 458.0, |
| | | | | | | | 517.1, 535.1 |
| 128 | 0.99 | ± 0.15 | 2Me-L-Phe | 5F-L-Trp | 471.1674 | 471.1663 (Δ 2.3 ppm) | 148.1, 261.1, 394.1, |
| | | | | | | | 453.2, 471.2 |
| 129 | 1.08 | ± 0.07 | 4Me-L-Phe | 5F-L-Trp | 411.1674 | 471.1662 (Δ 2.5 ppm) | 148.1, 261.1, 394.1, |
| | | | | | | | 453.2, 471.2 |
| 130 | 0.70 | ± 0 | L-Phe | 6F-D,L-Trp | 457.1518 | 457.1507 (Δ 2.4 ppm) | 148.1, 247.1, 380.1, |
| | | | | | | | 439.1, 457.2 |
| 131 | 0.23 | ± 0.01 | 2F-L-Phe | 6F-D,L-Trp | 475.1424 | 475.1409 (Δ 3.2 ppm) | 148.2, 265.1, 398.1, |
| | | | | | | | 457.1, 475.1 |
| 132 | 2.04 | ± 0.02 | 3F-L-Phe | 6F-D,L-Trp | 475.1424 | 475.1408 (Δ 3.4 ppm) | 148.1, 265.1, 398.1, |
| | | | | | | | 457.1, 475.1 |
| 133 | 0.77 | ± 0.41 | 4F-L-Phe | 6F-D,L-Trp | 475.1424 | 475.1416 (Δ 1.7 ppm) | 148.1, 265.1, 398.1, |
| | | | | | | | 457.1, 475.1 |
| 134 | 0.23 | ± 0.02 | 4Me-L-Phe | 6F-D,L-Trp | 471.1674 | 471.1664 (Δ 2.1 ppm) | 147.9, 261.1, 394.1, |
| | | | | | | | 453.2, 471.1 |

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A compound, wherein the compound has a formula represented by a structure: 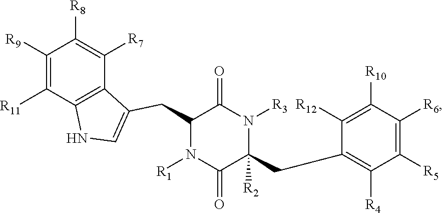
wherein each of R₁ and R₃ is independently selected from hydrogen and C1-C3 alkyl;
wherein R₂ is selected from hydrogen and hydroxy; and
wherein each of R₄, R₅, R₆, R₇, R₈, R₉, R₁₀, R₁₁, and R₁₂ is independently selected from hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl;
or an agriculturally acceptable salt thereof.

2. The compound of claim 1, wherein R₁ is selected from hydrogen, methyl, and ethyl, wherein R₂ is hydrogen, wherein R₃ is selected from hydrogen, methyl, and ethyl, wherein R₃ is selected from hydrogen, methyl, and ethyl, wherein each of R₁₁ and R₁₂ is hydrogen.

3. The compound of claim 1, wherein each of R₄, R₅, R₆, R₇, R₈, R₉, R₁₀, R₁₁, and R₁₂ is independently selected from azido, hydroxy, hydrazino, sulfhydryl, amino, isocyano, cyano, nitro, —F, —Cl, —Br, methyl, ethyl, —CH₂F, —CH₂Cl, —CH₂Br, —CH₂CH₂F, —CH₂CH2Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —OCH₃, —OCH₂CH₃, —NHCH₃, —NHCH₂CH₃, —N(CH3)₂, —N(CH₃)CH₂CH₃, —CH₂OH, —(CH₂)₂OH, —(CHOH)CH₃, —(CHOH)CH₂CH₃, —CH₂OCH₃, —CH₂OCH₂CH₃, —CH₂CH₂OCH₃, —CH₂NH₂, —CH₂NHCH₃, —CH₂NHCH₂CH₃, and —CH₂CH₂NHCH₃.

4. The compound of claim 2, wherein each of R₄, R₅, R₆, R₇, R₈, R₉, R₁₀, R₁₁, and R₁₂ is independently selected from hydrogen, halo, hydroxy, azido, and C1-C3 alkyl.

5. The compound of claim 2, wherein each of R₄, R₅, R₆, R₇, R₈, R₉, R₁₀, R₁₁, and R₁₂ is independently selected from hydrogen, halo, hydroxy, azido, and methyl.

6. The compound of claim 2, wherein each of R₄, R₅, R₆, R₇, R₈, R₉, R₁₀, R₁₁, and R₁₂ is independently selected from hydrogen, hydroxy, azido, and methyl.

7. The compound of claim 1, wherein the compound has a formula represented by a structure:

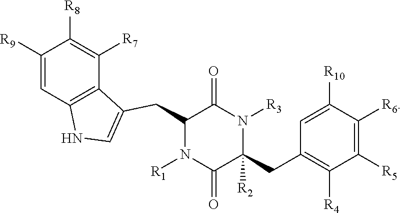

8. The compound of claim 7, wherein each of R₁ and R₃ is independently selected from hydrogen and C1-C3 alkyl;
wherein each of R₂ and R₁₀ is independently selected from hydrogen and hydroxy;
wherein each of R₄, R₅, and R₆ is independently selected from hydrogen, halo, hydroxy, azido, and C1-C3 alkyl;
wherein R₇ is selected from hydrogen, azido, halo, hydroxy, hydrazino, sulfhydryl, amino, isocyano, cyano, nitro, C1-C3 alkyl, C1-C3 haloalkyl, C1-C3 alkoxy, C1-C3 alkylamino, hydoxy(C1-C3 alkanediyl), C1-C3 alkoxy-C1-C3 alkanediyl, and C1-C3 alkyl-NH—C1-C3 alkanediyl;
wherein each of R₈ and R₉ is independently selected from hydrogen and halo; and
wherein R₁₀ is selected from hydrogen and hydroxyl.

9. The compound of claim 8, wherein each of R₁ and R₃ is independently selected from hydrogen or methyl

10. The compound of claim 8, wherein R₁ is methyl; and wherein R₃ is selected from hydrogen or methyl.

11. The compound of claim 8, wherein R₂ is hydrogen.

12. The compound of claim 8, wherein R₂ is hydroxy.

13. The compound of claim 8, wherein each of R₄, R₅, and R₆ is independently selected from hydrogen, fluoro, chloro, bromo, hydroxy, and methyl.

14. The compound of claim 8, wherein each of R₄, R₅, and R₆ is independently selected from hydrogen and hydroxy.

15. The compound of claim 8, wherein each of R₄, R₅, and R₆ is hydrogen.

16. The compound of claim 8, wherein R₄ is hydrogen; and where each of R₅ and R₆ is independently selected from hydrogen, fluoro, chloro, bromo, hydroxy, and methyl; or wherein R₅ is hydrogen and where each of R₅ and R₆ is independently selected from hydrogen, fluoro, chloro, bromo, hydroxy, and methyl; or wherein R₆ is hydrogen and where each of R₄ and R₅ is independently selected from hydrogen, fluoro, chloro, bromo, hydroxy, and methyl or wherein R₇ is selected from hydrogen, nitro, and fluoro.

17. The compound of claim 16, wherein each of R₈ and R₉ is independently selected from hydrogen and fluoro.

18. A herbicidal composition comprising an herbicidally effective amount of a compound of claim 1, in a mixture with an agriculturally acceptable adjuvant or carrier.

19. The herbicidal composition of claim 18, further comprising at least one herbicidal agent.

20. The herbicidal composition of claim 19, wherein the at least one herbicidal agent is selected from an amide herbicide, anilide herbicide, arylalanine herbicide, chloroacetanilide herbicide, sulfonanilide herbicide, sulfonamide herbicide, antibiotic herbicide, benzoic add herbicide, pyrimidinyloxybenzoic acid herbicide, pyrimidinylthiobenzoic acid herbicide, quinolinecarboxylic acid herbicide, arsenical herbicide, benzoylcyclohexanedione herbicide, benzofuranyl alkylsulfonate herbicide, carbamate herbicide, carbanilate herbicide, cyclohexene oxime herbicide, cyclopropylisoxazole herbicide, dicarboximide herbicide, dinitroaniline herbicide, dinitrophenol herbicide, diphenyl ether herbicide, nitrophenyl ether herbicide, dithiocarbamate herbicide, halogenated aliphatic herbicide, imidazolinone herbicide, inorganic herbicide, nitrile herbicide, organophosphorus herbicide, phenoxy herbicide, phenoxyacetic herbicide, phenoxybutyric herbicide, phenoxypropionic herbicide, aryloxyphenoxypropionic herbicide, phenylenediamine herbicide, pyrazolyl herbicide, pyrazolylphenyl herbicide, pyridazine herbicide, pyridazinone herbicide, pyridine herbicide, pyrimidinediamine herbicide, quaternary ammonium herbicide, thiocarbamate herbicide, thiocarbonate herbicide, triazine herbicide, chlorotriazine herbicide, methoxytriazine herbicide, methylthiotriazine herbicide, triazinone herbicide, triazole herbicide, triazolone herbicide, triazolopyrimidine herbicide, urea herbicide, phenylurea herbicide, pyrimidinylsulfonylurea herbicide, triazinylsulfonylurea herbicide, thiadiazolylurea herbicide, and unclassified herbicide, and combinations thereof.
